(19) United States
(12) Patent Application Publication
(10) Pub. No.: US 2023/0111853 A1
(43) Pub. Date: Apr. 13, 2023

(54) COMPOUNDS MODULATING PROTEIN RECRUITMENT AND/OR DEGRADATION

(71) Applicant: Orionis Biosciences, Inc., Waltham, MA (US)

(72) Inventors: Nikolai KLEY, Waltham, MA (US); Riccardo SABATINI, Waltham, MA (US); Edward SUH, Waltham, MA (US)

(21) Appl. No.: 17/786,161

(22) PCT Filed: Dec. 16, 2020
(86) PCT No.: PCT/US2020/065303

§ 371 Date: Jun. 16, 2022

Related U.S. Application Data
(60) Provisional application No. 62/949,027, filed on Dec. 17, 2019.

(51) Int. Cl. C07D 401/04; C07D 417/04; C07D 211/40; C07D 471/04; C07D 401/14; C07D 495/04; C07D 491/056; C07D 487/04; C07D 491/048; C07D 405/14; A61P 43/00

(52) U.S. Cl. CPC C07D 401/04; C07D 417/04; C07D 211/40; C07D 471/04; C07D 401/14; C07D 495/04; C07D 491/056; C07D 487/04; C07D 491/048; C07D 405/14; A61P 43/00

(57) ABSTRACT

The invention provides cereblon binders for the degradation of proteins by the ubiquitin proteasome pathway for therapeutic applications.

### CROSS-REFERENCE TO RELATED APPLICATIONS

This application claims the benefit of U.S. Provisional Application No. 62/949,027, filed on Dec. 17, 2019, the entire contents of which are incorporated herein.

### FIELD OF THE INVENTION

The invention provides cereblon binders for the degradation of proteins by the ubiquitin proteasome pathway for therapeutic applications as described further herein.

### BACKGROUND OF THE INVENTION

Protein degradation is a highly regulated and essential process that maintains cellular homeostasis. The selective identification and removal of damaged, misfolded, or excess proteins is achieved via the ubiquitin-proteasome pathway (UPP). The UPP is essential to the regulation of almost all cellular processes.

Covalent attachment of multiple ubiquitin molecules by an E3 ubiquitin ligase to a terminal lysine residue marks the protein for proteasome degradation, where the protein is digested into small peptides and eventually into its constituent amino acids that serve as building blocks for new proteins.

Thalidomide and its analogues have been found to bind to the ubiquitin ligase cereblon and redirect its ubiquitination activity (Ito, T. et al., Science, 2010, 327: 1345). Cereblon forms part of an E3 ubiquitin ligase complex which interacts with damaged DNA binding protein, forming an E3 ubiquitin ligase complex with Cullin 4 and the E2-binding protein ROC1 (known as RBX1) where it functions as a substrate receptor to select proteins for ubiquitination. The binding of lenalidomide to cereblon facilitates subsequent binding of cereblon to Ikaros and Aiolos, leading to their ubiquitination and degradation by the proteasome (Lu, G. et al., Science, 2014, 343:305-309; Kronke, J. et al., Science, 2014, 343:301-305).

It is an object of the present invention to provide new compounds binding to cereblon and the use thereof for the treatment of various diseases and disorders, e.g. by modulating protein degradation.

### SUMMARY OF THE INVENTION

New compounds that bind cereblon are provided, along with their uses and manufacture. It is believed, without wishing to be bound by theory, that binding of the disclosed compounds to cereblon results in increased or reduced interactions of cereblon with one or more of IKZF1, SALL4, and ASS1, leading to their subsequent ubiquitination and degradation in the proteasome. The selected compounds are found to be both potent binders of cereblon as well as showing potential therapeutic use. Accordingly, in various embodiments, the compounds are “molecular glues,” and therefore can bind protein surfaces or interfaces, e.g. on cereblon, and stabilize interaction(s) with another protein, potentially resulting in the activation or suppression of a cellular response (e.g. ubiquitination and degradation in the proteasome).

Compounds disclosed herein, pharmaceutically acceptable salts thereof, or pharmaceutically acceptable compositions thereof can be used to treat a disorder mediated by cereblon, IKZF1, SALL4, or ASS1, e.g. various cancers and autoimmune diseases or disorders.

In one aspect, the compound of the present invention is selected from Formula I:

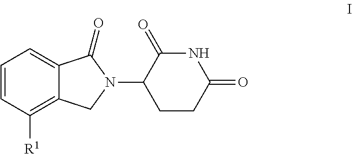

or a pharmaceutically acceptable salt thereof, wherein:

R¹ is aryl, —N(R⁵)—X—R⁶, —SO₂R⁵, or —O(CH₂)ₘR⁵, any of which may be optionally substituted with 1 or more R^{w} groups as allowed by valence;

R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;

R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;

R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;

X is a bond, —SO₂—, —(CH₂)ₙC(O)(CH₂)ₘ—, —C(O)NH—, —C(O)N(R^{w})—, —NHC(O)NH—, or —(CH₂)ₙ—;

m is 0, 1, 2, 3, or 4;

n is 0, 1, 2, 3, or 4.

In one aspect, the compound of the present invention is selected from Formula II:

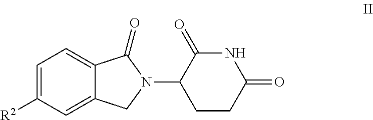

or a pharmaceutically acceptable salt thereof, wherein:

R² is aryl, —NH—(C₃-C₁₀) heteroaryl, or —N(R⁵)—(CH₂)ₘ—X—(CH₂)ₙ—R⁶, any of which may be optionally substituted with 1 or more R^{w} groups as allowed by valence;

R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;

R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;

R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;

X is a bond, —SO₂—, —(CH₂)ₙC(O)(CH₂)ₘ—, —C(O)NH—, —C(O)N(R^{w})—, —NHC(O)NH—, or —(CH₂)ₙ—;

m is 0, 1, 2, 3, or 4;

n is 0, 1, 2, 3, or 4.

In one aspect, the compound of the present invention is selected from Formula III:

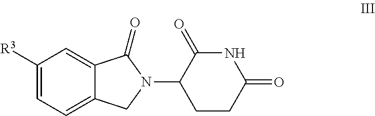

or a pharmaceutically acceptable salt thereof, wherein:

R³ is cyano, aryl, —NH—(C₃-C₁₀) heteroaryl, (C₃-C₁₀)heterocyclo, or —N(R⁵)—(CH₂)ₘ—X—(CH₂)ₙ—R⁶, any of which may be optionally substituted with 1 or more R^{w} groups as allowed by valence;

R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;

R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;

R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;

X is a bond, —SO₂—, —(CH₂)ₙC(O)(CH₂)ₘ—, —C(O)NH—, —C(O)N(R^{w})—, —NHC(O)NH—, or —(CH₂)ₙ—;

m is 0, 1, 2, 3, or 4;

n is 0, 1, 2, 3, or 4.

In one aspect, the compound of the present invention is selected from Formula IV:

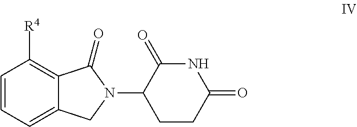

or a pharmaceutically acceptable salt thereof, wherein:

R⁴ is halo, cyano, aryl, OR⁵, or —N(R⁵)—(CH₂)ₘ—X—(CH₂)ₙ—R⁶, any of which may be optionally substituted with 1 or more R^{w} groups as allowed by valence;

R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;

R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;

R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;

X is a bond, —SO₂—, —(CH₂)ₙC(O)(CH₂)ₘ—, —C(O)NH—, —C(O)N(R^{w})—, —NHC(O)NH—, or —(CH₂)ₙ—;

m is 0, 1, 2, 3, or 4;

n is 0, 1, 2, 3, or 4.

In one aspect, the compound of the present invention is selected from Formula V:

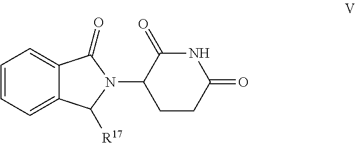

or a pharmaceutically acceptable salt thereof, wherein:

R¹⁷ is cyano, heteroaryl, —(CH₂)ₘ—C(O)O—R⁶, or —N(R⁵)—(CH₂)ₘ—X—(CH₂)ₙ—R⁶, any of which may be optionally substituted with 1 or more R^{w} groups as allowed by valence;

R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;

R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;

R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;

X is a bond, —SO₂—, —(CH₂)ₙC(O)(CH₂)ₘ—, —C(O)NH—, —C(O)N(R^{w})—, —NHC(O)NH—, or —(CH₂)ₙ—;

m is 0, 1, 2, 3, or 4;

n is 0, 1, 2, 3, or 4.

In one aspect, the compound of the present invention is selected from Formula VI:

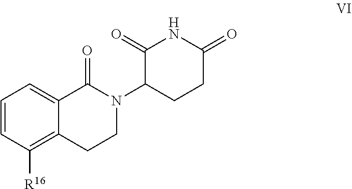

or a pharmaceutically acceptable salt thereof, wherein:

R¹⁶ is NH₂ or —N(R⁵)—(CH₂)ₘ—X—(CH₂)ₙ—R⁶, any of which may be optionally substituted with 1 or more R^{w} groups as allowed by valence;

R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;

R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;

R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;

X is a bond, —SO₂—, —(CH₂)ₙC(O)(CH₂)ₘ—, —C(O)NH—, —C(O)N(R^{w})—, —NHC(O)NH—, or —(CH₂)ₙ—;

m is 0, 1, 2, 3, or 4;

n is 0, 1, 2, 3, or 4.

In one aspect, the compound of the present invention is selected from Formula VII:

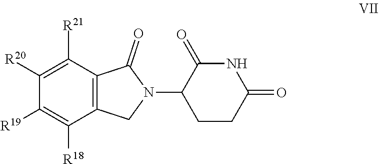

or a pharmaceutically acceptable salt thereof, wherein:

R¹⁸, R¹⁹, R²⁰, R²¹ each independently is H, halo, (C₁-C₃)alkyl, or —N(R⁵)—X—R⁶, with the proviso that no more than two substituents of R¹⁸, R¹⁹, R²⁰, R²¹ are H; or

R¹⁸, R¹⁹ taken together with the carbons they are attached to forming a (C₃-C₁₀)cycloalkyl or a (C₃-C₁₀)heterocyclo, or R¹⁹, R²⁰ taken together with the carbons they are attached to forming a (C₃-C₁₀)cycloalkyl or a (C₃-C₁₀)heterocyclo, or R²⁰, R²¹ taken together with the carbons they are attached to forming a (C₃-C₁₀)cycloalkyl or a (C₃-C₁₀)heterocyclo, any of which may be optionally substituted with 1 or more R^{w} groups as allowed by valence;

R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;

R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;

R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;

X is a bond, —SO₂—, —(CH₂)ₙC(O)(CH₂)ₘ—, —C(O)NH—, —C(O)N(R^{w})—, —NHC(O)NH—, or —(CH₂)ₙ—;

m is 0, 1, 2, 3, or 4;

n is 0, 1, 2, 3, or 4.

In one aspect, the compound of the present invention is selected from Formula VIII:

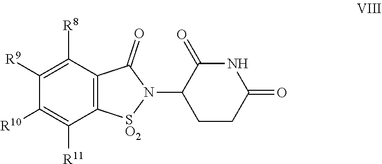

or a pharmaceutically acceptable salt thereof, wherein:

R⁸, R⁹, R¹⁰, R¹¹ each independently is H, halo, OH, cyano, (C₁-C₃)alkyl, (C₁-C₃)alkoxy, aryl, or heteroaryl, any of which may be optionally substituted with 1 or more R^{w} groups as allowed by valence;

R^{w} at each occurrence is independently, H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;

n is 0, 1, 2, 3, or 4.

In one aspect, the compound of the present invention is selected from Formula IX:

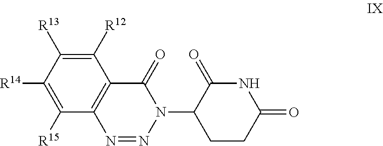

or a pharmaceutically acceptable salt thereof, wherein:

R¹², R¹³, R¹⁴, R¹⁵ each is independently H, NH₂, (C₁-C₃)alkyl, —N(R⁵)—(CH₂)ₘ—N(R⁵)—X—R⁶, with proviso that no more than three substituents out of R¹², R¹³, R¹⁴, and R¹⁵ are H, any of which may be optionally substituted with 1 or more R^{w} groups as allowed by valence;

R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;

R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;

R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;

X is a bond, —SO₂—, —(CH₂)ₙC(O)(CH₂)ₘ—, —C(O)NH—, —C(O)N(R^{w})—, —NHC(O)NH—, or —(CH₂)ₙ—;

m is 0, 1, 2, 3, or 4;

n is 0, 1, 2, 3, or 4.

In one aspect, the compound of the present invention is selected from Formula X:

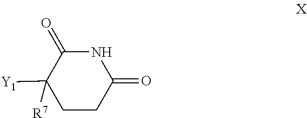

or a pharmaceutically acceptable salt thereof, wherein:

Y is —NHR³³, —NHC(O)R³³, or —CHR³³R³⁴;

R⁷ is H, (C₁-C₃)alkyl, or R⁷ and R³⁴ taken together with the carbons they are attached to forming a carbon carbon double bond;

R³³ is aryl, heteroaryl, or (C₃-C₁₀)heterocyclo, any of which may be optionally substituted with 1 or more R^{w} groups as allowed by valence;

R^{w} at each occurrence is independently, H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;

n is 0, 1, 2, 3, or 4.

In one aspect, the compound of the present invention is selected from the group consisting of.
3-[1-oxo-5-(quinazolin-4-ylamino)isoindolin-2-yl]piperidine-2,6-dione;
3-[5-[(4-aminothieno[2,3-d]pyrimidin-2-yl)amino]-1-oxo-isoindolin-2-yl]piperidine-2,6-dione;
N-[2-(2,6-dioxo-3-piperidyl)-1-oxo-isoindolin-5-yl]acetamide;
3-[5-[(2-aminopyrimidin-4-yl)amino]-1-oxo-isoindolin-2-yl]piperidine-2,6-dione;
6-[[2-(2,6-dioxo-3-piperidyl)-1-oxo-isoindolin-5-yl]amino]pyridazine-3-carbonitrile;
3-[[2-[[2-(2,6-dioxo-3-piperidyl)-1-oxo-isoindolin-5-yl]amino]acetyl]amino]benzamide;
2-[[2-(2,6-dioxo-3-piperidyl)-1-oxo-isoindolin-5-yl]amino]acetic acid;
N-[2-(2,6-dioxo-3-piperidyl)-3-oxo-isoindolin-5-yl]quinoline-2-carboxamide;
3-[6-[[2-(2-methyl-1-piperidyl)-2-oxo-ethyl]amino]-1-oxo-isoindolin-2-yl]piperidine-2,6-dione;
3-[6-[(2-isoindolin-2-yl-2-oxo-ethyl)amino]-1-oxo-isoindolin-2-yl]piperidine-2,6-dione;
N-(cyclopropylmethyl)-2-[[2-(2,6-dioxo-3-piperidyl)-3-oxo-isoindolin-5-yl]amino]-N-methyl-acetamide;
acetic acid; 3-[1-oxo-6-(quinazolin-4-ylamino)isoindolin-2-yl]piperidine-2,6-dione;
3-[6-[[2-(3-methyl-1-piperidyl)-2-oxo-ethyl]amino]-1-oxo-isoindolin-2-yl]piperidine-2,6-dione;
3-[6-[(4-methyl-3-oxo-pyrazin-2-yl)amino]-1-oxo-isoindolin-2-yl]piperidine-2,6-dione;
3-[1-oxo-6-(quinoxalin-2-ylamino)isoindolin-2-yl]piperidine-2,6-dione;
3-[6-[(1-methylpyrazolo[3,4-d]pyrimidin-4-yl)amino]-1-oxo-isoindolin-2-yl]piperidine-2,6-dione;
3-[6-(5,7-dihydrofuro[3,4-d]pyrimidin-2-ylamino)-1-oxo-isoindolin-2-yl]piperidine-2,6-dione;
3-[6-[(6-methylpyrimidin-4-yl)amino]-1-oxo-isoindolin-2-yl]piperidine-2,6-dione;
2-[[2-(2,6-dioxo-3-piperidyl)-3-oxo-isoindolin-5-yl]amino]-N-phenyl-acetamide;
3-[6-[[2-(2,4-dimethylpiperazin-1-yl)-2-oxo-ethyl]amino]-1-oxo-isoindolin-2-yl]piperidine-2,6-dione;
3-[6-(dimethylamino)-1-oxo-isoindolin-2-yl]piperidine-2,6-dione;
3-(1-oxo-6-phenyl-isoindolin-2-yl)piperidine-2,6-dione;
2-[[2-(2,6-dioxo-3-piperidyl)-3-oxo-isoindolin-5-yl]amino]-N,N-dimethyl-acetamide;
3-[6-[[2-(2-methylmorpholin-4-yl)-2-oxo-ethyl]amino]-1-oxo-isoindolin-2-yl]piperidine-2,6-dione;
2-[[2-(2,6-dioxo-3-piperidyl)-3-oxo-isoindolin-5-yl]amino]-N-methyl-N-[(1-methylpyrazol-4-yl)methyl]acetamide;
N-benzyl-2-[[2-(2,6-dioxo-3-piperidyl)-3-oxo-isoindolin-5-yl]amino]acetamide;
6-[[2-(2,6-dioxo-3-piperidyl)-3-oxo-isoindolin-5-yl]amino]pyridazine-3-carbonitrile;
3-[6-[(6-methylpyrrolo[3,2-d]pyrimidin-4-yl)amino]-1-oxo-isoindolin-2-yl]piperidine-2,6-dione;
2-(dimethylamino)-N-[2-(2,6-dioxo-3-piperidyl)-3-oxo-isoindolin-5-yl]acetamide;
N-[2-(2,6-dioxo-3-piperidyl)-3-oxo-isoindolin-5-yl]-5H-pyrrolo[2,3-b]pyridine-4-carboxamide;
N-cyclopropyl-2-[[2-(2,6-dioxo-3-piperidyl)-3-oxo-isoindolin-5-yl]amino]acetamide;
3-[1-oxo-6-(2-oxoimidazolidin-1-yl)isoindolin-2-yl]piperidine-2,6-dione;
2-(2,6-dioxo-3-piperidyl)-3-oxo-isoindoline-5-carbonitrile;
2-[[2-(2,6-dioxo-3-piperidyl)-3-oxo-isoindolin-5-yl]amino]propanoic acid;
2-acetamido-N-[2-(2,6-dioxo-3-piperidyl)-3-oxo-isoindolin-5-yl]acetamide;
2-[[2-(2,6-dioxo-3-piperidyl)-3-oxo-isoindolin-5-yl]amino]acetamide;
3-[6-[[2-(3-methyl-5-oxo-piperazin-1-yl)-2-oxo-ethyl]amino]-1-oxo-isoindolin-2-yl]piperidine-2,6-dione;
N-[2-(2,6-dioxo-3-piperidyl)-3-oxo-isoindolin-5-yl]acetamide;
3-[6-[[2-(4-methyl-3-oxo-piperazin-1-yl)-2-oxo-ethyl]amino]-1-oxo-isoindolin-2-yl]piperidine-2,6-dione;
N-[2-(2,6-dioxo-3-piperidyl)-3-oxo-isoindolin-5-yl]-3H-imidazo[4,5-b]pyridine-6-carboxamide;
2-[[2-(2,6-dioxo-3-piperidyl)-3-oxo-isoindolin-5-yl]amino]-N-tetrahydropyran-4-yl-acetamide;
2-[[2-(2,6-dioxo-3-piperidyl)-3-oxo-isoindolin-5-yl]amino]acetic acid;
3-[1-oxo-6-[[2-oxo-2-(1-piperidyl)ethyl]amino]isoindolin-2-yl]piperidine-2,6-dione;
3-(1-oxo-7-phenyl-isoindolin-2-yl)piperidine-2,6-dione;
2-(2,6-dioxo-3-piperidyl)-3-oxo-isoindoline-4-carbonitrile;
2-[[2-(2,6-dioxo-3-piperidyl)-3-oxo-isoindolin-4-yl]amino]acetic acid;
3-(7-fluoro-1-oxo-isoindolin-2-yl)piperidine-2,6-dione;
3-(5-amino-1-oxo-3,4-dihydroisoquinolin-2-yl)piperidine-2,6-dione;
t-butyl 2-[2-(2,6-dioxo-3-piperidyl)-3-oxo-isoindolin-1-yl]acetate;
3-[1-(2H-indol-3-yl)-3-oxo-isoindolin-2-yl]piperidine-2,6-dione;
2-(2,6-dioxo-3-piperidyl)-3-oxo-isoindoline-1-carbonitrile;
3-[1-(dimethylamino)-3-oxo-isoindolin-2-yl]piperidine-2,6-dione;
3-(2-oxopyrrolidin-1-yl)piperidine-2,6-dione;
3-(quinazolin-2-ylamino)piperidine-2,6-dione;
(3Z)-3-benzylidenepiperidine-2,6-dione;
3-(quinoxalin-2-ylamino)piperidine-2,6-dione;
3-(pyrimidin-2-ylamino)piperidine-2,6-dione;
N-(2,6-dioxo-3-piperidyl)-2-oxo-3H-pyridine-6-carboxamide;
3-(4-methyl-1,1,3-trioxo-1,2-benzothiazol-2-yl)piperidine-2,6-dione;
3-(8-amino-4-oxo-1,2,3-benzotriazin-3-yl)piperidine-2,6-dione;
3-(5-amino-4-oxo-1,2,3-benzotriazin-3-yl)piperidine-2,6-dione;
3-(4-oxo-1,2,3-benzotriazin-3-yl)piperidine-2,6-dione;
3-(5-methyl-4-oxo-1,2,3-benzotriazin-3-yl)piperidine-2,6-dione;
3-(6-methyl-4-oxo-1,2,3-benzotriazin-3-yl)piperidine-2,6-dione; and
3-(8-methyl-4-oxo-1,2,3-benzotriazin-3-yl)piperidine-2,6-dione.

In one aspect, the present invention relates to a composition comprising a pharmaceutically effective amount of the compound described herein and a pharmaceutically acceptable carrier.

In one aspect, the present invention relates to a method of treating a disease comprising administering the composition comprising a pharmaceutically effective amount of the compound described herein and a pharmaceutically acceptable carrier to a subject in need thereof.

In one aspect, the present invention relates to a method of treating or preventing a cancer comprising administering the composition comprising a pharmaceutically effective amount of the compound described herein and a pharmaceutically acceptable carrier to a subject in need thereof. In an embodiment, the cancer is selected from the group consisting of squamous-cell carcinoma, basal cell carcinoma, adenocarcinoma, hepatocellular carcinomas, renal cell carcinomas, bladder cancer, bowel cancer, breast cancer, cervical cancer, colon cancer, esophageal cancer, head cancer, kidney cancer, liver cancer, lung cancer, neck cancer, ovarian cancer, pancreatic cancer, prostate cancer, stomach cancer, uterine cancer, leukemias, lymphomas, Burkitt's lymphoma, Non-Hodgkin's lymphoma, melanomas, myeloproliferative diseases, multiple myeloma, sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, Schwannomas, testicular cancer, thyroid cancer, astrocytoma, Hodgkin's disease, Wilms' tumor, and teratocarcinomas. In an embodiment, the subject is a human.

In another aspect, the present invention relates to a method of treating or preventing one or more autoimmune diseases or disorders comprising administering a composition comprising a pharmaceutically effective amount of the compound described herein and a pharmaceutically acceptable carrier to a subject in need thereof. In an embodiment, the autoimmune disease or disorder is selected from, such as multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, and other autoimmune diseases or disorders.

### BRIEF DESCRIPTION OF THE DRAWINGS

The foregoing summary, as well as the following detailed description of the invention, will be better understood when read in conjunction with the appended drawings. References to various compounds are made with reference to Table 1 in Example 4.

FIG. 1 illustrates the change of cereblon's ability to recruit ASS1, IKZF1, and SALL4 after binding with 5HPP-3 in comparison to lenalidomide (LEN).

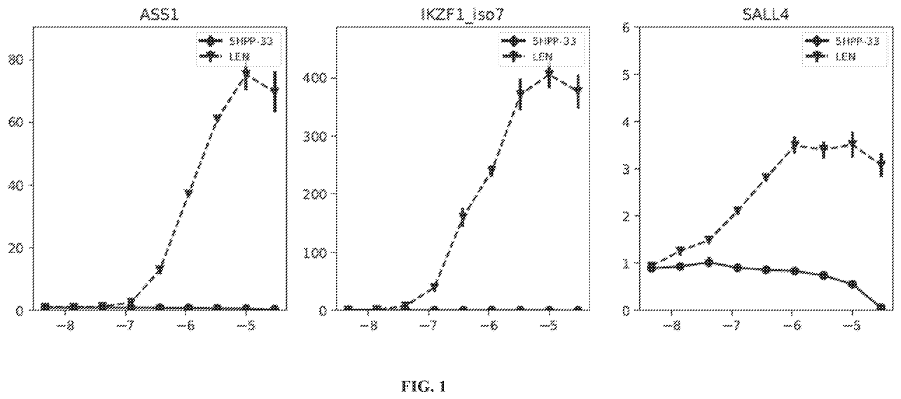

FIG. 2 illustrates the change of cereblon's ability to recruit ASS1, IKZF1, and SALL4 after binding with C98696 in comparison to LEN. Lenalidomide is the top curve of the right panel.

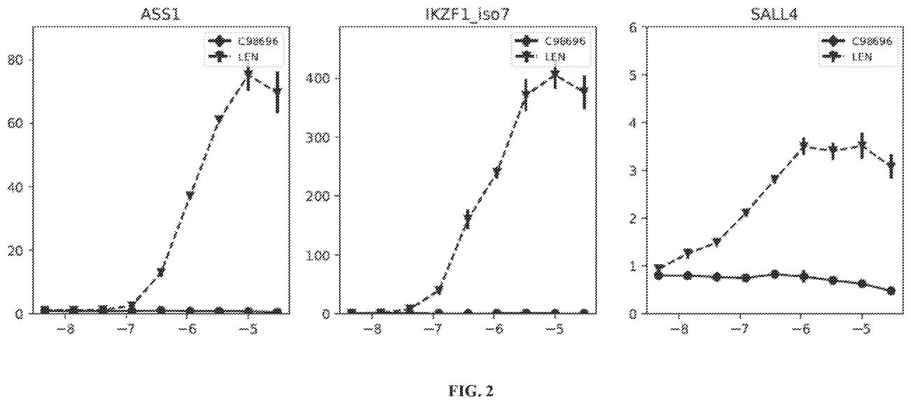

FIG. 3 illustrates the change of cereblon's ability to recruit ASS1, IKZF1, and SALL4 after binding with C36126 in comparison to LEN.

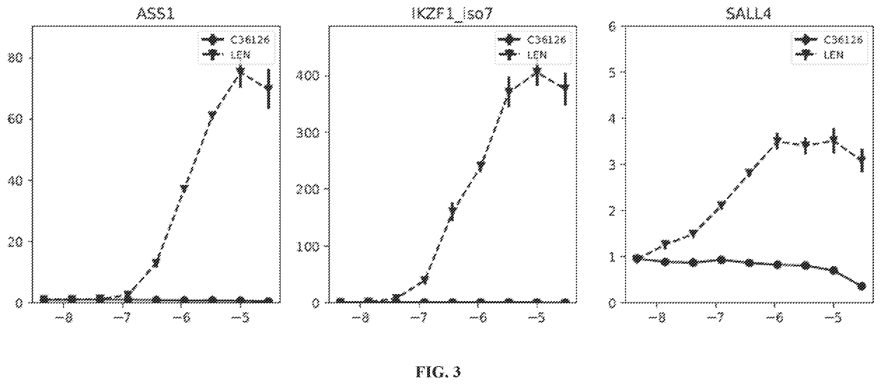

FIG. 4 illustrates the change of cereblon's ability to recruit ASS1, IKZF1, and SALL4 after binding with C44292 in comparison to LEN.

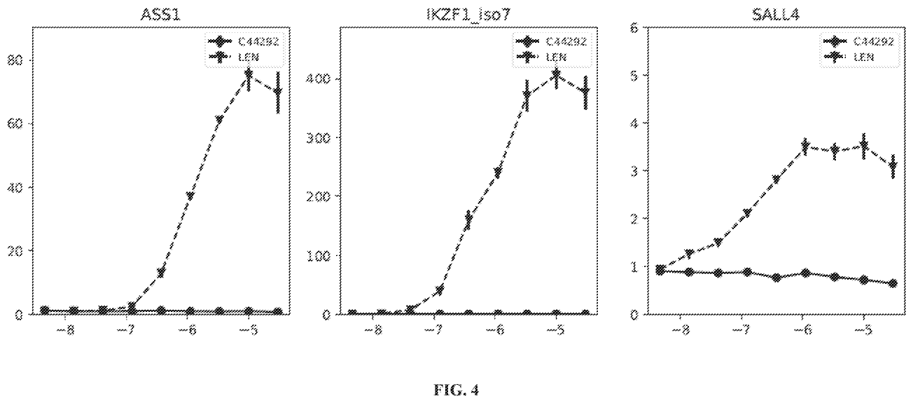

FIG. 5 illustrates the change of cereblon's ability to recruit ASS1, IKZF1, and SALL4 after binding with C97402 in comparison to LEN.

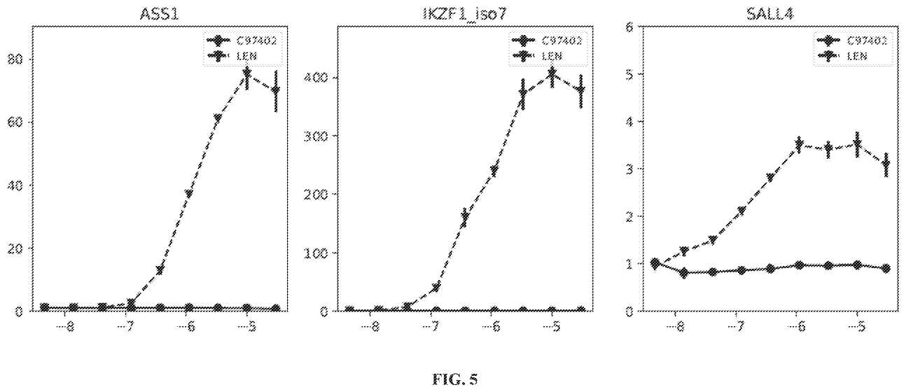

FIG. 6 illustrates the change of cereblon's ability to recruit ASS1, IKZF1, and SALL4 after binding with C51830 in comparison to LEN.

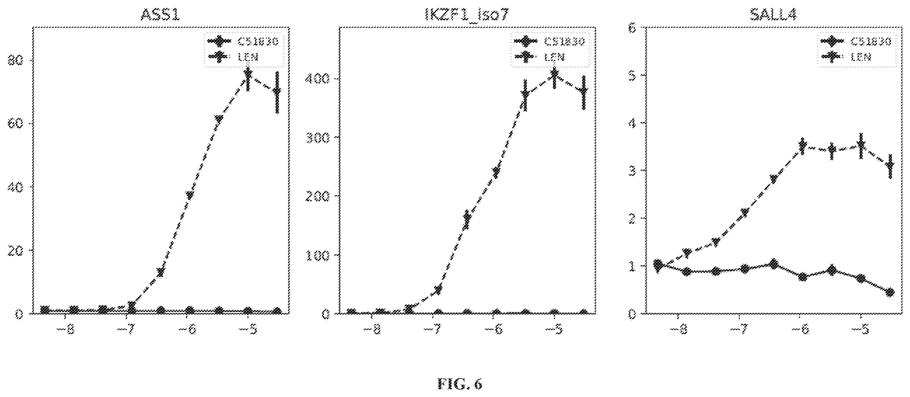

FIG. 7 illustrates the change of cereblon's ability to recruit ASS1, IKZF1, and SALL4 after binding with C55468 in comparison to LEN.

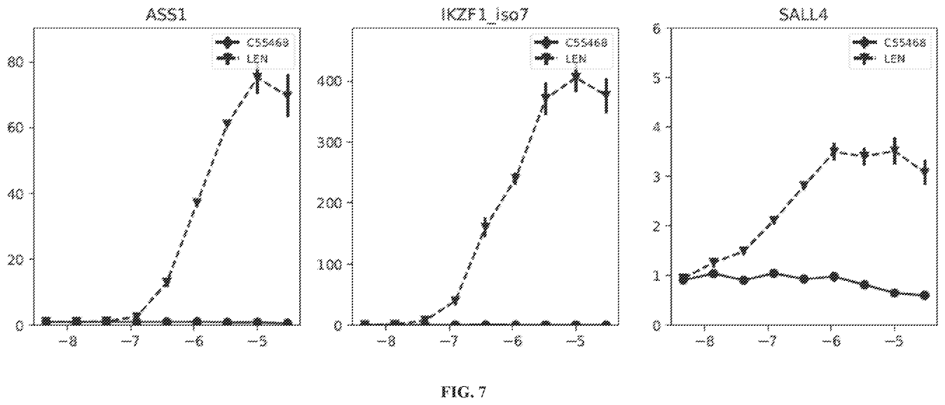

FIG. 8 illustrates the change of cereblon's ability to recruit ASS1, IKZF1, and SALL4 after binding with C28661 in comparison to LEN.

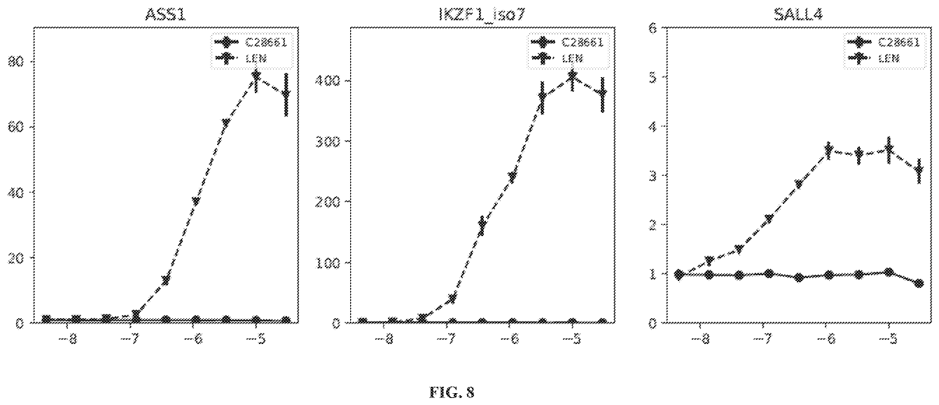

FIG. 9 illustrates the change of cereblon's ability to recruit ASS1, IKZF1, and SALL4 after binding with C29137 in comparison to LEN.

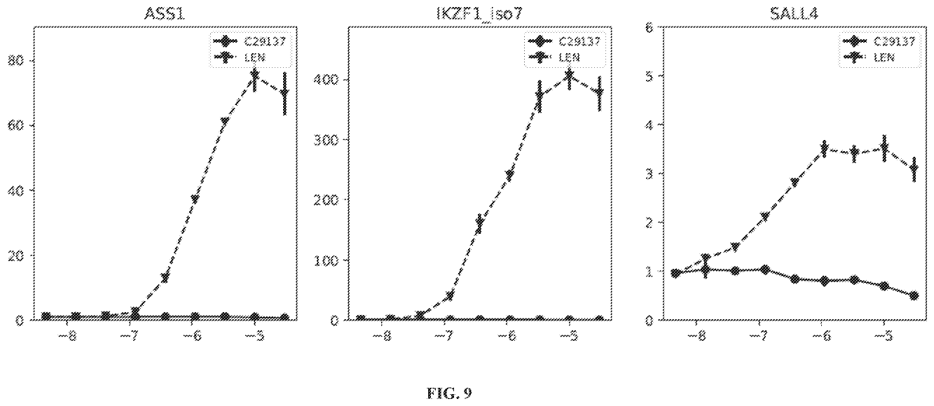

FIG. 10 illustrates the change of cereblon's ability to recruit ASS1, IKZF1, and SALL4 after binding with C29408 in comparison to LEN.

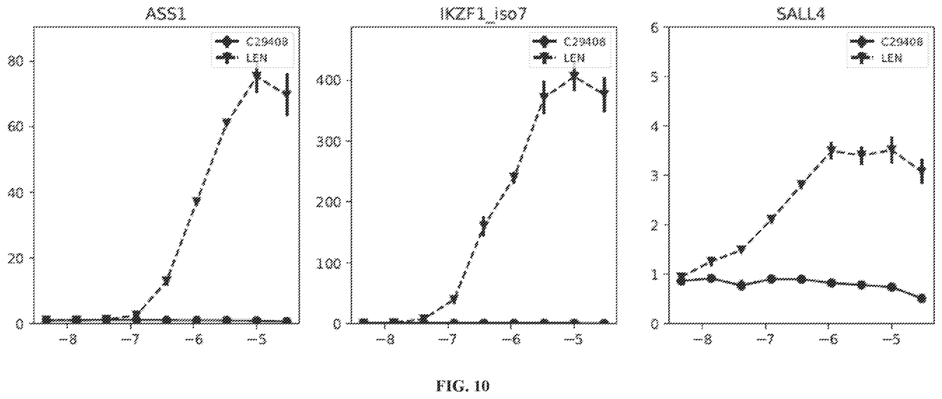

FIG. 11 illustrates the change of cereblon's ability to recruit ASS1, IKZF1, and SALL4 after binding with C47997 in comparison to LEN.

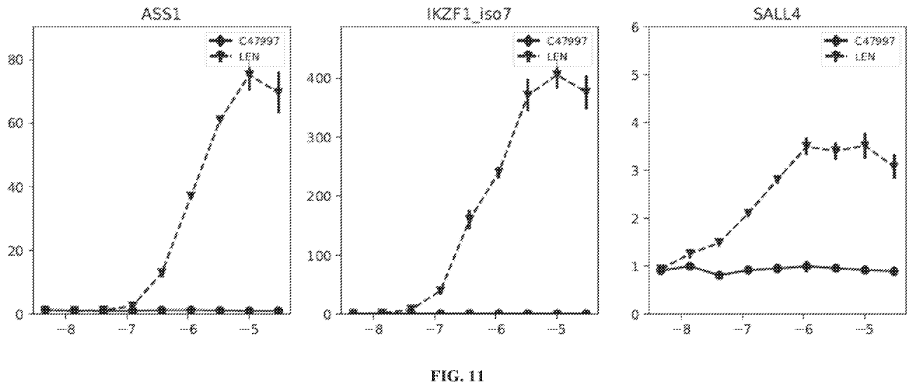

FIG. 12 illustrates the change of cereblon's ability to recruit ASS1, IKZF1, and SALL4 after binding with C48009 in comparison to LEN.

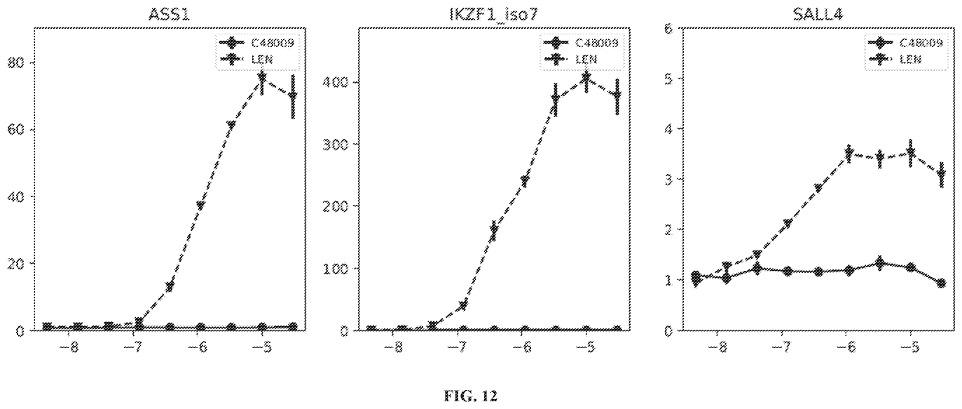

FIG. 13 illustrates the change of cereblon's ability to recruit ASS1, IKZF1, and SALL4 after binding with C48020 in comparison to LEN.

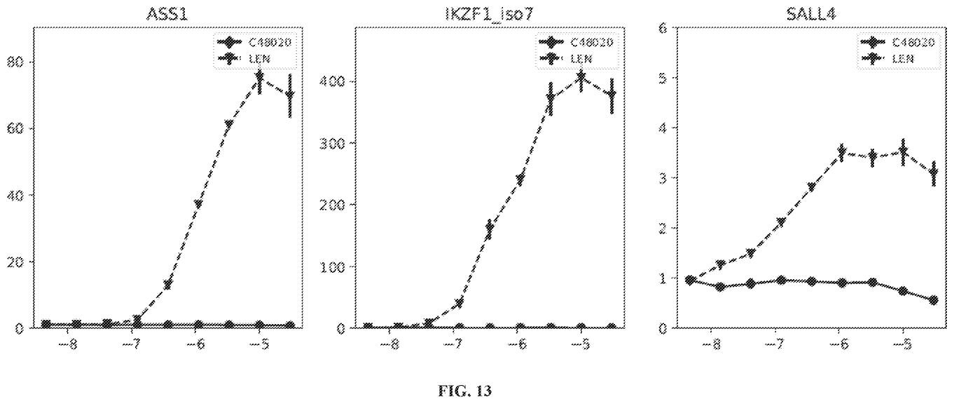

FIG. 14 illustrates the change of cereblon's ability to recruit ASS1, IKZF1, and SALL4 after binding with C66979 in comparison to LEN.

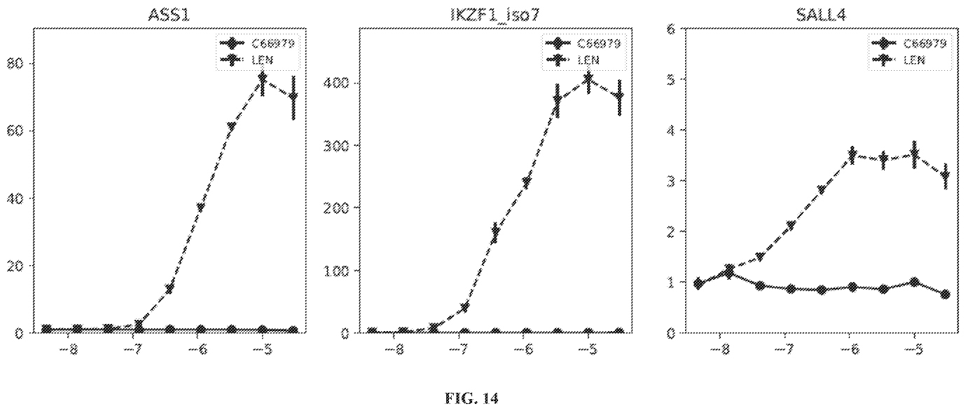

FIG. 15 illustrates the change of cereblon's ability to recruit ASS1, IKZF1, and SALL4 after binding with C68121 in comparison to LEN.

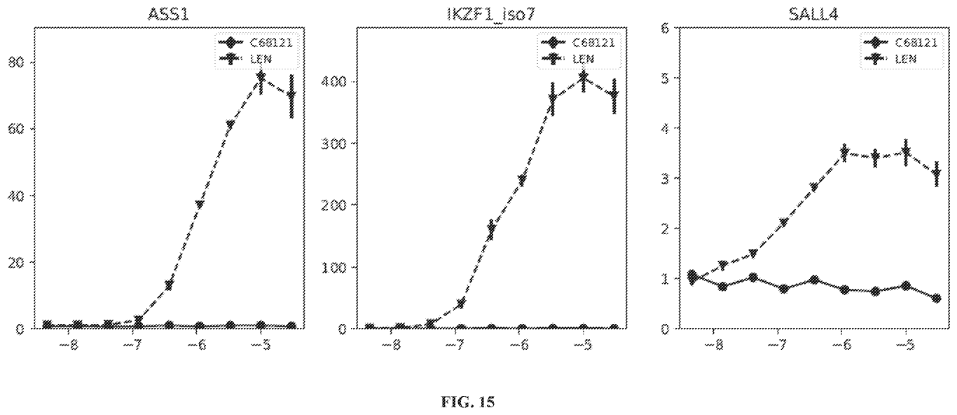

FIG. 16 illustrates the change of cereblon's ability to recruit ASS1, IKZF1, and SALL4 after binding with C49708 in comparison to LEN.

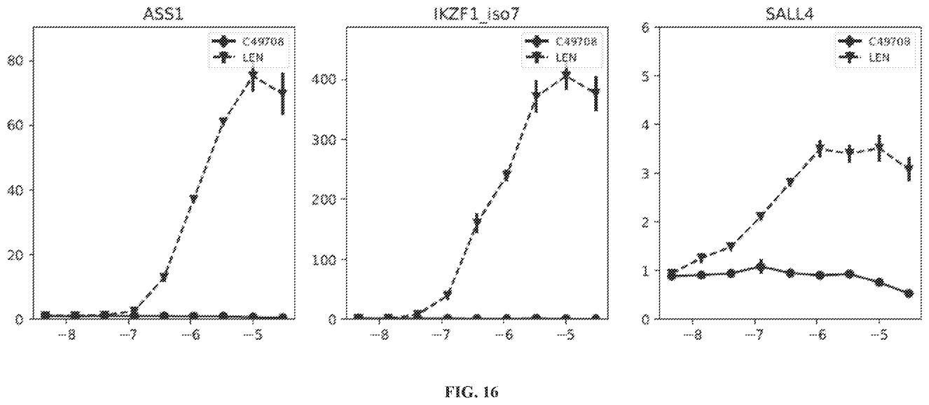

FIG. 17 illustrates the change of cereblon's ability to recruit ASS1, IKZF1, and SALL4 after binding with C96622 in comparison to LEN.

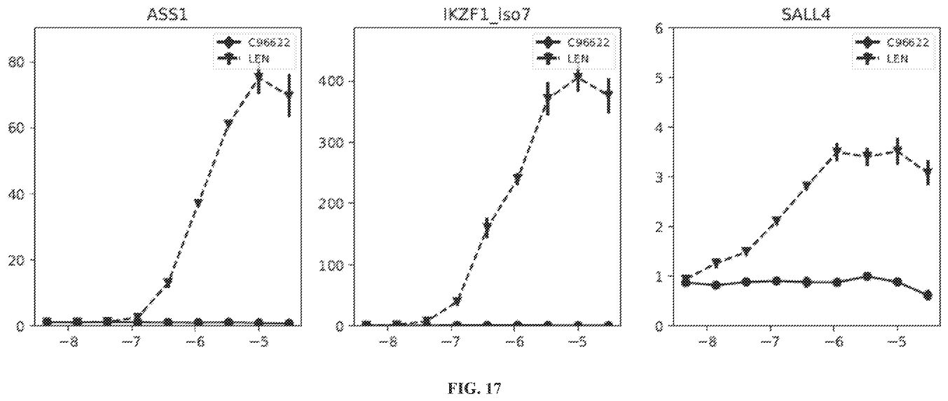

FIG. 18 illustrates the change of cereblon's ability to recruit ASS1, IKZF1, and SALL4 after binding with C45748 in comparison to LEN.

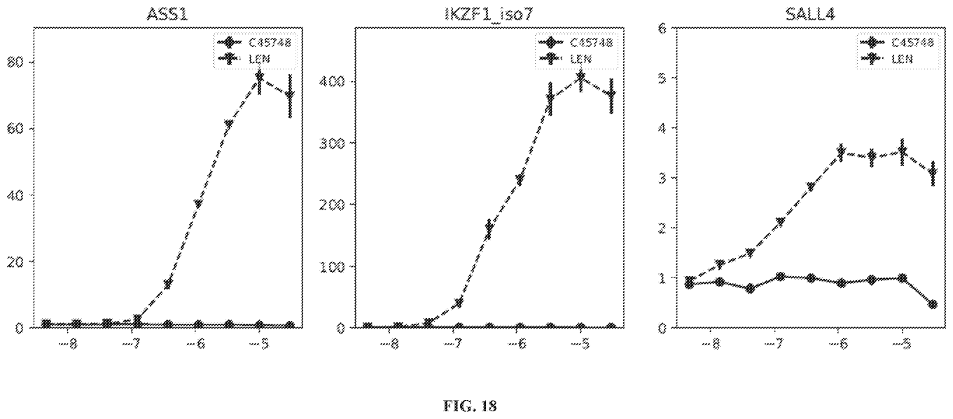

FIG. 19 illustrates the change of cereblon's ability to recruit ASS1, IKZF1, and SALL4 after binding with C84961 in comparison to LEN.

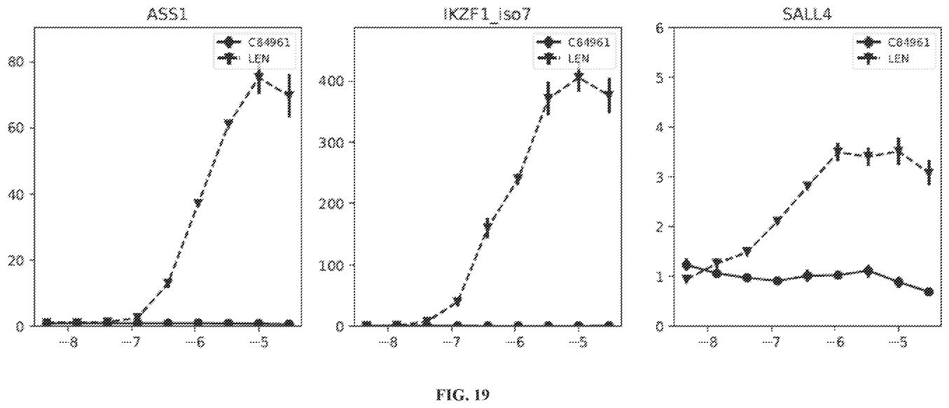

FIG. 20 illustrates the change of cereblon's ability to recruit ASS1, IKZF1, and SALL4 after binding with C84964 in comparison to LEN.

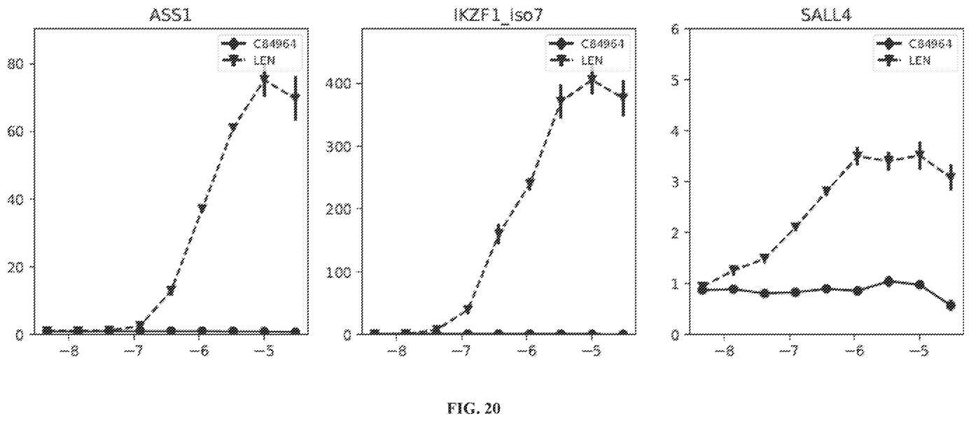

FIG. 21 illustrates the change of cereblon's ability to recruit ASS1, IKZF1, and SALL4 after binding with C84966 in comparison to LEN.

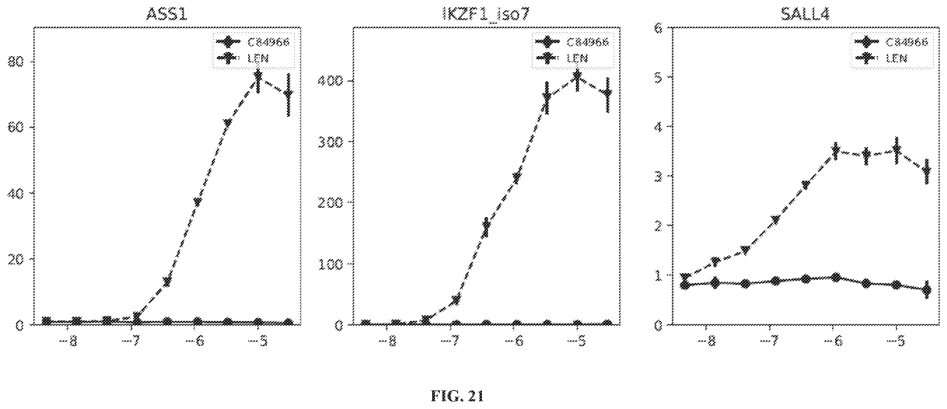

FIG. 22 illustrates the change of cereblon's ability to recruit ASS1, IKZF1, and SALL4 after binding with C84971 in comparison to LEN.

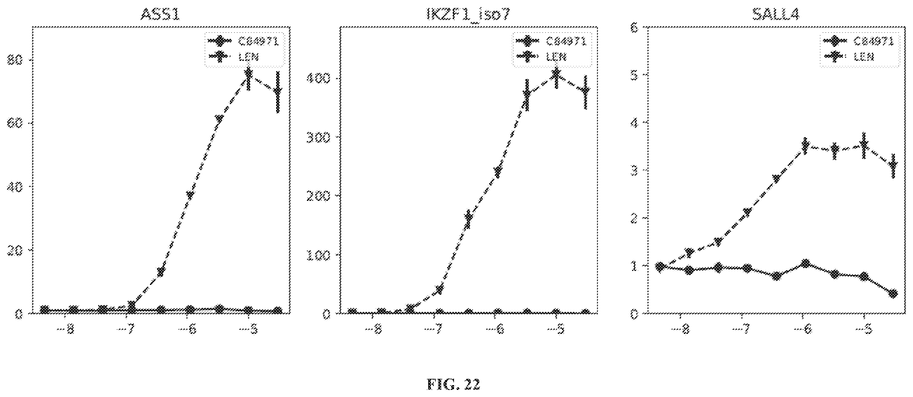

FIG. 23 illustrates the change of cereblon's ability to recruit ASS1, IKZF1, and SALL4 after binding with C56572 in comparison to LEN.

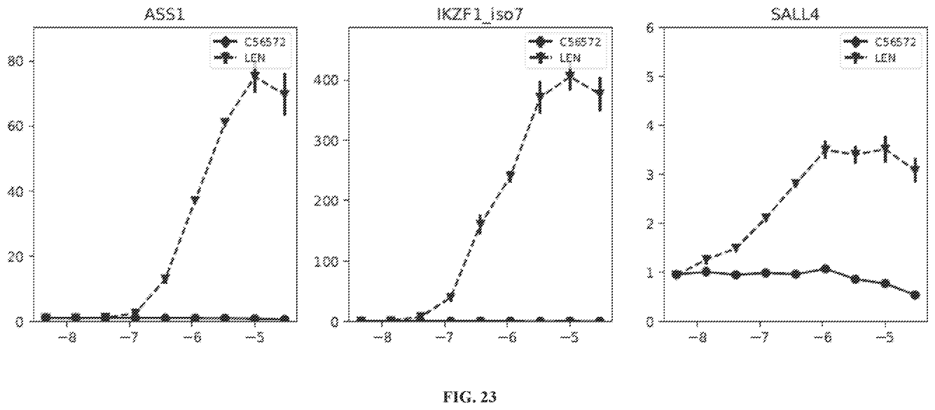

FIG. 24 illustrates the change of cereblon's ability to recruit ASS1, IKZF1, and SALL4 after binding with C64324 in comparison to LEN.

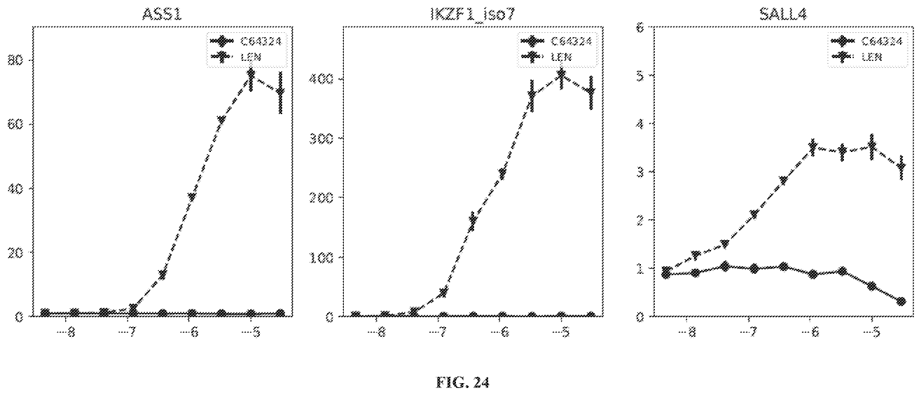

FIG. 25 illustrates the change of cereblon's ability to recruit ASS1, IKZF1, and SALL4 after binding with C64376 in comparison to LEN.

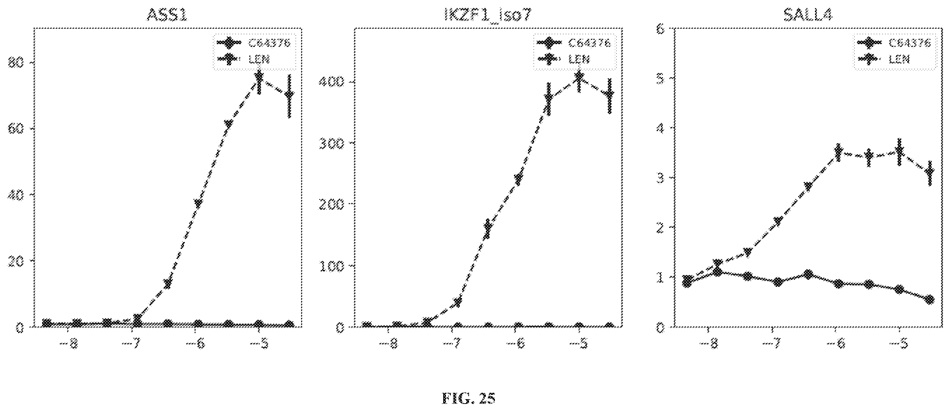

FIG. 26 illustrates the change of cereblon's ability to recruit ASS1, IKZF1, and SALL4 after binding with C80370 in comparison to LEN.

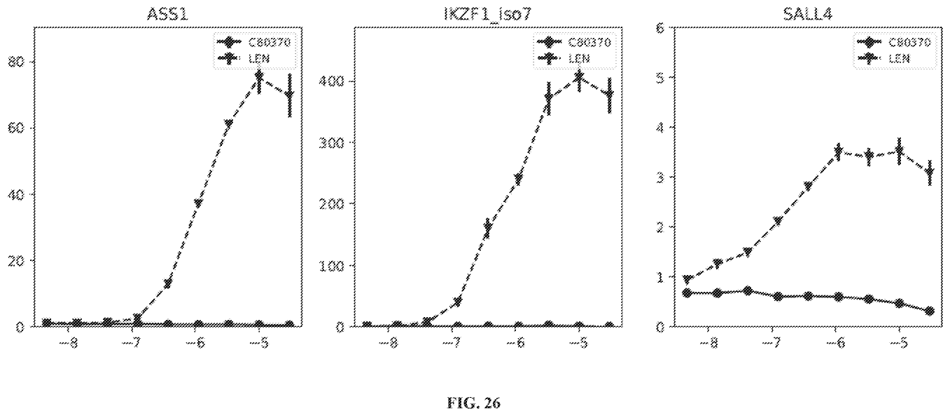

FIG. 27 illustrates the change of cereblon's ability to recruit ASS1, IKZF1, and SALL4 after binding with C80375 in comparison to LEN.

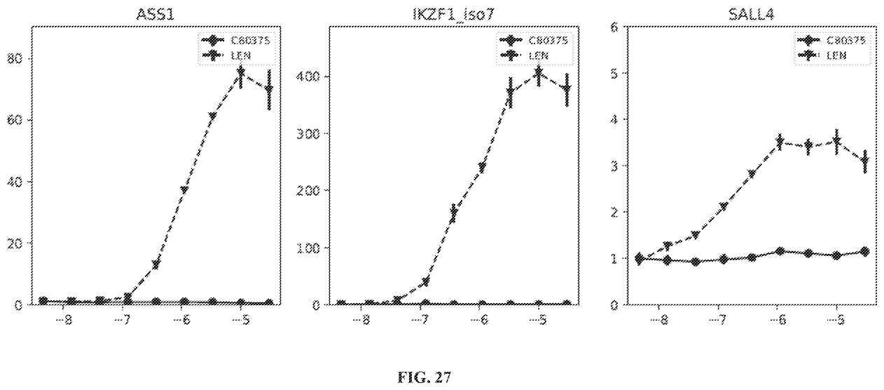

FIG. 28 illustrates the change of cereblon's ability to recruit ASS1, IKZF1, and SALL4 after binding with C80382 in comparison to LEN.

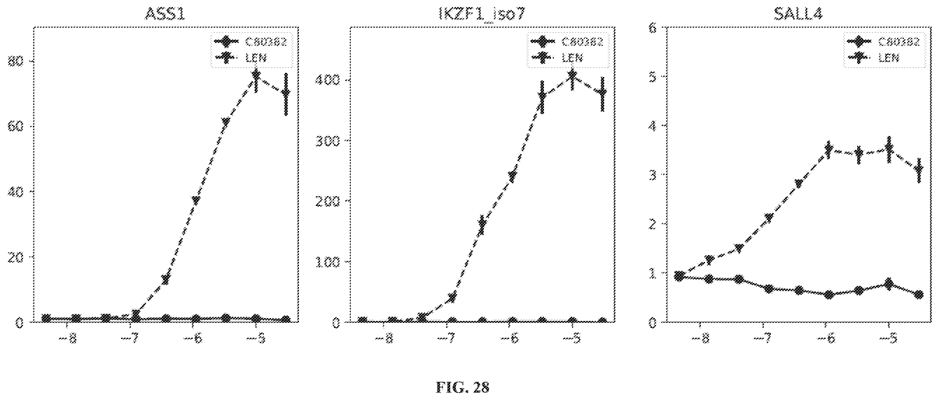

FIG. 29 illustrates the change of cereblon's ability to recruit ASS1, IKZF1, and SALL4 after binding with C80383 in comparison to LEN.

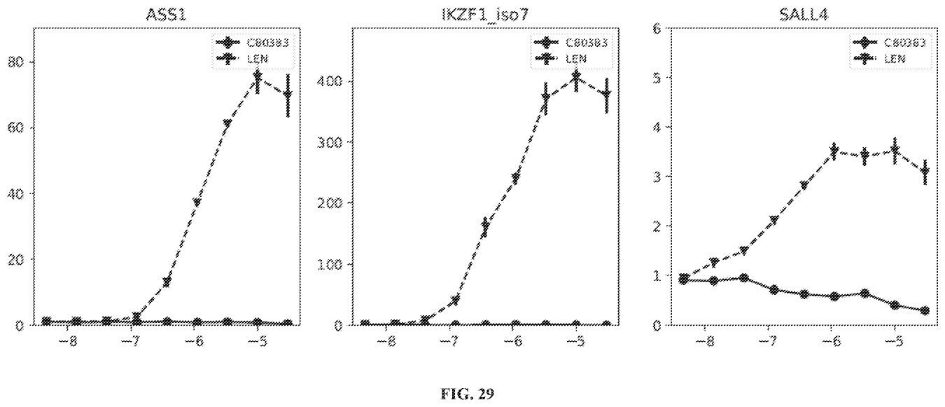

FIG. 30 illustrates the change of cereblon's ability to recruit ASS1, IKZF1, and SALL4 after binding with C80384 in comparison to LEN.

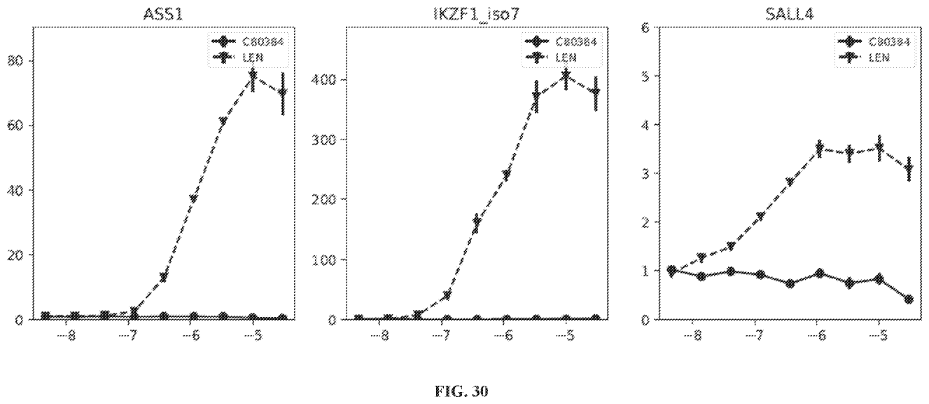

FIG. 31 illustrates the change of cereblon's ability to recruit ASS1, IKZF1, and SALL4 after binding with C80386 in comparison to LEN.

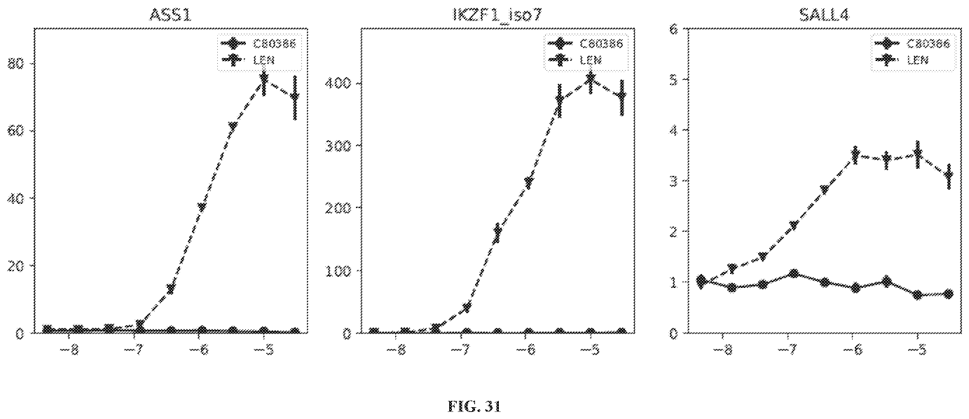

FIG. 32 illustrates the change of cereblon's ability to recruit ASS1, IKZF1, and SALL4 after binding with C80387 in comparison to LEN.

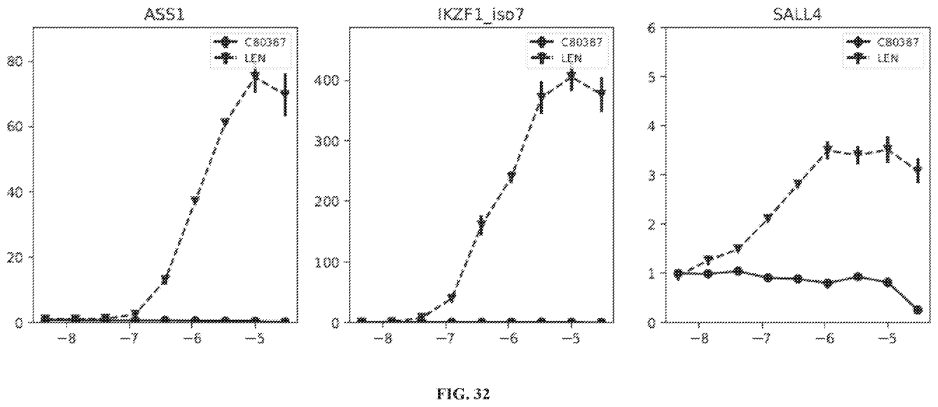

FIG. 33 illustrates the change of cereblon's ability to recruit ASS1, IKZF1, and SALL4 after binding with C80389 in comparison to LEN.

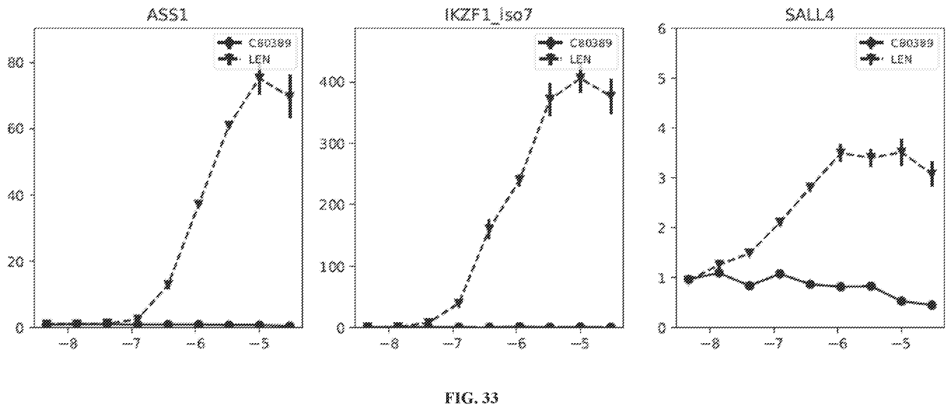

FIG. 34 illustrates the change of cereblon's ability to recruit ASS1, IKZF1, and SALL4 after binding with C80392 in comparison to LEN.

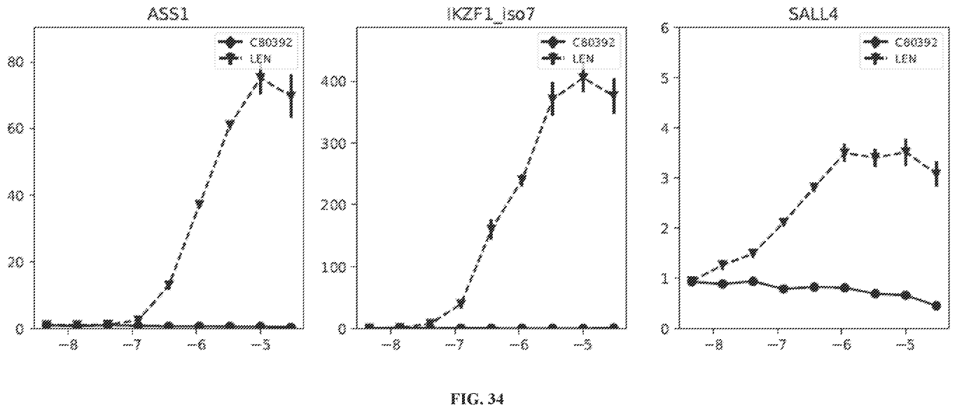

FIG. 35 illustrates the change of cereblon's ability to recruit ASS1, IKZF1, and SALL4 after binding with C98053 in comparison to LEN.

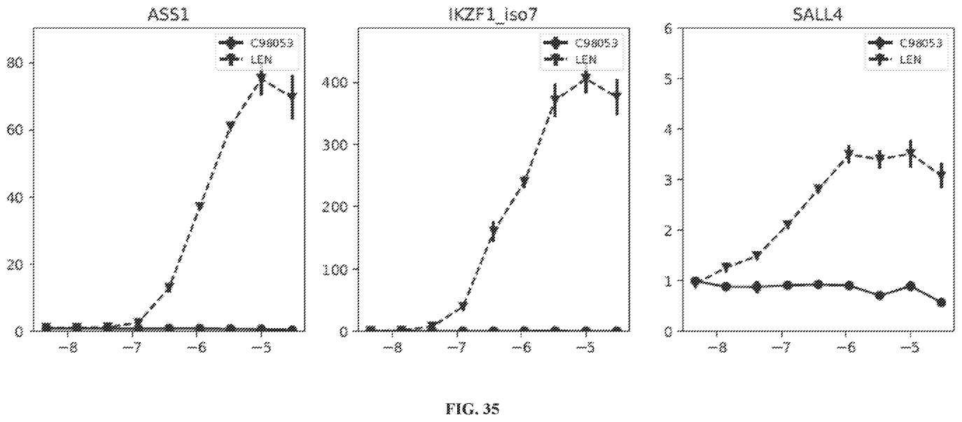

FIG. 36 illustrates the change of cereblon's ability to recruit ASS1, IKZF1, and SALL4 after binding with C49713 in comparison to LEN.

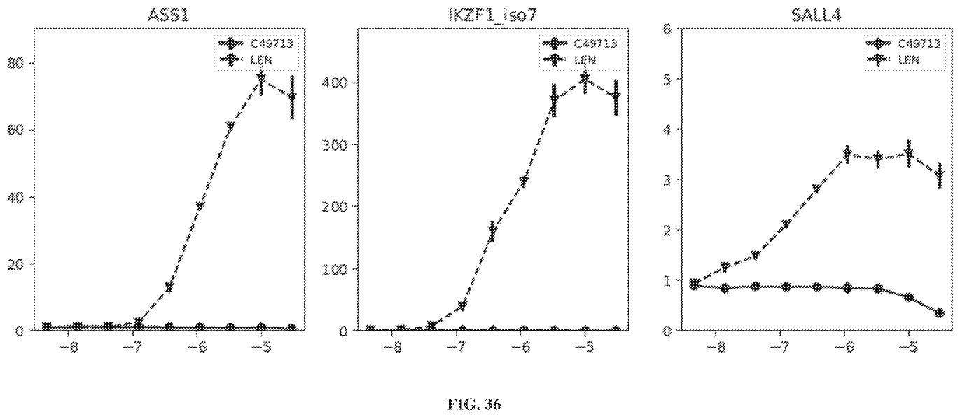

FIG. 37 illustrates the change of cereblon's ability to recruit ASS1, IKZF1, and SALL4 after binding with C11892 in comparison to LEN.

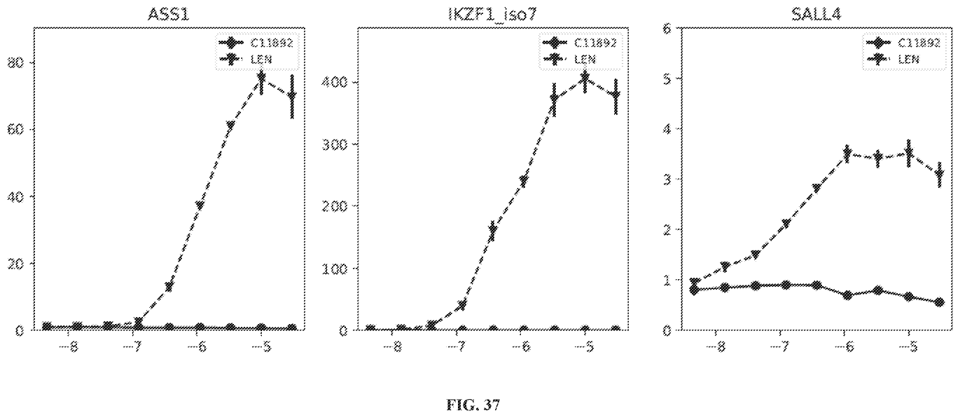

FIG. 38 illustrates the change of cereblon's ability to recruit ASS1, IKZF1, and SALL4 after binding with C12581 in comparison to LEN.

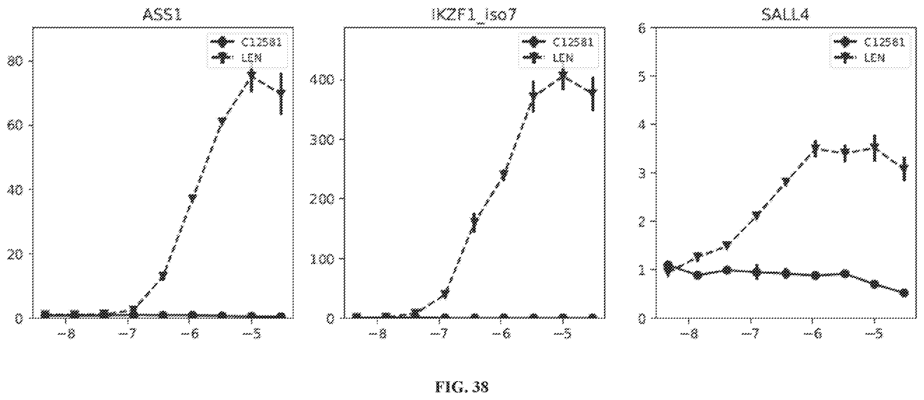

FIG. 39 illustrates the change of cereblon's ability to recruit ASS1, IKZF1, and SALL4 after binding with C12583 in comparison to LEN.

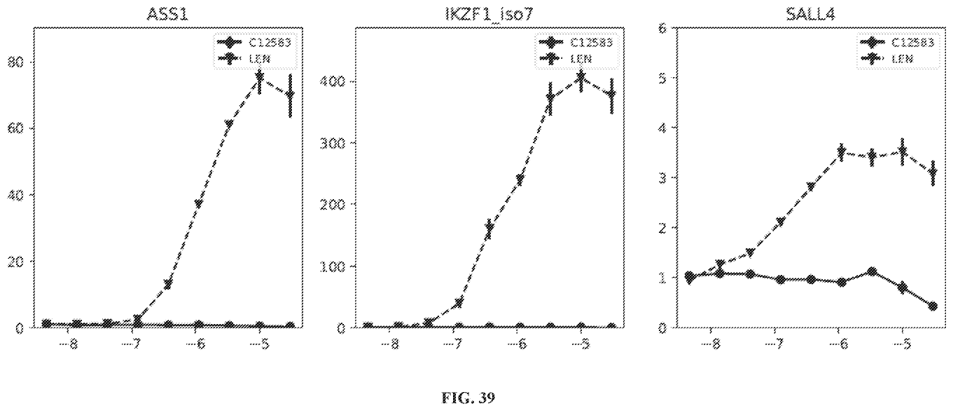

FIG. 40 illustrates the change of cereblon's ability to recruit ASS1, IKZF1, and SALL4 after binding with C12584 in comparison to LEN.

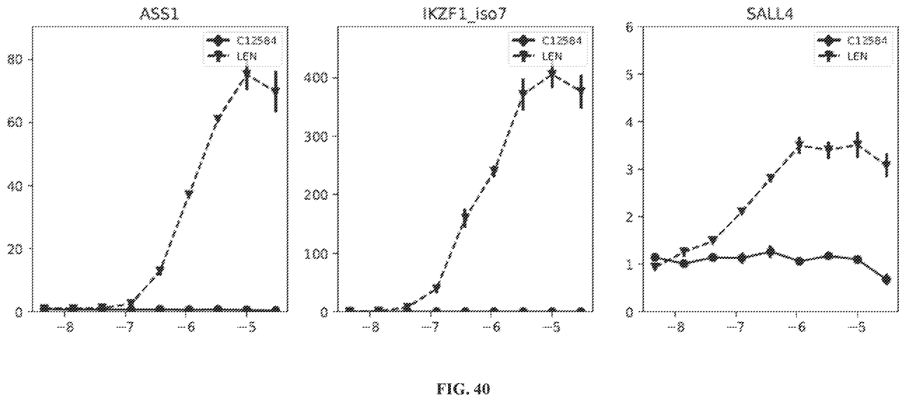

FIG. 41 illustrates the change of cereblon's ability to recruit ASS1, IKZF1, and SALL4 after binding with C12586 in comparison to LEN.

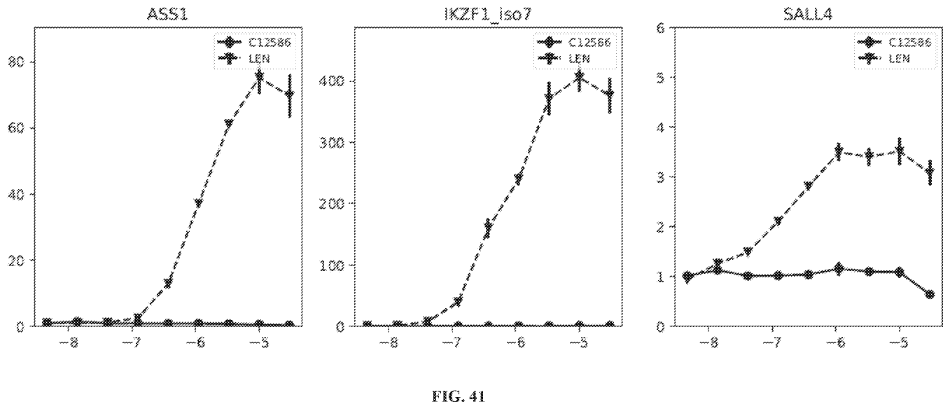

FIG. 42 illustrates the change of cereblon's ability to recruit ASS1, IKZF1, and SALL4 after binding with C12595 in comparison to LEN.

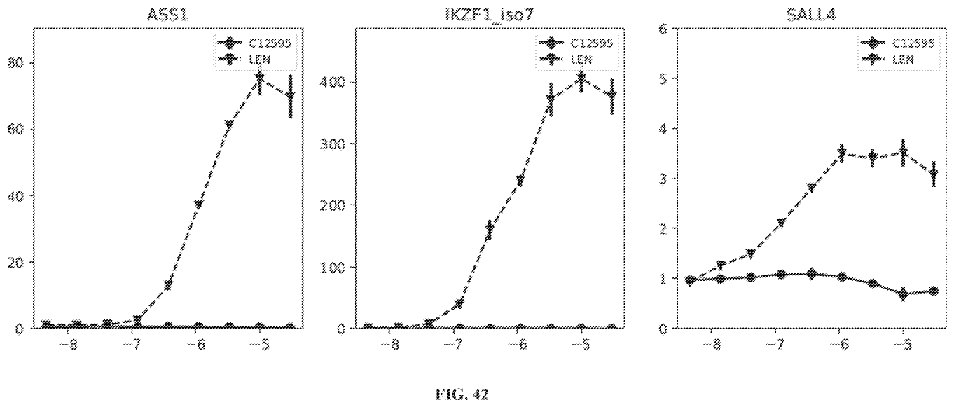

FIG. 43 illustrates the change of cereblon's ability to recruit ASS1, IKZF1, and SALL4 after binding with C12597 in comparison to LEN.

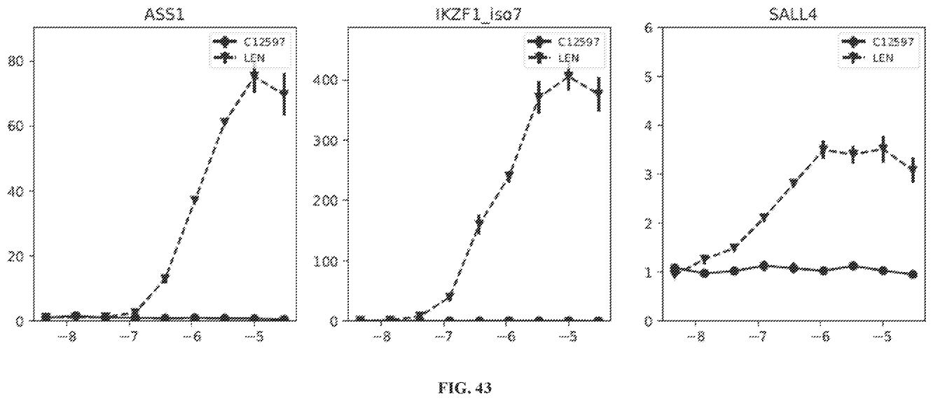

FIG. 44 illustrates the change of cereblon's ability to recruit ASS1, IKZF1, and SALL4 after binding with C12598 in comparison to LEN.

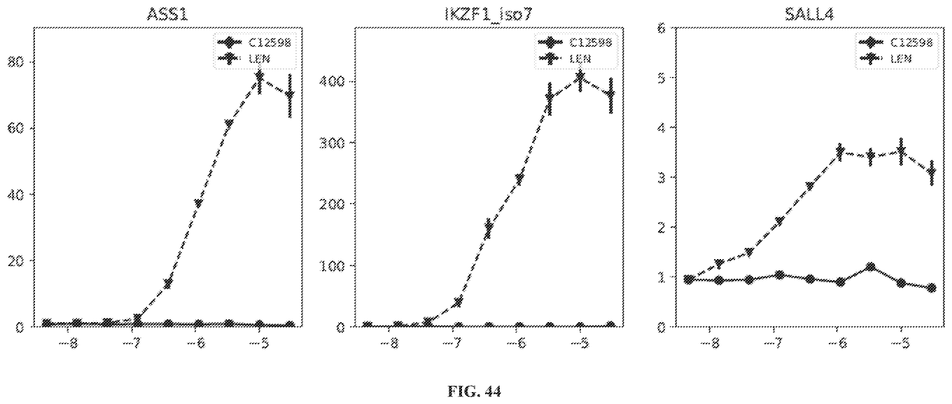

FIG. 45 illustrates the change of cereblon's ability to recruit ASS1, IKZF1, and SALL4 after binding with C95330 in comparison to LEN.

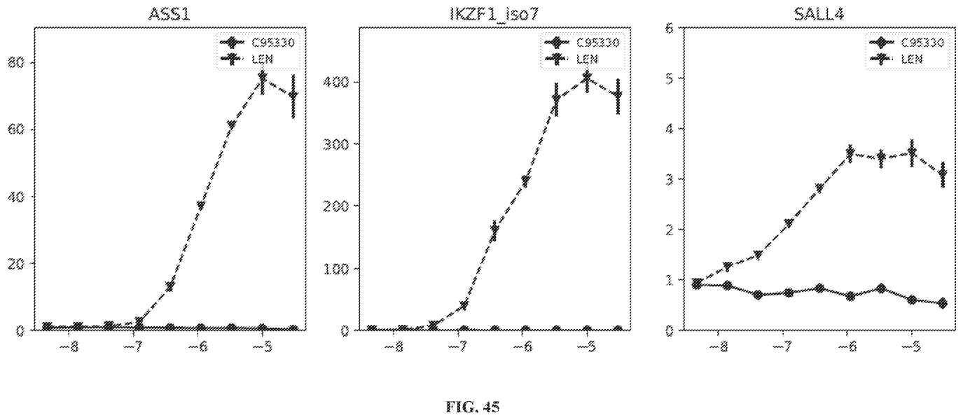

FIG. 46 illustrates the change of cereblon's ability to recruit ASS1, IKZF1, and SALL4 after binding with C95333 in comparison to LEN.

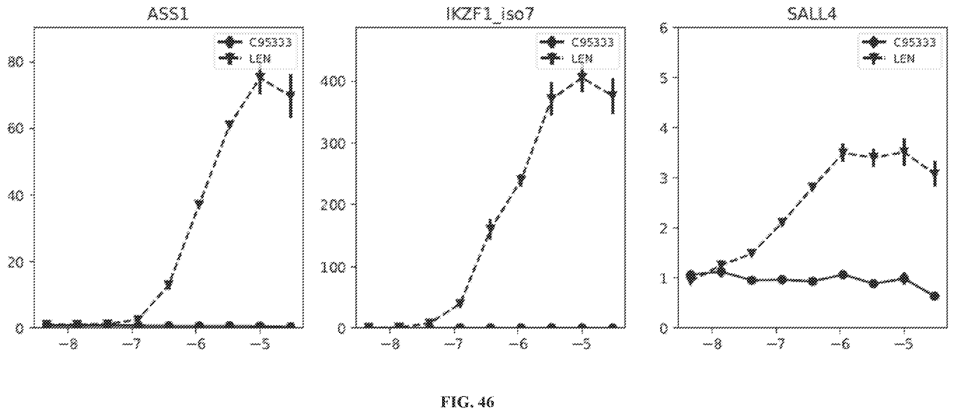

FIG. 47 illustrates the change of cereblon's ability to recruit ASS1, IKZF1, and SALL4 after binding with C95338 in comparison to LEN.

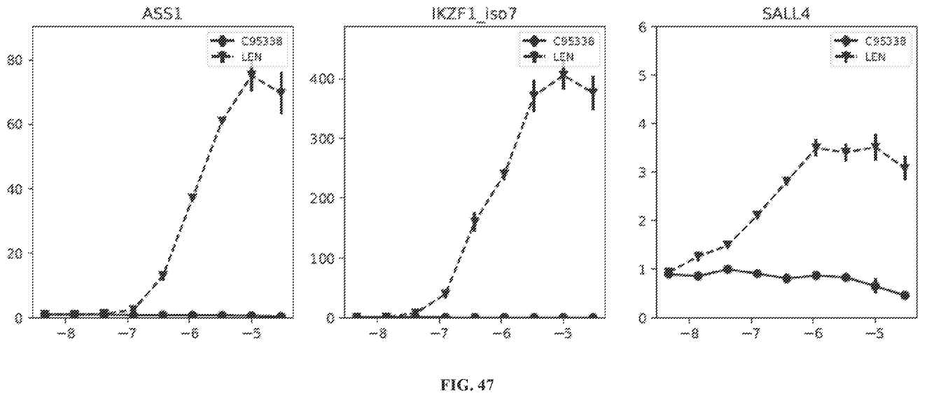

FIG. 48 illustrates the change of cereblon's ability to recruit ASS1, IKZF1, and SALL4 after binding with C74668 in comparison to LEN.

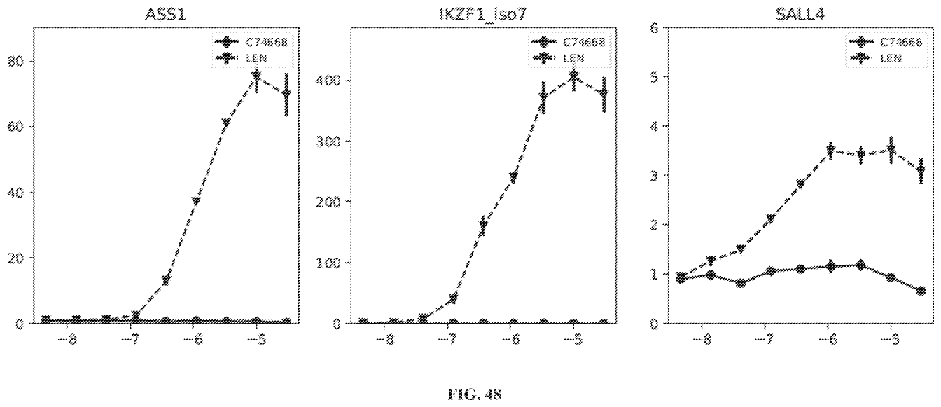

FIG. 49 illustrates the change of cereblon's ability to recruit ASS1, IKZF1, and SALL4 after binding with C73349 in comparison to LEN.

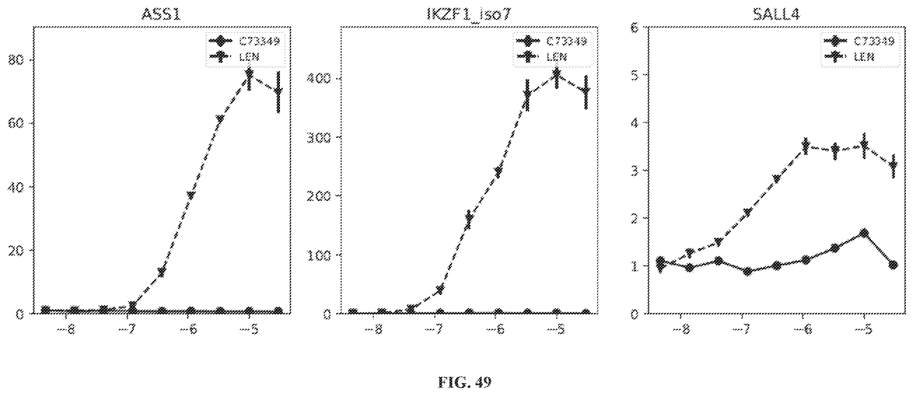

FIG. 50 illustrates the change of cereblon's ability to recruit ASS1, IKZF1, and SALL4 after binding with C35833 in comparison to LEN.

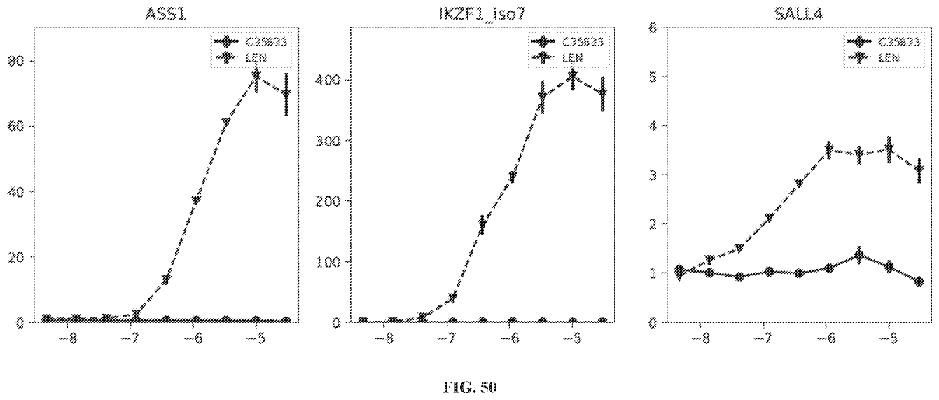

FIG. 51 illustrates the change of cereblon's ability to recruit ASS1, IKZF1, and SALL4 after binding with 5′-OH-THL in comparison to LEN.

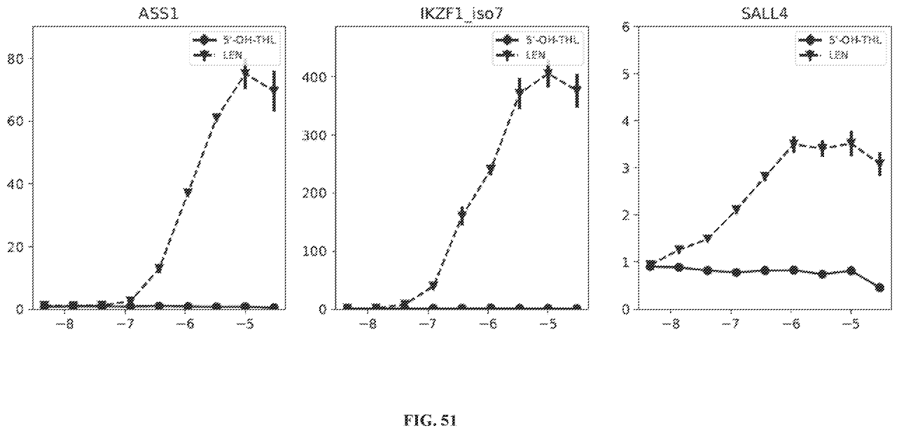

FIG. 52 illustrates the change of cereblon's ability to recruit ASS1, IKZF1, and SALL4 after binding with C60651 in comparison to LEN.

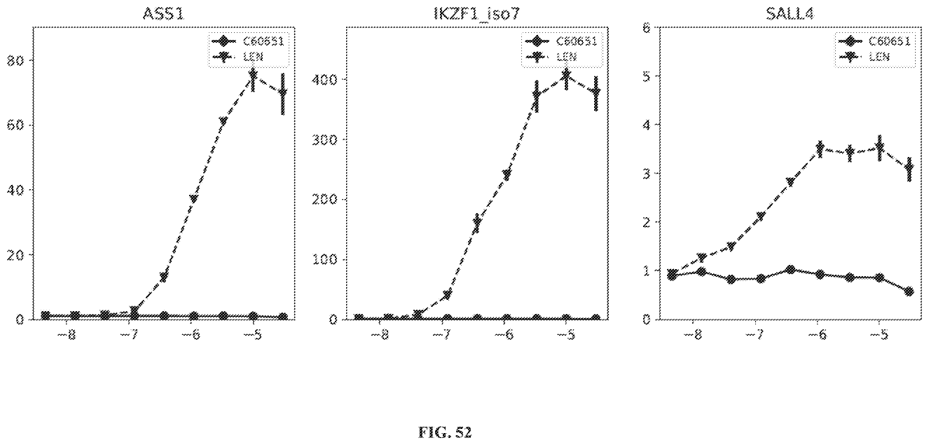

FIG. 53 illustrates the change of cereblon's ability to recruit ASS1, IKZF1, and SALL4 after binding with C58181 in comparison to LEN.

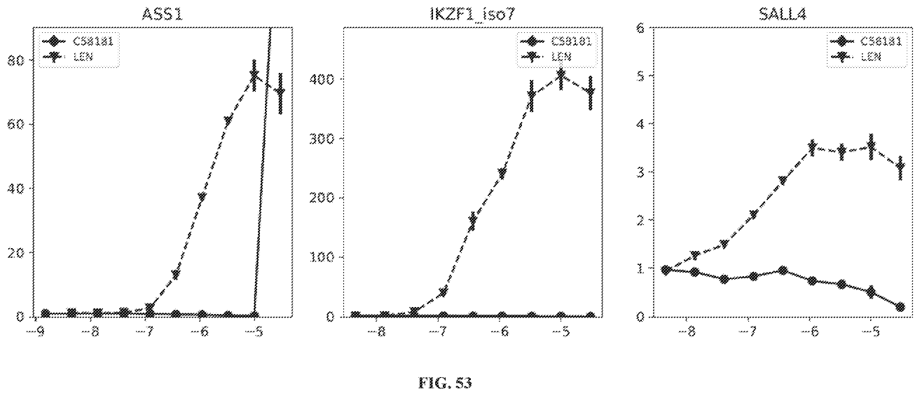

FIG. 54 illustrates the change of cereblon's ability to recruit ASS1, IKZF1, and SALL4 after binding with CC-122 in comparison to LEN.

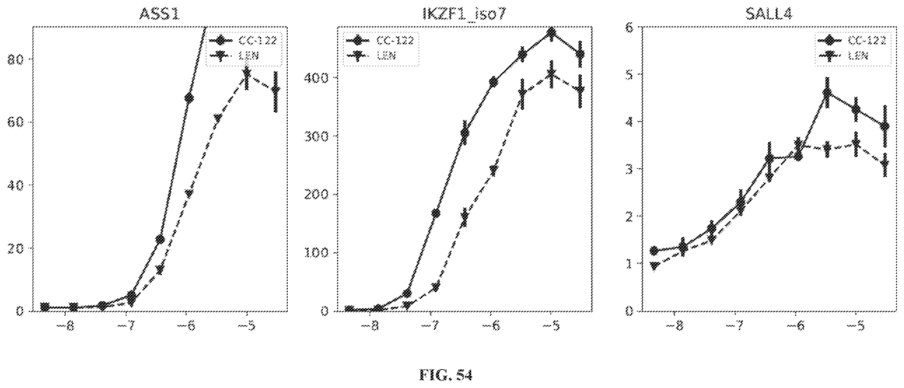

FIG. 55 illustrates the change of cereblon's ability to recruit ASS1, IKZF1, and SALL4 after binding with CC-220 in comparison to LEN.

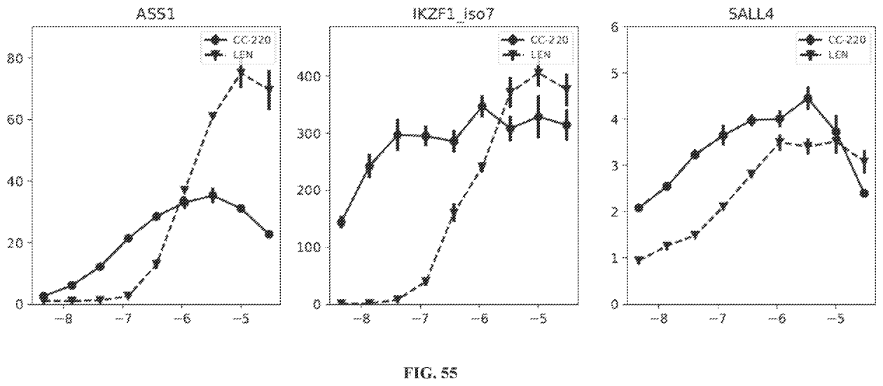

FIG. 56 illustrates the change of cereblon's ability to recruit ASS1, IKZF1, and SALL4 after binding with CC-885 in comparison to LEN.

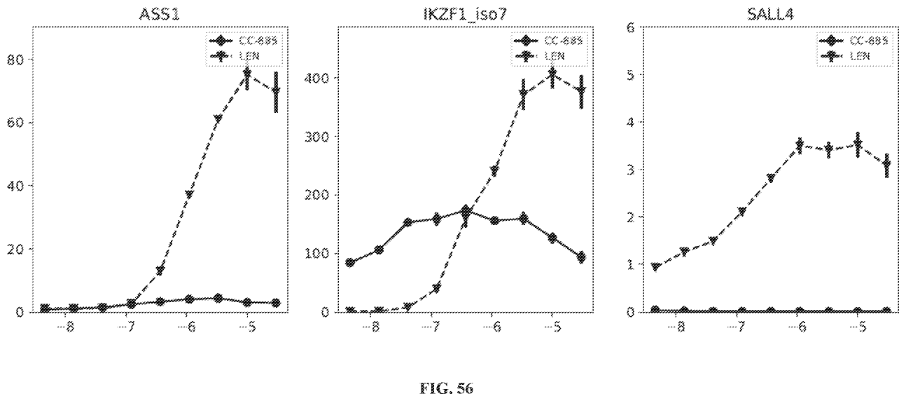

FIG. 57 illustrates the change of cereblon's ability to recruit ASS1, IKZF1, and SALL4 after binding with C15352 in comparison to LEN.

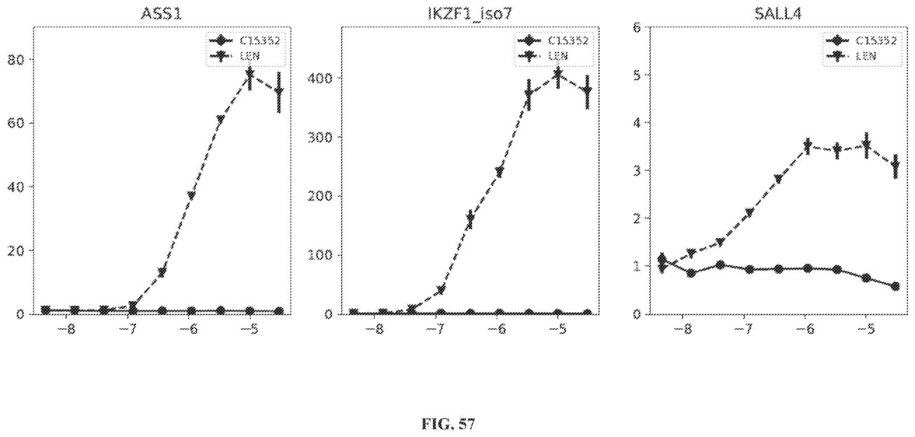

FIG. 58 illustrates the change of cereblon's ability to recruit ASS1, IKZF1, and SALL4 after binding with C36124 in comparison to LEN.

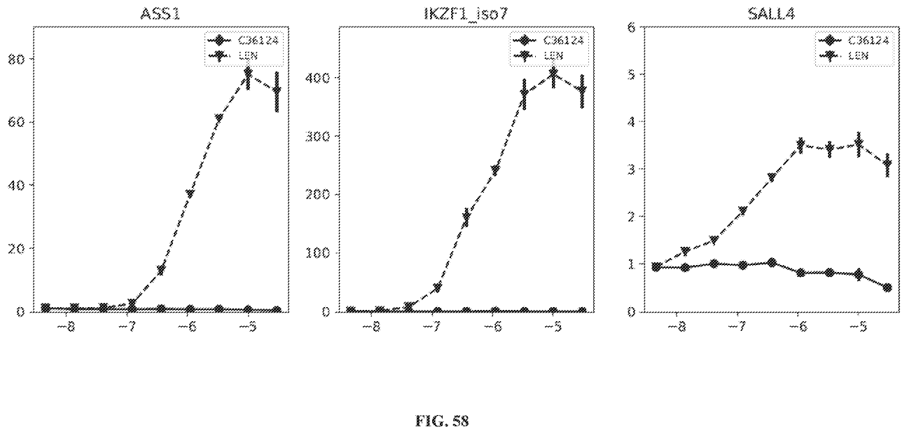

FIG. 59 illustrates the change of cereblon's ability to recruit ASS1, IKZF1, and SALL4 after binding with C36128 in comparison to LEN.

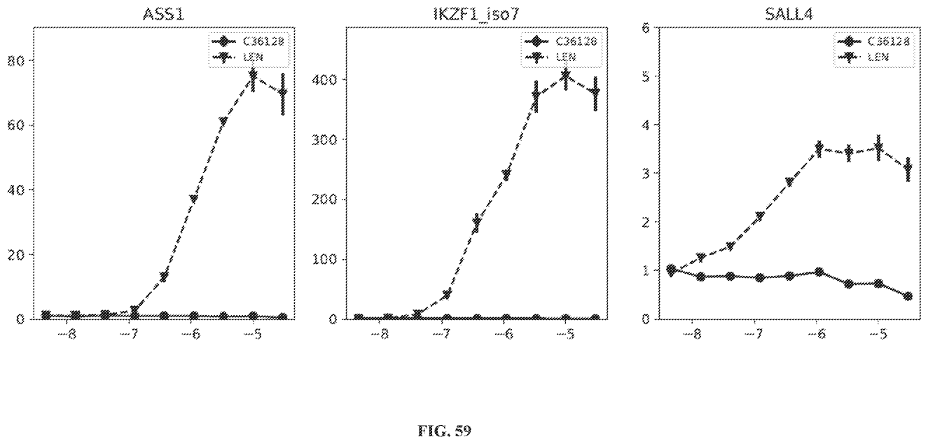

FIG. 60 illustrates the change of cereblon's ability to recruit ASS1, IKZF1, and SALL4 after binding with glutarimide in comparison to LEN.

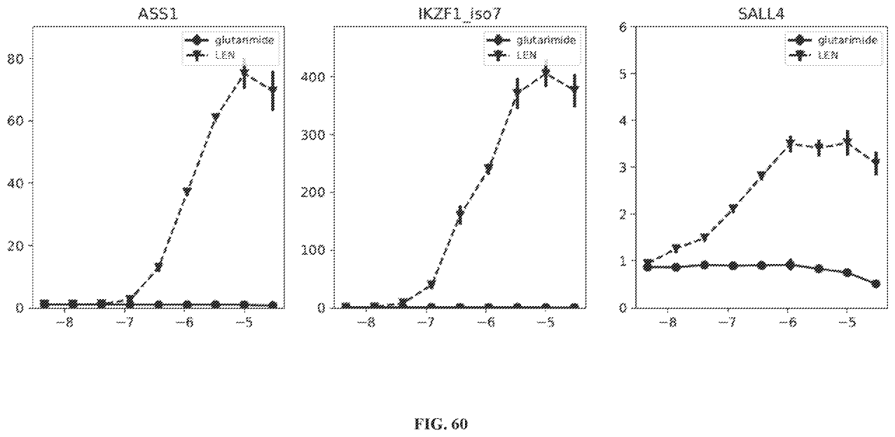

FIG. 61 illustrates LEN's effects on cereblon's ability to recruit ASS1, IKZF1, and SALL4 after binding.

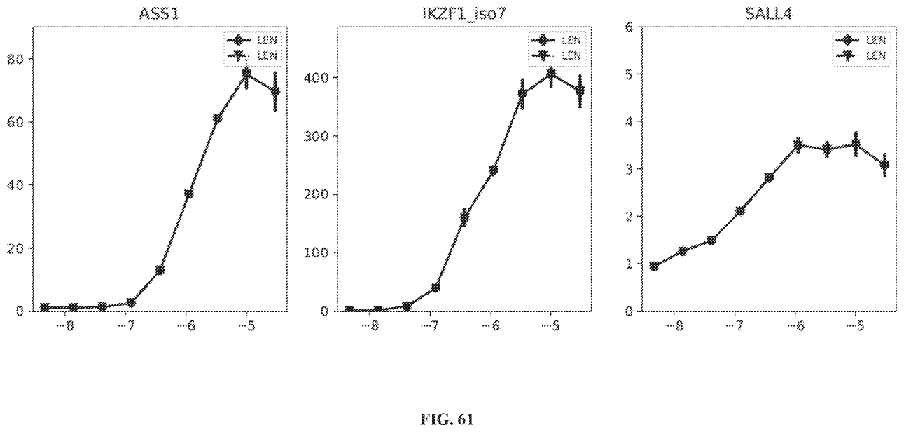

FIG. 62 illustrates the change of cereblon's ability to recruit ASS1, IKZF1, and SALL4 after binding with C10001 in comparison to LEN.

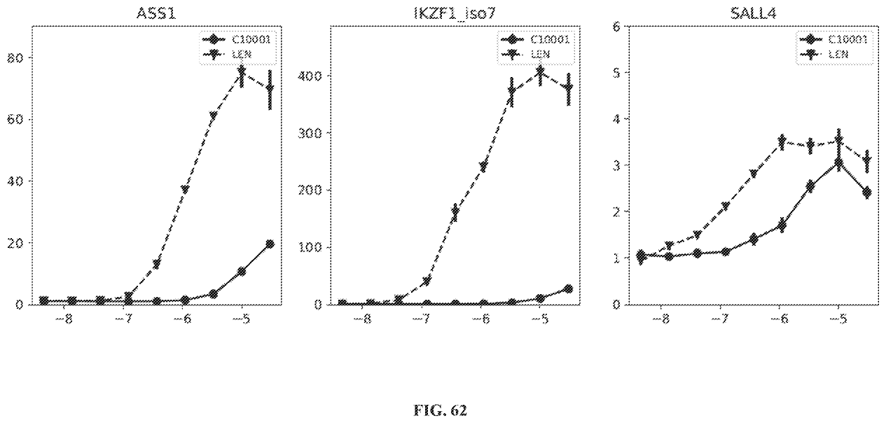

FIG. 63 illustrates the change of cereblon's ability to recruit ASS1, IKZF1, and SALL4 after binding with POM in comparison to LEN.

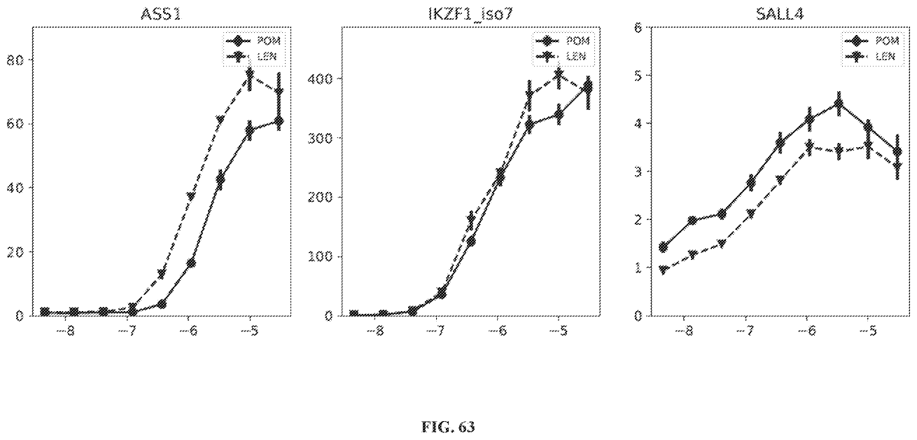

FIG. 64 illustrates the change of cereblon's ability to recruit ASS1, IKZF1, and SALL4 after binding with THL in comparison to LEN.

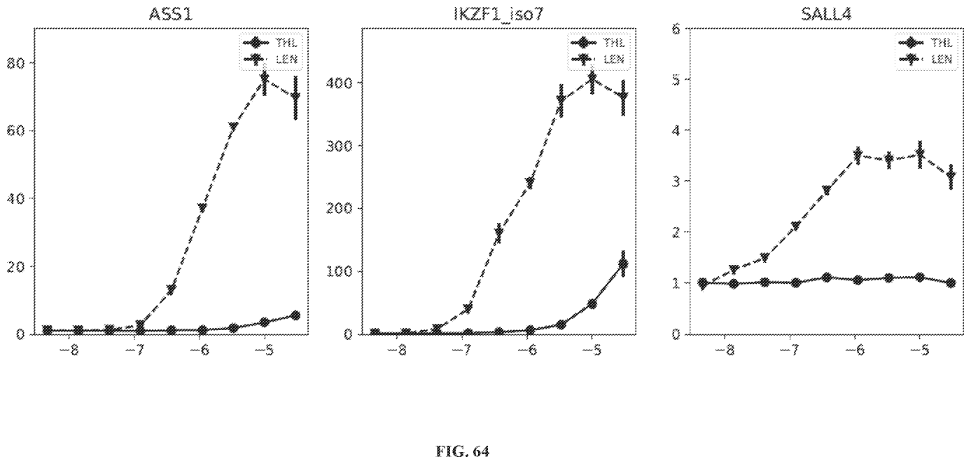

FIG. 65 illustrates the change of cereblon's ability to recruit ASS1, IKZF1, and SALL4 after binding with CC07128 in comparison to LEN.

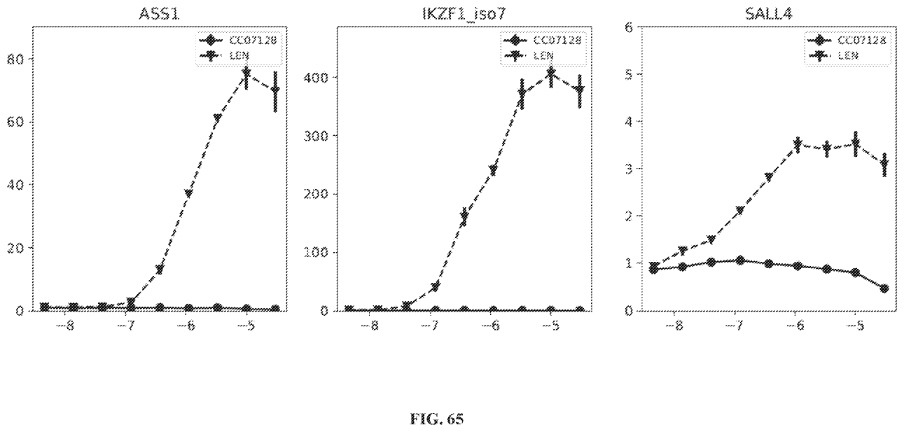

FIG. 66 illustrates the change of cereblon's ability to recruit ASS1, IKZF1, and SALL4 after binding with C38930 in comparison to LEN.

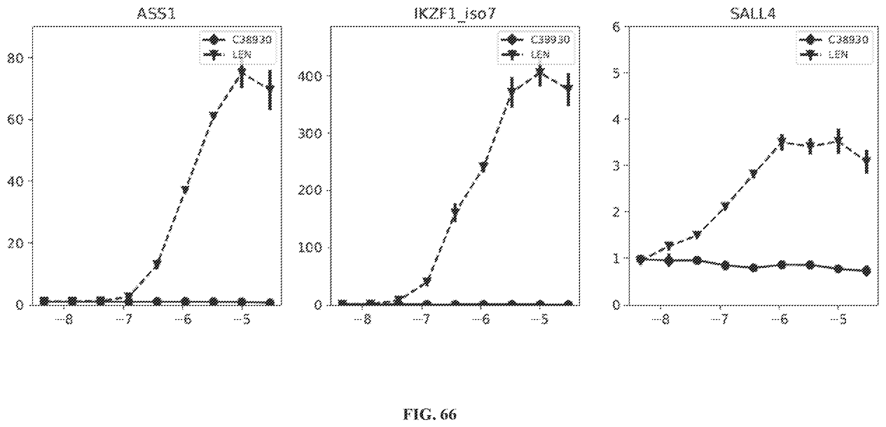

FIG. 67 illustrates the change of cereblon's ability to recruit ASS1, IKZF1, and SALL4 after binding with C38935 in comparison to LEN.

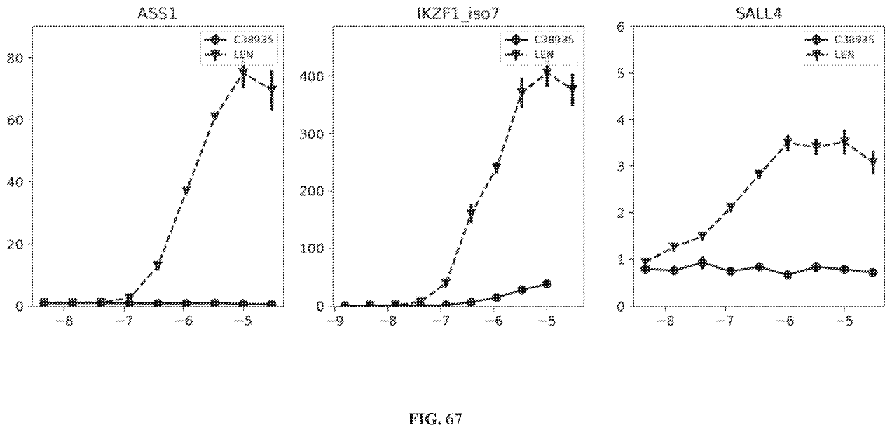

FIG. 68 illustrates the change of cereblon's ability to recruit ASS1, IKZF1, and SALL4 after binding with C75987 in comparison to LEN.

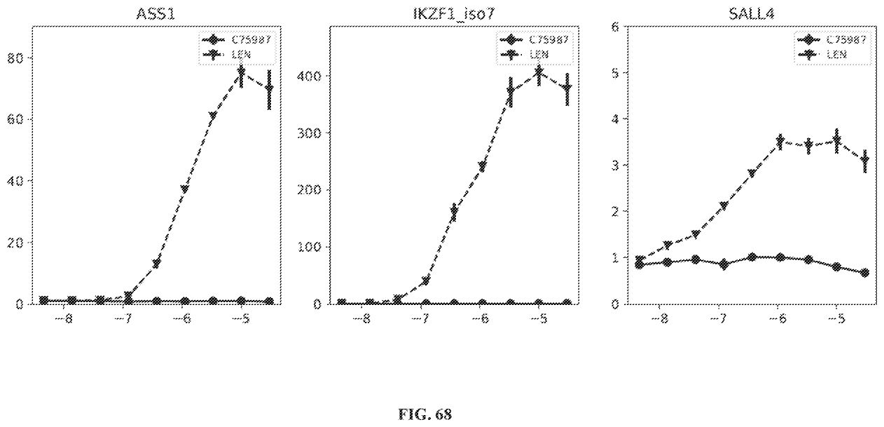

FIG. 69 illustrates the change of cereblon's ability to recruit ASS1, IKZF1, and SALL4 after binding with C87699 in comparison to LEN.

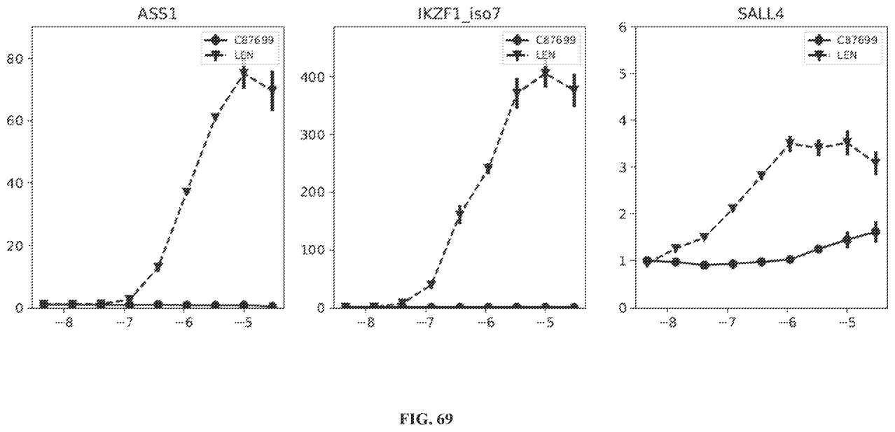

FIG. 70 illustrates the change of cereblon's ability to recruit ASS1, IKZF1, and SALL4 after binding with C24031 in comparison to LEN.

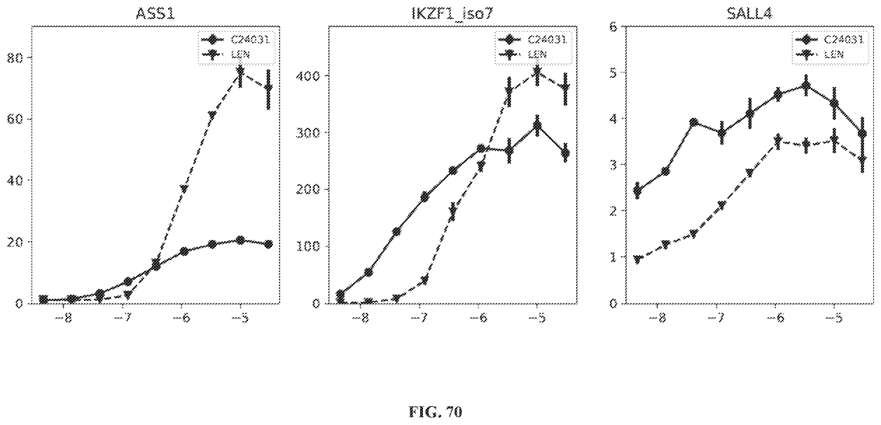

FIG. 71 illustrates the change of cereblon's ability to recruit ASS1, IKZF1, and SALL4 after binding with C07207 in comparison to LEN.

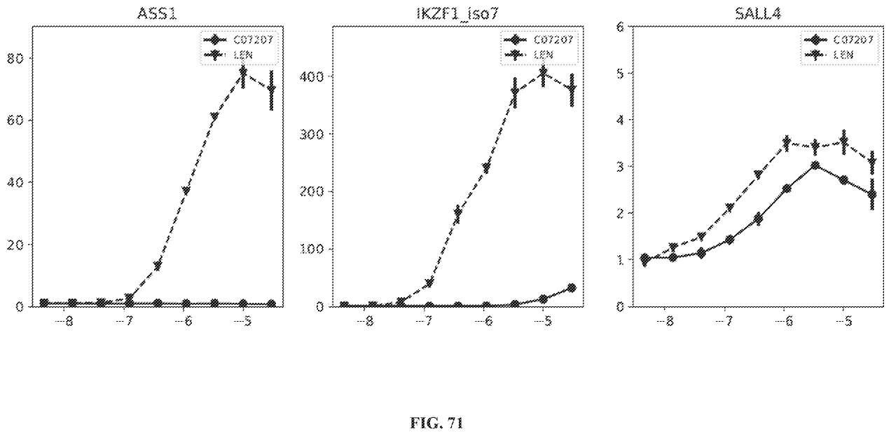

FIG. 72 illustrates the change of cereblon's ability to recruit ASS1, IKZF1, and SALL4 after binding with C89676 in comparison to LEN.

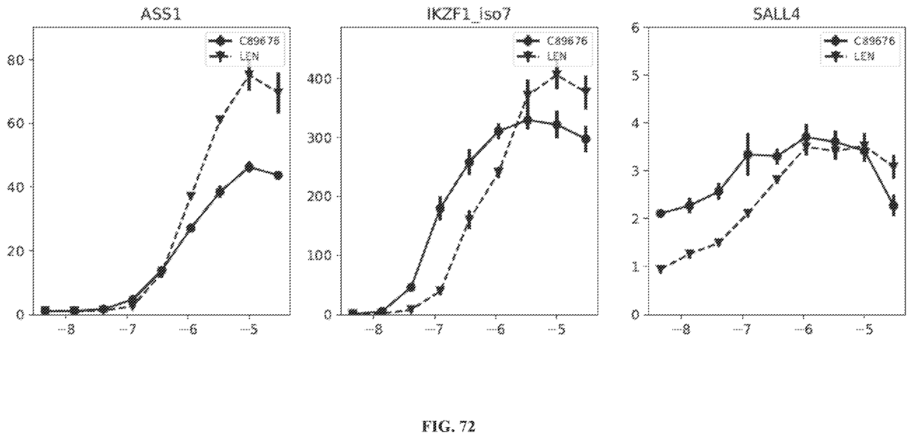

FIG. 73 illustrates the change of cereblon's ability to recruit ASS1, IKZF1, and SALL4 after binding with C28558 in comparison to LEN.

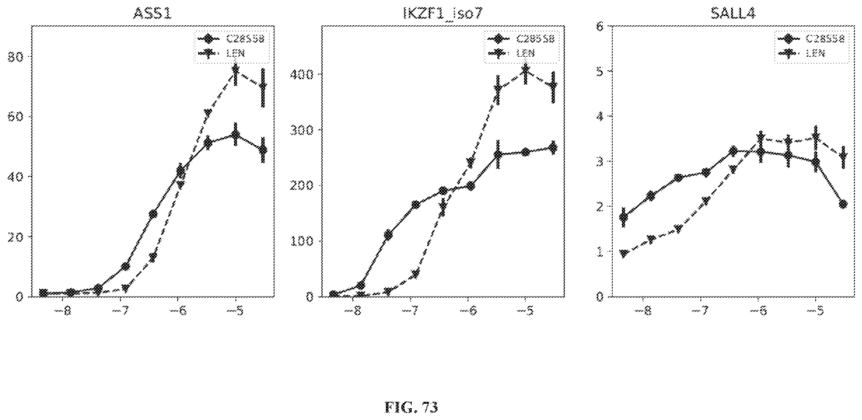

FIG. 74 illustrates the change of cereblon's ability to recruit ASS1, IKZF1, and SALL4 after binding with C28577 in comparison to LEN.

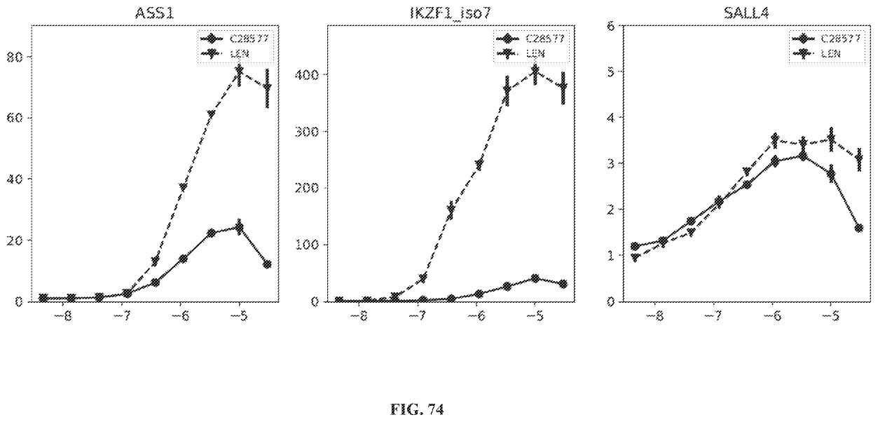

FIG. 75 illustrates the change of cereblon's ability to recruit ASS1, IKZF1, and SALL4 after binding with C28620 in comparison to LEN.

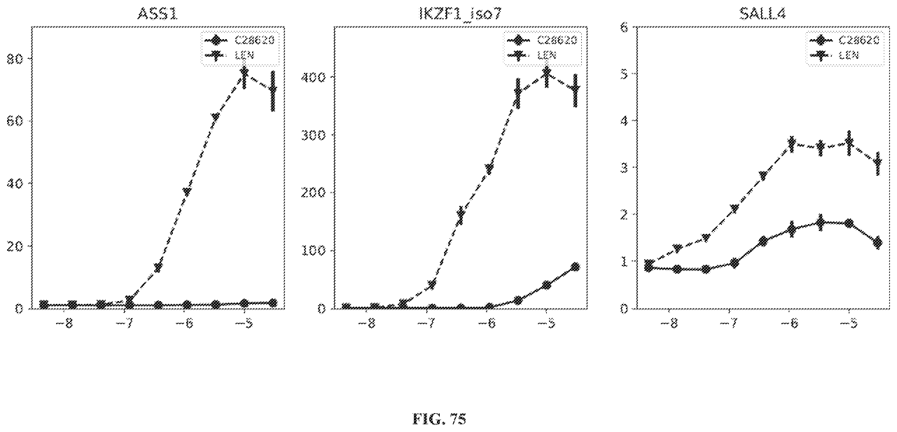

FIG. 76 illustrates the change of cereblon's ability to recruit ASS1, IKZF1, and SALL4 after binding with C28891 in comparison to LEN.

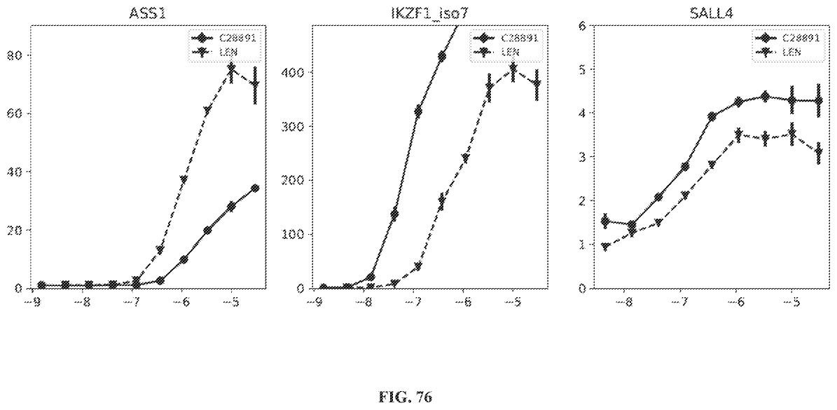

FIG. 77 illustrates the change of cereblon's ability to recruit ASS1, IKZF1, and SALL4 after binding with C28928 in comparison to LEN.

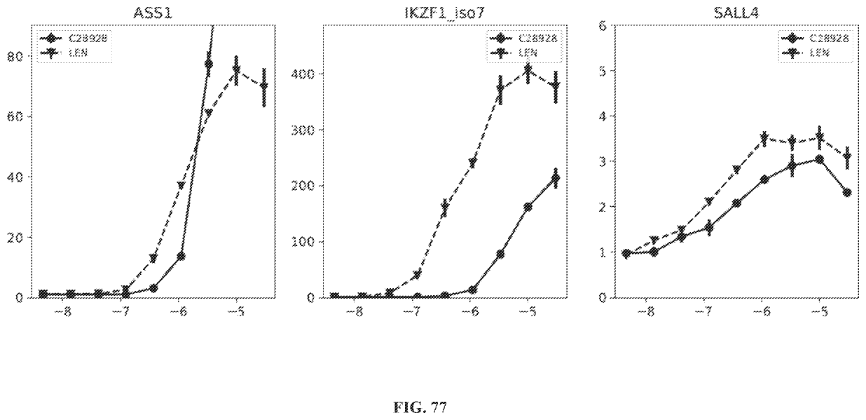

FIG. 78 illustrates the change of cereblon's ability to recruit ASS1, IKZF1, and SALL4 after binding with C28973 in comparison to LEN.

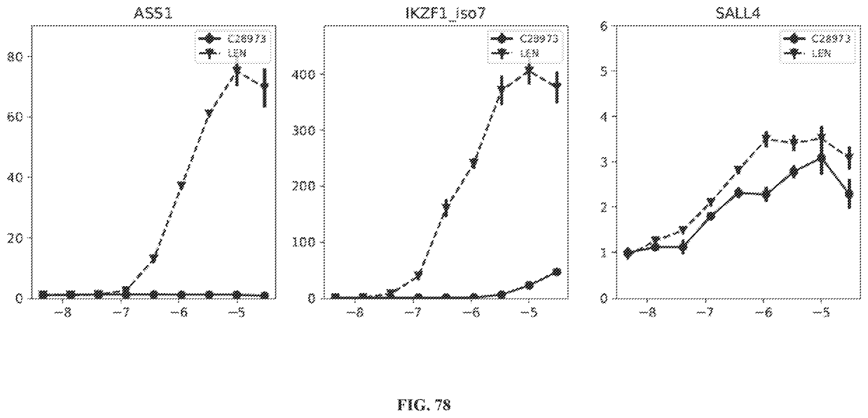

FIG. 79 illustrates the change of cereblon's ability to recruit ASS1, IKZF1, and SALL4 after binding with C29330 in comparison to LEN.

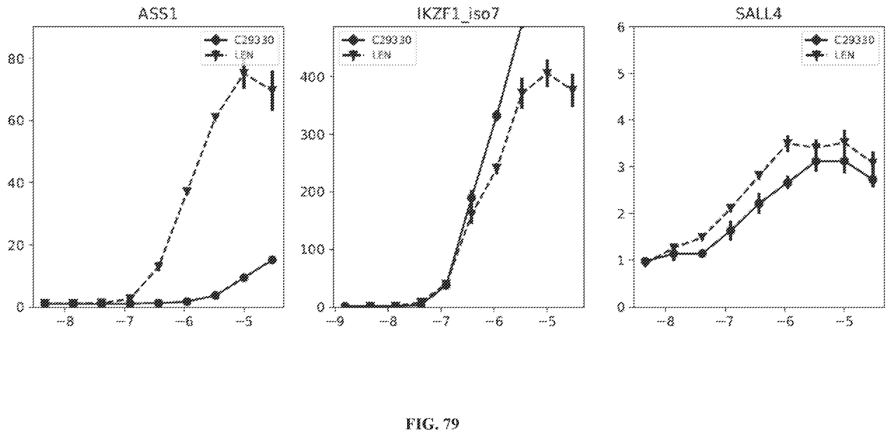

FIG. 80 illustrates the change of cereblon's ability to recruit ASS1, IKZF1, and SALL4 after binding with C29361 in comparison to LEN.

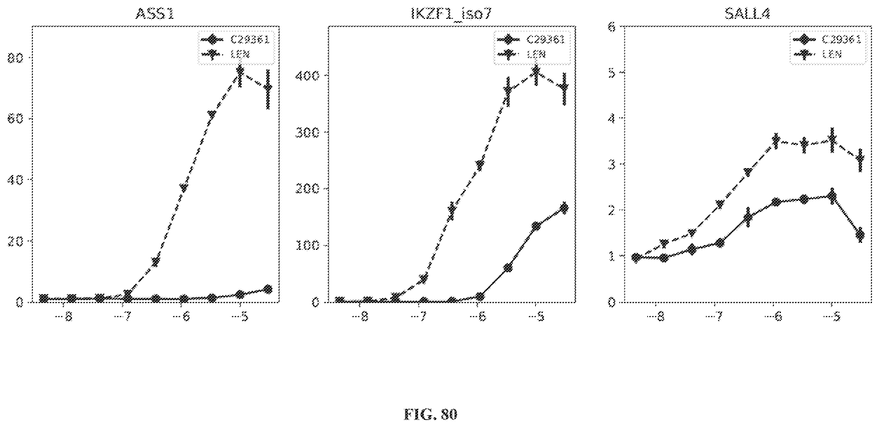

FIG. 81 illustrates the change of cereblon's ability to recruit ASS1, IKZF1, and SALL4 after binding with C29457 in comparison to LEN.

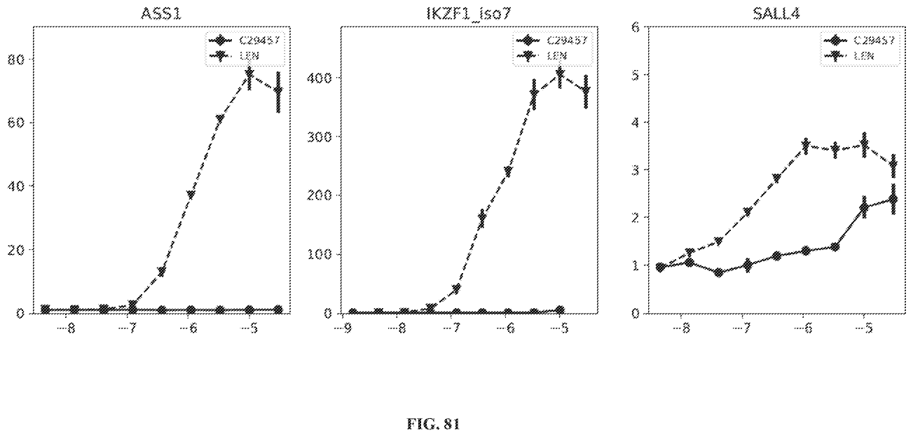

FIG. 82 illustrates the change of cereblon's ability to recruit ASS1, IKZF1, and SALL4 after binding with C29490 in comparison to LEN.

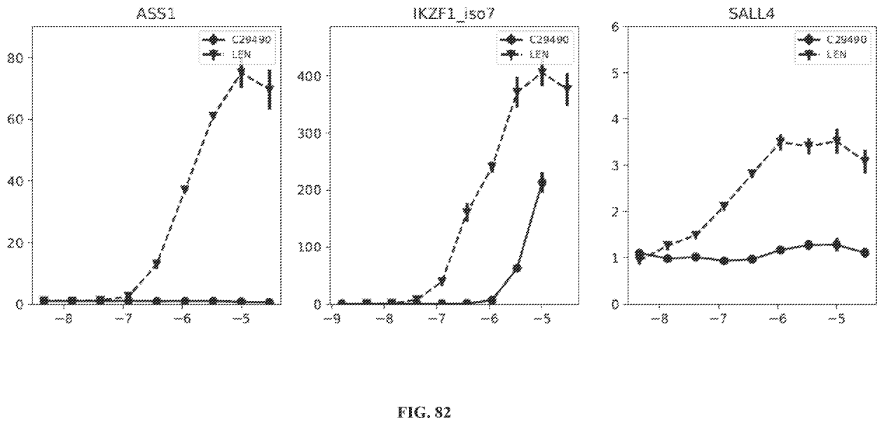

FIG. 83 illustrates the change of cereblon's ability to recruit ASS1, IKZF1, and SALL4 after binding with C14950 in comparison to LEN.

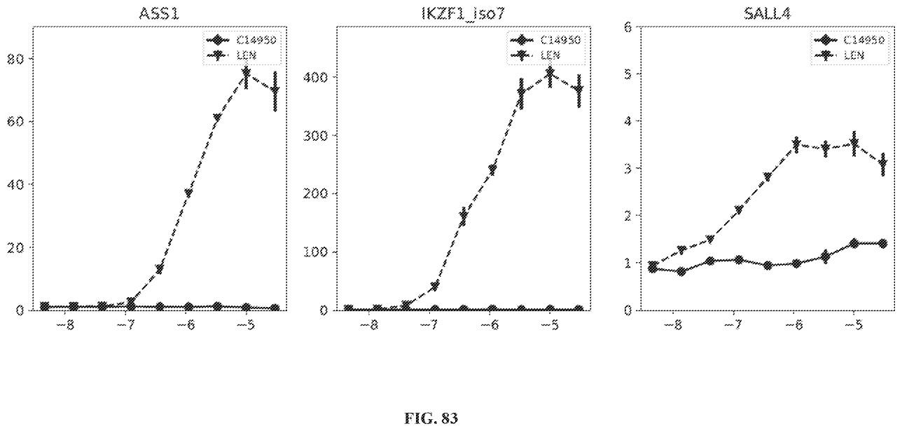

FIG. 84 illustrates the change of cereblon's ability to recruit ASS1, IKZF1, and SALL4 after binding with C24191 in comparison to LEN.

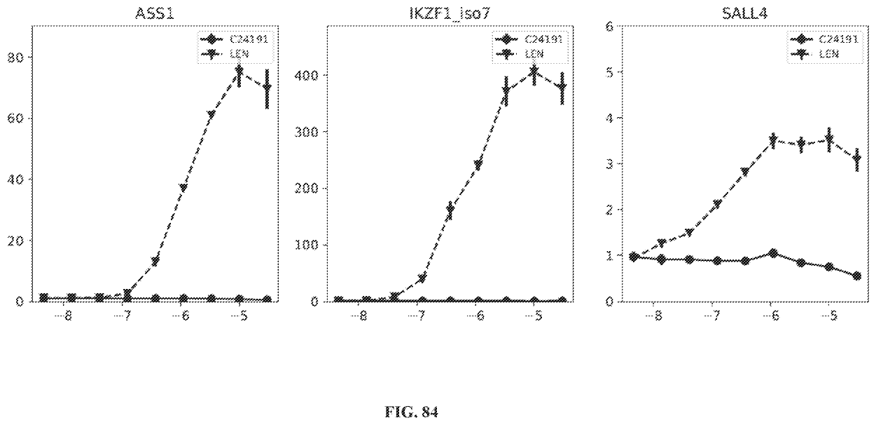

FIG. 85 illustrates the change of cereblon's ability to recruit ASS1, IKZF1, and SALL4 after binding with C02896 in comparison to LEN.

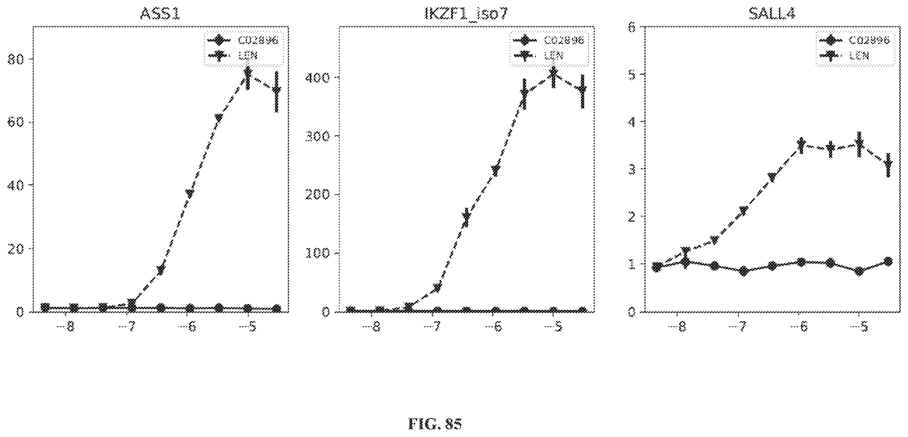

FIG. 86 illustrates the change of cereblon's ability to recruit ASS1, IKZF1, and SALL4 after binding with C47927 in comparison to LEN.

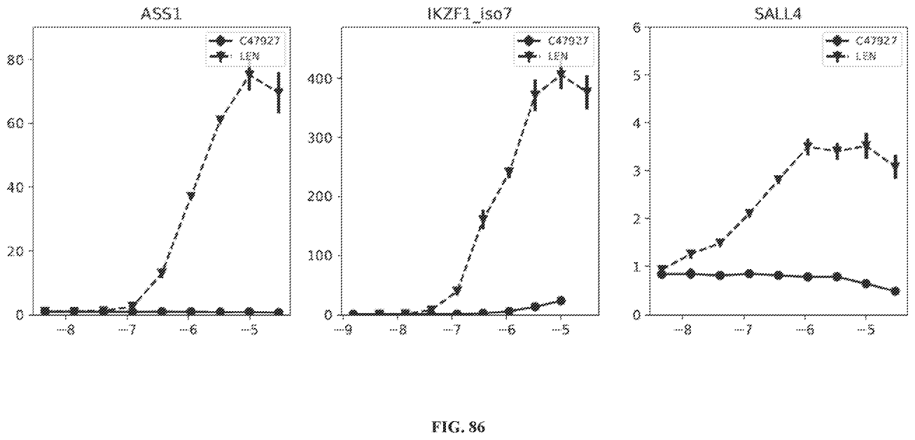

FIG. 87 illustrates the change of cereblon's ability to recruit ASS1, IKZF1, and SALL4 after binding with C47928 in comparison to LEN.

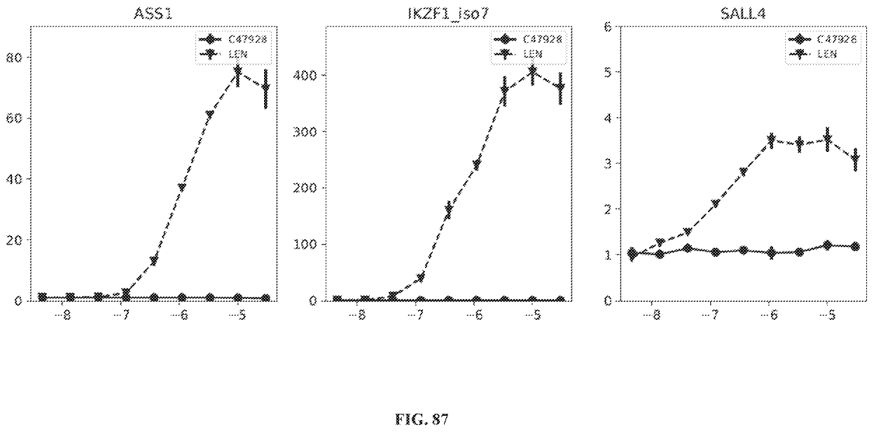

FIG. 88 illustrates the change of cereblon's ability to recruit ASS1, IKZF1, and SALL4 after binding with C47930 in comparison to LEN.

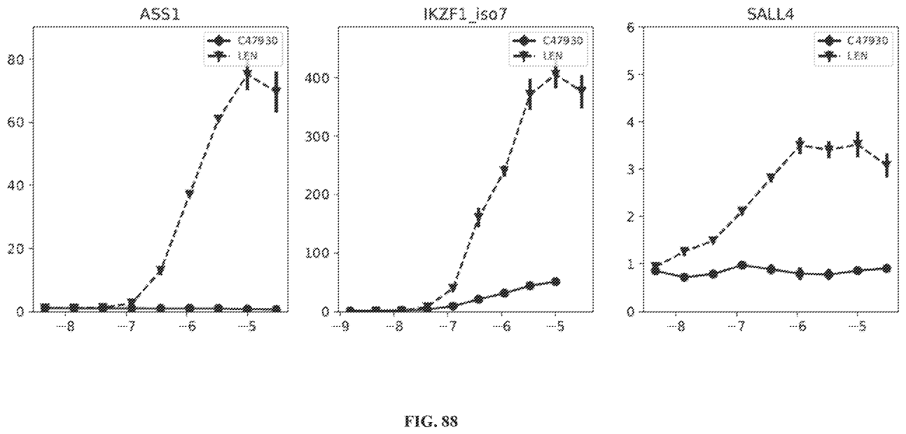

FIG. 89 illustrates the change of cereblon's ability to recruit ASS1, IKZF1, and SALL4 after binding with C47932 in comparison to LEN.

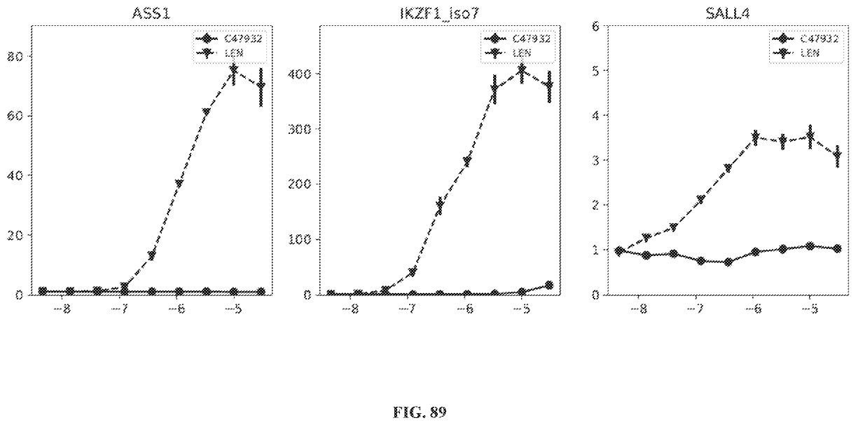

FIG. 90 illustrates the change of cereblon's ability to recruit ASS1, IKZF1, and SALL4 after binding with C47933 in comparison to LEN.

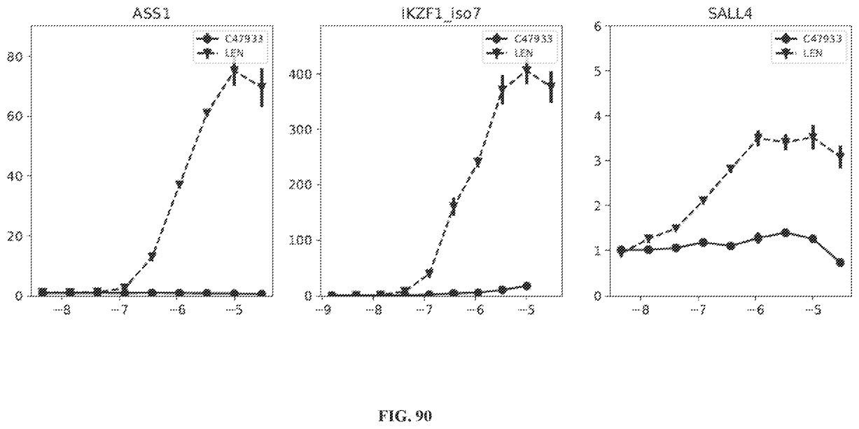

FIG. 91 illustrates the change of cereblon's ability to recruit ASS1, IKZF1, and SALL4 after binding with C47959 in comparison to LEN.

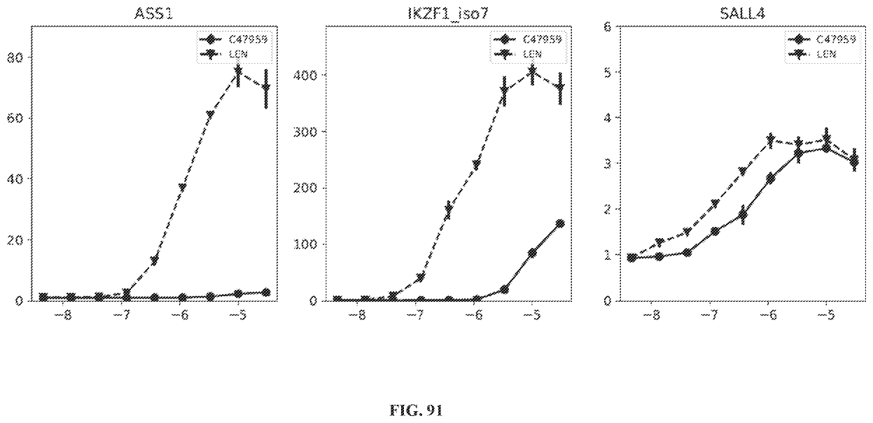

FIG. 92 illustrates the change of cereblon's ability to recruit ASS1, IKZF1, and SALL4 after binding with C47995 in comparison to LEN.

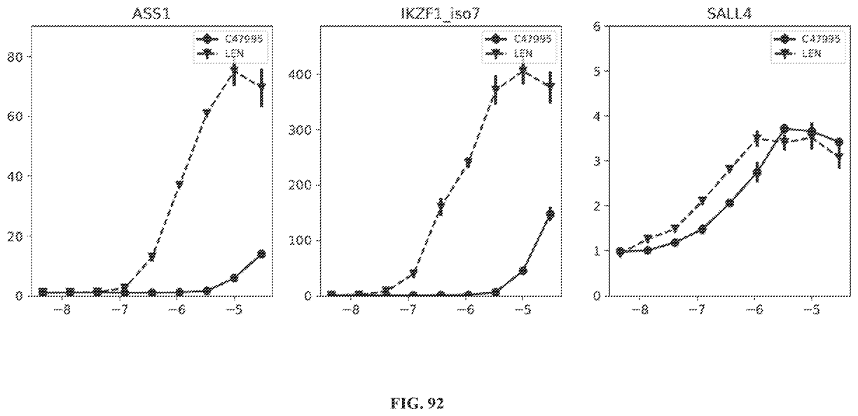

FIG. 93 illustrates the change of cereblon's ability to recruit ASS1, IKZF1, and SALL4 after binding with C47998 in comparison to LEN.

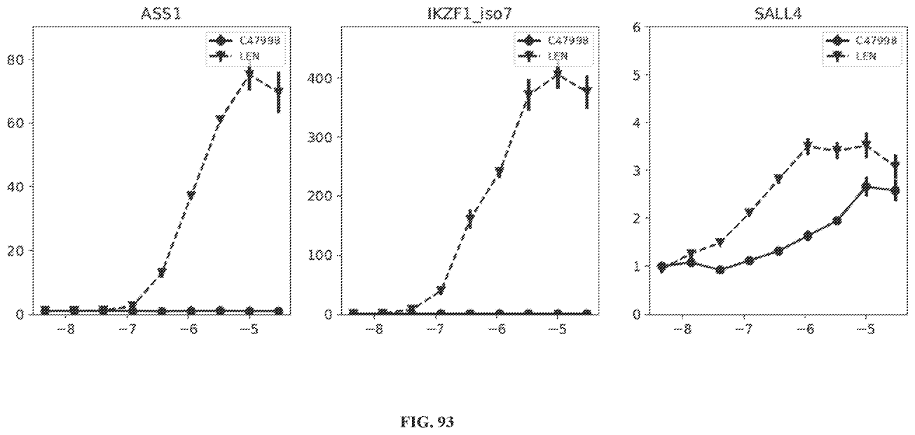

FIG. 94 illustrates the change of cereblon's ability to recruit ASS1, IKZF1, and SALL4 after binding with C48003 in comparison to LEN.

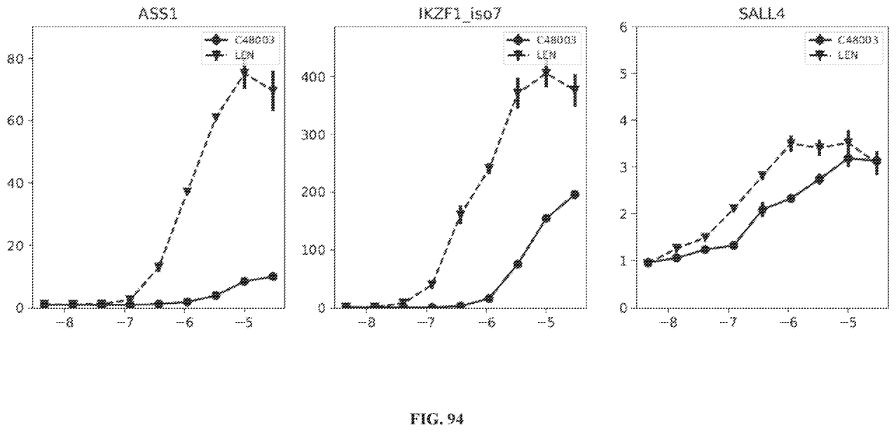

FIG. 95 illustrates the change of cereblon's ability to recruit ASS1, IKZF1, and SALL4 after binding with C48005 in comparison to LEN.

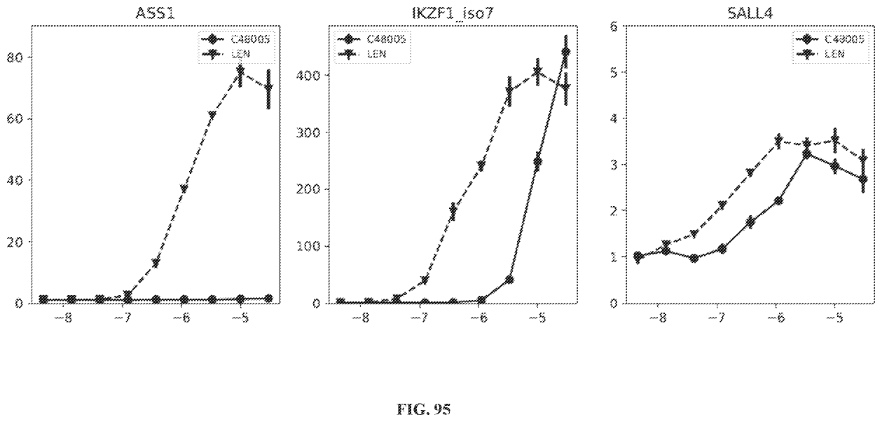

FIG. 96 illustrates the change of cereblon's ability to recruit ASS1, IKZF1, and SALL4 after binding with C48007 in comparison to LEN.

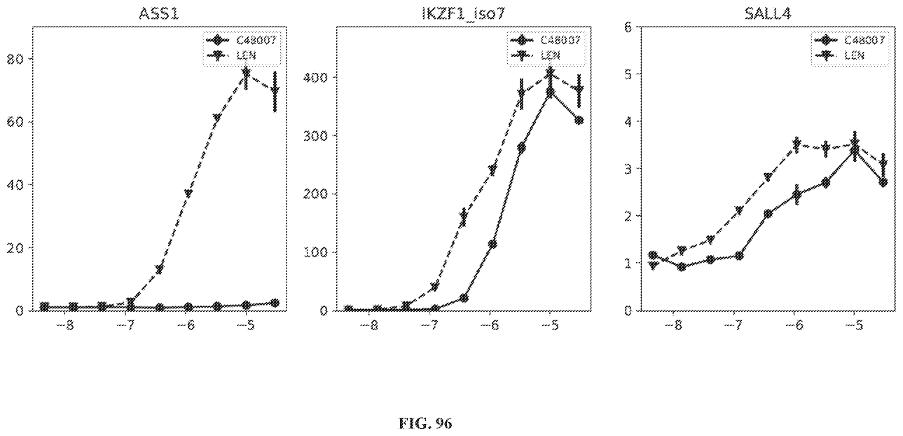

FIG. 97 illustrates the change of cereblon's ability to recruit ASS1, IKZF1, and SALL4 after binding with C48014 in comparison to LEN.

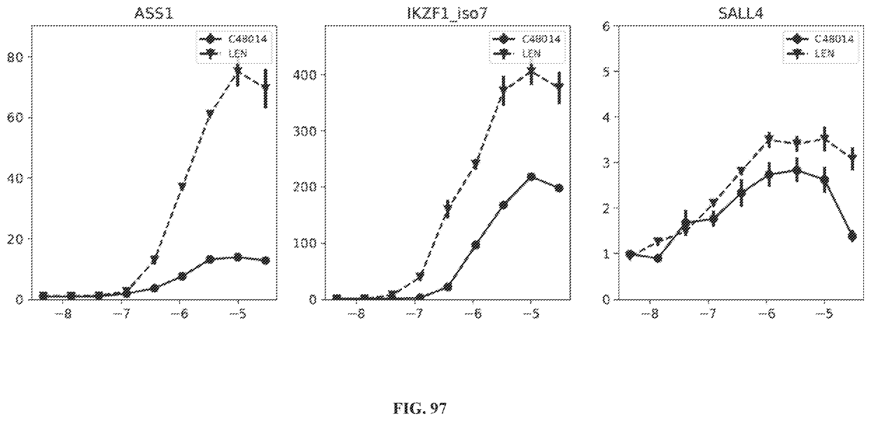

FIG. 98 illustrates the change of cereblon's ability to recruit ASS1, IKZF1, and SALL4 after binding with C48016 in comparison to LEN.

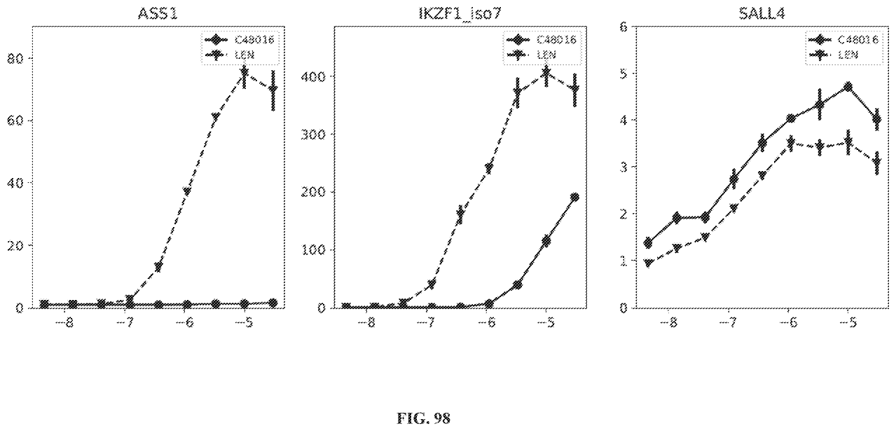

FIG. 99 illustrates the change of cereblon's ability to recruit ASS1, IKZF1, and SALL4 after binding with C48018 in comparison to LEN.

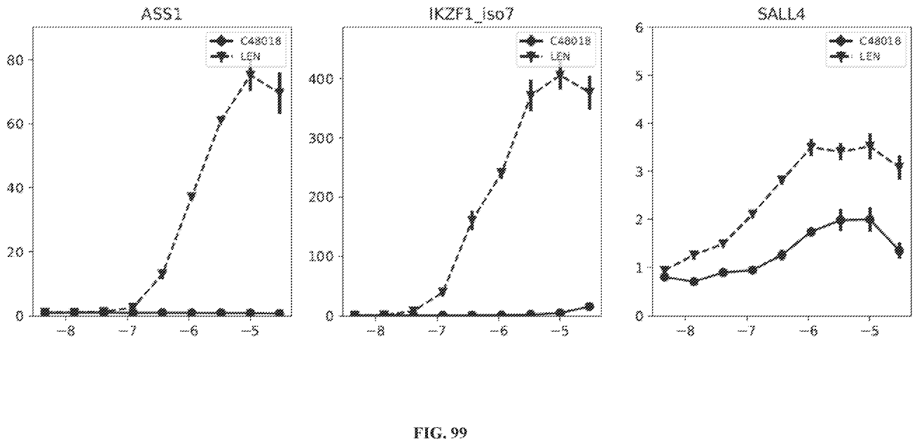

FIG. 100 illustrates the change of cereblon's ability to recruit ASS1, IKZF1, and SALL4 after binding with C22548 in comparison to LEN.

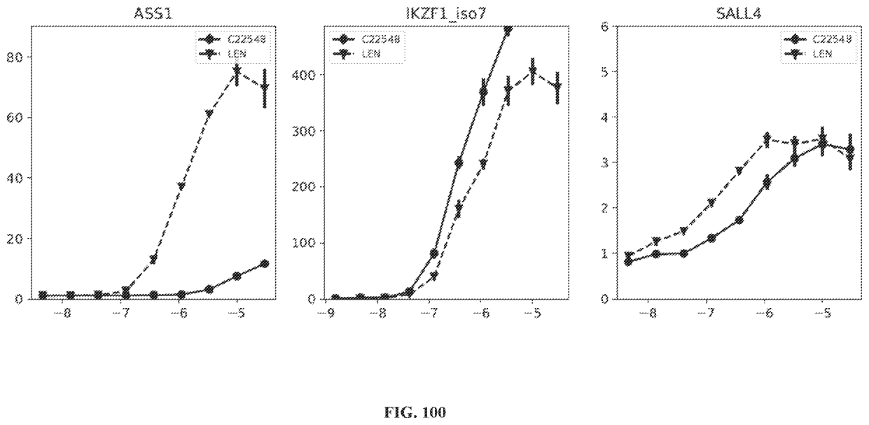

FIG. 101 illustrates the change of cereblon's ability to recruit ASS1, IKZF1, and SALL4 after binding with C22564 in comparison to LEN.

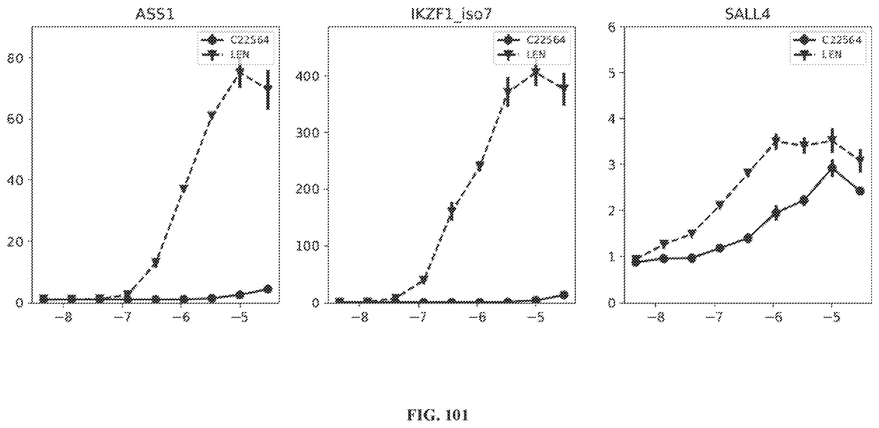

FIG. 102 illustrates the change of cereblon's ability to recruit ASS1, IKZF1, and SALL4 after binding with C22586 in comparison to LEN.

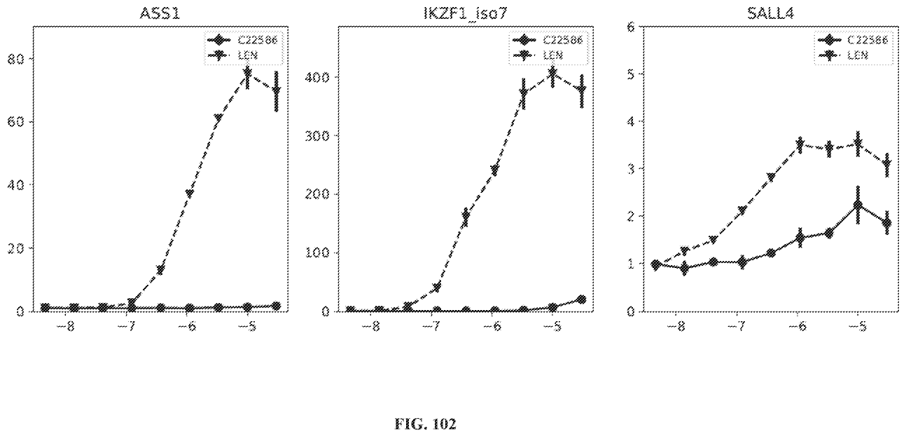

FIG. 103 illustrates the change of cereblon's ability to recruit ASS1, IKZF1, and SALL4 after binding with C22594 in comparison to LEN.

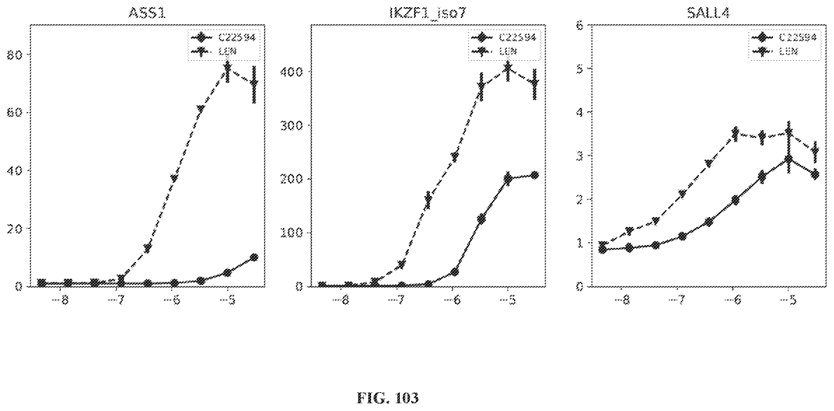

FIG. 104 illustrates the change of cereblon's ability to recruit ASS1, IKZF1, and SALL4 after binding with C22622 in comparison to LEN.

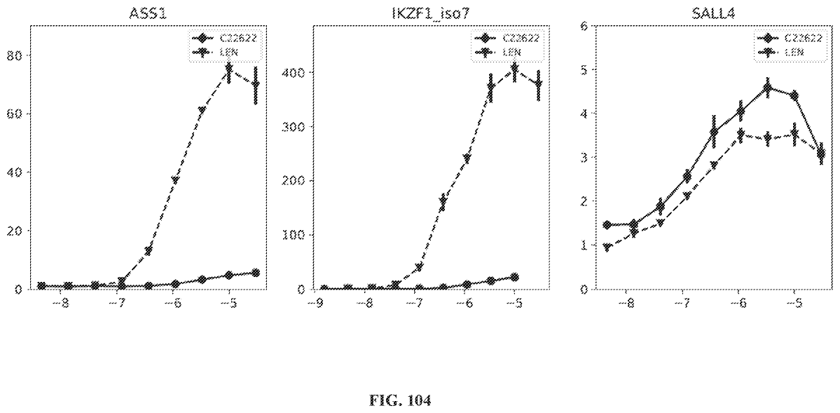

FIG. 105 illustrates the change of cereblon's ability to recruit ASS1, IKZF1, and SALL4 after binding with C30231 in comparison to LEN.

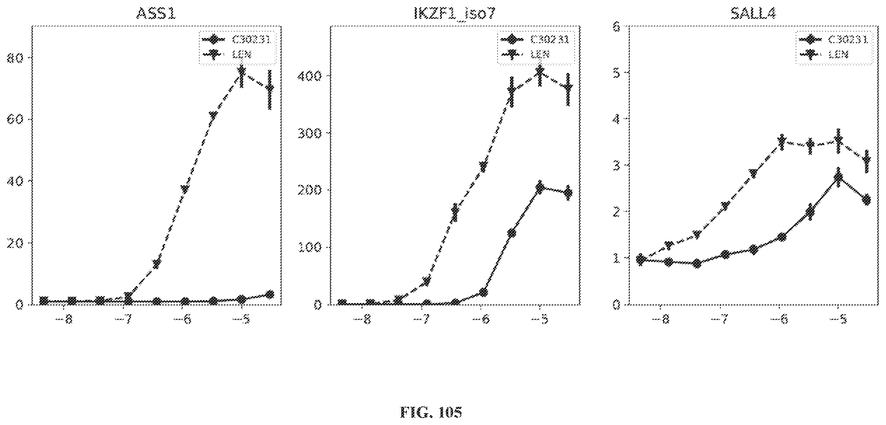

FIG. 106 illustrates the change of cereblon's ability to recruit ASS1, IKZF1, and SALL4 after binding with C29737 in comparison to LEN.

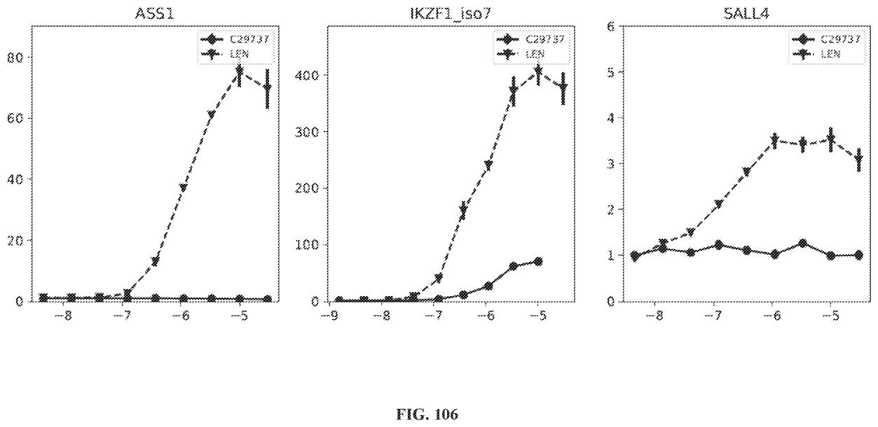

FIG. 107 illustrates the change of cereblon's ability to recruit ASS1, IKZF1, and SALL4 after binding with C67858 in comparison to LEN.

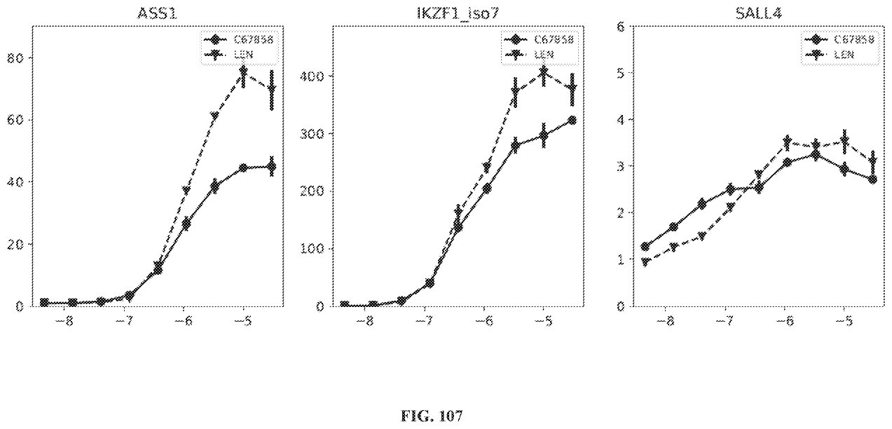

FIG. 108 illustrates the change of cereblon's ability to recruit ASS1, IKZF1, and SALL4 after binding with C68126 in comparison to LEN.

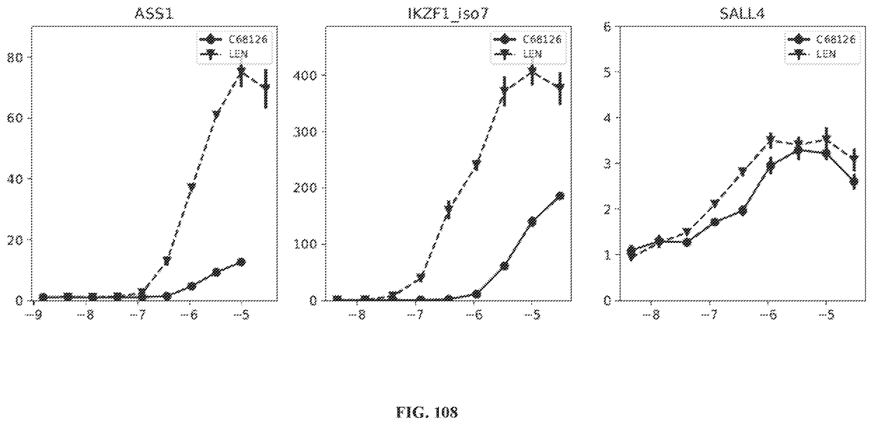

FIG. 109 illustrates the change of cereblon's ability to recruit ASS1, IKZF1, and SALL4 after binding with C55859 in comparison to LEN.

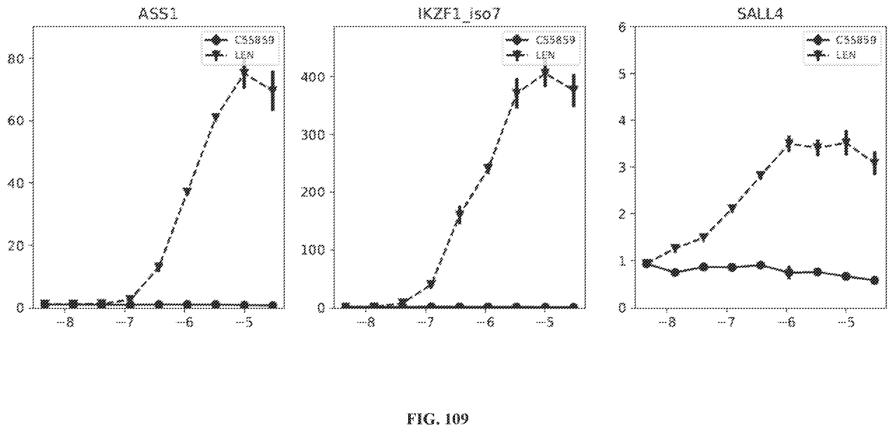

FIG. 110 illustrates the change of cereblon's ability to recruit ASS1, IKZF1, and SALL4 after binding with C23258 in comparison to LEN.

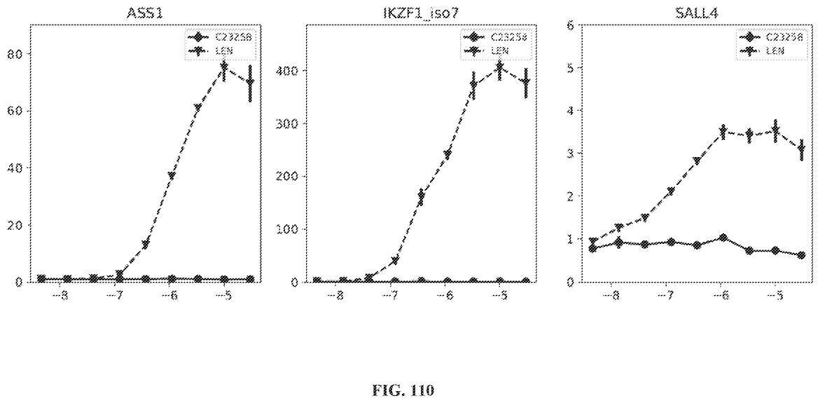

FIG. 111 illustrates the change of cereblon's ability to recruit ASS1, IKZF1, and SALL4 after binding with C47935 in comparison to LEN.

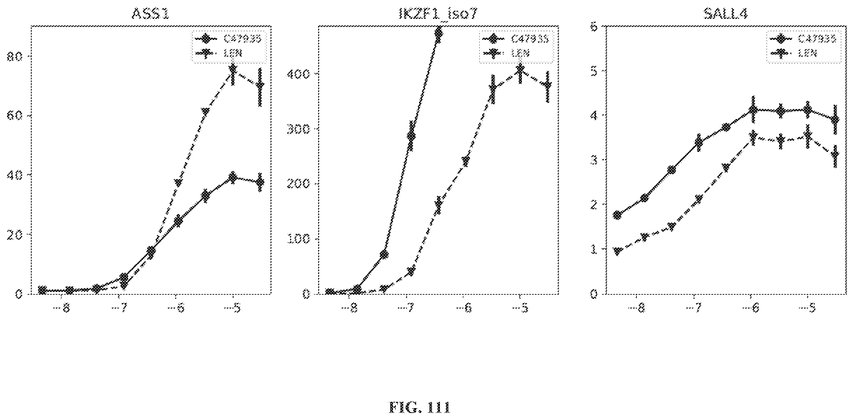

FIG. 112 illustrates the change of cereblon's ability to recruit ASS1, IKZF1, and SALL4 after binding with C05955 in comparison to LEN.

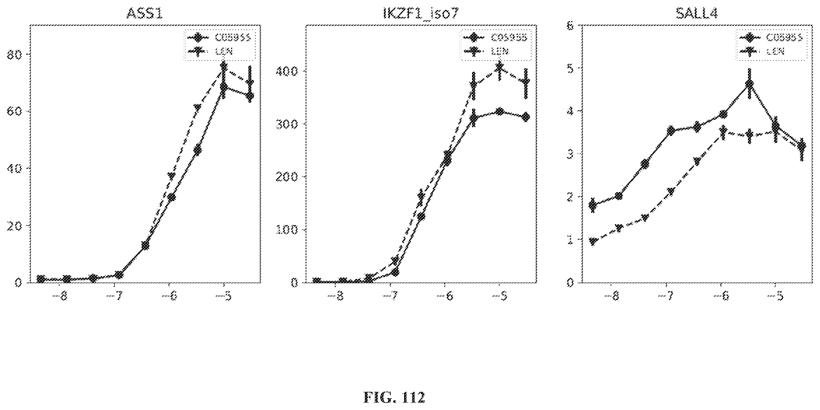

FIG. 113 illustrates the change of cereblon's ability to recruit ASS1, IKZF1, and SALL4 after binding with C49278 in comparison to LEN.

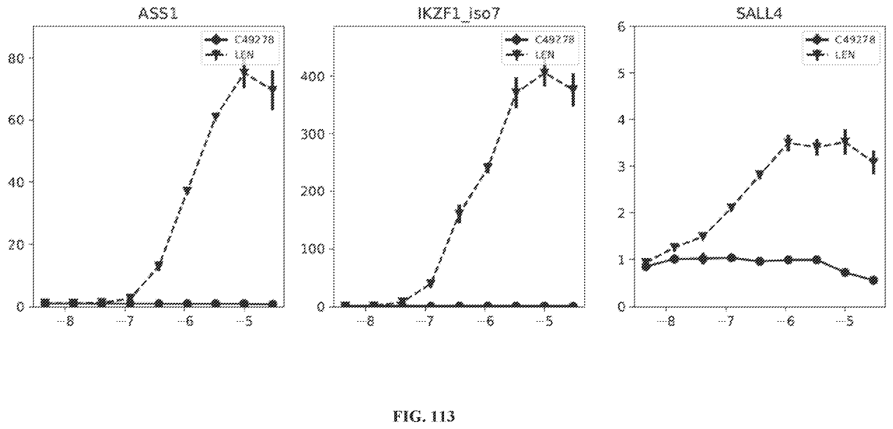

FIG. 114 illustrates the change of cereblon's ability to recruit ASS1, IKZF1, and SALL4 after binding with C39453 in comparison to LEN.

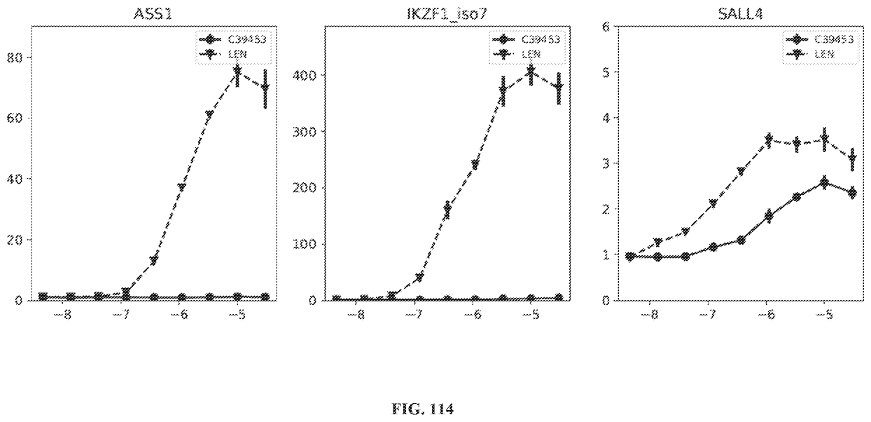

FIG. 115 illustrates the change of cereblon's ability to recruit ASS1, IKZF1, and SALL4 after binding with C51383 in comparison to LEN.

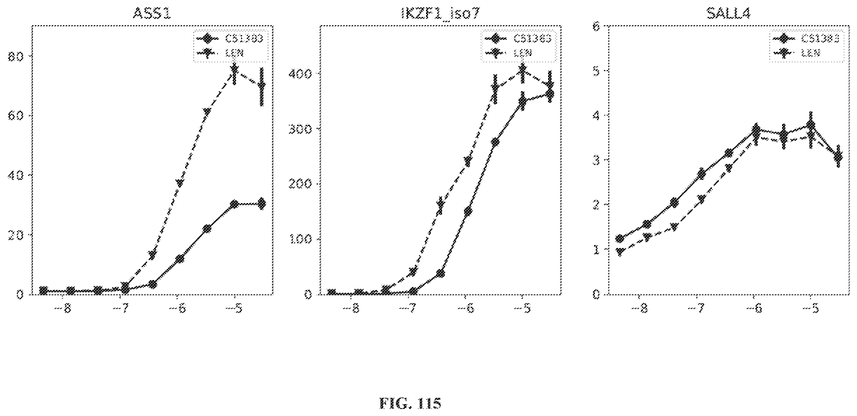

FIG. 116 illustrates the change of cereblon's ability to recruit ASS1, IKZF1, and SALL4 after binding with C99884 in comparison to LEN.

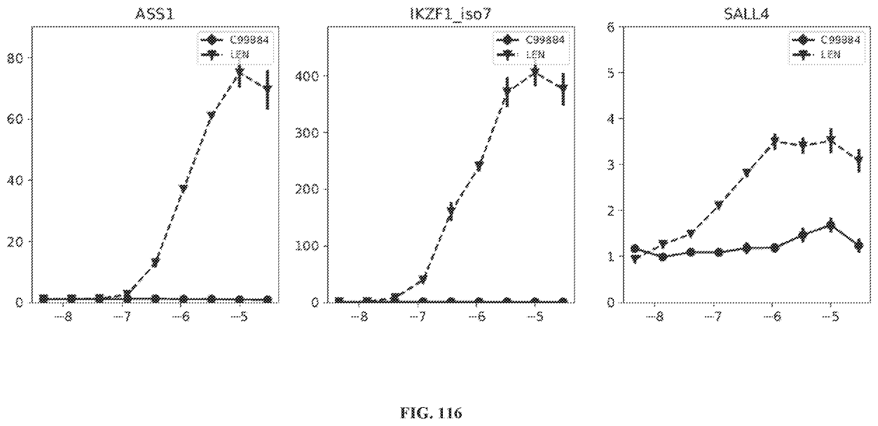

FIG. 117 illustrates the change of cereblon's ability to recruit ASS1, IKZF1, and SALL4 after binding with C34491 in comparison to LEN.

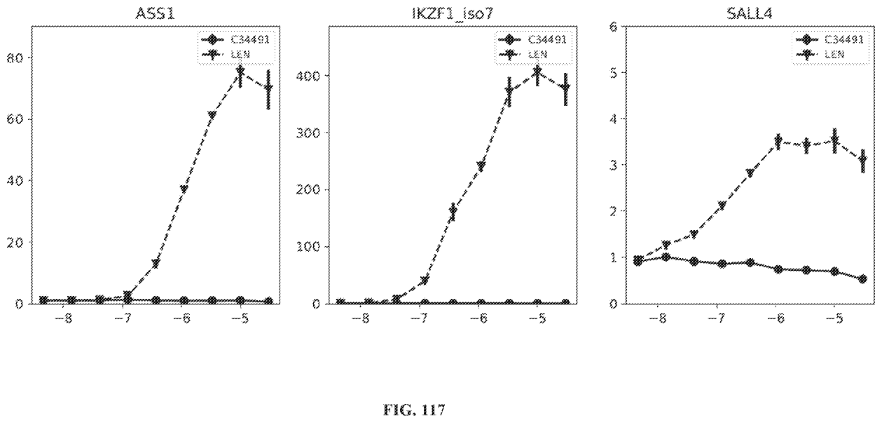

FIG. 118 illustrates the change of cereblon's ability to recruit ASS1, IKZF1, and SALL4 after binding with C98103 in comparison to LEN.

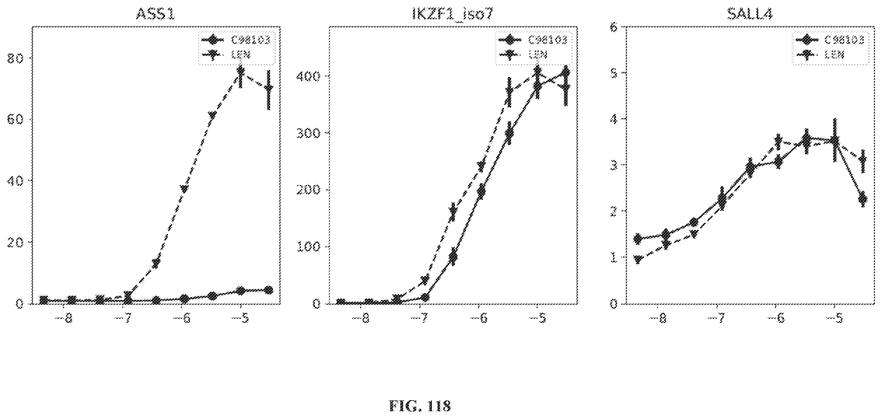

FIG. 119 illustrates the change of cereblon's ability to recruit ASS1, IKZF1, and SALL4 after binding with C96413 in comparison to LEN.

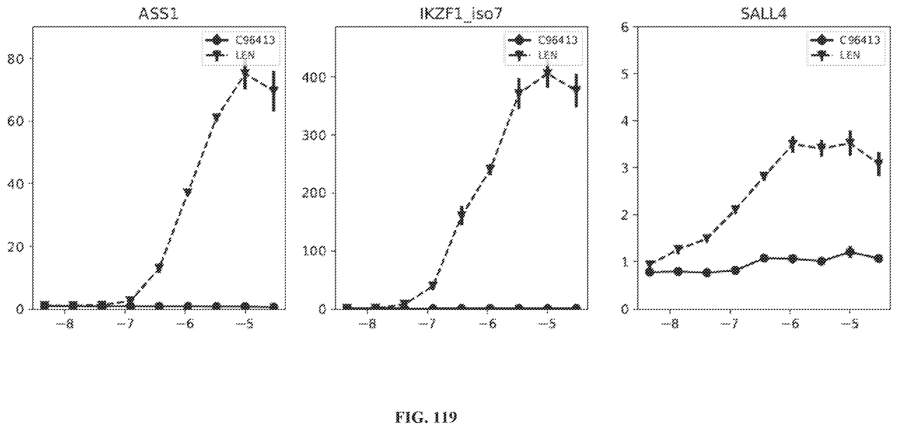

FIG. 120 illustrates the change of cereblon's ability to recruit ASS1, IKZF1, and SALL4 after binding with C35745 in comparison to LEN.

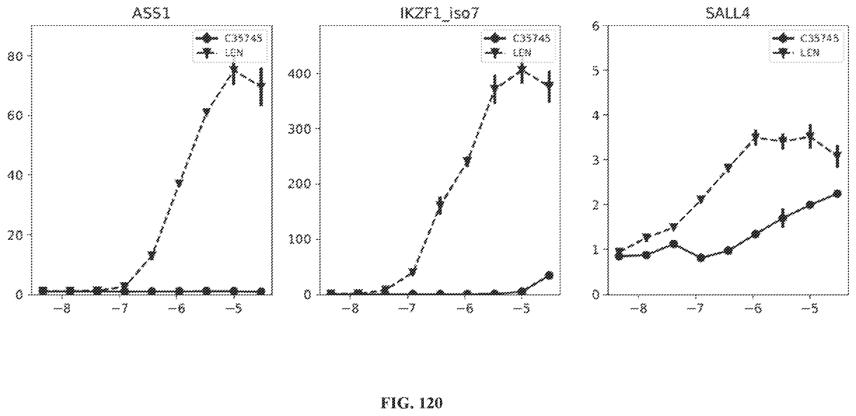

FIG. 121 illustrates the change of cereblon's ability to recruit ASS1, IKZF1, and SALL4 after binding with C35751 in comparison to LEN.

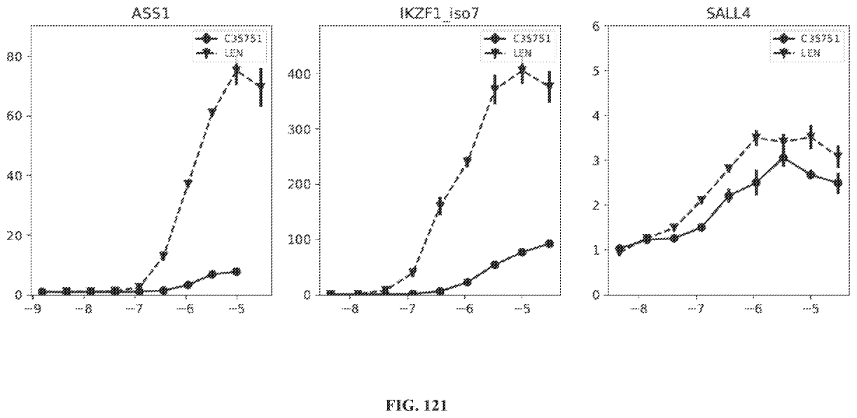

FIG. 122 illustrates the change of cereblon's ability to recruit ASS1, IKZF1, and SALL4 after binding with C35754 in comparison to LEN.

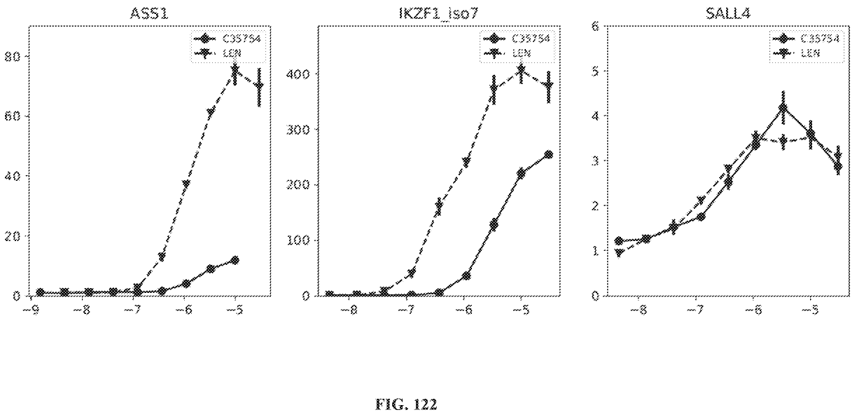

FIG. 123 illustrates the change of cereblon's ability to recruit ASS1, IKZF1, and SALL4 after binding with C35797 in comparison to LEN.

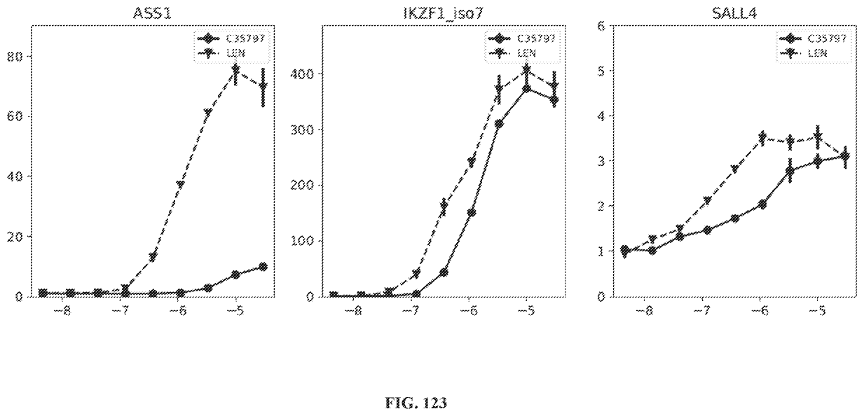

FIG. 124 illustrates the change of cereblon's ability to recruit ASS1, IKZF1, and SALL4 after binding with C35811 in comparison to LEN.

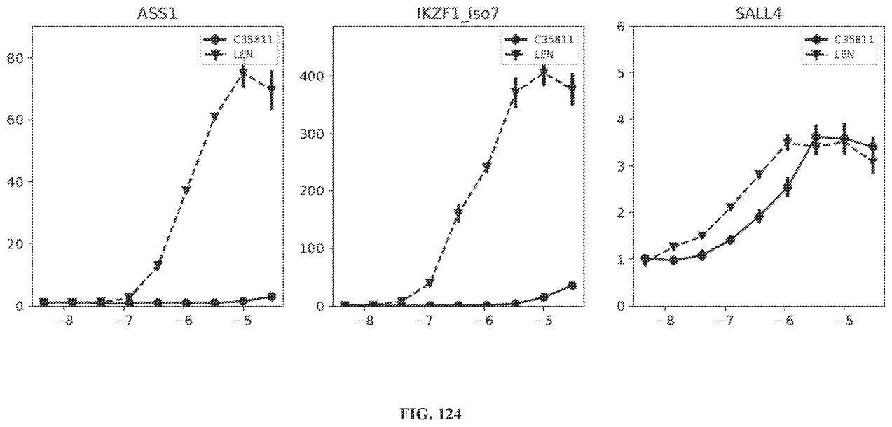

FIG. 125 illustrates the change of cereblon's ability to recruit ASS1, IKZF1, and SALL4 after binding with C35856 in comparison to LEN.

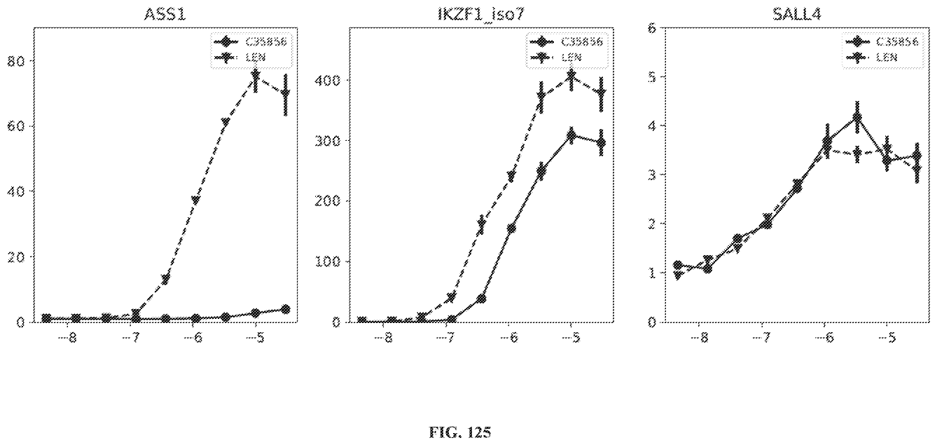

FIG. 126 illustrates the change of cereblon's ability to recruit ASS1, IKZF1, and SALL4 after binding with C23066 in comparison to LEN.

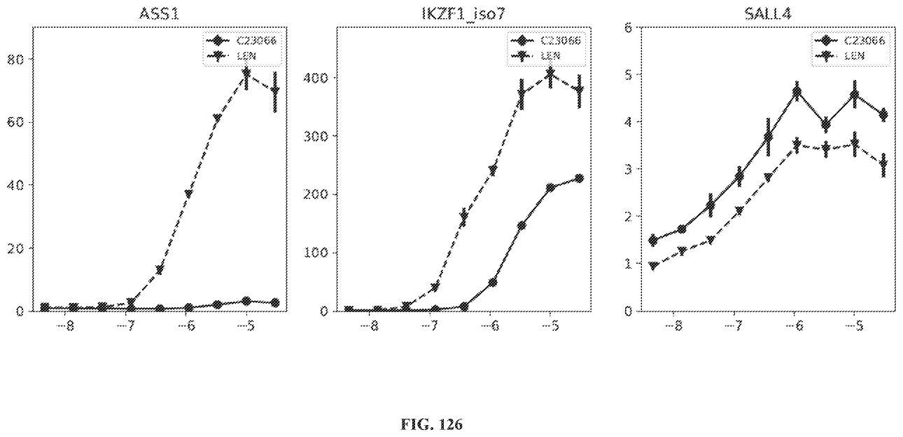

FIG. 127 illustrates the change of cereblon's ability to recruit ASS1, IKZF1, and SALL4 after binding with C39772 in comparison to LEN.

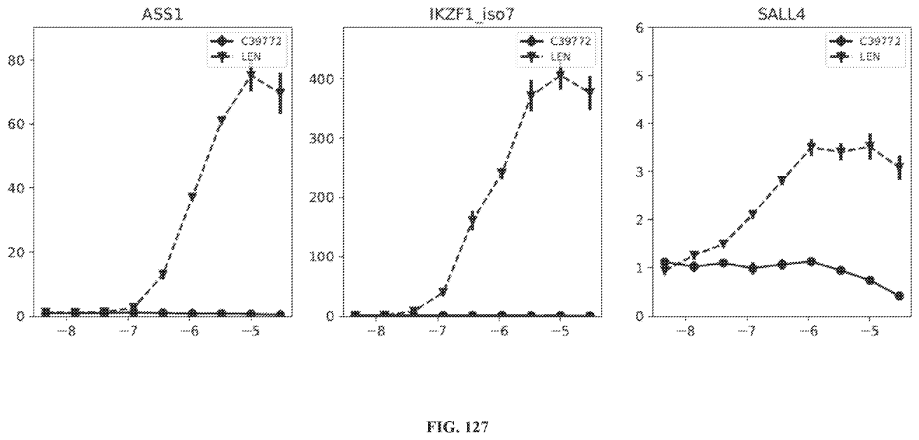

FIG. 128 illustrates the change of cereblon's ability to recruit ASS1, IKZF1, and SALL4 after binding with C51647 in comparison to LEN.

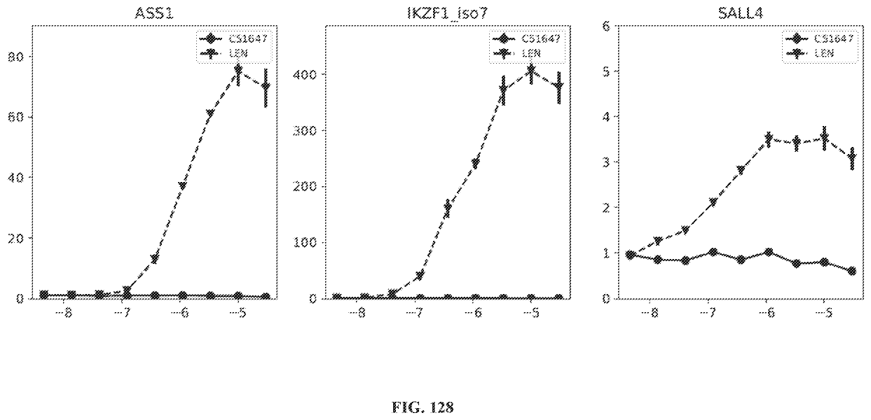

FIG. 129 illustrates the change of cereblon's ability to recruit ASS1, IKZF1, and SALL4 after binding with C40531 in comparison to LEN.

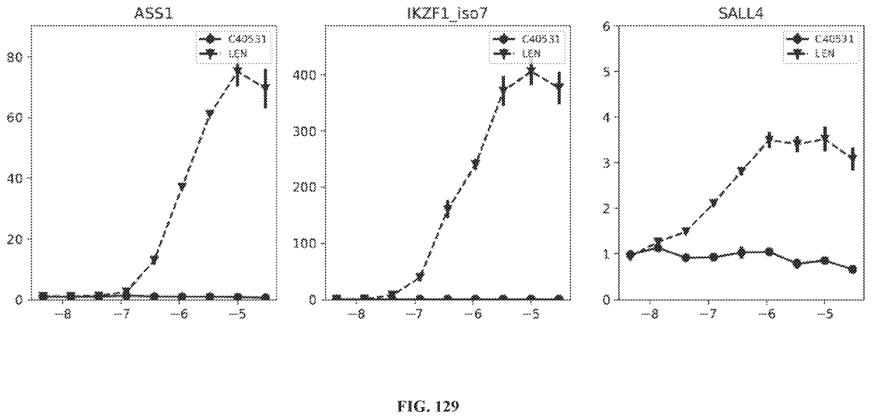

FIG. 130 illustrates the change of cereblon's ability to recruit ASS1, IKZF1, and SALL4 after binding with C84963 in comparison to LEN.

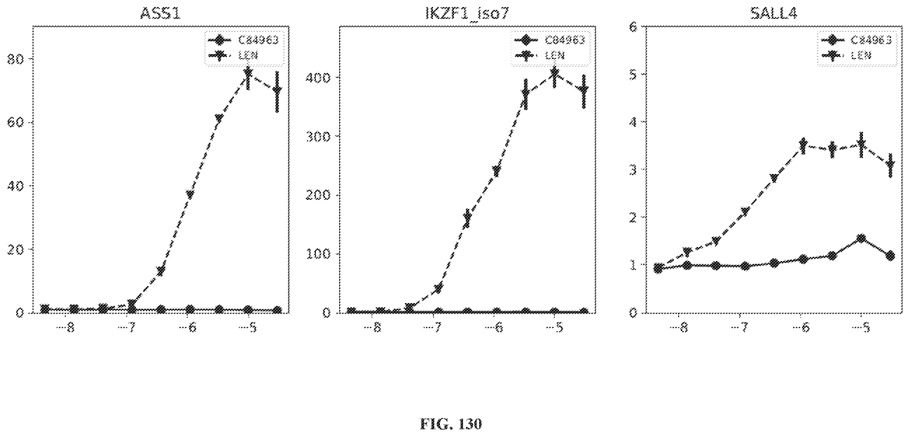

FIG. 131 illustrates the change of cereblon's ability to recruit ASS1, IKZF1, and SALL4 after binding with C84965 in comparison to LEN.

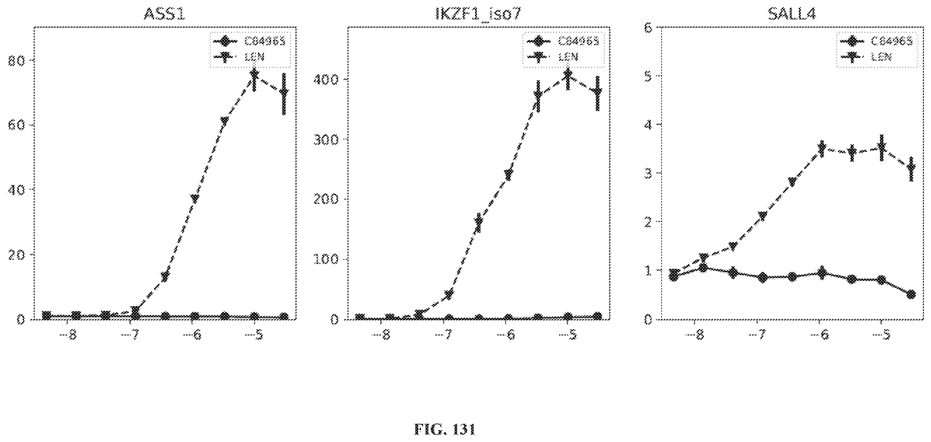

FIG. 132 illustrates the change of cereblon's ability to recruit ASS1, IKZF1, and SALL4 after binding with C84967 in comparison to LEN.

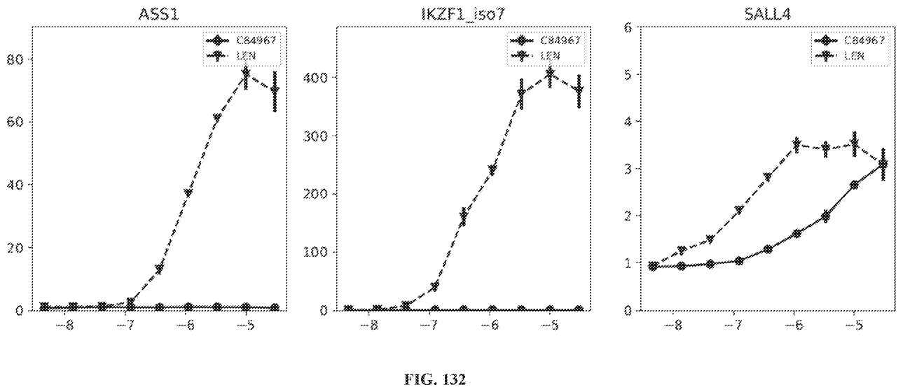

FIG. 133 illustrates the change of cereblon's ability to recruit ASS1, IKZF1, and SALL4 after binding with C84970 in comparison to LEN.

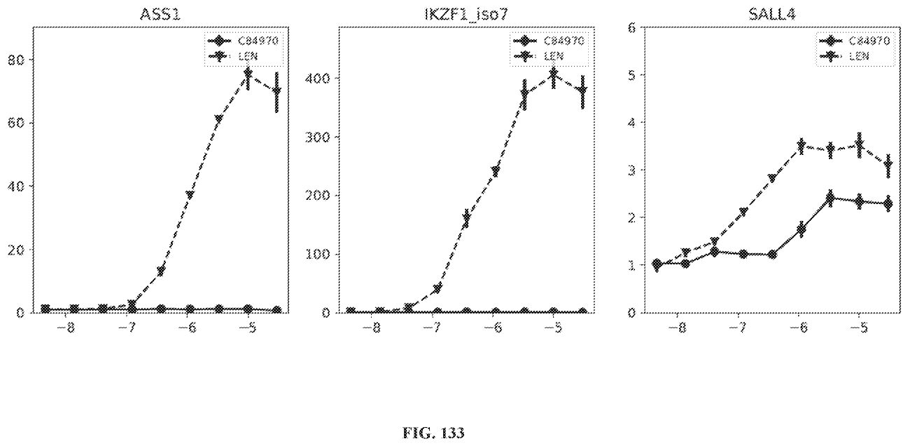

FIG. 134 illustrates the change of cereblon's ability to recruit ASS1, IKZF1, and SALL4 after binding with C64319 in comparison to LEN.

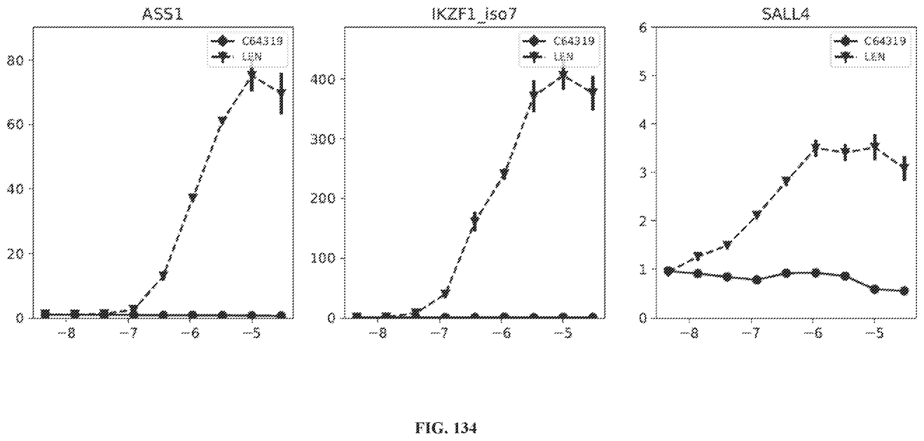

FIG. 135 illustrates the change of cereblon's ability to recruit ASS1, IKZF1, and SALL4 after binding with C64329 in comparison to LEN.

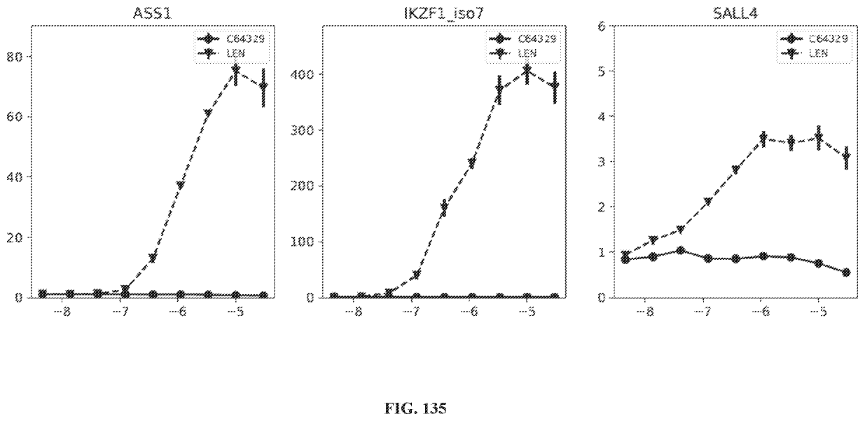

FIG. 136 illustrates the change of cereblon's ability to recruit ASS1, IKZF1, and SALL4 after binding with C64340 in comparison to LEN.

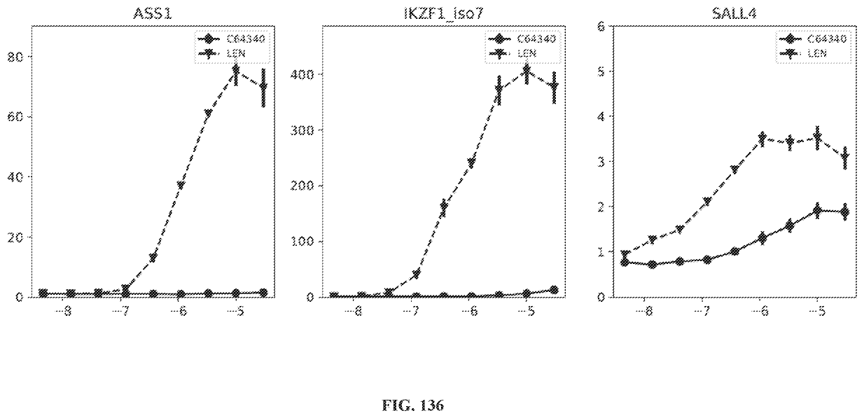

FIG. 137 illustrates the change of cereblon's ability to recruit ASS1, IKZF1, and SALL4 after binding with C64344 in comparison to LEN.

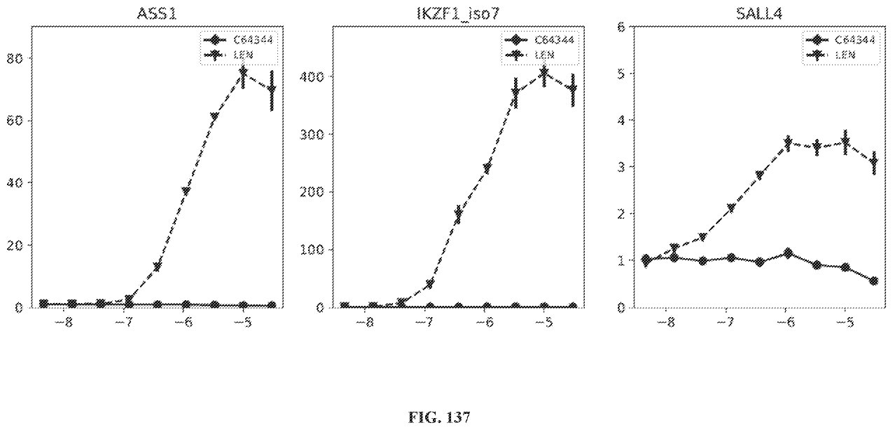

FIG. 138 illustrates the change of cereblon's ability to recruit ASS1, IKZF1, and SALL4 after binding with C64348 in comparison to LEN.

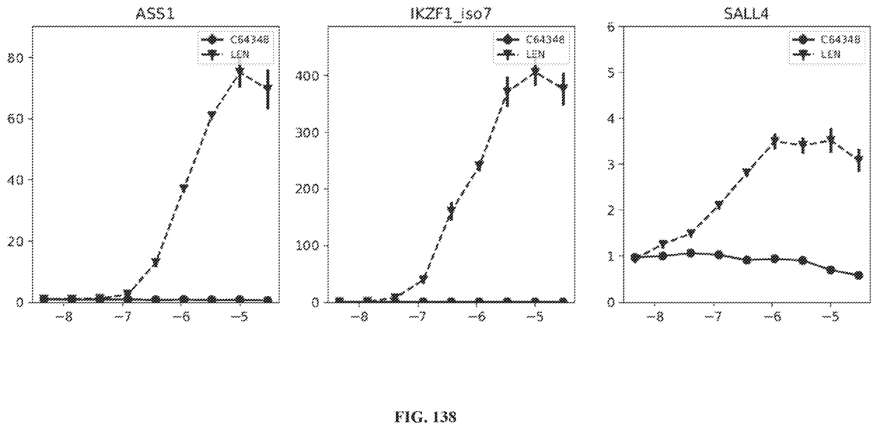

FIG. 139 illustrates the change of cereblon's ability to recruit ASS1, IKZF1, and SALL4 after binding with C64372 in comparison to LEN.

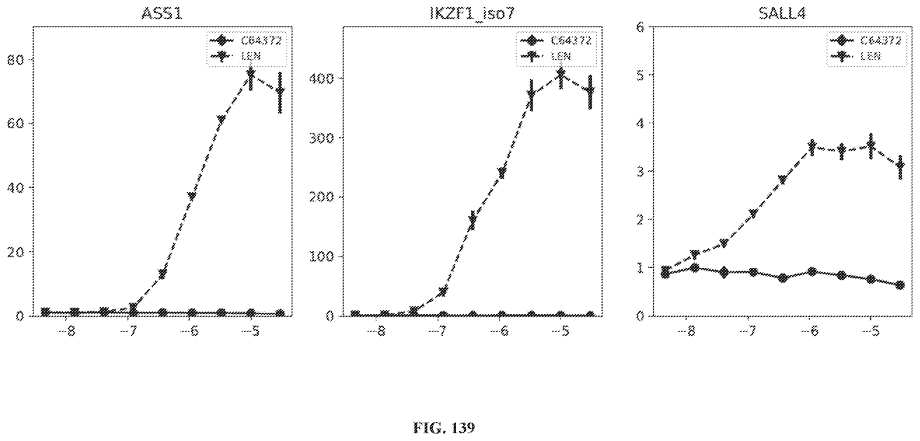

FIG. 140 illustrates the change of cereblon's ability to recruit ASS1, IKZF1, and SALL4 after binding with C89940 in comparison to LEN.

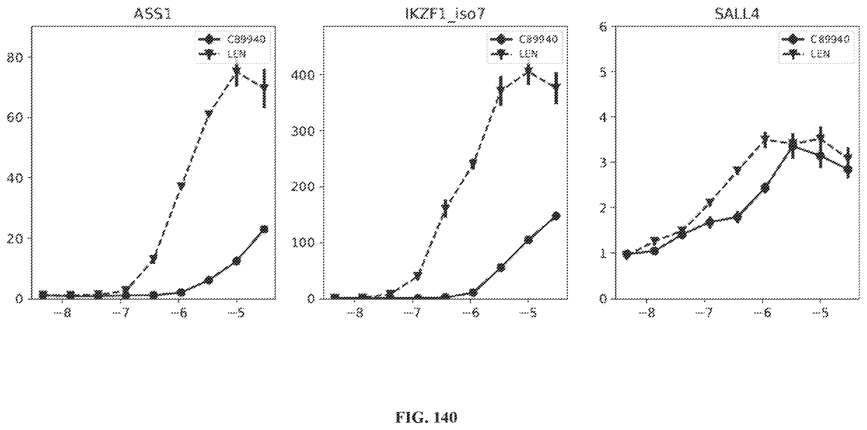

FIG. 141 illustrates the change of cereblon's ability to recruit ASS1, IKZF1, and SALL4 after binding with C12697 in comparison to LEN.

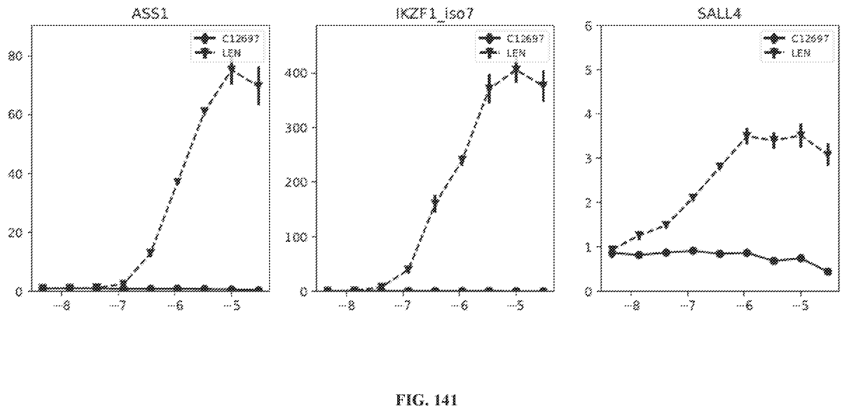

FIG. 142 illustrates the change of cereblon's ability to recruit ASS1, IKZF1, and SALL4 after binding with C16899 in comparison to LEN.

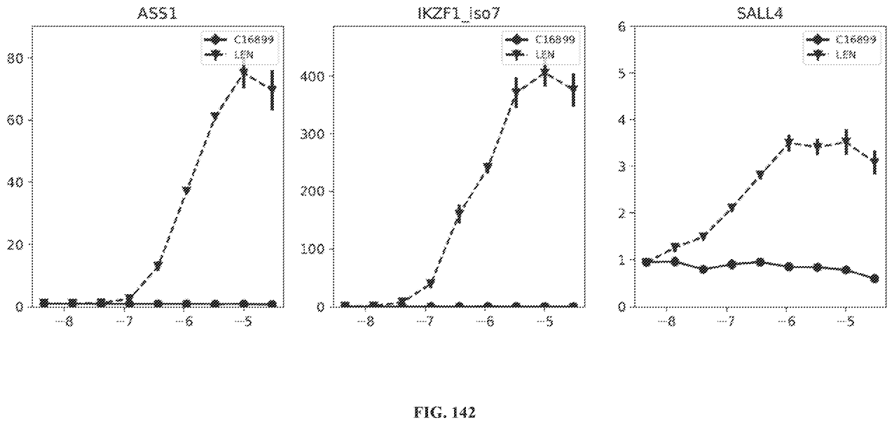

FIG. 143 illustrates the change of cereblon's ability to recruit ASS1, IKZF1, and SALL4 after binding with C80369 in comparison to LEN.

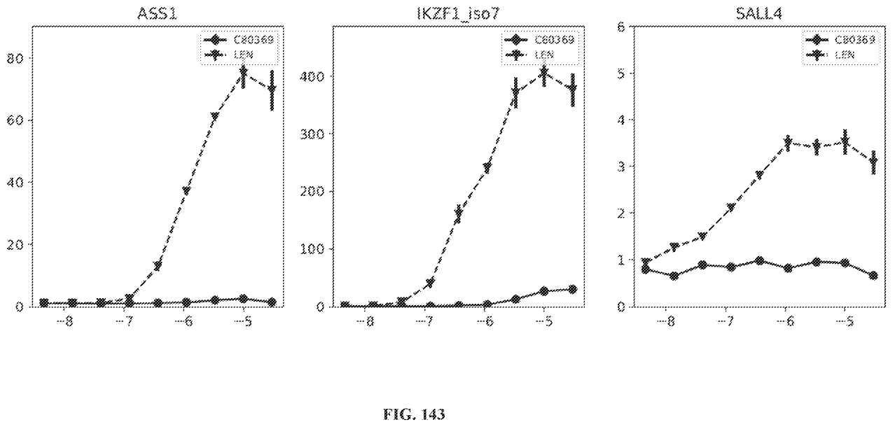

FIG. 144 illustrates the change of cereblon's ability to recruit ASS1, IKZF1, and SALL4 after binding with C80373 in comparison to LEN.

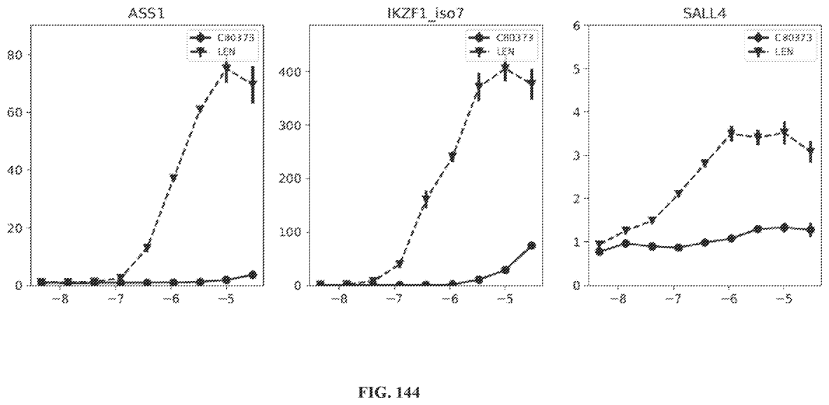

FIG. 145 illustrates the change of cereblon's ability to recruit ASS1, IKZF1, and SALL4 after binding with C80374 in comparison to LEN.

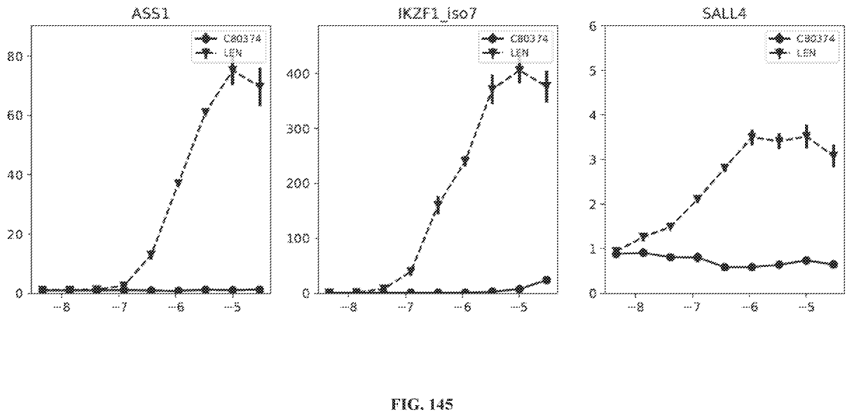

FIG. 146 illustrates the change of cereblon's ability to recruit ASS1, IKZF1, and SALL4 after binding with C80376 in comparison to LEN.

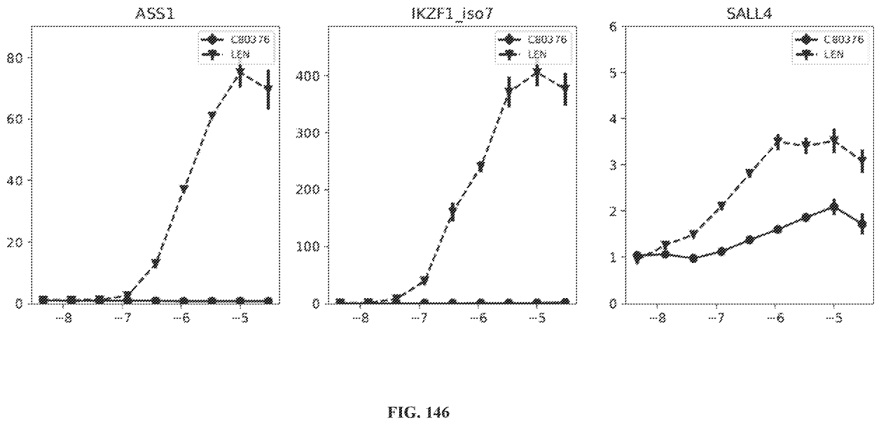

FIG. 147 illustrates the change of cereblon's ability to recruit ASS1, IKZF1, and SALL4 after binding with C80378 in comparison to LEN.

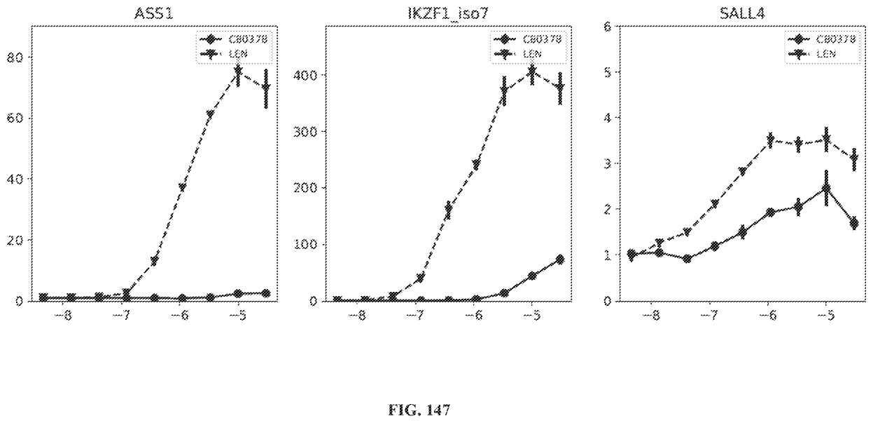

FIG. 148 illustrates the change of cereblon's ability to recruit ASS1, IKZF1, and SALL4 after binding with C80379 in comparison to LEN.

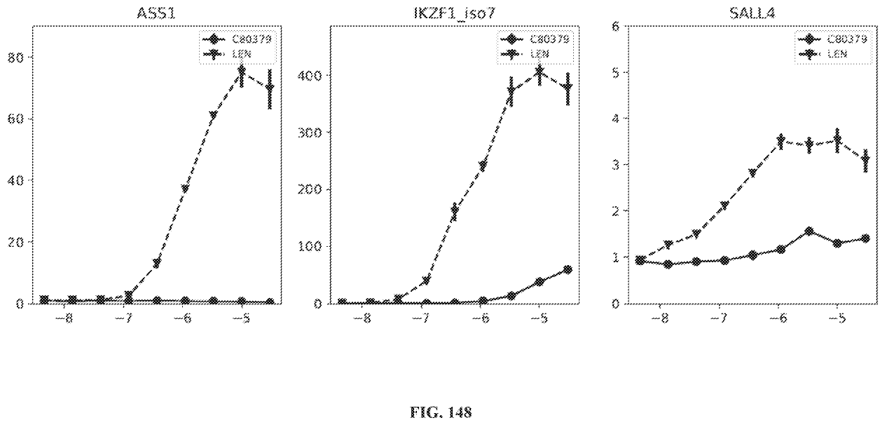

FIG. 149 illustrates the change of cereblon's ability to recruit ASS1, IKZF1, and SALL4 after binding with C80380 in comparison to LEN.

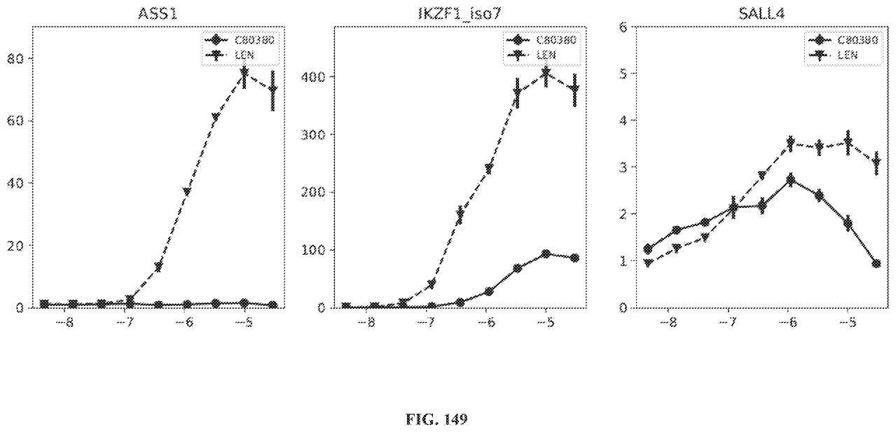

FIG. 150 illustrates the change of cereblon's ability to recruit ASS1, IKZF1, and SALL4 after binding with C80385 in comparison to LEN.

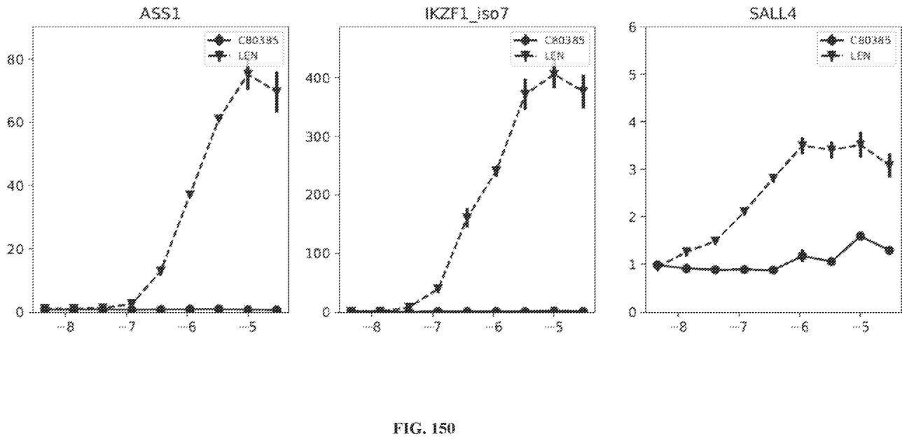

FIG. 151 illustrates the change of cereblon's ability to recruit ASS1, IKZF1, and SALL4 after binding with C80390 in comparison to LEN.

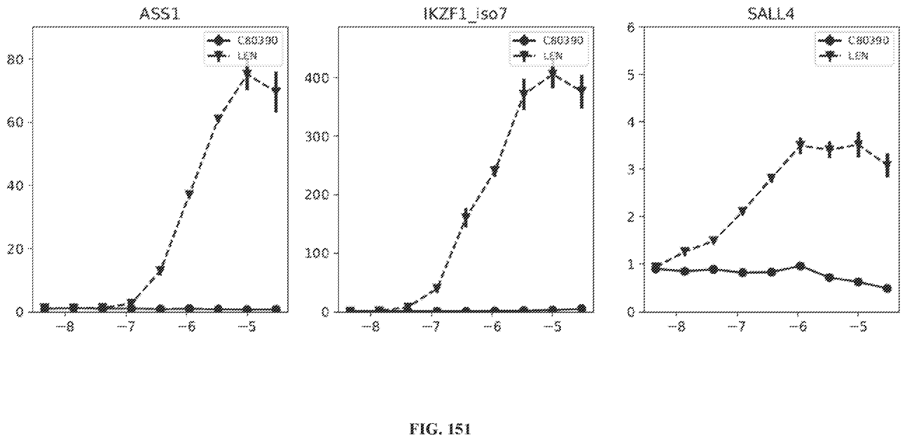

FIG. 152 illustrates the change of cereblon's ability to recruit ASS1, IKZF1, and SALL4 after binding with C80391 in comparison to LEN.

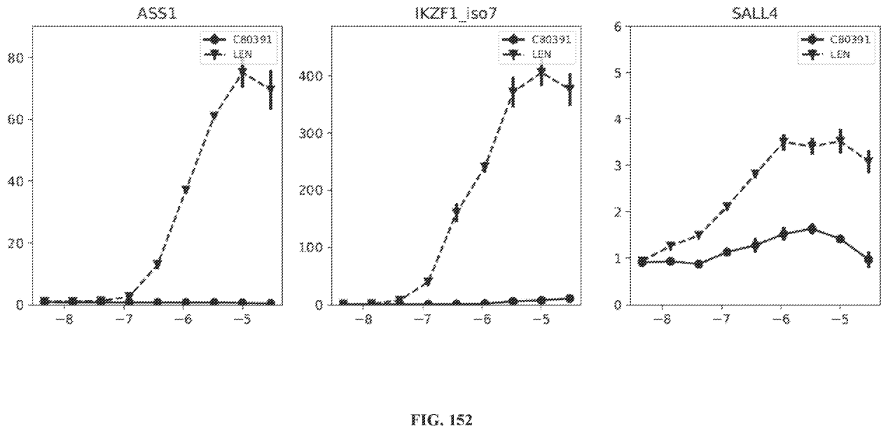

FIG. 153 illustrates the change of cereblon's ability to recruit ASS1, IKZF1, and SALL4 after binding with C80393 in comparison to LEN.

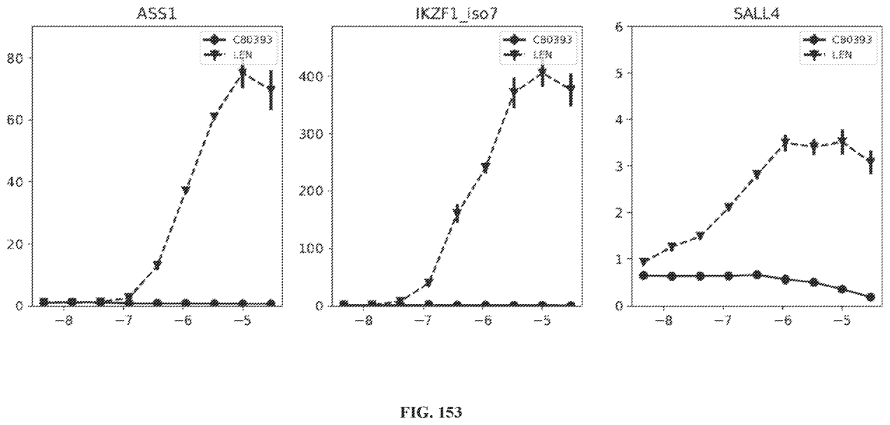

FIG. 154 illustrates the change of cereblon's ability to recruit ASS1, IKZF1, and SALL4 after binding with C80394 in comparison to LEN.

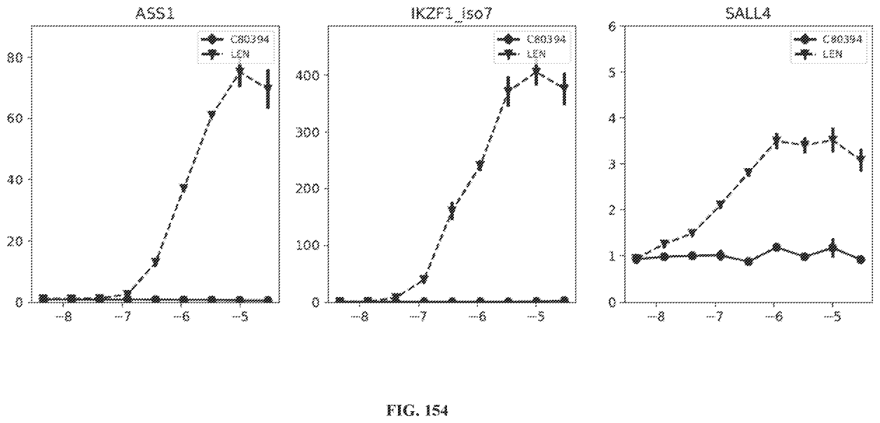

FIG. 155 illustrates the change of cereblon's ability to recruit ASS1, IKZF1, and SALL4 after binding with C80395 in comparison to LEN.

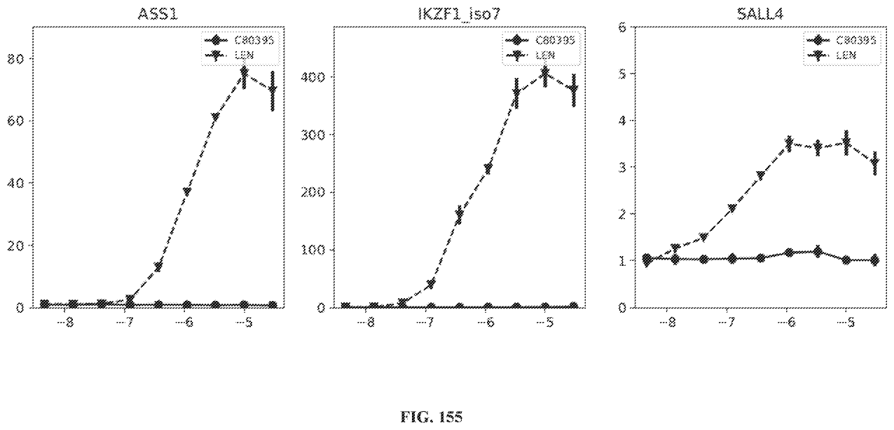

FIG. 156 illustrates the change of cereblon's ability to recruit ASS1, IKZF1, and SALL4 after binding with C80396 in comparison to LEN.

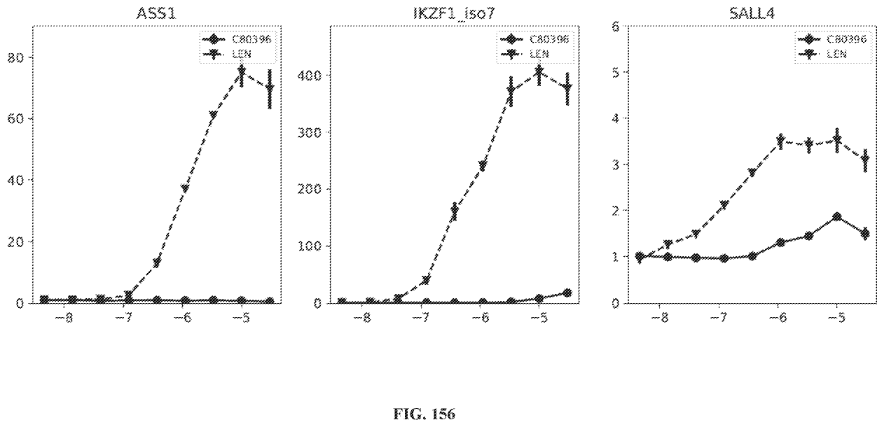

FIG. 157 illustrates the change of cereblon's ability to recruit ASS1, IKZF1, and SALL4 after binding with C33779 in comparison to LEN.

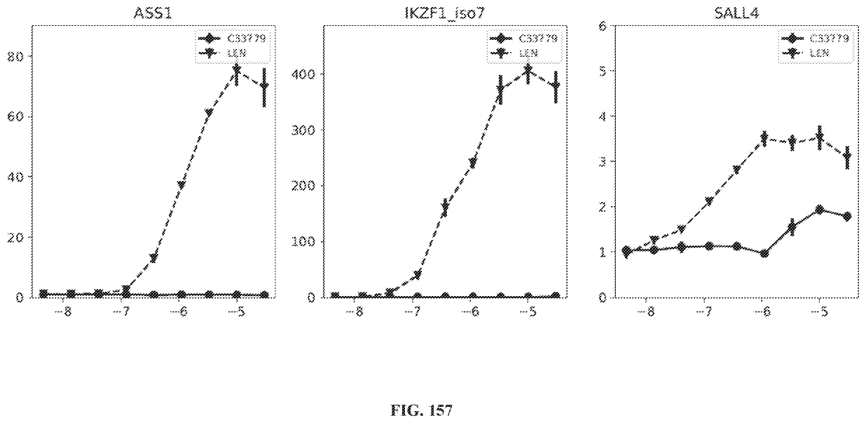

FIG. 158 illustrates the change of cereblon's ability to recruit ASS1, IKZF1, and SALL4 after binding with C46003 in comparison to LEN.

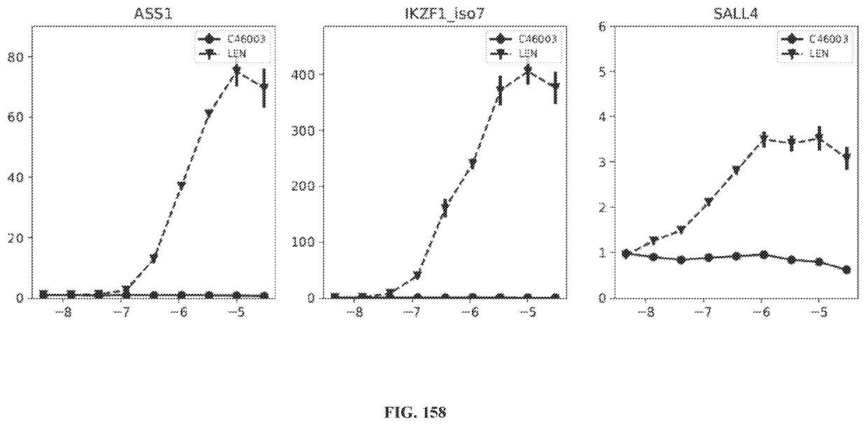

FIG. 159 illustrates the change of cereblon's ability to recruit ASS1, IKZF1, and SALL4 after binding with CC08493 in comparison to LEN.

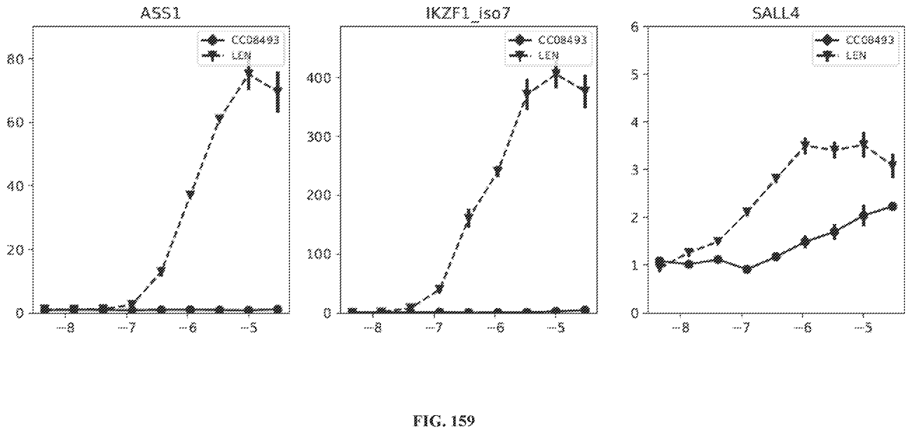

FIG. 160 illustrates the change of cereblon's ability to recruit ASS1, IKZF1, and SALL4 after binding with C09063 in comparison to LEN.

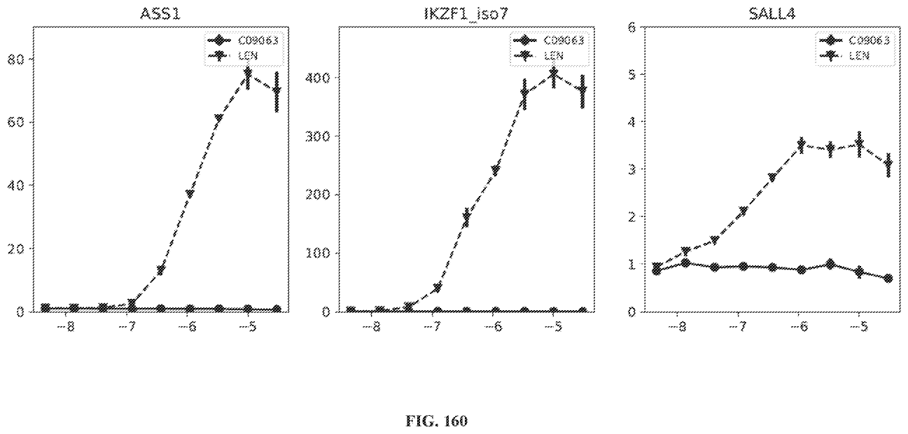

FIG. 161 illustrates the change of cereblon's ability to recruit ASS1, IKZF1, and SALL4 after binding with C09563 in comparison to LEN.

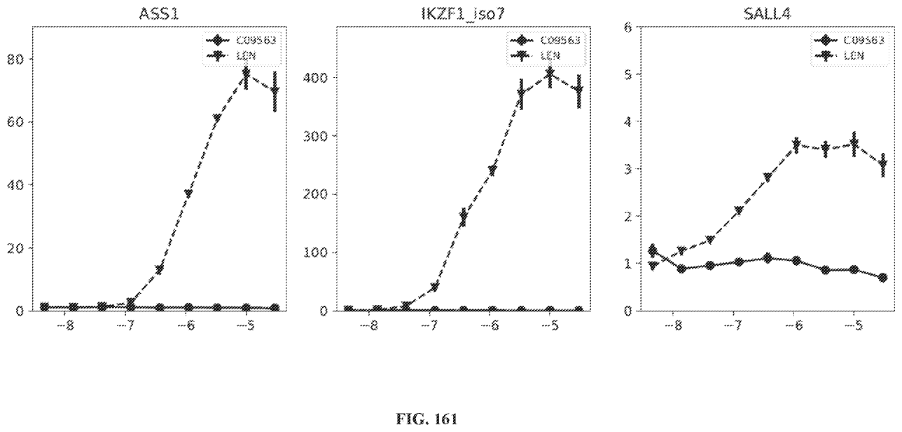

FIG. 162 illustrates the change of cereblon's ability to recruit ASS1, IKZF1, and SALL4 after binding with C10239 in comparison to LEN.

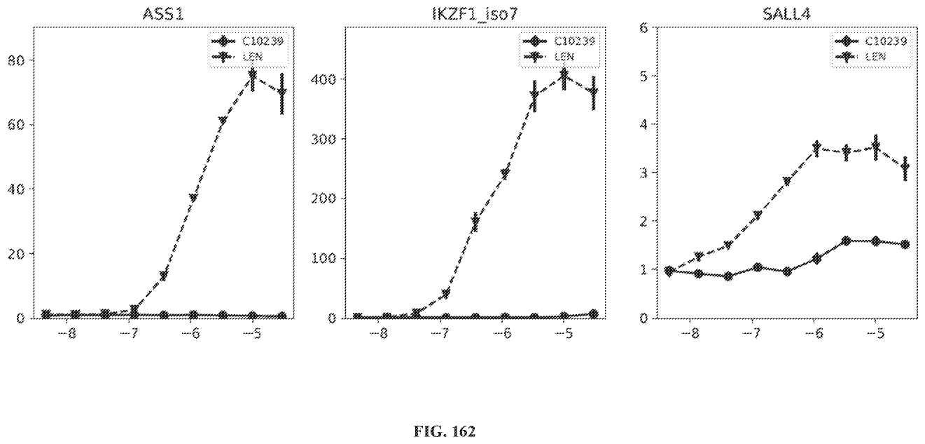

FIG. 163 illustrates the change of cereblon's ability to recruit ASS1, IKZF1, and SALL4 after binding with C10537 in comparison to LEN.

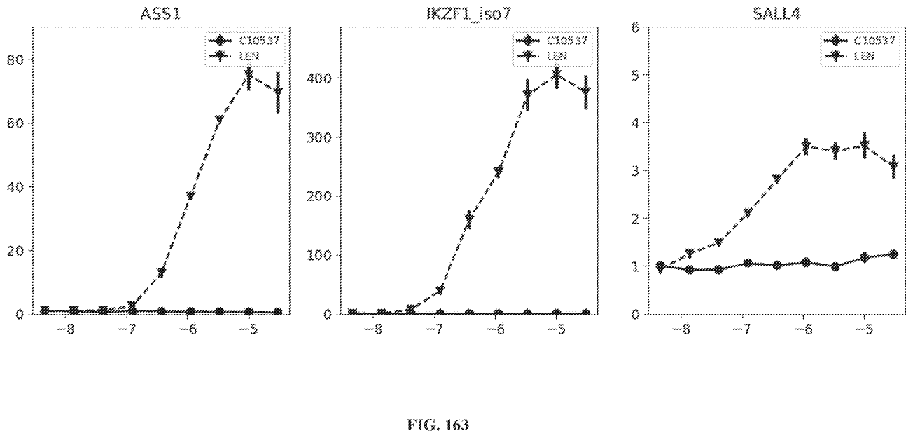

FIG. 164 illustrates the change of cereblon's ability to recruit ASS1, IKZF1, and SALL4 after binding with C10981 in comparison to LEN.

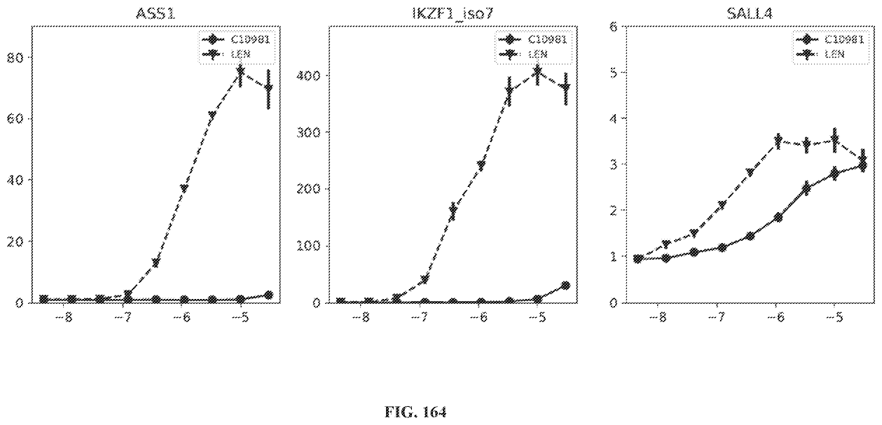

FIG. 165 illustrates the change of cereblon's ability to recruit ASS1, IKZF1, and SALL4 after binding with C12582 in comparison to LEN.

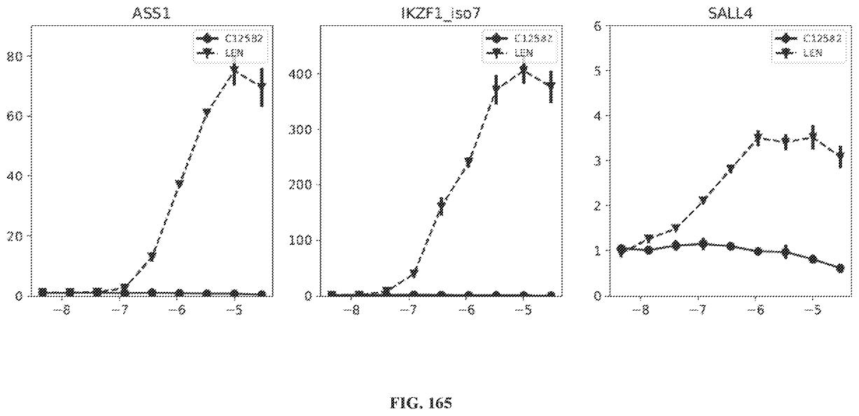

FIG. 166 illustrates the change of cereblon's ability to recruit ASS1, IKZF1, and SALL4 after binding with C12589 in comparison to LEN.

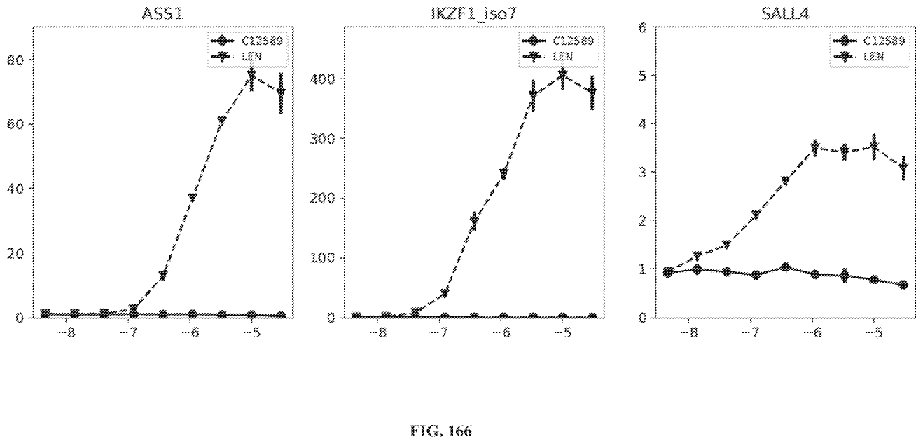

FIG. 167 illustrates the change of cereblon's ability to recruit ASS1, IKZF1, and SALL4 after binding with C12693 in comparison to LEN.

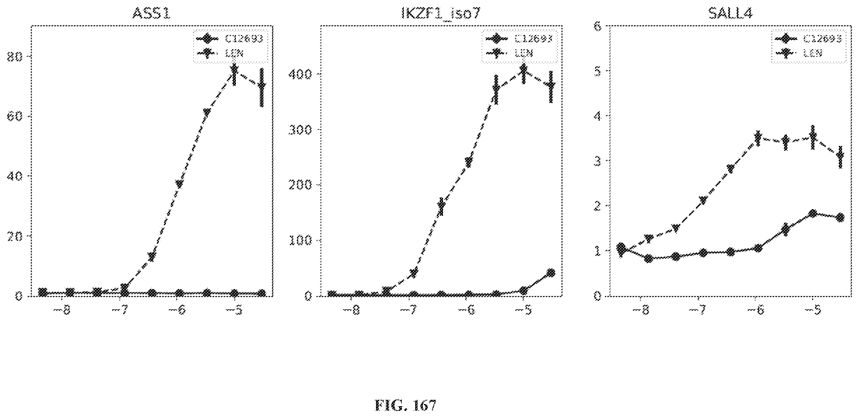

FIG. 168 illustrates the change of cereblon's ability to recruit ASS1, IKZF1, and SALL4 after binding with C12694 in comparison to LEN.

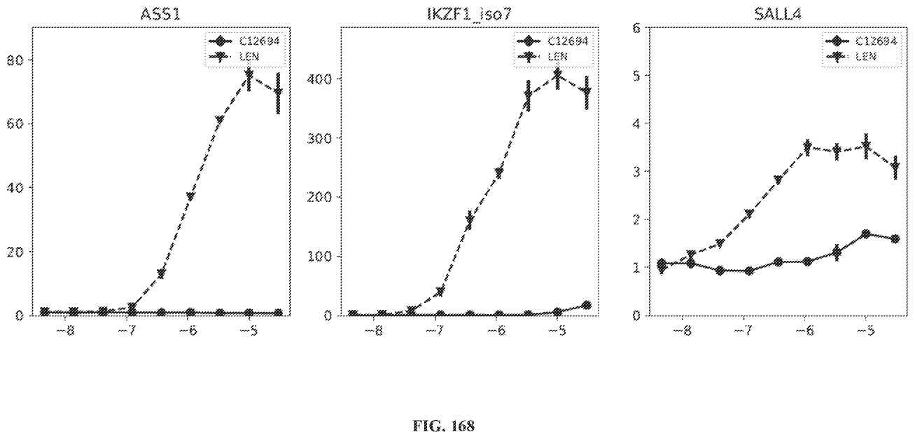

FIG. 169 illustrates the change of cereblon's ability to recruit ASS1, IKZF1, and SALL4 after binding with C12695 in comparison to LEN.

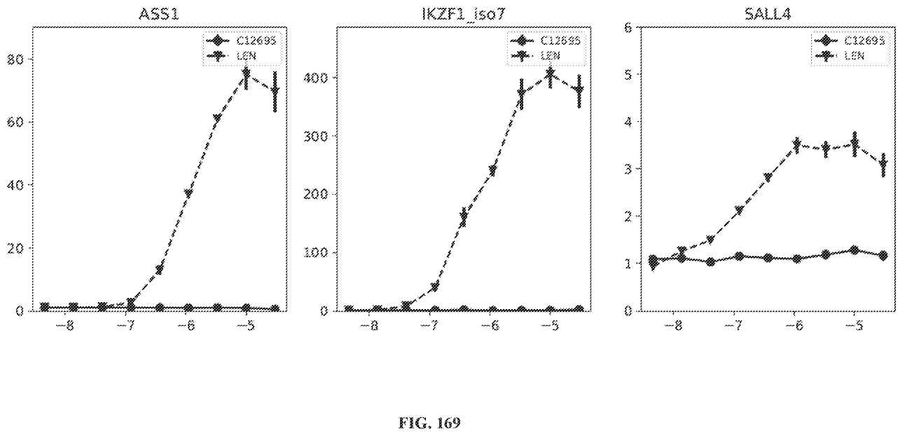

FIG. 170 illustrates the change of cereblon's ability to recruit ASS1, IKZF1, and SALL4 after binding with C95329 in comparison to LEN.

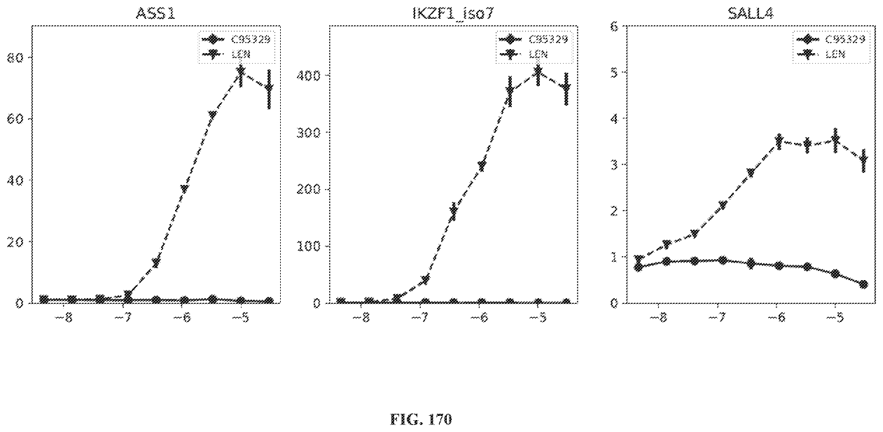

FIG. 171 illustrates the change of cereblon's ability to recruit ASS1, IKZF1, and SALL4 after binding with C95334 in comparison to LEN.

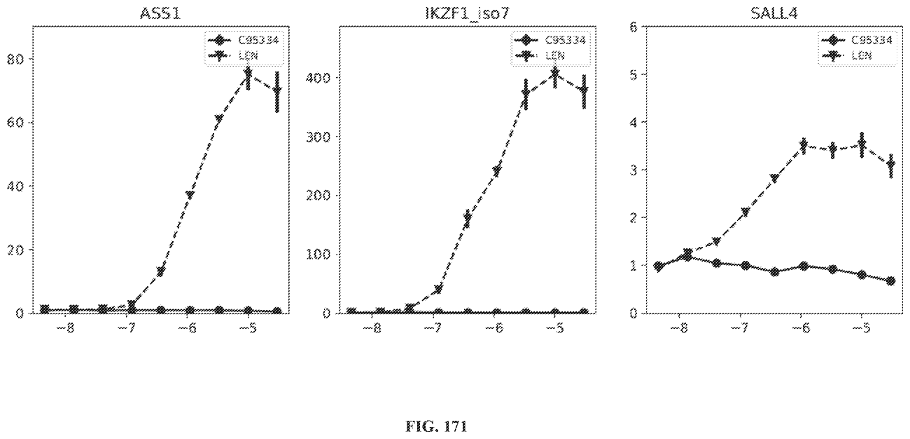

FIG. 172 illustrates the change of cereblon's ability to recruit ASS1, IKZF1, and SALL4 after binding with C95336 in comparison to LEN.

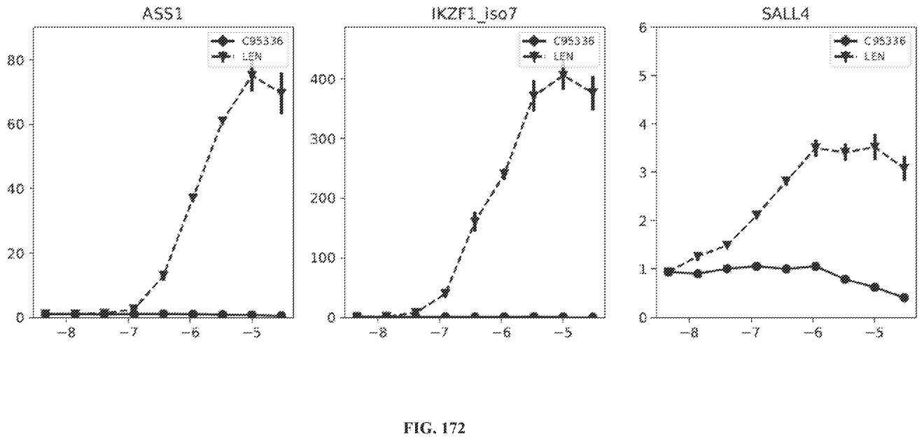

FIG. 173 illustrates the change of cereblon's ability to recruit ASS1, IKZF1, and SALL4 after binding with C59904 in comparison to LEN.

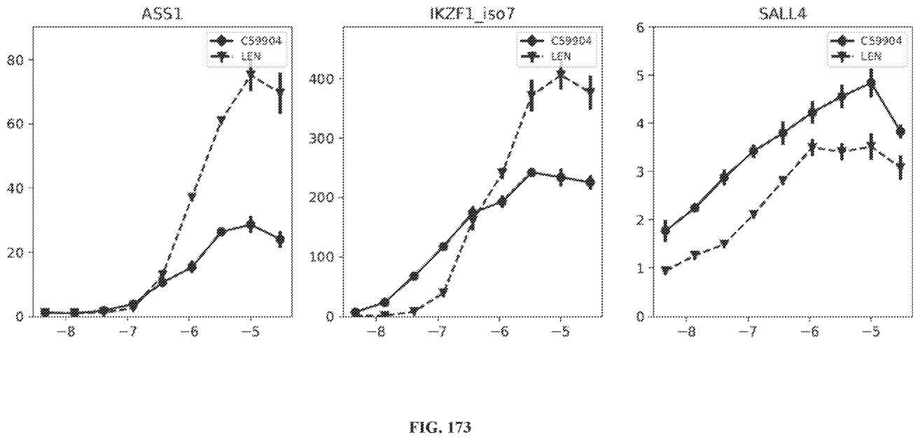

FIG. 174 illustrates the change of cereblon's ability to recruit ASS1, IKZF1, and SALL4 after binding with C75688 in comparison to LEN.

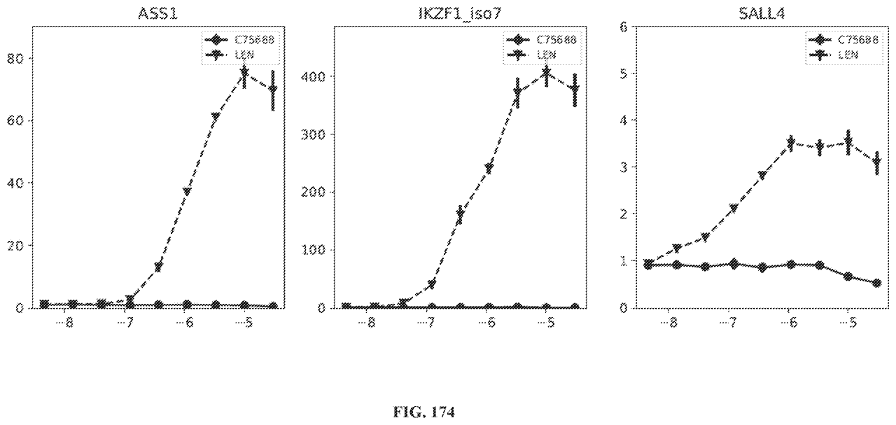

FIG. 175 illustrates the change of cereblon's ability to recruit ASS1, IKZF1, and SALL4 after binding with C35830 in comparison to LEN.

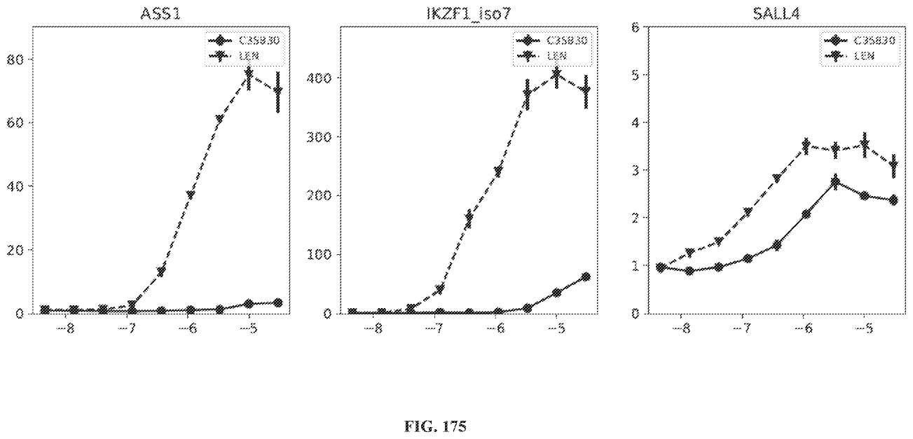

FIG. 176 illustrates the change of cereblon's ability to recruit ASS1, IKZF1, and SALL4 after binding with C13247 in comparison to LEN.

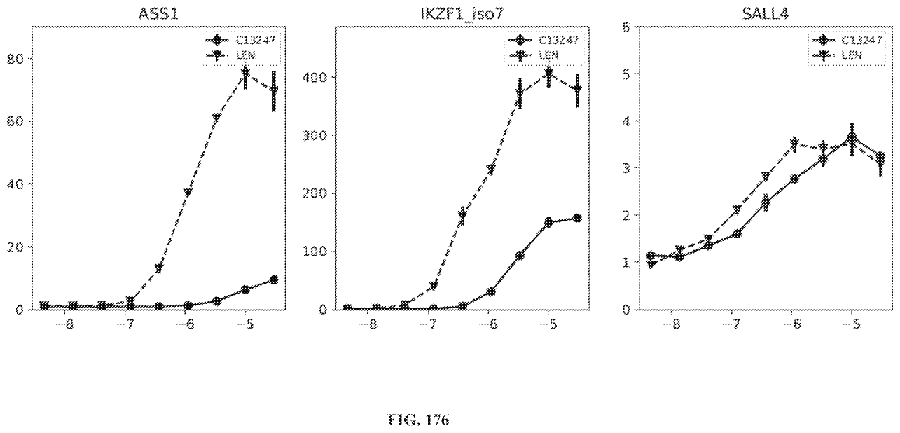

FIG. 177 illustrates the change of cereblon's ability to recruit ASS1, IKZF1, and SALL4 after binding with C16463 in comparison to LEN.

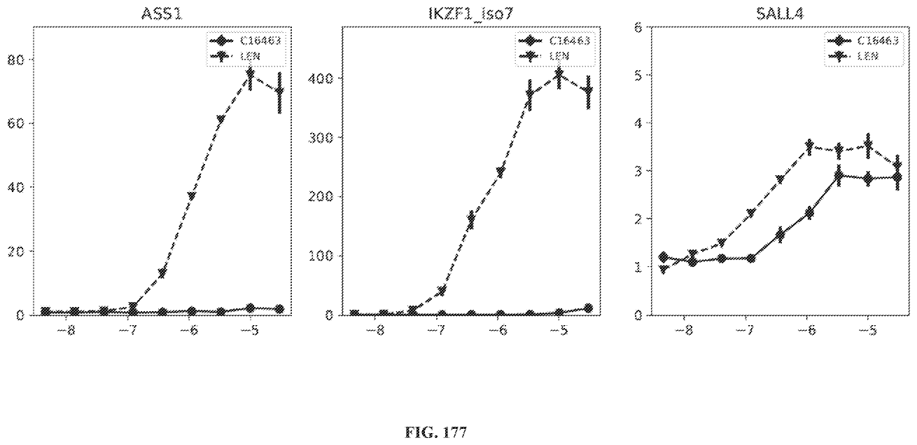

FIG. 178 illustrates the change of cereblon's ability to recruit ASS1, IKZF1, and SALL4 after binding with C17800 in comparison to LEN.

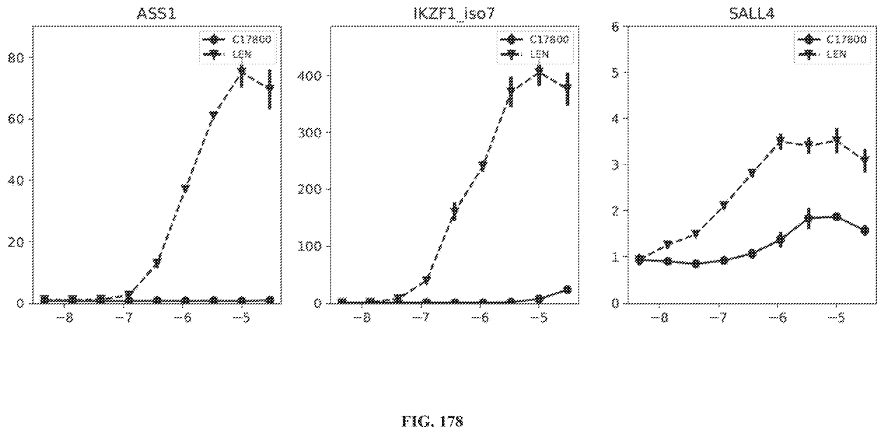

FIG. 179 illustrates the change of cereblon's ability to recruit ASS1, IKZF1, and SALL4 after binding with C21223 in comparison to LEN.

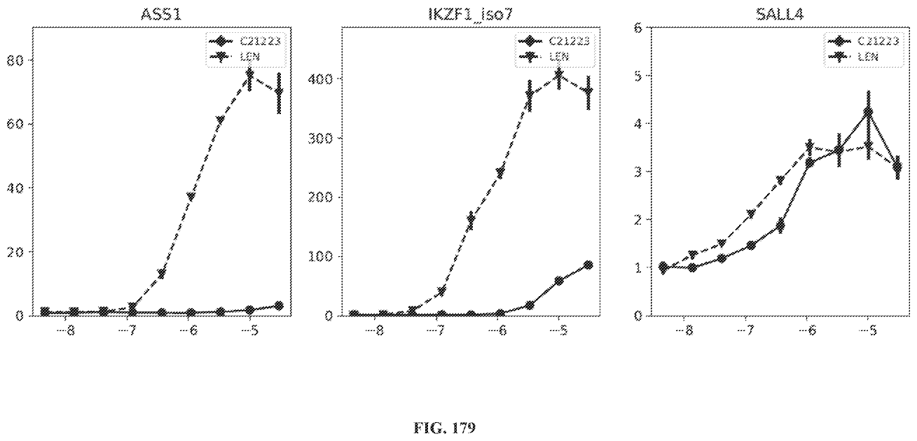

FIG. 180 illustrates the change of cereblon's ability to recruit ASS1, IKZF1, and SALL4 after binding with ZE26-0001 in comparison to LEN.

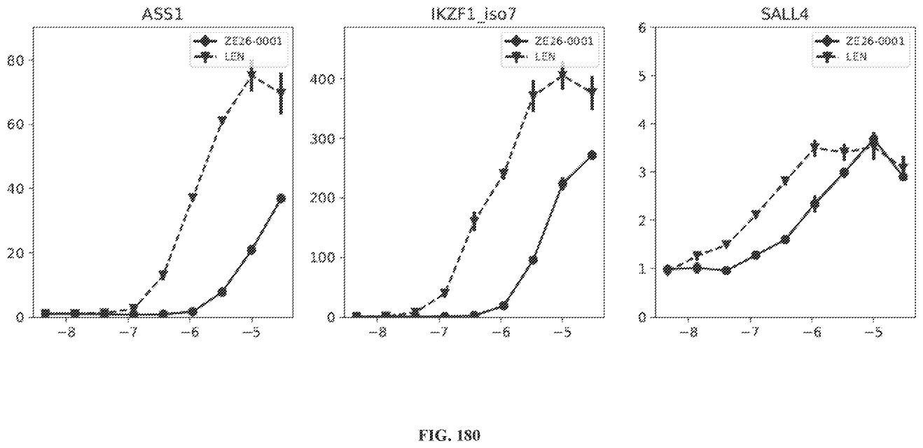

### DETAILED DESCRIPTION OF THE INVENTION

### Definitions

The term “H” denotes a single hydrogen atom. This radical may be attached, for example, to an oxygen atom to form a hydroxyl radical.

“ [symbol] ” indicates the double bond in E or Z configuration.

Where the term “alkyl” is used, either alone or within other terms such as “haloalkyl” or “alkylamino”, it embraces linear or branched radicals having one to about twelve carbon atoms. More preferred alkyl radicals are “lower alkyl” radicals having one to about six carbon atoms. Examples of such radicals include methyl, ethyl, n-propyl, isopropyl, n-butyl, isobutyl, sec-butyl, tert-butyl, pentyl, isoamyl, hexyl and the like. Even more preferred are lower alkyl radicals having one or two carbon atoms. The term “alkylenyl” or “alkylene” embraces bridging divalent alkyl radicals such as methylenyl or ethylenyl. The term “lower alkyl substituted with R²” does not include an acetal moiety. The term “alkyl” further includes alkyl radicals wherein one or more carbon atoms in the chain is substituted with a heteroatom selected from oxygen, nitrogen, or sulfur.

The term “alkenyl” embraces linear or branched radicals having at least one carbon-carbon double bond of two to about twelve carbon atoms. More preferred alkenyl radicals are “lower alkenyl” radicals having two to about six carbon atoms. Most preferred lower alkenyl radicals are radicals having two to about four carbon atoms. Examples of alkenyl radicals include ethenyl, propenyl, allyl, propenyl, butenyl and 4-methylbutenyl. The terms “alkenyl” and “lower alkenyl”, embrace radicals having “cis” and “trans” orientations, or alternatively, “E” and “Z” orientations.

The term “alkynyl” denotes linear or branched radicals having at least one carbon-carbon triple bond and having two to about twelve carbon atoms. More preferred alkynyl radicals are “lower alkynyl” radicals having two to about six carbon atoms. Most preferred are lower alkynyl radicals having two to about four carbon atoms. Examples of such radicals include propargyl, and butynyl, and the like.

Alkyl, alkylenyl, alkenyl, and alkynyl radicals may be optionally substituted with one or more functional groups such as halo, hydroxy, nitro, amino, cyano, haloalkyl, aryl, heteroaryl, and heterocyclo and the like.

The term “halo” means halogens such as fluorine, chlorine, bromine or iodine atoms.

The term “haloalkyl” embraces radicals wherein any one or more of the alkyl carbon atoms is substituted with halo as defined above. Specifically embraced are monohaloalkyl, dihaloalkyl and polyhaloalkyl radicals including perhaloalkyl. A monohaloalkyl radical, for example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1 to 6 carbon atoms. Even more preferred are lower haloalkyl radicals having one to three carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “perfluoroalkyl” means alkyl radicals having all hydrogen atoms replaced with fluoro atoms. Examples include trifluoromethyl and pentafluoroethyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. Even more preferred are lower hydroxyalkyl radicals having one to three carbon atoms.

The term “alkoxy” embraces linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. Even more preferred are lower alkoxy radicals having one to three carbon atoms. Alkoxy radicals may be further substituted with one or more halo atoms, such as fluoro, chloro or bromo, to provide “haloalkoxy” radicals. Even more preferred are lower haloalkoxy radicals having one to three carbon atoms. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one or two rings, wherein such rings may be attached together in a fused manner. The term “aryl” embraces aromatic radicals such as phenyl, naphthyl, indenyl, tetrahydronaphthyl, and indanyl. More preferred aryl is phenyl. An “aryl” group may have 1 or more substituents such as lower alkyl, hydroxyl, halo, haloalkyl, nitro, cyano, alkoxy, and lower alkylamino, and the like. Phenyl substituted with —O—CH₂—O— forms the aryl benzodioxolyl substituent.

The term “heterocyclyl” (or “heterocyclo”) embraces saturated, partially saturated and unsaturated heteroatom-containing ring radicals, where the heteroatoms may be selected from nitrogen, sulfur and oxygen. It does not include rings containing —O—O—, —O—S— or —S—S— portions. The “heterocyclyl” group may have 1 to 4 substituents such as hydroxyl, Boc, halo, haloalkyl, cyano, lower alkyl, lower aralkyl, oxo, lower alkoxy, amino and lower alkylamino.

Examples of saturated heterocyclic radicals include saturated 3 to 6-membered heteromonocyclic groups containing 1 to 4 nitrogen atoms [e.g., pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocyclyl radicals include dihydrothienyl, dihydropyranyl, dihydrofuryl and dihydrothiazolyl.

Examples of unsaturated heterocyclic radicals, also termed “heteroaryl” radicals, include unsaturated 5 to 6 membered heteromonocyclyl group containing 1 to 4 nitrogen atoms, for example, pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 5- to 6-membered heteromonocyclic group containing an oxygen atom, for example, pyranyl, 2-furyl, 3-furyl, etc.; unsaturated 5 to 6-membered heteromonocyclic group containing a sulfur atom, for example, 2-thienyl, 3-thienyl, etc.; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic group containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl].

The term heterocyclyl, (or heterocyclo) also embraces radicals where heterocyclic radicals are fused/condensed with aryl radicals: unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo [1,5-b]pyridazinyl]; unsaturated condensed heterocyclic group containing 1 to 2 oxygen atoms and 1 to 3 nitrogen atoms [e.g. benzoxazolyl, benzoxadiazolyl]; unsaturated condensed heterocyclic group containing 1 to 2 sulfur atoms and 1 to 3 nitrogen atoms [e.g., benzothiazolyl, benzothiadiazolyl]; and saturated, partially unsaturated and unsaturated condensed heterocyclic group containing 1 to 2 oxygen or sulfur atoms [e.g. benzofuryl, benzothienyl, 2,3-dihydro-benzo[1,4]dioxinyl and dihydrobenzofuryl]. Preferred heterocyclic radicals include five to ten membered fused or unfused radicals. More preferred examples of heteroaryl radicals include quinolyl, isoquinolyl, imidazolyl, pyridyl, thienyl, thiazolyl, oxazolyl, furyl and pyrazinyl. Other preferred heteroaryl radicals are 5- or 6-membered heteroaryl, containing one or two heteroatoms selected from sulfur, nitrogen and oxygen, selected from thienyl, furyl, pyrrolyl, indazolyl, pyrazolyl, oxazolyl, triazolyl, imidazolyl, pyrazolyl, isoxazolyl, isothiazolyl, pyridyl, piperidinyl and pyrazinyl.

Particular examples of non-nitrogen containing heteroaryl include pyranyl, 2-furyl, 3-furyl, 2-thienyl, 3-thienyl, benzofuryl, and benzothienyl, and the like.

Particular examples of partially saturated and saturated heterocyclyl include pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl, and the like.

The term “heterocyclo” thus encompasses the following ring systems:

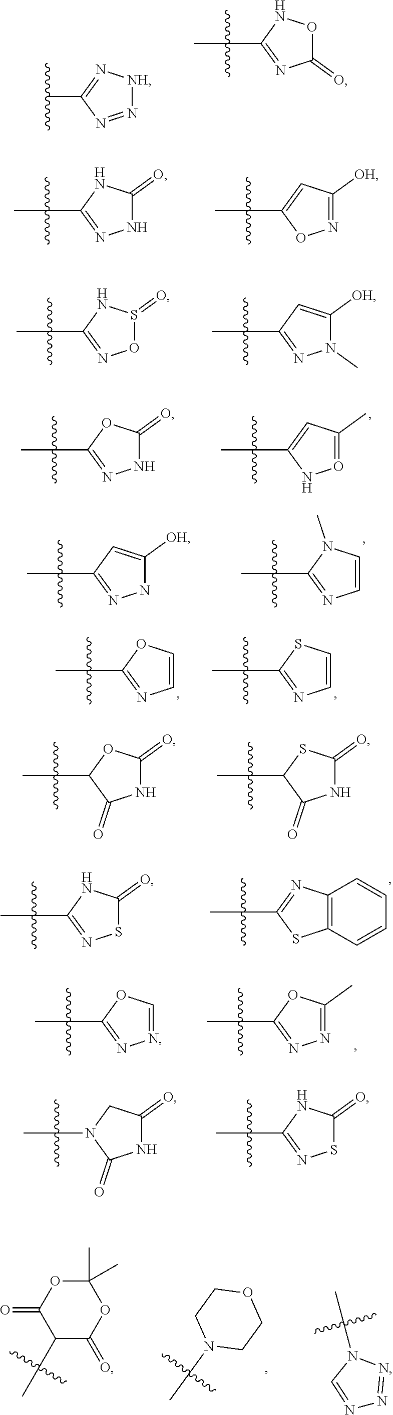

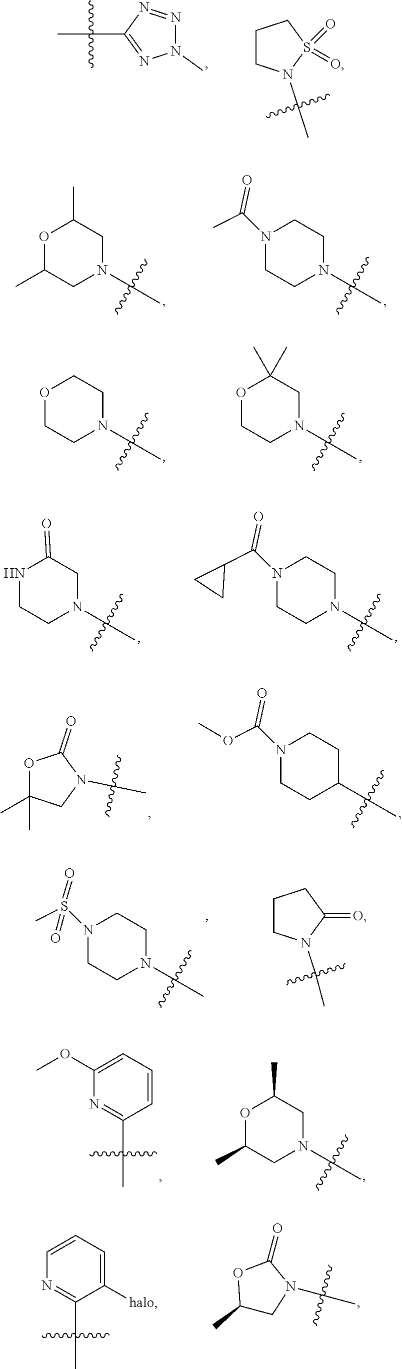

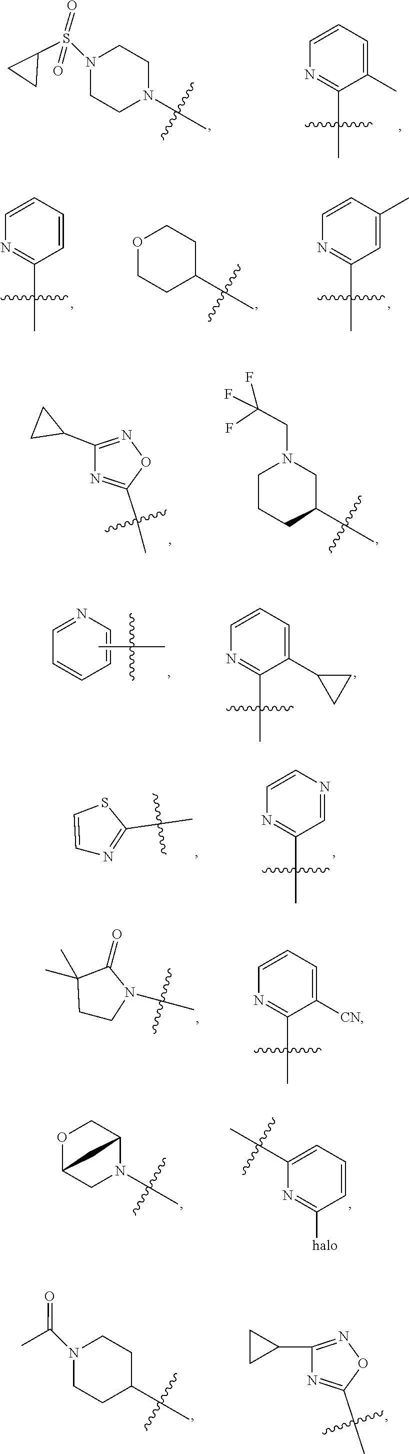

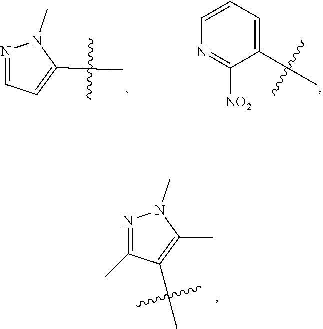

and the like.

The term “sulfonyl”, whether used alone or linked to other terms such as alkylsulfonyl, denotes respectively divalent radicals —SO₂—.

The terms “sulfamyl,” “aminosulfonyl” and “sulfonamidyl,” denotes a sulfonyl radical substituted with an amine radical, forming a sulfonamide (—SO₂NH₂).

The term “alkylaminosulfonyl” includes “N-alkylaminosulfonyl” where sulfamyl radicals are independently substituted with one or two alkyl radical(s). More preferred alkylaminosulfonyl radicals are “lower alkylaminosulfonyl” radicals having one to six carbon atoms. Even more preferred are lower alkylaminosulfonyl radicals having one to three carbon atoms. Examples of such lower alkylaminosulfonyl radicals include N-methylaminosulfonyl, and N-ethylaminosulfonyl.

The terms “carboxy” or “carboxyl,” whether used alone or with other terms, such as “carboxyalkyl,” denotes —CO₂H.

The term “carbonyl,” whether used alone or with other terms, such as “aminocarbonyl,” denotes —(C═O)—.

The term “aminocarbonyl” denotes an amide group of the formula C(═O)NH₂.

The terms “N-alkylaminocarbonyl” and “N,N-dialkylaminocarbonyl” denote aminocarbonyl radicals independently substituted with one or two alkyl radicals, respectively. More preferred are “lower alkylaminocarbonyl” having lower alkyl radicals as described above attached to an aminocarbonyl radical.

The terms “N-arylaminocarbonyl” and “N-alkyl-N-arylaminocarbonyl” denote aminocarbonyl radicals substituted, respectively, with one aryl radical, or one alkyl and one aryl radical.

The terms “heterocyclylalkylenyl” and “heterocyclylalkyl” embrace heterocyclic-substituted alkyl radicals. More preferred heterocyclylalkyl radicals are “5- or 6-membered heteroarylalkyl” radicals having alkyl portions of one to six carbon atoms and a 5- or 6-membered heteroaryl radical. Even more preferred are lower heteroarylalkylenyl radicals having alkyl portions of one to three carbon atoms. Examples include such radicals as pyridylmethyl and thienylmethyl.

The term “aralkyl” embraces aryl-substituted alkyl radicals. Preferable aralkyl radicals are “lower aralkyl” radicals having aryl radicals attached to alkyl radicals having one to six carbon atoms. Even more preferred are “phenylalkylenyl” attached to alkyl portions having one to three carbon atoms. Examples of such radicals include benzyl, diphenylmethyl and phenylethyl. The aryl in said aralkyl may be additionally substituted with halo, alkyl, alkoxy, halkoalkyl and haloalkoxy.

The term “alkylthio” embraces radicals containing a linear or branched alkyl radical, of one to ten carbon atoms, attached to a divalent sulfur atom. Even more preferred are lower alkylthio radicals having one to three carbon atoms. An example of “alkylthio” is methylthio, (CH₃S—).

The term “haloalkylthio” embraces radicals containing a haloalkyl radical, of one to ten carbon atoms, attached to a divalent sulfur atom. Even more preferred are lower haloalkylthio radicals having one to three carbon atoms. An example of “haloalkylthio” is trifluoromethylthio.

The term “alkylamino” embraces “N-alkylamino” and “N,N-dialkylamino” where amino groups are independently substituted with one alkyl radical and with two alkyl radicals, respectively. More preferred alkylamino radicals are “lower alkylamino” radicals having one or two alkyl radicals of one to six carbon atoms, attached to a nitrogen atom. Even more preferred are lower alkylamino radicals having one to three carbon atoms. Suitable alkylamino radicals may be mono or dialkylamino such as N-methylamino, N-ethylamino, N,N-dimethylamino, and N,N-diethylamino, and the like.

The term “arylamino” denotes amino groups, which have been substituted with one or two aryl radicals, such as N-phenylamino. The arylamino radicals may be further substituted on the aryl ring portion of the radical.

The term “heteroarylamino” denotes amino groups, which have been substituted with one or two heteroaryl radicals, such as N-thienylamino. The “heteroarylamino” radicals may be further substituted on the heteroaryl ring portion of the radical.

The term “aralkylamino” denotes amino groups, which have been substituted with one or two aralkyl radicals. More preferred are phenyl-C₁-C₃-alkylamino radicals, such as N-benzylamino. The aralkylamino radicals may be further substituted on the aryl ring portion.

The terms “N-alkyl-N-arylamino” and “N-aralkyl-N-alkylamino” denote amino groups, which have been independently substituted with one aralkyl and one alkyl radical, or one aryl and one alkyl radical, respectively, to an amino group.

The term “aminoalkyl” embraces linear or branched alkyl radicals having one to about ten carbon atoms any one of which may be substituted with one or more amino radicals. More preferred aminoalkyl radicals are “lower aminoalkyl” radicals having one to six carbon atoms and one or more amino radicals. Examples of such radicals include aminomethyl, aminoethyl, aminopropyl, aminobutyl and aminohexyl. Even more preferred are lower aminoalkyl radicals having one to three carbon atoms.

The term “alkylaminoalkyl” embraces alkyl radicals substituted with alkylamino radicals. More preferred alkylaminoalkyl radicals are “lower alkylaminoalkyl” radicals having alkyl radicals of one to six carbon atoms. Even more preferred are lower alkylaminoalkyl radicals having alkyl radicals of one to three carbon atoms. Suitable alkylaminoalkyl radicals may be mono or dialkyl substituted, such as N-methylaminomethyl, N,N-dimethyl-aminoethyl, and N,N-diethylaminomethyl, and the like.

The term “alkylaminoalkoxy” embraces alkoxy radicals substituted with alkylamino radicals. More preferred alkylaminoalkoxy radicals are “lower alkylaminoalkoxy” radicals having alkoxy radicals of one to six carbon atoms. Even more preferred are lower alkylaminoalkoxy radicals having alkyl radicals of one to three carbon atoms. Suitable alkylaminoalkoxy radicals may be mono or dialkyl substituted, such as N-methylaminoethoxy, N,N-dimethylaminoethoxy, and N,N-diethylaminoethoxy, and the like.

The term “alkylaminoalkoxyalkoxy” embraces alkoxy radicals substituted with alkylaminoalkoxy radicals. More preferred alkylaminoalkoxyalkoxy radicals are “lower alkylaminoalkoxyalkoxy” radicals having alkoxy radicals of one to six carbon atoms. Even more preferred are lower alkylaminoalkoxyalkoxy radicals having alkyl radicals of one to three carbon atoms. Suitable alkylaminoalkoxyalkoxy radicals may be mono or dialkyl substituted, such as N-methylaminomethoxyethoxy, N-methylaminoethoxyethoxy, N,N-dimethylaminoethoxyethoxy, and N,N-diethylaminomethoxymethoxy, and the like.

The term “carboxyalkyl” embraces linear or branched alkyl radicals having one to about ten carbon atoms any one of which may be substituted with one or more carboxy radicals. More preferred carboxyalkyl radicals are “lower carboxyalkyl” radicals having one to six carbon atoms and one carboxy radical. Examples of such radicals include carboxymethyl, and carboxypropyl, and the like. Even more preferred are lower carboxyalkyl radicals having one to three CH₂ groups.

The term “halosulfonyl” embraces sulfonyl radicals substituted with a halogen radical. Examples of such halosulfonyl radicals include chlorosulfonyl and fluorosulfonyl.

The term “arylthio” embraces aryl radicals of six to ten carbon atoms, attached to a divalent sulfur atom. An example of “arylthio” is phenylthio.

The term “aralkylthio” embraces aralkyl radicals as described above, attached to a divalent sulfur atom. More preferred are phenyl-C₁-C₃-alkylthio radicals. An example of “aralkylthio” is benzylthio.

The term “aryloxy” embraces optionally substituted aryl radicals, as defined above, attached to an oxygen atom. Examples of such radicals include phenoxy.

The term “aralkoxy” embraces oxy-containing aralkyl radicals attached through an oxygen atom to other radicals. More preferred aralkoxy radicals are “lower aralkoxy” radicals having optionally substituted phenyl radicals attached to lower alkoxy radical as described above.

The term “heteroaryloxy” embraces optionally substituted heteroaryl radicals, as defined above, attached to an oxygen atom.

The term “heteroarylalkoxy” embraces oxy-containing heteroarylalkyl radicals attached through an oxygen atom to other radicals. More preferred heteroarylalkoxy radicals are “lower heteroarylalkoxy” radicals having optionally substituted heteroaryl radicals attached to lower alkoxy radical as described above.

The term “cycloalkyl” includes saturated carbocyclic groups. Preferred cycloalkyl groups include C₃-C₆ rings. More preferred compounds include, cyclopentyl, cyclopropyl, and cyclohexyl.

The term “cycloalkylalkyl” embraces cycloalkyl-substituted alkyl radicals. Preferable cycloalkylalkyl radicals are “lower cycloalkylalkyl” radicals having cycloalkyl radicals attached to alkyl radicals having one to six carbon atoms. Even more preferred are “5 to 6-membered cycloalkylalkyl” attached to alkyl portions having one to three carbon atoms. Examples of such radicals include cyclohexylmethyl. The cycloalkyl in said radicals may be additionally substituted with halo, alkyl, alkoxy and hydroxy.

The term “cycloalkenyl” includes carbocyclic groups having one or more carbon-carbon double bonds including “cycloalkyldienyl” compounds. Preferred cycloalkenyl groups include C₃-C₆ rings. More preferred compounds include, for example, cyclopentenyl, cyclopentadienyl, cyclohexenyl and cycloheptadienyl.

The term “comprising” is meant to be open ended, including the indicated component but not excluding other elements.

A group or atom that replaces a hydrogen atom is also called a substituent.

Any particular molecule or group can have one or more substituent depending on the number of hydrogen atoms that can be replaced.

The symbol “—” represents a covalent bond and can also be used in a radical group to indicate the point of attachment to another group. In chemical structures, the symbol is commonly used to represent a methyl group in a molecule.

The term “therapeutically effective amount” means an amount of a compound that ameliorates, attenuates or eliminates one or more symptom of a particular disease or condition, or prevents or delays the onset of one of more symptom of a particular disease or condition.

The terms “patient” and “subject” may be used interchangeably and mean animals, such as dogs, cats, cows, horses, sheep and humans. Particular patients are mammals. The term patient includes males and females.

The term “pharmaceutically acceptable” means that the referenced substance, such as a compound of Formula I, or a salt of a compound of Formula I, or a formulation containing a compound of Formula I, or a particular excipient, are suitable for administration to a patient.

The terms “treating”, “treat” or “treatment” and the like include preventative (e.g., prophylactic) and palliative treatment.

The term “excipient” means any pharmaceutically acceptable additive, carrier, diluent, adjuvant, or other ingredient, other than the active pharmaceutical ingredient (API), which is typically included for formulation and/or administration to a patient.

The term “cancer” means a physiological condition in mammals that is characterized by unregulated cell growth. General classes of cancers include carcinomas, lymphomas, sarcomas, and blastomas.

### Composition

The compounds of the present invention are administered to a patient in a therapeutically effective amount. The compounds can be administered alone or as part of a pharmaceutically acceptable composition or formulation. In addition, the compounds or compositions can be administered all at once, as for example, by a bolus injection, multiple times, such as by a series of tablets, or delivered substantially uniformly over a period of time, as for example, using transdermal delivery. It is also noted that the dose of the compound can be varied over time.

The compounds of the present invention, if desired, can be administered to a patient either orally, rectally, parenterally, (for example, intravenously, intramuscularly, or subcutaneously) intracistemally, intravaginally, intraperitoneally, intravesically, locally (for example, powders, ointments or drops), or as a buccal or nasal spray. All methods that are used by those skilled in the art to administer a pharmaceutically active agent are contemplated.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quatemary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents.

Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. They may also contain opacifying agents, and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferable suppositories, which can be prepared by mixing the compounds of the present invention with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

Dosage forms for topical administration of a compound of the present invention include ointments, powders, sprays and inhalants. The active compound or fit compounds are admixed under sterile condition with a physiologically acceptable carrier, and any preservatives, buffers, or propellants that may be required. Opthalmic formulations, eye ointments, powders, and solutions are also contemplated as being within the scope of this invention.

The compounds of the present invention can be administered to a patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that can be used depends on a number of factors, including the requirements of the patient, the severity of the condition or disease being treated, and the pharmacological activity of the compound being administered. The determination of dosage ranges and optimal dosages for a particular patient is within the ordinary skill in the art.

The compounds of the present invention can be administered as pharmaceutically acceptable salts, esters, amides or prodrugs. The term “salts” refers to inorganic and organic salts of compounds of the present invention. The salts can be prepared in situ during the final isolation and purification of a compound, or by separately reacting a purified compound in its free base or acid form with a suitable organic or inorganic base or acid and isolating the salt thus formed. Representative salts include the hydrobromide, hydrochloride, sulfate, bisulfate, nitrate, acetate, oxalate, palmitiate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. The salts may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66: 1-19 (1977).

Examples of pharmaceutically acceptable esters of the compounds of the present invention include C₁-C₈ alkyl esters. Acceptable esters also include C₅-C₇ cycloalkyl esters, as well as arylalkyl esters such as benzyl. C₁-C₄ alkyl esters are commonly used. Esters of compounds of the present invention may be prepared according to methods that are well known in the art.

Examples of pharmaceutically acceptable amides of the compounds of the present invention include amides derived from ammonia, primary C₁-C₈ alkyl amines, and secondary C₁-C₈ dialkyl amines. In the case of secondary amines, the amine may also be in the form of a 5 or 6 membered heterocycloalkyl group containing at least one nitrogen atom. Amides derived from ammonia, C₁-C₃ primary alkyl amines and C₁-C₂ dialkyl secondary amines are commonly used. Amides of the compounds of the present invention may be prepared according to methods well known to those skilled in the art.

The term “prodrug” means compounds that are transformed in vivo to yield a compound of the present invention. The transformation may occur by various mechanisms, such as through hydrolysis in blood. A discussion of the use of prodrugs is provided by T. Higuchi and W. Stella, “Prodrugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

To illustrate, if the compound of the invention contains a carboxylic acid functional group, a prodrug can comprise an ester formed by the replacement of the hydrogen atom of the acid group with a group such as (C₁-C₈ alkyl, (C₂-C₁₂)alkanoyloxymethyl, 1-(alkanoyloxy)ethyl having from 4 to 9 carbon atoms, 1-methyl-1-(alkanoyloxy)ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)aminomethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if a compound of the present invention comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, —P(O)(OH)2, —P(O)(O(C₁-C₆)alkyl)2 or glycosyl (the radical resulting from the removal of a hydroxyl group of the hemiacetal form of a carbohydrate).

The compounds of the present invention may contain asymmetric or chiral centers, and therefore, exist in different stereoisomeric forms. It is contemplated that all stereoisomeric forms of the compounds as well as mixtures thereof, including racemic mixtures, form part of the present invention. In addition, the present invention contemplates all geometric and positional isomers. For example, if the compound contains a double bond, both the cis and trans forms (designated as S and E, respectively), as well as mixtures, are contemplated.

Mixture of stereoisomers, such as diastereomeric mixtures, can be separated into their individual stereochemical components on the basis of their physical chemical differences by known methods such as chromatography and/or fractional crystallization. Enantiomers can also be separated by converting the enantiomeric mixture into a diastereomeric mixture by reaction with an appropriate optically active compound (e.g., an alcohol), separating the diastereomers and converting (e.g., hydrolyzing) the individual diastereomers to the corresponding pure enantiomers. Also, some compounds may be atropisomers (e.g., substituted biaryls).

The compounds of the present invention may exist in unsolvated as well as solvated forms with pharmaceutically acceptable solvents such as water (hydrate), ethanol, and the like. The present invention contemplates and encompasses both the solvated and unsolvated forms.

It is also possible that compounds of the present invention may exist in different tautomeric forms. All tautomers of compounds of the present invention are contemplated. For example, all of the tautomeric forms of the tetrazole moiety are included in this invention. Also, for example, all keto-enol or imine-enamine forms of the compounds are included in this invention.

Those skilled in the art will recognize that the compound names and structures contained herein may be based on a particular tautomer of a compound. While the name or structure for only a particular tautomer may be used, it is intended that all tautomers are encompassed by the present invention, unless stated otherwise.

It is also intended that the present invention encompass compounds that are synthesized in vitro using laboratory techniques, such as those well known to synthetic chemists; or synthesized using in vivo techniques, such as through metabolism, fermentation, digestion, and the like. It is also contemplated that the compounds of the present invention may be synthesized using a combination of in vitro and in vivo techniques.

The present invention also includes isotopically-labelled compounds, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁶O, ¹⁷O ¹⁸O, ³¹P, ³²p, ³⁵S, ¹⁸F, and ³⁶Cl. In one aspect, the present invention relates to compounds wherein one or more hydrogen atom is replaced with deuterium (2H) atoms.

Compounds of the present invention that contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detection. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of this invention can generally be prepared by substituting a readily available isotopically labelled reagent for a non-isotopically labelled reagent.

The compounds of the present invention may exist in various solid states including crystalline states and as an amorphous state. The different crystalline states, also called polymorphs, and the amorphous states of the present compounds are contemplated as part of this invention.

All patents, published patent applications and other publications recited herein are hereby incorporated by reference.

### Compounds

The present invention relates to novel compounds that can bind cereblon (CRBN), but variously have reduced ability to recruit liability substrate proteins, such as SALL4 and ASS1 and have varied ability recruit more common substrates, such as IKZF1 to CRBN. Differential recruitment profiles among CRBN binding molecules create novel starting points for selective small molecule substrate degrader glues, either alone, or as a modular component of a CRBN-binding heterobifunctional molecule (HBM). Recruitment profiles of key liability substrates offer an objective means to prioritize and select promising small molecules. In one embodiment, some CRBN binding compounds described herein, after binding with CRBN, reduce CRBN's ability to recruit selected liability substrates, such as SALL4 and ASS1, and common substrates, such as IKZF1. These compounds is referred as “silent” compounds. In one embodiment, some CRBN binding compounds described herein, after binding with CRBN, increase CRBN's ability to recruit selected liability substrates.

CRBN acts as a substrate receptor protein within an E3 ligase complex to direct substrate protein degradation via the proteasome, forming functional interactions with DNA Damage Binding Protein 1 (DDB1), Cullin4 (4A or 4B) and Regulator of Culling 1 (RoC1) as well as an E2 ligase protein, such as UBE2G1, to substrate ubiquitination and subsequent degradation.

Small molecules deriving from the glutarimide-containing drug (IMiD), thalidomide, have been shown to bind CRBN and, as a complex, recruit novel neosubstrates to the E3 ligase complex.

The consequence of the IMiD-induced recruitment of substrate proteins to CRBN is that such proteins are then degraded by the Ubiquitin Proteasome Pathway (UPP).

New compounds are provided, along with their uses and manufacture that bind cereblon. It is believed that binding of the disclosed compounds to cereblon results in increased or reduced interactions of cereblon with IKZF1, SALL4, or ASS1, leading to their subsequent ubiquitination and degradation in the proteasome. The selected compounds are found to be both potent binders of cereblon as well as showing potential therapeutic use.

Compounds disclosed herein, pharmaceutically acceptable salts thereof, or pharmaceutically acceptable compositions can be used to treat a disorder mediated by one or more of cereblon, IKZF1, SALL4, and ASS1.

In one aspect, the compound of the present invention is selected from Formula I:

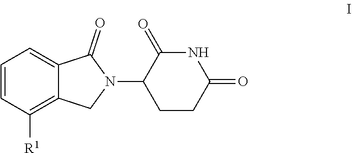

or a pharmaceutically acceptable salt thereof, wherein:
R¹ is aryl, —N(R⁵)—X—R⁶, —SO₂R⁵, or —O(CH₂)ₘR⁵, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

In an embodiment, R¹ is phenyl optionally substituted with 1 or more R^{w} groups as allowed by valence.

In an embodiment, R¹ is phenyl.

In an embodiment, R¹ is phenyl optionally substituted with one or more (C₁-C₃)alkyl, (C₁-C₃)alkoxy, or OH.

In one aspect, the compound of the present invention is selected from Formula II:

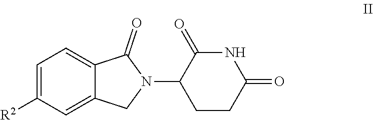

or a pharmaceutically acceptable salt thereof, wherein:
R² is aryl, —NH—(C₃-C₁₀) heteroaryl, or —N(R⁵)—(CH₂)ₘ—X—(CH₂)ₙ—R⁶, any of which may be optionally substituted with 1 or more R^{w} groups as allowed by valence; R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

In an embodiment, R² is phenyl optionally substituted with 1 or more R^{w} groups as allowed by valence.

In an embodiment, R² is —NH—(C₃-C₁₀) heteroaryl.

In an embodiment, R² is phenyl optionally substituted with one or more (C₁-C₃)alkyl, (C₁-C₃)alkoxy, or OH.

In an embodiment, R² is —N(R⁵)—(CH₂)ₘ—X—(CH₂)ₙ—R⁶; R⁵ is H; R⁶ is OH, (C₁-C₃)alkyl, —(C₁-C₃)alkoxy, —NR⁵R⁵, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl.

In one aspect, the compound of the present invention is selected from Formula III:

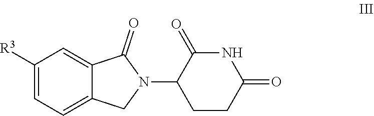

or a pharmaceutically acceptable salt thereof, wherein:
R³ is cyano, aryl, —NH—(C₃-C₁₀) heteroaryl, (C₃-C₁₀)heterocyclo, or —N(R⁵)—(CH₂)ₘ—X—(CH₂)ₙ—R⁶, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

In an embodiment, R³ is —N(R⁵)—(CH₂)ₘ—X—(CH₂)ₙ—R⁶; R⁵ is H; R⁶ is OH, (C₁-C₃)alkyl, —(C₁-C₃)alkoxy, —NR⁵R⁵, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl.

In one aspect, the compound of the present invention is selected from Formula IV:

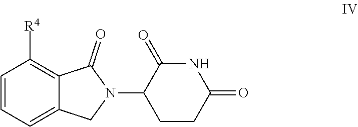

or a pharmaceutically acceptable salt thereof, wherein:
R⁴ is halo, cyano, aryl, OR⁵, or —N(R⁵)—(CH₂)ₘ—X—(CH₂)ₙ—R⁶, any of which may be optionally substituted with 1 or more R^{w} groups as allowed by valence; R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

In an embodiment, R⁴ is —N(R⁵)—(CH₂)ₘ—X—(CH₂)ₙ—R⁶; R⁵ is H; R⁶ is OH, (C₁-C₃)alkyl, —(C₁-C₃)alkoxy, —NR⁵R⁵, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl.

In one aspect, the compound of the present invention is selected from Formula V:

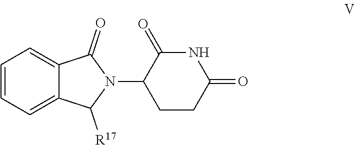

or a pharmaceutically acceptable salt thereof, wherein:
R¹⁷ is cyano, heteroaryl, —(CH₂)ₘ—C(O)O—R⁶, or —N(R⁵)—(CH₂)ₘ—X—(CH₂)ₙ—R⁶, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

In an embodiment, R¹⁷ is heteroaryl optionally substituted with OH, halo, (C₁-C₃)alkyl or (C₁-C₃)alkoxy.

In one aspect, the compound of the present invention is selected from Formula VI:

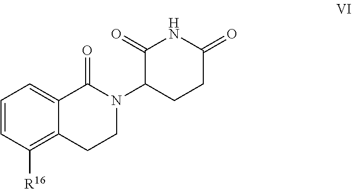

or a pharmaceutically acceptable salt thereof, wherein:
R¹⁶ is NH₂ or —N(R⁵)—(CH₂)ₘ—X—(CH₂)ₙ—R⁶, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

In one aspect, the compound of the present invention is selected from Formula VII:

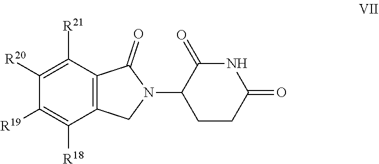

or a pharmaceutically acceptable salt thereof, wherein:
R¹⁸, R¹⁹, R²⁰, R²¹ each independently is H, halo, (C₁-C₃)alkyl, or —N(R⁵)—X—R⁶, with the proviso that no more than two substituents of R¹⁸, R¹⁹, R²⁰, R²¹ are H; or
R¹⁸, R¹⁹ taken together with the carbons they are attached to forming a (C₃-C₁₀)cycloalkyl or a (C₃-C₁₀)heterocyclo, or R¹⁹, R²⁰ taken together with the carbons they are attached to forming a (C₃-C₁₀)cycloalkyl or a (C₃-C₁₀)heterocyclo, or R²⁰, R²¹ taken together with the carbons they are attached to forming a (C₃-C₁₀)cycloalkyl or a (C₃-C₁₀)heterocyclo, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

In one aspect, the compound of the present invention is selected from Formula VIII:

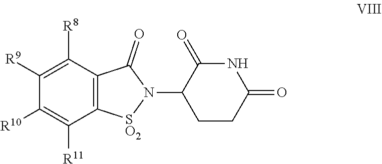

or a pharmaceutically acceptable salt thereof, wherein:
R⁸, R⁹, R¹⁰, R¹¹ each independently is H, halo, OH, cyano, (C₁-C₃)alkyl, (C₁-C₃)alkoxy, aryl, or heteroaryl, any of which may be optionally substituted with 1 or more R^{w} groups as allowed by valence;
R^{w} at each occurrence is independently, H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
n is 0, 1, 2, 3, or 4.

In one aspect, the compound of the present invention is selected from Formula IX:

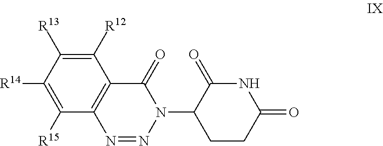

or a pharmaceutically acceptable salt thereof, wherein:
R¹², R¹³, R¹⁴, R¹⁵ each is independently H, NH₂, (C₁-C₃)alkyl, —N(R⁵)—(CH₂)ₘ—N(R⁵)—X—R⁶, with proviso that no more than three substituents out of R¹², R¹³, R¹⁴, and R¹⁵ are H, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

In one aspect, the compound of the present invention is selected from Formula X:

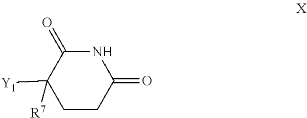

or a pharmaceutically acceptable salt thereof, wherein:
Y is —NHR³³, —NHC(O)R³³, or —CHR³³R³⁴;
R⁷ is H, (C₁-C₃)alkyl, or R⁷ and R³⁴ taken together with the carbons they are attached to forming a carbon double bond;
R³³ is aryl, heteroaryl, or (C₃-C₁₀)heterocyclo, any of which may be optionally substituted with 1 or more R^{w} groups as allowed by valence;
R^{w} at each occurrence is independently, H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
n is 0, 1, 2, 3, or 4.

In one aspect, the compound of the present invention is a compound of Formula XI

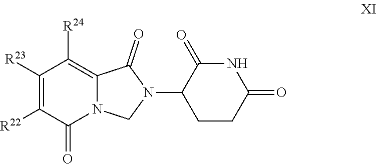

or a pharmaceutically acceptable salt thereof, wherein:
R²² is H, halo, OH, —NR⁵R⁵, (C₁-C₃)alkyl, (C₁-C₃)alkoxy, (hydroxy)(C₁-C₃)alkyl, cyano, —N(R⁵)—X—R⁶, —N(R⁵)—(CH₂)ₘ—N(R⁵)—X—R⁶, aryl, or heteroaryl, any of which may be optionally substituted with 1 or more R^{w} groups as allowed by valence;
R²³ is H, halo, OH, —NR⁵R⁵, —(CH₂)ₙ—NR⁵R⁵, (C₁-C₃)alkyl, (C₁-C₃)alkoxy, —C(O)NR⁵R⁶, (hydroxy)(C₁-C₃)alkyl, cyano, —N(R⁵)—X—R⁶, —N(R⁵)—(CHR⁵)ₘ—X—R⁶, —N(R⁵)—(CH₂)ₘ—N(R⁵)—X—R⁶, aryl, heteroaryl, or R²² and R²³ taken together with the carbons they are attached to forming a (C₃-C₁₀)cycloalkyl ring or a (C₃-C₁₀)heterocyclic ring, any of which may be optionally substituted with 1 or more R^{w} groups as allowed by valence;
R²⁴ is H, halo, OH, —NR⁵R⁵, —(CH₂)ₙ—NR⁵R⁵, (C₁-C₃)alkyl, (C₁-C₃)alkoxy, (halo)(C₁-C₃)alkyl, (hydroxy)(C₁-C₃)alkyl, cyano, —NO₂, —N(R⁵)—X—R⁶, —N(R⁵)—(CH₂)ₘ—N(R⁵)—X—R⁶, aryl, heteroaryl, or R²³ and R²⁴ taken together with the carbons they are attached to forming a (C₃-C₁₀)cycloalkyl ring or a (C₃-C₁₀)heterocyclic ring, any of which may be optionally substituted with 1 or more R^{w} groups as allowed by valence; R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;

R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;

R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;

X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

In an embodiment, R²² is H; R²³ is H; R²⁴ is halo.

In one aspect, the present invention relates to a compound of Formula XII, XIII, XIV, XV, XVI, XVII, or XVIII

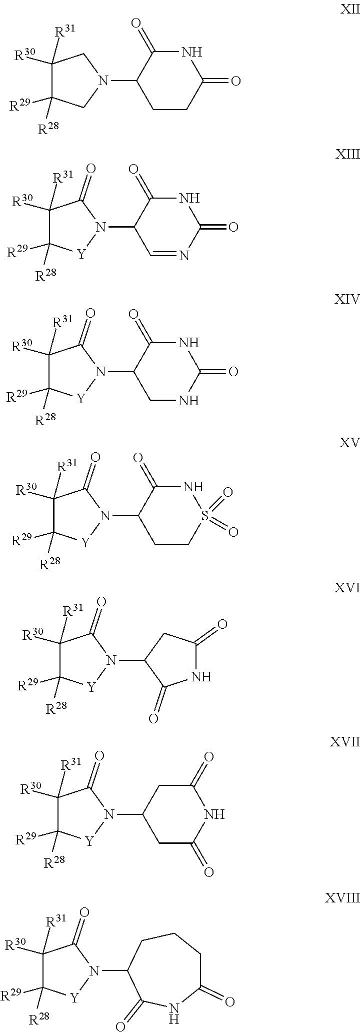

or a pharmaceutically acceptable salt thereof, wherein:
R²⁸, R²⁹, R³⁰, R³¹ is independently H, halo, OH, —NR⁵R⁵, —(CH₂)ₙ—NR⁵R⁵, (C₁-C₃)alkyl, (C₁-C₃)alkoxy, (halo)(C₁-C₃)alkyl, (hydroxy)(C₁-C₃)alkyl, cyano, —NO₂, —N(R⁵)—X—R⁶, —N(R⁵)—(CH₂)ₘ—N(R⁵)—X—R⁶, aryl, heteroaryl, or R²⁸, R²⁹ taken together with the carbons to which they are attached to form a (C₃-C₁₀)cycloalkyl or a (C₃-C₁₀)heterocyclo, or R³⁰, R³¹ taken together with the carbons to which they are attached to form a (C₃-C₁₀)cycloalkyl or a (C₃-C₁₀)heterocyclo, or R²⁹, R³⁰ taken together to form a bond, or R²⁹, R³⁰ taken together with the carbons to which they are attached to form a (C₃-C₁₀)cycloalkyl or a (C₃-C₁₀)heterocyclo or an aryl or a heteroaryl, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

In one aspect, the compound of the present invention is selected from the compounds listed in Table 1 in Example 4.

In one aspect, the compound of the present invention is selected from the group consisting of:
3-[1-oxo-5-(quinazolin-4-ylamino)isoindolin-2-yl]piperidine-2,6-dione;
3-[5-[(4-aminothieno[2,3-d]pyrimidin-2-yl)amino]-1-oxo-isoindolin-2-yl]piperidine-2,6-dione;
N-[2-(2,6-dioxo-3-piperidyl)-1-oxo-isoindolin-5-yl]acetamide;
3-[5-[(2-aminopyrimidin-4-yl)amino]-1-oxo-isoindolin-2-yl]piperidine-2,6-dione;
6-[[2-(2,6-dioxo-3-piperidyl)-1-oxo-isoindolin-5-yl]amino]pyridazine-3-carbonitrile;
3-[[2-[[2-(2,6-dioxo-3-piperidyl)-1-oxo-isoindolin-5-yl]amino]acetyl]amino]benzamide;
2-[[2-(2,6-dioxo-3-piperidyl)-1-oxo-isoindolin-5-yl]amino]acetic acid;
N-[2-(2,6-dioxo-3-piperidyl)-3-oxo-isoindolin-5-yl]quinoline-2-carboxamide;
3-[6-[[2-(2-methyl-1-piperidyl)-2-oxo-ethyl]amino]-1-oxo-isoindolin-2-yl]piperidine-2,6-dione;
3-[6-[(2-isoindolin-2-yl-2-oxo-ethyl)amino]-1-oxo-isoindolin-2-yl]piperidine-2,6-dione;
N-(cyclopropylmethyl)-2-[[2-(2,6-dioxo-3-piperidyl)-3-oxo-isoindolin-5-yl]amino]-N-methyl-acetamide;
acetic acid; 3-[1-oxo-6-(quinazolin-4-ylamino)isoindolin-2-yl]piperidine-2,6-dione;
3-[6-[[2-(3-methyl-1-piperidyl)-2-oxo-ethyl]amino]-1-oxo-isoindolin-2-yl]piperidine-2,6-dione;
3-[6-[(4-methyl-3-oxo-pyrazin-2-yl)amino]-1-oxo-isoindolin-2-yl]piperidine-2,6-dione;
3-[1-oxo-6-(quinoxalin-2-ylamino)isoindolin-2-yl]piperidine-2,6-dione;
3-[6-[(1-methylpyrazolo[3,4-d]pyrimidin-4-yl)amino]-1-oxo-isoindolin-2-yl]piperidine-2,6-dione;
3-[6-(5,7-dihydrofuro[3,4-d]pyrimidin-2-ylamino)-1-oxo-isoindolin-2-yl]piperidine-2,6-dione;
3-[6-[(6-methylpyrimidin-4-yl)amino]-1-oxo-isoindolin-2-yl]piperidine-2,6-dione;
2-[[2-(2,6-dioxo-3-piperidyl)-3-oxo-isoindolin-5-yl]amino]-N-phenyl-acetamide;
3-[6-[[2-(2,4-dimethylpiperazin-1-yl)-2-oxo-ethyl]amino]-1-oxo-isoindolin-2-yl]piperidine-2,6-dione;
3-[6-(dimethylamino)-1-oxo-isoindolin-2-yl]piperidine-2,6-dione;
3-(1-oxo-6-phenyl-isoindolin-2-yl)piperidine-2,6-dione;
2-[[2-(2,6-dioxo-3-piperidyl)-3-oxo-isoindolin-5-yl]amino]-N,N-dimethyl-acetamide;
3-[6-[[2-(2-methylmorpholin-4-yl)-2-oxo-ethyl]amino]-1-oxo-isoindolin-2-yl]piperidine-2,6-dione;
2-[[2-(2,6-dioxo-3-piperidyl)-3-oxo-isoindolin-5-yl]amino]-N-methyl-N-[(1-methylpyrazol-4-yl)methyl]acetamide;
N-benzyl-2-[[2-(2,6-dioxo-3-piperidyl)-3-oxo-isoindolin-5-yl]amino]acetamide;
6-[[2-(2,6-dioxo-3-piperidyl)-3-oxo-isoindolin-5-yl]amino]pyridazine-3-carbonitrile;
3-[6-[(6-methylpyrrolo[3,2-d]pyrimidin-4-yl)amino]-1-oxo-isoindolin-2-yl]piperidine-2,6-dione;
2-(dimethylamino)-N-[2-(2,6-dioxo-3-piperidyl)-3-oxo-isoindolin-5-yl]acetamide;
N-[2-(2,6-dioxo-3-piperidyl)-3-oxo-isoindolin-5-yl]-5H-pyrrolo[2,3-b]pyridine-4-carboxamide;
N-cyclopropyl-2-[[2-(2,6-dioxo-3-piperidyl)-3-oxo-isoindolin-5-yl]amino]acetamide;
3-[1-oxo-6-(2-oxoimidazolidin-1-yl)isoindolin-2-yl]piperidine-2,6-dione;
2-(2,6-dioxo-3-piperidyl)-3-oxo-isoindoline-5-carbonitrile;
2-[[2-(2,6-dioxo-3-piperidyl)-3-oxo-isoindolin-5-yl]amino]propanoic acid;
2-acetamido-N-[2-(2,6-dioxo-3-piperidyl)-3-oxo-isoindolin-5-yl]acetamide;
2-[[2-(2,6-dioxo-3-piperidyl)-3-oxo-isoindolin-5-yl]amino]acetamide;
3-[6-[[2-(3-methyl-5-oxo-piperazin-1-yl)-2-oxo-ethyl]amino]-1-oxo-isoindolin-2-yl]piperidine-2,6-dione;
N-[2-(2,6-dioxo-3-piperidyl)-3-oxo-isoindolin-5-yl]acetamide;
3-[6-[[2-(4-methyl-3-oxo-piperazin-1-yl)-2-oxo-ethyl]amino]-1-oxo-isoindolin-2-yl]piperidine-2,6-dione;
N-[2-(2,6-dioxo-3-piperidyl)-3-oxo-isoindolin-5-yl]-3H-imidazo[4,5-b]pyridine-6-carboxamide;
2-[[2-(2,6-dioxo-3-piperidyl)-3-oxo-isoindolin-5-yl]amino]-N-tetrahydropyran-4-yl-acetamide;
2-[[2-(2,6-dioxo-3-piperidyl)-3-oxo-isoindolin-5-yl]amino]acetic acid;
3-[1-oxo-6-[[2-oxo-2-(1-piperidyl)ethyl]amino]isoindolin-2-yl]piperidine-2,6-dione;
3-(1-oxo-7-phenyl-isoindolin-2-yl)piperidine-2,6-dione;
2-(2,6-dioxo-3-piperidyl)-3-oxo-isoindoline-4-carbonitrile;
2-[[2-(2,6-dioxo-3-piperidyl)-3-oxo-isoindolin-4-yl]amino]acetic acid;
3-(7-fluoro-1-oxo-isoindolin-2-yl)piperidine-2,6-dione;
3-(5-amino-1-oxo-3,4-dihydroisoquinolin-2-yl)piperidine-2,6-dione;
t-butyl 2-[2-(2,6-dioxo-3-piperidyl)-3-oxo-isoindolin-1-yl]acetate;
3-[1-(2H-indol-3-yl)-3-oxo-isoindolin-2-yl]piperidine-2,6-dione;
2-(2,6-dioxo-3-piperidyl)-3-oxo-isoindoline-1-carbonitrile;
3-[1-(dimethylamino)-3-oxo-isoindolin-2-yl]piperidine-2,6-dione;
3-(2-oxopyrrolidin-1-yl)piperidine-2,6-dione;
3-(quinazolin-2-ylamino)piperidine-2,6-dione;
(3Z)-3-benzylidenepiperidine-2,6-dione;
3-(quinoxalin-2-ylamino)piperidine-2,6-dione;
3-(pyrimidin-2-ylamino)piperidine-2,6-dione;
N-(2,6-dioxo-3-piperidyl)-2-oxo-3H-pyridine-6-carboxamide;
3-(4-methyl-1,1,3-trioxo-1,2-benzothiazol-2-yl)piperidine-2,6-dione;
3-(8-amino-4-oxo-1,2,3-benzotriazin-3-yl)piperidine-2,6-dione;
3-(5-amino-4-oxo-1,2,3-benzotriazin-3-yl)piperidine-2,6-dione;
3-(4-oxo-1,2,3-benzotriazin-3-yl)piperidine-2,6-dione;
3-(5-methyl-4-oxo-1,2,3-benzotriazin-3-yl)piperidine-2,6-dione;
3-(6-methyl-4-oxo-1,2,3-benzotriazin-3-yl)piperidine-2,6-dione; and
3-(8-methyl-4-oxo-1,2,3-benzotriazin-3-yl)piperidine-2,6-dione.

In one aspect, the present invention relates to a composition comprising a pharmaceutically effective amount of the compound described herein and a pharmaceutically acceptable carrier.

In one aspect, the present invention relates to a method of treating a disease comprising administering the composition comprising a pharmaceutically effective amount of the compound described herein and a pharmaceutically acceptable carrier to a subject in need thereof.

In one aspect, the present invention relates to a method of treating a cancer comprising administering the composition comprising a pharmaceutically effective amount of the compound described herein and a pharmaceutically acceptable carrier to a subject in need thereof. In an embodiment, the cancer is selected from the group consisting of squamous-cell carcinoma, basal cell carcinoma, adenocarcinoma, hepatocellular carcinomas, renal cell carcinomas, bladder cancer, bowel cancer, breast cancer, cervical cancer, colon cancer, esophageal cancer, head cancer, kidney cancer, liver cancer, lung cancer, neck cancer, ovarian cancer, pancreatic cancer, prostate cancer, stomach cancer, uterine cancer, leukemias, lymphomas, Burkitt's lymphoma, Non-Hodgkin's lymphoma, melanomas, myeloproliferative diseases, multiple myeloma, sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, Schwannomas, testicular cancer, thyroid cancer, astrocytoma, Hodgkin's disease, Wilms' tumor, and teratocarcinomas.

In another aspect, the present invention relates to a method of treating or preventing one or more autoimmune diseases or disorders comprising administering a composition comprising a pharmaceutically effective amount of the compound described herein and a pharmaceutically acceptable carrier to a subject in need thereof. In an embodiment, the autoimmune disease or disorder is selected from, such as multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, and other autoimmune diseases or disorders.

In an embodiment, the subject is a human.

In another aspect, the present invention relates to a method of modulating cereblon comprising administering the composition comprising the compounds of Formulas I-XVIII to a subject in need thereof. In an embodiment, the present invention relates to a method of modulating cereblon comprising administering the composition comprising the compounds selected from the compounds listed in Table 1 in Example 4 to a subject in need thereof.

In another aspect, the present invention relates to a method of modulating proteasomal degradation of a protein comprising administering the composition comprising the compounds of Formulas I-XVIII to a subject in need thereof. In an embodiment, the present invention relates to a method of modulating proteasomal degradation of a protein comprising administering the composition comprising the compounds selected from the compounds listed in Table 1 in Example 4 to a subject in need thereof.

In another aspect, the present invention relates to a method of modulating sequestration of a protein to the proteasome comprising administering the composition comprising the compounds of Formulas I-XVIII to a subject in need thereof. In an embodiment, the present invention relates to a method of modulating sequestration of a protein to the proteasome comprising administering the composition comprising the compounds selected from the compounds listed in Table 1 in Example 4 to a subject in need thereof.

### Examples

### General Synthetic Schemes

Compounds of the present invention generally can be prepared beginning with commercially available starting materials and using synthetic techniques known to those of skill in the art. Outlined below are some reaction schemes suitable for preparing compounds of the present invention. Further exemplification is found in the specific examples provided.

### Example 1: Competition Assay

CRBN binding was assessed with a MAPPIT-like assay by determining the ability of test compounds to compete with a trimethoprim-lenalidomide hybrid ligand for binding to CRBN in cells. The traditional MAPPIT assay, as described for example in Lemmens, et al. “MAPPIT, a mammalian two-hybrid method for in-cell detection of protein-protein interactions,” Methods Mol Biol. 2015; 1278:447-55, has been used to monitor protein-protein interactions. A bait protein (protein A) is expressed as a fusion protein in which it is genetically fused to an engineered intracellular receptor domain of the leptin receptor, which is itself fused to the extracellular domain of the erythropoietin (Epo) receptor. Binding of Epo ligand to the EpoR component results in activation of receptor-associated intracellular JAK2. However, activated JAK2 cannot activate the leptin receptor to trigger STAT3 binding and its phosphorylation because its tyrosine residues, normally phosphorylated by activated JAK2, have been mutated. Reconstitution of a JAK2 phosphorylatable STAT3 docking site is instead created through interaction of a protein B with protein A, whereby protein B is fused to a cytoplasmic domain of the gp130 receptor (which now harbors appropriate tyrosine resides recognized by the activated JAK2 kinase). Thus, physical interaction of protein A with protein B reconstitutes and Epo triggers JAK2-STAT3 signaling pathway activation. Activation of STAT3 can be monitored by introduction of a STAT3-responsive reporter gene, including a luciferase-encoding gene or a gene encoding a fluorescent marker such as GFP or some other type of fluorescent protein (EGF etc.). In this manner, the MAPPIT assay provides a versatile assay to assess such recombinant protein-protein interactions, or compound- or hybrid ligand-induced protein-protein interactions, in intact cells.

Here, a similar MAPPIT-like assay was used to determine the ability of test compounds to compete with the trimpethoprim-lenalidomide-induced binding between DHFR and CRBN. Therefore, HEK293 cells transfected with the appropriate cDNAs encoding transgenes (encoding DHFR and CRBN fusion proteins), were used to generate a positive assay signal as a result of ternary protein/compound complex formation, including a DHFR-fusion protein, a trimethoprim(TMP)-lenalidomide hybrid ligand (TMP is a ligand for DHFR), and a CRBN-gp130 fusion protein (CRBN binds the ligand lenalidomide)—thus, a DHFR-TMP-LEN-CRBN complex formation. Formation of the complex resulted in activation of a STAT-responsive luciferase reporter gene. That signal was set to 100% luciferase activity. In a separate sample set up, cells were prepared in the same manner but, in addition, co-incubated with a test compound whose interaction with CRBN was investigated. Binding to the CRBN fusion protein would compete with binding of the hybrid ligand to the same CRBN protein, hence inhibiting the assay signal due to prevention of ternary complex formation, which was required to generate an assay signal. Increasing concentrations of test compound were assessed to determine CRBN binding efficiency as determined in this type of ligand competition experiment in living cells. Specificity of signal inhibition was assessed by a parallel experimental set up in which test compound effect was assessed for inhibition of signal generated by a control gp130 fusion protein (CTRL) that directly bound to the DHFR-fusion protein in the absence of hybrid ligand (i.e. a direct interaction of the proteins).

In more detail, HEK293T cells were cultured in Dulbecco's modified Eagle's medium supplemented with 10% fetal calf serum, incubated at 37° C., 8% CO₂. Cells were transfected with a plasmid encoding E. coli Dihydrofolate Reductase (DHFR) fused to the tails of the cytoplasmic domain of a mutated leptin receptor (pCLG-eDHFR), a plasmid encoding a CRBN prey fused to gp130 cytoplasmic domain (pMG1-CRBN) or a plasmid encoding a REM2 control prey that could directly interact with the leptin receptor of the DHFR fusion protein (pMG1-REM2), and the STAT3 responsive pXP2d2-rPAPI-luciferase reporter plasmid—using a standard transfection method, as described (Lievens, et al. “Array MAPPIT: high-throughput interactome analysis in mammalian cells.” Journal of Proteome Research 8.2 (2009): 877-886). Cells were treated with leptin to activate the leptin receptor fusion protein and supplemented with 300 nM trimethoprim-lenalidomide fusion compound (hybrid ligand, where trimethoprim interacts with DHFR and lenalidomide with CRBN) without or with the indicated dose of test compound at 24 hours after transfection. Luciferase activity, induced by formation of the ternary complex including DHFR-trimethoprim-lenalidomide-CRBN, and consequential activation of STAT3 signaling, was measured 24 hours after compound treatment using the Luciferase Assay System kit (PROMEGA, Madison, Wis.) with an Ensight plate reader (PERKIN ELMER LIFE SCIENCES, Waltham, Mass.). Data points represented the average luciferase activity of triplicate samples derived from cells treated with leptin+test compound for the REM2 control (CTRL) or cells treated with leptin+hybrid ligand+test compound (CRBN) relative to leptin (CTRL) or leptin+hybrid ligand (CRBN) only treated samples (the signals obtained in absence of added test compound for both cases is set at 100% of luciferase activity on y-axis). Error bars represented standard deviations. Curves were fit using 4-parameter nonlinear regression in GRAPHPAD PRISM software

### Example 2: Recruitment Assays

In this Example 2, a similar MAPPIT-like assay was applied as described in Example 1 to determine test compound-induced binding of a particular substrate protein of interest to CRBN. In this experimental set-up, cells were transfected with a construct encoding a CRBN-fusion protein and another one encoding a substrate-fusion protein. Test compound activity was assessed with increasing concentrations of test compounds (dose-response studies) to monitor the ability to promote CRBN-ligand-induced protein interaction.

Specifically, HEK293T cells were transfected with a plasmid encoding the MAPPIT receptor fusion wherein the protein of interest (CRBN or substrate protein) is genetically linked to a cytoplasmic domain of the leptin receptor, which itself is fused to the extracellular domain of the erythropoietin (Epo) receptor (pSEL-X, where X represents either CRBN or any of the tested substrate proteins of interest), a plasmid encoding the MAPPIT gp130 fusion (pMG1-Y, Y being either any of the tested substrate proteins or CRBN) and a STAT3-responsive luciferase-encoding reporter plasmid (pXP2d2-rPAPI-luciferase reporter plasmid), as described (Lievens, et al. “Array MAPPIT: high-throughput interactome analysis in mammalian cells.” Journal of Proteome Research 8.2 (2009): 877-886). Full size proteins were fused for each of the target proteins tested, except in the case of IKZF1 where isoform 7 was used. For the study, the following were used: IKZF1 recruitment: pSEL-CRBN+pMG1-IKZF1(isoform 7); ASS1 recruitment: pSEL-CRBN+pMG1-ASS1. SALL4 recruitment: pSEL-SALL4+pMG1-CRBN. Cells were treated with erythropoietin (Epo) without or with the indicated dose of test compound at 24 hours after transfection. Luciferase activity was measured 24 hours after test compound treatment using the Luciferase Assay System kit (PROMEGA, Madison, Wis.) with an Ensight plate reader (PERKIN ELMER LIFE SCIENCES, Waltham, Mass.). Data points depicted fold induction of the average luciferase activity of triplicate samples from EPO+test compound treated cells versus EPO only treated cells. Error bars represent standard deviations. Curves were fit using 4-parameter nonlinear regression in GRAPHPAD PRISM software.

### Example 3: Preparation of the Compounds

The compounds of the present invention can be prepared by methods well known in the art of organic chemistry. See, for example, J. March, ‘Advanced Organic Chemistry’ 4th Edition, John Wiley and Sons. During synthetic sequences it may be necessary and/or desirable to protect sensitive or reactive groups on any of the molecules concerned. This is achieved by means of conventional protecting groups, such as those described in T. W. Greene and P. G. M. Wutts ‘Protective Groups in Organic Synthesis’ 3rd Edition, John Wiley and Sons, 1999. The protective groups are optionally removed at a convenient subsequent stage using methods well known in the art. The products of the reactions are optionally isolated and purified, if desired, using conventional techniques, but not limited to, filtration, distillation, crystallization, chromatography and the like. Such materials are optionally characterized using conventional means, including physical constants and spectral data.

In synthesizing compounds of the present invention, it may be desirable to use certain leaving groups. The term “leaving groups” (“LG”) generally refer to groups that are displaceable by a nucleophile. Such leaving groups are known in the art. Examples of leaving groups include, but are not limited to, halides (e.g., I, Br, F, Cl), sulfonates (e.g., mesylate, tosylate), sulfides (e.g., SCH3), N-hydroxsuccinimide, N-hydroxybenzotriazole, and the like. Examples of nucleophiles include, but are not limited to, amines, thiols, alcohols, Grignard reagents, anionic species (e.g., alkoxides, amides, carbanions) and the like.

### HPLC Purification

Purification was performed using HPLC (H₂O—MeOH; Agilent 1260 Infinity systems equipped with DAD and mass-detectors. Waters Sunfire C18 OBD Prep Column, 100 Å, 5 μm, 19 mm×100 mm with SunFire C18 Prep Guard Cartridge, 100 Å, 10 μm, 19 mm×10 mm) The material was dissolved in 0.7 mL DMSO. Flow: 30 mL/min. Purity of the obtained fractions was checked via the analytical LCMS. Spectra were recorded for each fraction as it was obtained straight after chromatography in the solution form. The solvent was evaporated in the flow of N₂ at 80° C. On the basis of post-chromatography LCMS analysis fractions were united. Solid fractions were dissolved in 0.5 mL MeOH and transferred into a pre-weighted marked vials. Obtained solutions were again evaporated in the flow of N₂ at 80° C. After drying, products were finally characterized by LCMS and ¹H NMR. For clarity, in the synthetic schemes of this section, hydrogen atom is not shown for simplicity, for example: “—NH₂” is shown as “—N”; “—OH” is shown as “—O”.

### Analytical Methods

### NMR

Instrument specifications:

Bruker AVANCE DRX 500

Varian UNITYplus 400

### LCMS

Instrument specifications:
Agilent 1100 Series LC/MSD system with DAD\ELSD and Agilent LC\MSD VL (G1956 Å), SL (G1956B) mass-spectrometer.
Agilent 1200 Series LC/MSD system with DAD\ELSD and Agilent LC\MSD SL (G6130 Å), SL (G6140 Å) mass-spectrometer.
All the LC/MS data were obtained using positive/negative mode switching.
Column Zorbax SB-C18 1.8 μm 4.6×15 mm Rapid Resolution cartridge (PN 821975-932)
Mobile phase A—acetonitrile, 0.1% formic acid
   B—water (0.1% formic acid)
Flow rate 3 ml/min
Gradient 0 min—100% B
0.01 min—100% B
1.5 min—0% B
1.8 min—0% B
1.81 min—100% B
Injection volume 1l
Ionization mode atmospheric pressure chemical ionization (APCI)
Scan range m/z 80-1000

### C24031

Synthesis of target compound was carried out following the scheme given below:

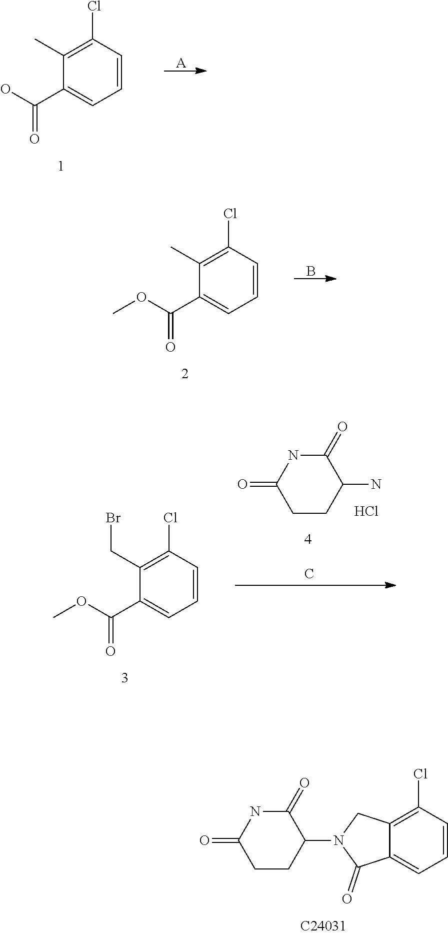

### General Procedure A:

Compound 1 (20 mmol) and K₂CO₃ (24 mmol) were suspended in DMF (20 mL). To the reaction mixture, was added iodomethane (24 mmol). The reaction was stirred at r.t. overnight, then diluted with water (150 mL). The solution was extracted with ethyl acetate (2×200 mL), organic phase was washed with brine (3×150 ml), dried with CaCl₂) and evaporated to give the methyl ester 2. Yield: 94%.

### General Procedure B:

To a solution of compound 2 (1.6 mmol) in CCl₄ (3 ml) was added AIBN (0.1 mmol) and NBS (2 mmol) during 15 min at 25° C. The reaction was heated for 12h at 60° C. and cooled to r.t. The solvent was then removed in vacuo. The crude residue was purified using LC. Yield: 64%.

### General Procedure C:

Compound 1 (1 mmol), DIPEA (2.1 mmol) and compound 2 hydrochloride (1.3 mmol) were dissolved in 5 ml DMF, the mixture was heated at 80° C. for 18 hours (TLC and LCMS control). The reaction mixture was cooled, filtered and the solvent was evaporated. The crude residue was purified using HPLC. Yield: 49%.

### C74668

Synthesis of target compound was carried out following the scheme given below:

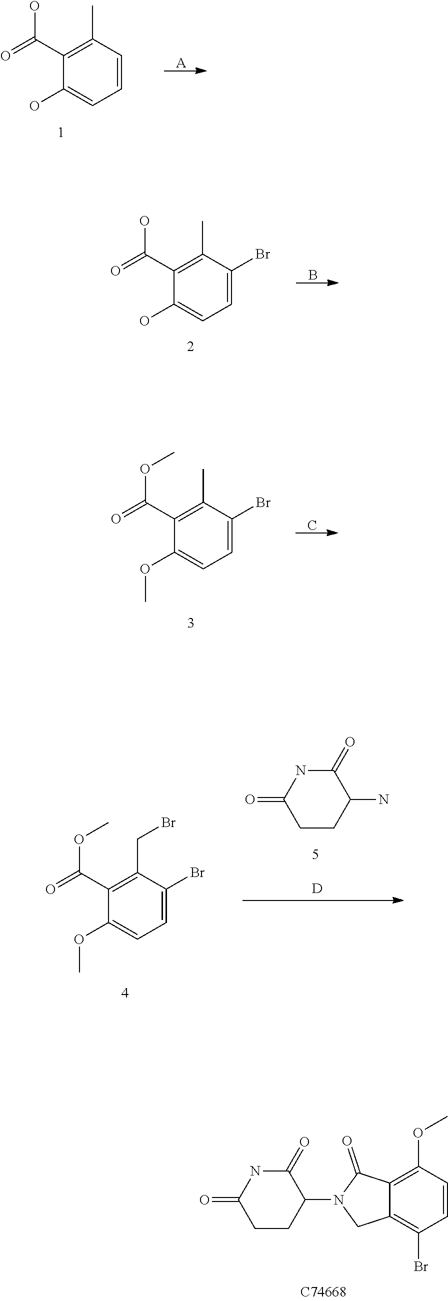

### Step A:

To a suspension of 1 (13.2 g, 86.8 mmol) in acetic acid (150 mL) at 0° C. was followed by portionwise N-bromosuccinimide (17 g, 95.4 mmol) over 30 min. The mixture was warmed to 20° C. and stirred for 3 h, then was treated with 40% aqueous sodium bisulfate (100 mL). The acetonitrile was removed under vacuum and the aqueous residue was extracted with EtOAc (3×40 mL). The combined organic layers were washed with water and brine, dried and concentrated to give 3-bromo-6-hydroxy-2-methylbenzoic acid as a white solid 16 g. Yield 82%.

### Step B:

This compound was prepared according to General Procedure A for C24031. Yield: 87%.

### Step C:

This compound was prepared according to General Procedure B for C24031. Yield: 92%.

### Step D:

This compound was prepared according to General Procedure C for C24031. Yield: 42%.

### C07207

Synthesis of target compound was carried out following the scheme given below:

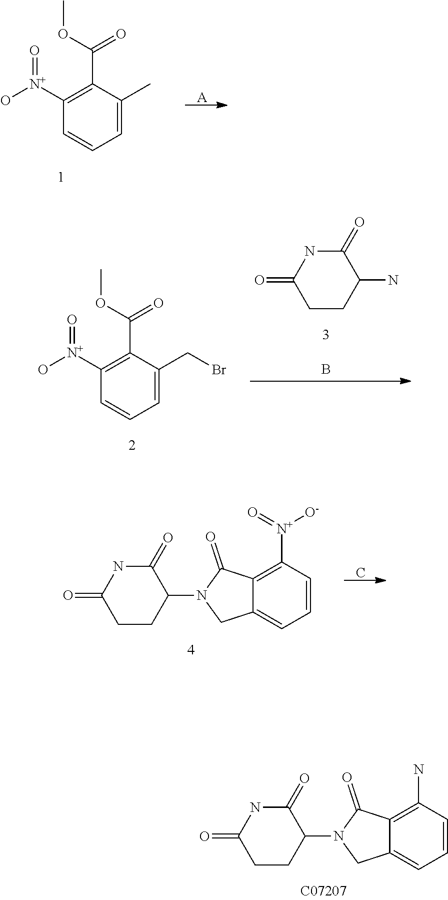

### Step A:

This compound was prepared according to General Procedure B for C24031. Yield: 84%.

### Step B:

This compound was prepared according to General Procedure C for C24031. Yield: 87%.

### Step C:

To a solution of compound 4 (0.5 mmol) in methanol (2 mL) was added palladium carbon (5%, 10 mg), and the mixture was stirred under a hydrogen atmosphere at room temperature for 10 h. The reaction mixture was filtered through celite. The filtrate was concentrated under reduced pressure and after was purified by using HPLC. Yield: 39%.

### C89676

Synthesis of target compound was carried out following the scheme given below:

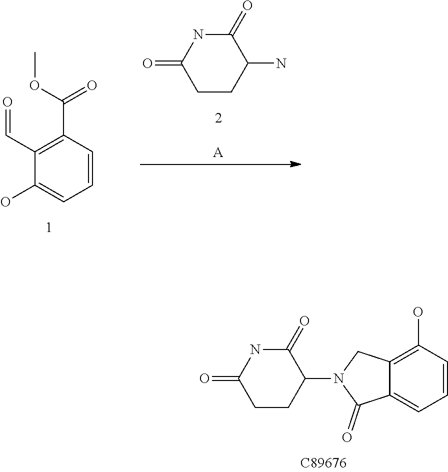 [SMILES: COC(=O)c1cccc(O)c1C=O ; NC1CCC(=O)NC1=O ; O=C1CCC(N2Cc3c(O)cccc3C2=O)C(=O)N1]

### General Procedure A:

Compound 1 (1 mmol), NaH(OAc)₃ (5 mmol), HOAc (1 mmol) and compound 2 (1.3 mmol) were dissolved in 5 ml of MeOH, the mixture was heated at 80° C. for 12 hours. TLC and LCMS control. The reaction mixture was cooled, then filtered and the solvent was evaporated. The crude residue was purified using HPLC. Yield 58%.

### C28558

Synthesis of target compound was carried out following the scheme given below:

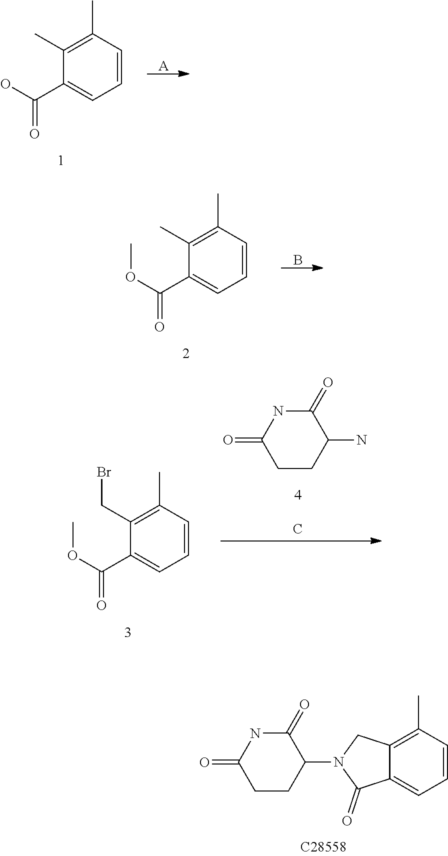

### Step A:

This compound was prepared according to General Procedure A for C24031. Yield: 98%.

### Step B:

This compound was prepared according to General Procedure B for C24031. Yield: 56%.

### Step C:

This compound was prepared according to General Procedure C for C24031. Yield: 67%.

C28577

Synthesis of target compound was carried out following the scheme given below:

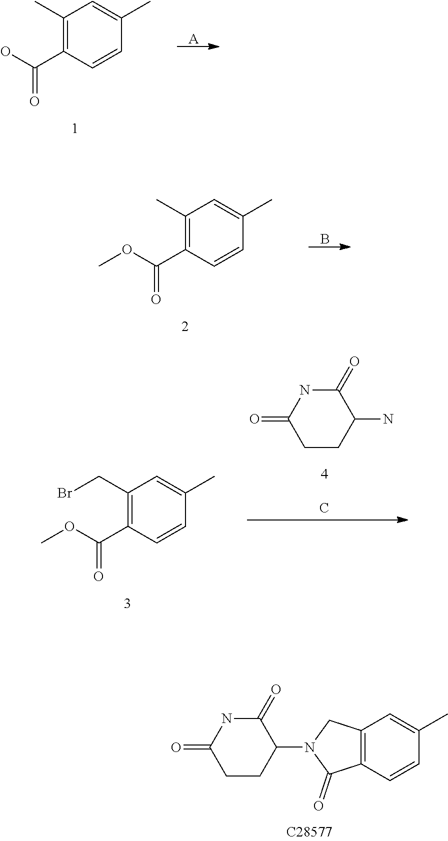

### Step A:

This compound was prepared according to General Procedure A for C24031. Yield:

98%.

### Step B:

This compound was prepared according to General Procedure B for C24031. Yield: 59%.

### Step C:

This compound was prepared according to General Procedure C for C24031. Yield: 81%.

C28620

Synthesis of target compound was carried out following the scheme given below:

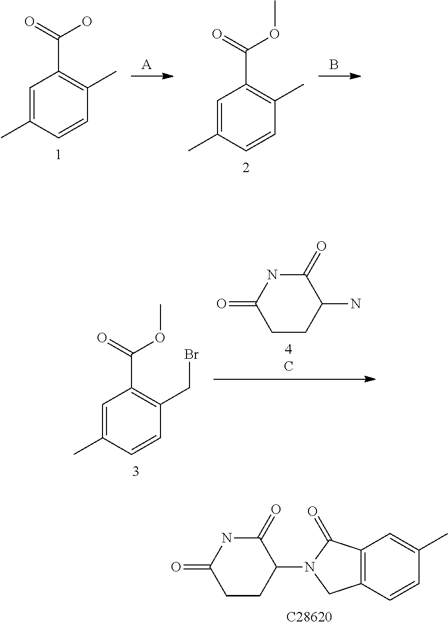

### Step A:

This compound was prepared according to General Procedure A for C24031. Yield: 95%.

### Step B:

This compound was prepared according to General Procedure B for C24031. Yield: 59%.

### Step C:

This compound was prepared according to General Procedure C for C24031. Yield: 72%.

### C28661

Synthesis of target compound was carried out following the scheme given below:

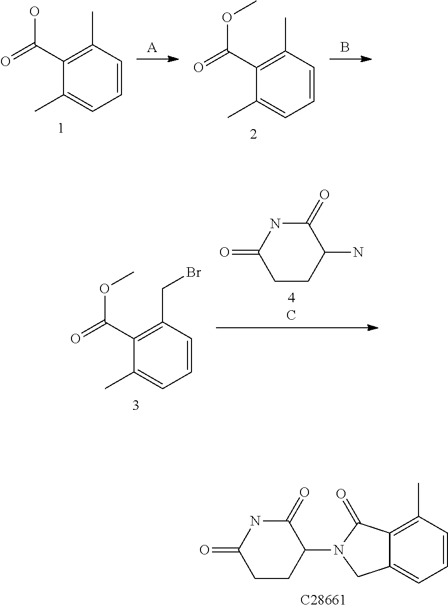

### Step A:

This compound was prepared according to General Procedure A for C24031. Yield: 95%.

### Step B:

This compound was prepared according to General Procedure B for C24031. Yield: 49%.

### Step C:

This compound was prepared according to General Procedure C for C24031. Yield: 64%.

### C28891

Synthesis of target compound was carried out following the scheme given below:

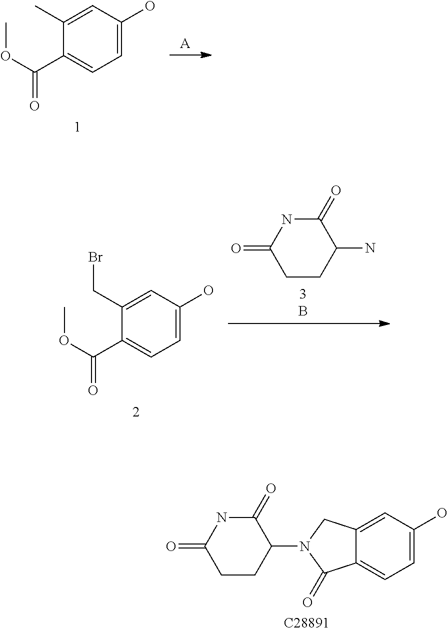

### Step A:

This compound was prepared according to General Procedure B for C24031. Yield: 69%.

### Step B:

This compound was prepared according to General Procedure C for C24031. Yield: 62%.

C28928

Synthesis of target compound was carried out following the scheme given below:

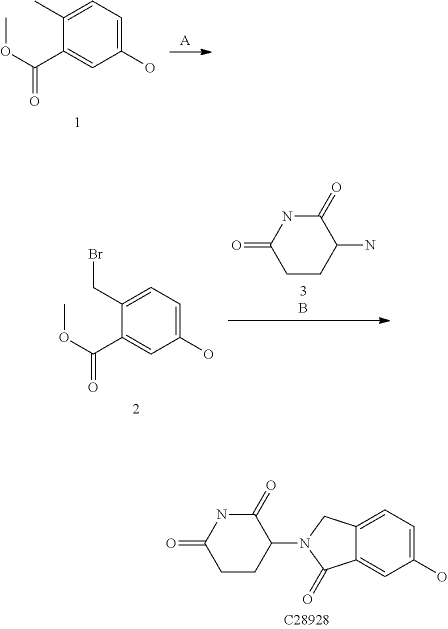

### Step A:

This compound was prepared according to General Procedure B for C24031. Yield: 37%.

### Step B:

This compound was prepared according to General Procedure C for C24031. Yield: 55%.

### C28973

Synthesis of target compound was carried out following the scheme given below:

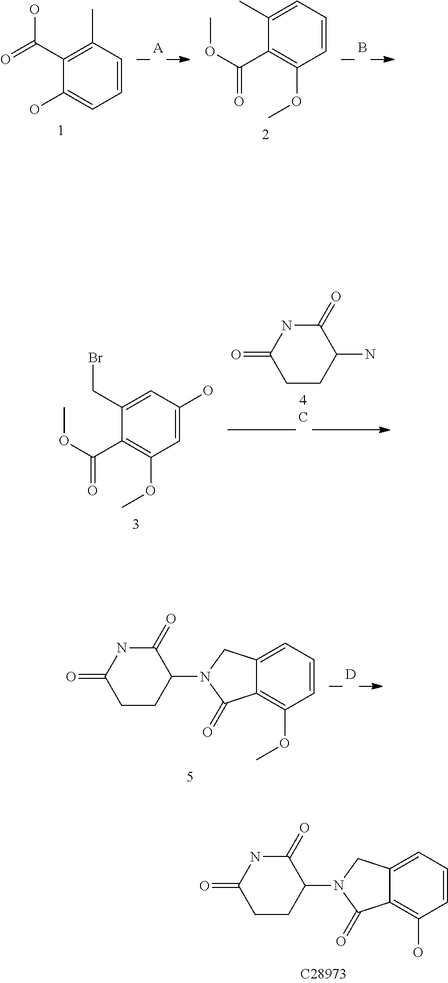

### Step A:

To a stirred solution of compound 1 (10 mmol) in acetone (10 mL) was added anhydrous powdered potassium carbonate (20 mmol). Dimethyl sulfate (12 mmol) was added in portions for about 10 min at room temperature. After the addition was complete, the solution was heated to reflux temperature on a water bath and maintained for 5 h. The solution was cooled to room temperature and then concentrated under reduced pressure. Distilled water (20 mL) was added to the reaction mixture, which was then extracted with ethyl acetate (50 mL). The organic layer was washed with distilled water (2×20 mL), dried over anhydrous sodium sulfate, and concentrated. Yield 86%.

### Step B:

This compound was prepared according to General Procedure B for C24031. Yield: 45%.

### Step C:

This compound was prepared according to General Procedure C for C24031. Yield: 74%.

### Step D:

To a solution of compound 5 (0.5 mmol) in dichloromethane (2.0 ml) was added TFA (2 mmol). The reaction stirred at room temperature for 0.5h. The reaction was poured into water and extracted (2×) with dichloromethane. The combined organic layers were dried over magnesium sulfate, filtered and concentrated. The crude residue was purified by HPLC. Yield: 26%.

### C47927

Synthesis of target compound was carried out following the scheme given below:

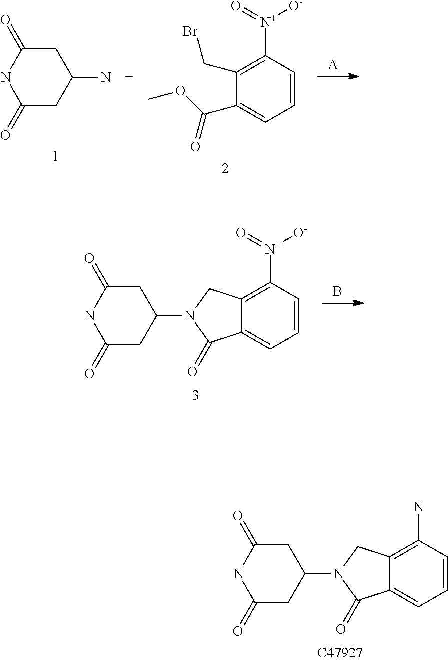

Step A:

This compound was prepared according to General Procedure C for C24031. Yield: 84%.

Step B:

To a solution of compound 4 (0.5 mmol) in methanol (2 mL) was added palladium carbon (5%, 10 mg), and the mixture was stirred under a hydrogen atmosphere at room temperature for 10 h. The reaction mixture was filtered through celite. The filtrate was concentrated under reduced pressure and after was purified by using HPLC. Yield: 79%.

### C47959

Synthesis of target compound was carried out following the scheme given below:

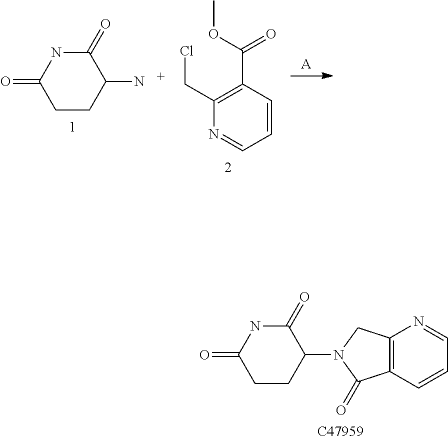

### General Procedure A:

Compound 1 (1 mmol), DIPEA (3.1 mmol) and compound 2 hydrochloride (1.3 mmol) were dissolved in 5 ml DMF, the mixture was heated at 80° C. for 18 hours (TLC and LCMS control). The reaction mixture was cooled, filtered and the solvent was evaporated. The crude residue was purified using HPLC. Yield: 29%.

### C48003

Synthesis of target compound was carried out following the scheme given below:

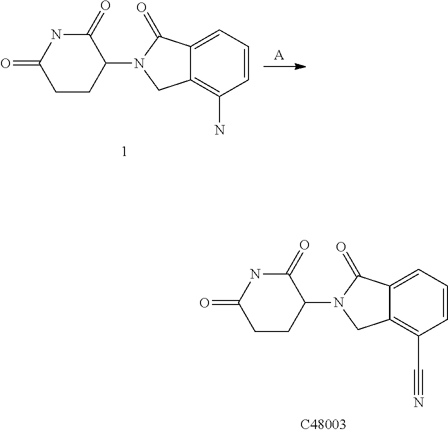

### General Procedure A:

Compound 1 (27 mmol) was treated with 50 ml of dilute (1:3) hydrochloric acid. The resulting suspension was cooled to 0-3° C. and a solution of NaNO₂ (108 mmol) in 50 ml of water was added during 20 min. The mixture was neutralized with NaHCO₃ and a suspension of CuCN (30 mmol) in 20 ml of toluene was added. The mixture was kept for 10-12h at room temperature and 100 ml of toluene was added. The toluene was evaporated, washed with water and recrystallization from mixture DMF/i-PrOH (⅓). Yield: 39%.

### C80392

Synthesis of target compound was carried out following the scheme given below:

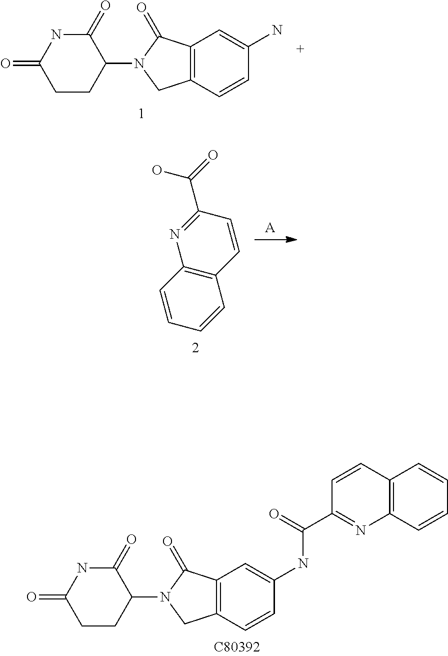

### General Procedure A:

Compound 1 (1 mmol) and 2 (1 mmol) were dissolved in 7 ml of dry DMF, DIPEA (3 mmol) and HATU (2 mmol) were added. The resulting mixture was stirred at 50° C. for 10-12h (LCMS control), cooled to room temperature, 15 ml of water and HOAc (5 mmol) were added. The mixture was extracted with EtOAc (3×25 ml), washed with brine (3×25 ml) and EtOAc was evaporated. The crude residue was purified using HPLC. Yield: 49%.

### C12584

Synthesis of target compound was carried out following the scheme given below:

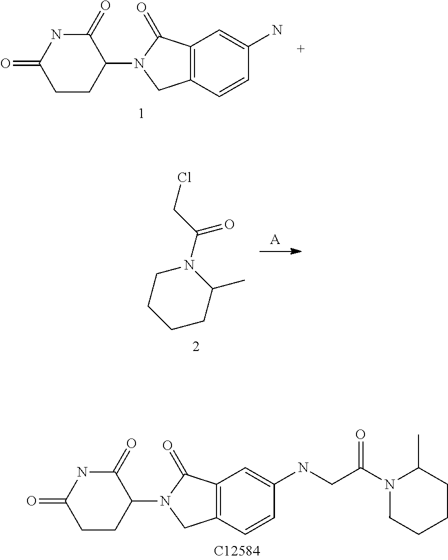

### General Procedure A:

Compound 1 (1 mmol) and 2 (1 mmol) were dissolved in 10 ml of dry DMF, DIPEA (3 mmol) was added. The resulting mixture was stirred at 90° C. for 3-5h (LCMS control), cooled to room temperature, 20 ml of water was added. The precipitate was filtered, washed with water (3×25 ml). The crude residue was purified using HPLC. Yield: 24%.

### C80370

Synthesis of target compound was carried out following the scheme given below:

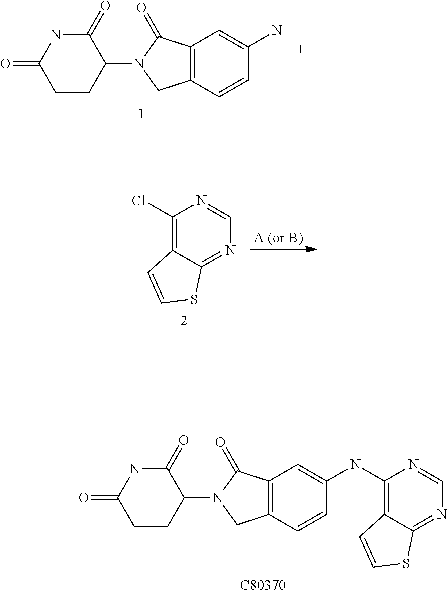

### General Procedure A:

Compound 1 (1 mmol) and 2 (1 mmol) were dissolved in 10 ml of dry DMF, DIPEA (2 mmol) was added. The resulting mixture was stirred at 80° C. for 10-12h (LCMS control), cooled to room temperature, 20 ml of water was added. The precipitate was filtered, washed with water (3×25 ml). The crude residue was purified using HPLC. Yield: 32%.

### General Procedure B:

Compound 1 (1 mmol) and 2 (1 mmol) were dissolved in 10 ml of dry HOAc. The resulting mixture was heated at 80° C. with stirring for 10-48h (LCMS control), cooled to room temperature, 20 ml of water was added. The precipitate was filtered, washed with water (3×25 ml). The crude residue was purified using HPLC. Yield: 38%.

### C48014

Synthesis of target compound was carried out following the scheme given below:

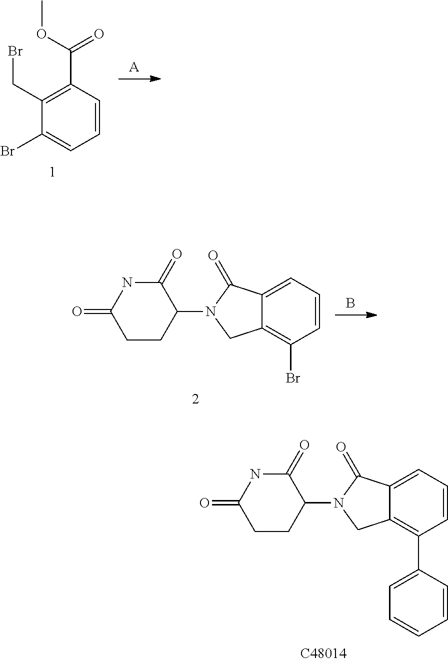

### Step A:

This compound was prepared according to General Procedure A for C47959. Yield: 82%.

### General Procedure B:

Compound 2 (1 mmol), PhB(OH)₂ (1.5 mmol), NaHCO₃ (2 mmol), Pd(PPh3)4 (0.1 mmol) and ×PhOS (0.05 mmol) were dissolved in 10 ml of dry Dioxane under Ar and 10 ml of water was added. The resulting mixture was heated at 80° C. with stirring for 10-12h (LCMS control), cooled to room temperature, solvent was evaporated. The crude residue was purified using HPLC. Yield: 24%.

### C95330

Synthesis of target compound was carried out following the scheme given below:

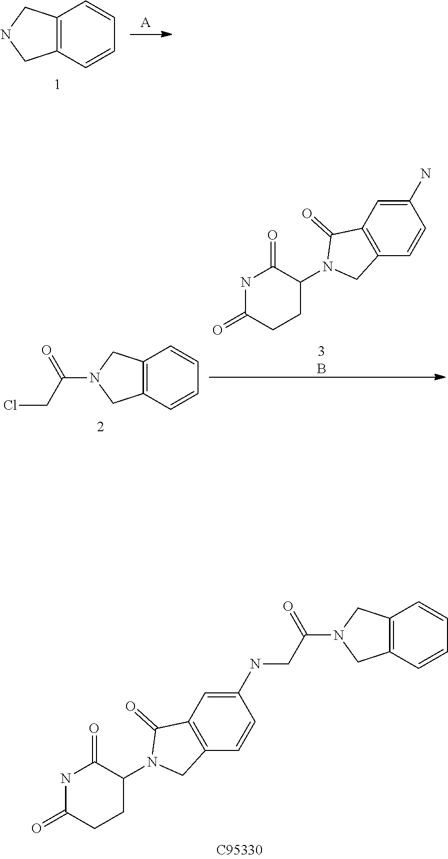

### General Procedure A:

Compound 1 (1 mmol) was dissolved in 20 ml of CH₂Cl₂, water (20 ml) and NaHCO₃ (2 mmol) were added. Chloroacetylchloride (1.1 mmol) was added dropwise during 15 min at 10-15° C. with stirring. The resulting mixture was stirred at room temperature for 3-5h. The organic phase was removed, washed with brine (3×25 ml), dried and solvent was evaporated. The crude residue was purified using FC. Yield: 88%.

### Step B:

This compound was prepared according to General Procedure A for C12584. Yield: 22%.

### C95338

Synthesis of target compound was carried out following the scheme given below:

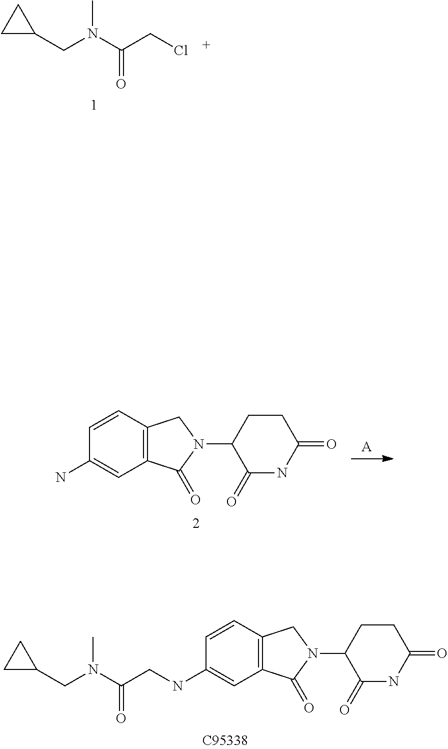

### Step A:

This compound was prepared according to General Procedure A for C12584. Yield: 29%.

### C80382

Synthesis of target compound was carried out following the scheme given below:

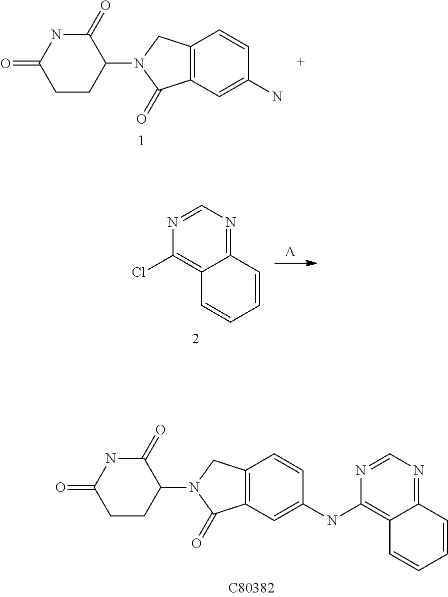

### Step A:

This compound was prepared according to General Procedure A for C80370. Yield: 31%.

### C80369

Synthesis of target compound was carried out following the scheme given below:

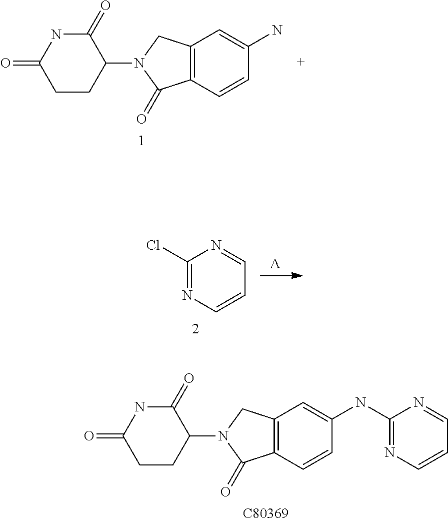

### Step A:

This compound was prepared according to General Procedure A for C80370. Yield: 41%.

### C47935

Synthesis of target compound was carried out following the scheme given below:

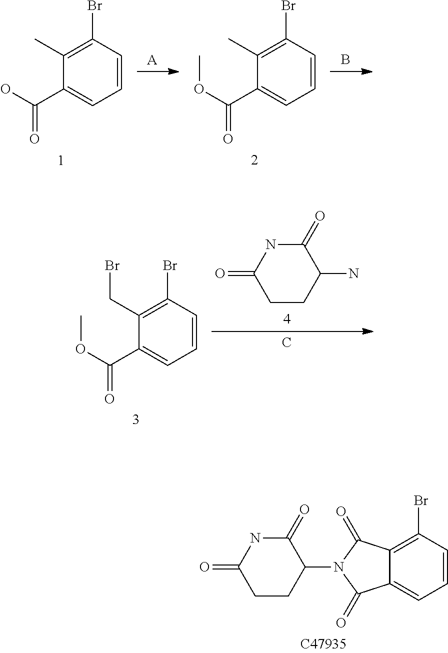

### General Procedure A:

Compound 1 (20 mmol) and K₂CO₃ (24 mmol) were suspended in DMF (20 mL). To the reaction mixture, was added iodomethane (24 mmol). The reaction was stirred at r.t. overnight, then diluted with water (150 mL). The solution was extracted with ethyl acetate (2×200 mL), organic phase was washed with brine (3×150 ml), dried with CaCl₂) and evaporated to give the methyl ester 2. Yield: 94%.

### General Procedure B:

To a solution of compound 2 (1.6 mmol) in CCl₄ (3 ml) was added AIBN (0.1 mmol) and NBS (2 mmol) during 15 min at 25° C. The reaction was heated for 12h at 60° C. and cooled to r.t. The solvent was then removed in vacuo. The crude residue was purified using LC. Yield: 64%.

### Step C:

This compound was prepared according to General Procedure A for C47959. Yield: 79%.

### C12583

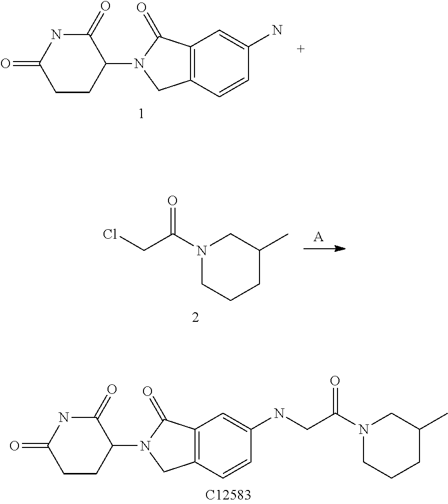

Synthesis of target compound was carried out following the scheme given below:

### Step A:

This compound was prepared according to General Procedure A for C12584. Yield: 32%.

### C80387

Synthesis of target compound was carried out following the scheme given below:

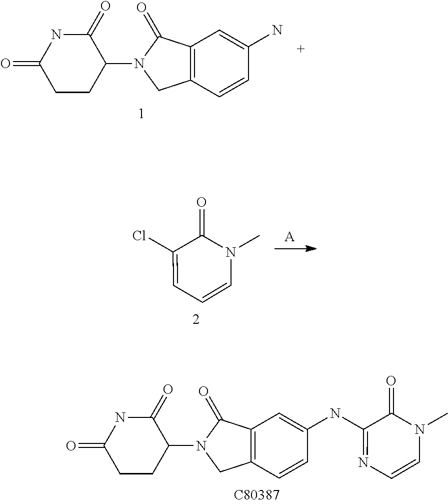

### Step A:

This compound was prepared according to General Procedure A for C80370. Yield: 44%.

### C80386

Synthesis of target compound was carried out following the scheme given below:

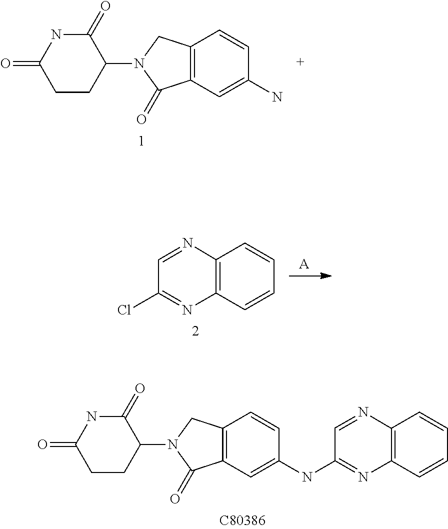

### Step A:

This compound was prepared according to General Procedure B for C80370. Yield: 46%.

### C67858

Synthesis of target compound was carried out following the scheme given below:

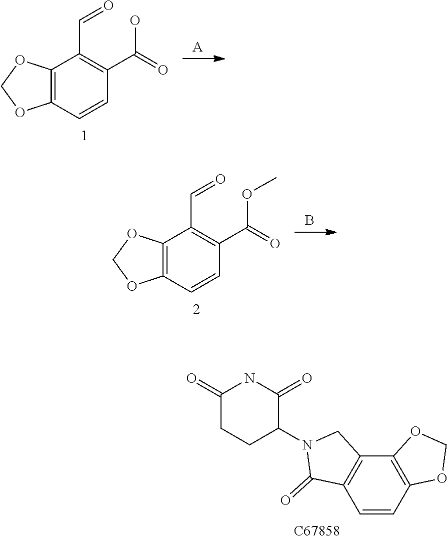

### Step A:

This compound was prepared according to General Procedure A for C47935. Yield: 69%.

### General Procedure B:

Compound 1 (1 mmol), NaH(OAc)₃ (5 mmol), HOAc (1 mmol) and compound 2 (1.3 mmol) were dissolved in 5 ml of MeOH, the mixture was heated at 80° C. for 12 hours. TLC and LCMS control. The reaction mixture was cooled, then filtered and the solvent was evaporated. The crude residue was purified using HPLC. Yield 48%.

### C98103

Synthesis of target compound was carried out following the scheme given below:

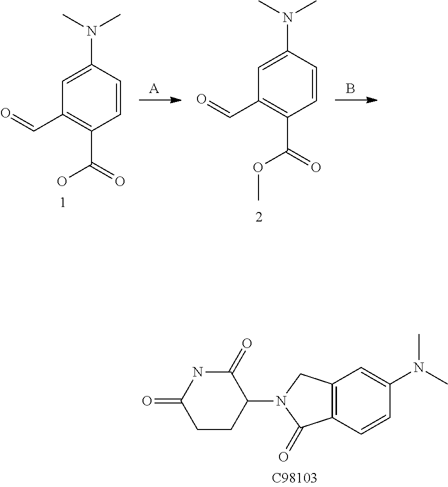

### Step A:

This compound was prepared according to General Procedure A for C47935. Yield: 71%.

### Step B:

This compound was prepared according to General Procedure B for C67858. Yield: 43%.

### C80383

Synthesis of target compound was carried out following the scheme given below:

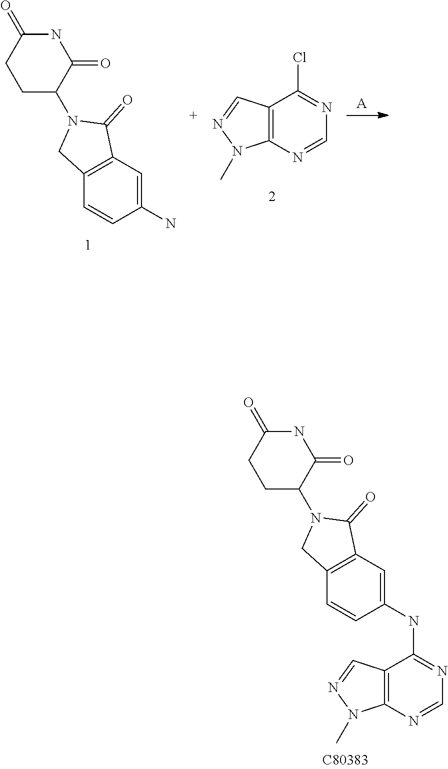

### Step A:

This compound was prepared according to General Procedure A for C80370. Yield: 39%.

### C80391

Synthesis of target compound was carried out following the scheme given below:

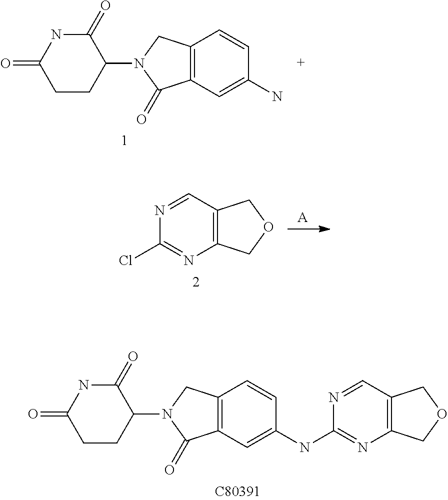

### Step A:

This compound was prepared according to General Procedure A for C80370. Yield: 49%.

### C48016

Synthesis of target compound was carried out following the scheme given below:

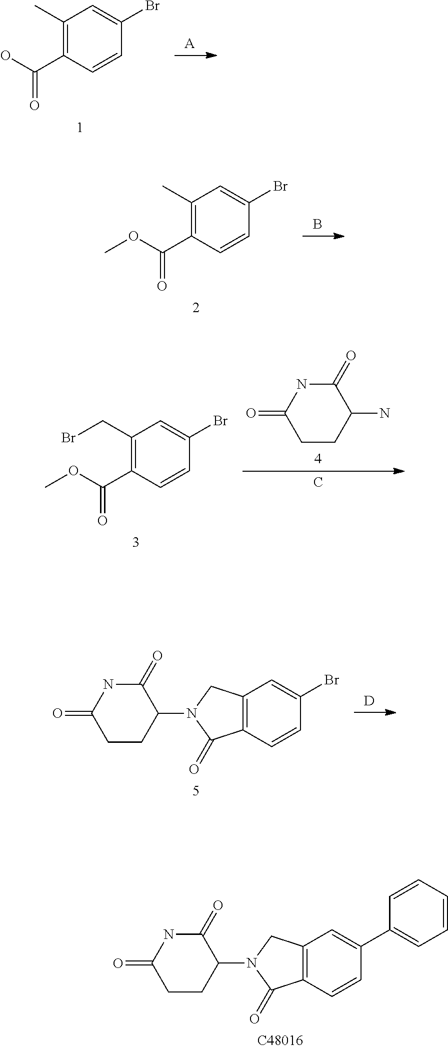

### Step B:

This compound was prepared according to General Procedure B for C47935. Yield: 64%.

### Step C:

This compound was prepared according to General Procedure A for C47959. Yield: 79%.

### Step D:

This compound was prepared according to General Procedure A for C48014. Yield: 33%.

### C05955

Synthesis of target compound was carried out following the scheme given below:

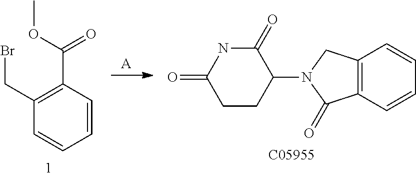

### Step A:

This compound was prepared according to General Procedure A for C47959. Yield: 91%.

### C80384

Synthesis of target compound was carried out following the scheme given below:

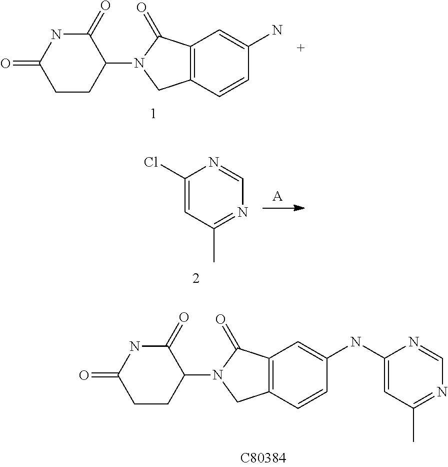

### Step A

This compound was prepared according to General Procedure A for C80370. Yield:

52%.

### C51383

Synthesis of target compound was carried out following the scheme given below:

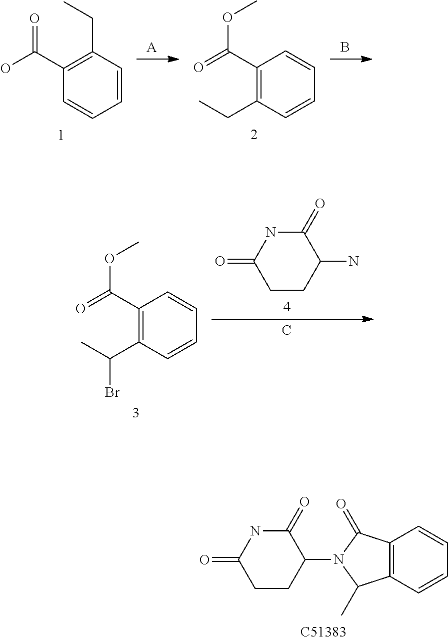

### Step A:

This compound was prepared according to General Procedure A for C47935. Yield: 89%.

### Step B:

This compound was prepared according to General Procedure B for C47935. Yield: 25%.

### Step C:

This compound was prepared according to General Procedure A for C47959. Yield: 22%.

### C84965

Synthesis of target compound was carried out following the scheme given below:

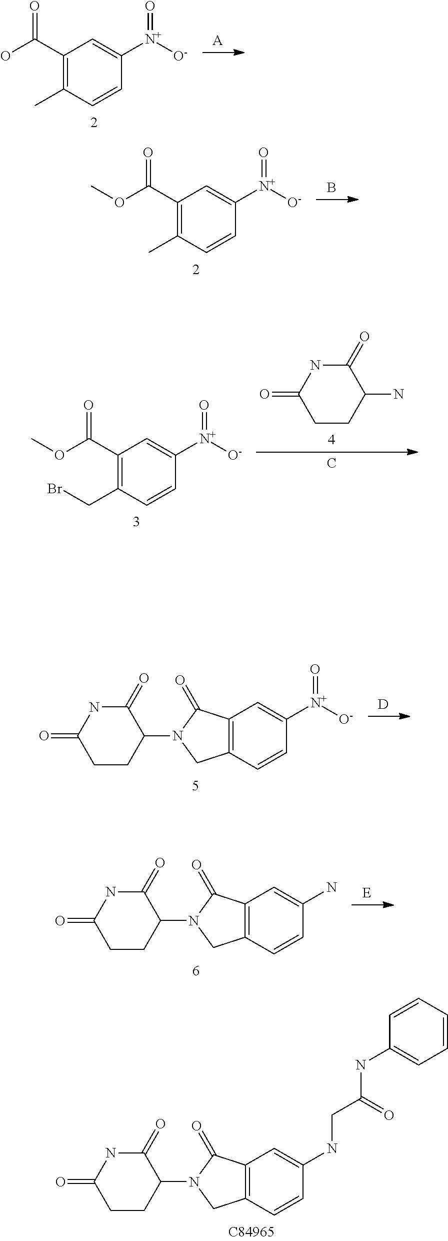

### Step A:

This compound was prepared according to General Procedure A for C47935. Yield: 93%.

### Step B:

This compound was prepared according to General Procedure B for C47935. Yield: 74%.

### Step C:

This compound was prepared according to General Procedure A for C47959. Yield: 82%.

### General Procedure D:

To a solution of compound 5 (0.5 mmol) in methanol (2 mL) was added palladium carbon (5%, 10 mg), and the mixture was stirred under a hydrogen atmosphere at room temperature for 10 h. The reaction mixture was filtered through celite. The filtrate was concentrated under reduced pressure and after was purified by using HPLC. Yield: 39%.

### Step E:

This compound was prepared according to General Procedure A for C12584. Yield: 34%.

### C12595

Synthesis of target compound was carried out following the scheme given below:

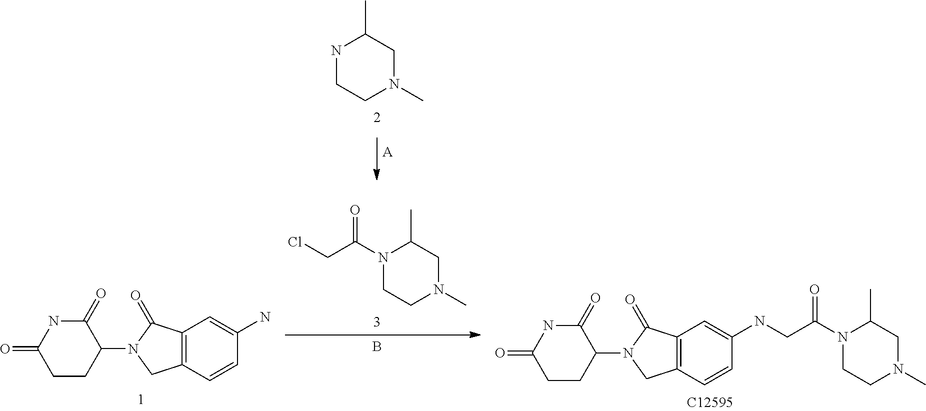

### Step A:

This compound was prepared according to General Procedure A for C95330. Yield: 82%.

### Step B:

This compound was prepared according to General Procedure A for C12584. Yield: 22%.

### C96622

Synthesis of target compound was carried out following the scheme given below:

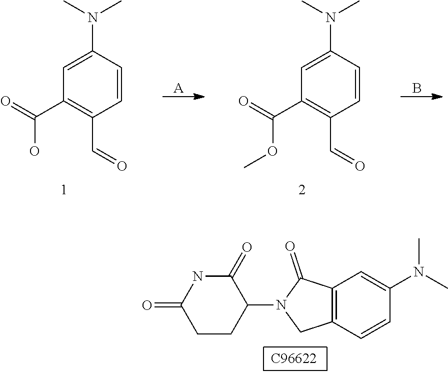

### Step A:

This compound was prepared according to General Procedure A for C47935. Yield: 88%.

### Step B:

This compound was prepared according to General Procedure B for C67858. Yield: 57%.

### C22622

Synthesis of target compound was carried out following the scheme given below:

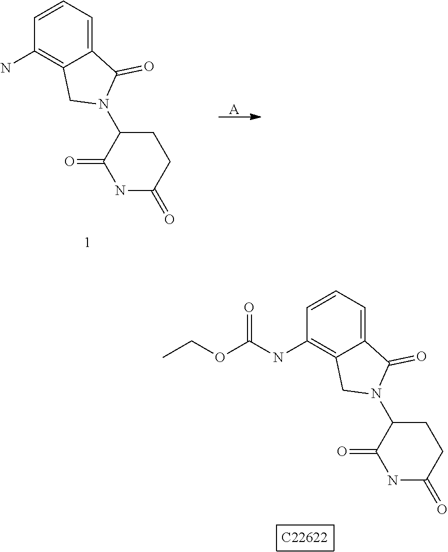

### Step A: C22622

This compound was prepared according to General Procedure A for C95330. Yield: 53%.

### C48018

Synthesis of target compound was carried out following the scheme given below:

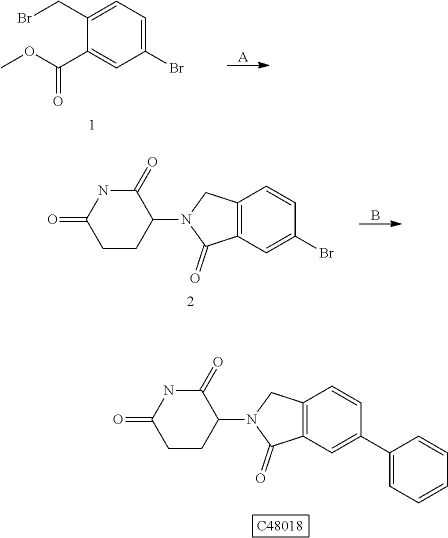

### Step A:

This compound was prepared according to General Procedure A for C47959. Yield: 79%.

### Step B:

This compound was prepared according to General Procedure A for C48014. Yield: 28%.

### C84964

Synthesis of target compound was carried out following the scheme given below:

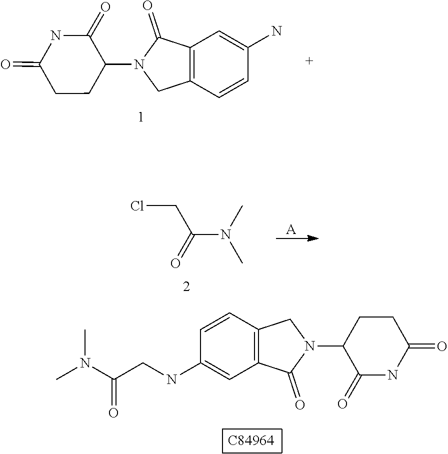

### Step A:

This compound was prepared according to General Procedure A for C12584. Yield: 39%.

### C29137

Synthesis of target compound was carried out following the scheme given below:

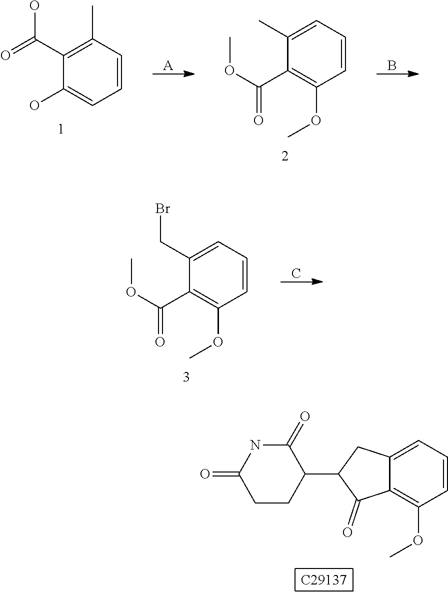

### Step A:

This compound was prepared according to General Procedure A for C47935. Yield: 97%.

### Step B:

This compound was prepared according to General Procedure B for C47935. Yield: 64%.

### Step C:

This compound was prepared according to General Procedure A for C47959. Yield: 62%.

### C12586

Synthesis of target compound was carried out following the scheme given below:

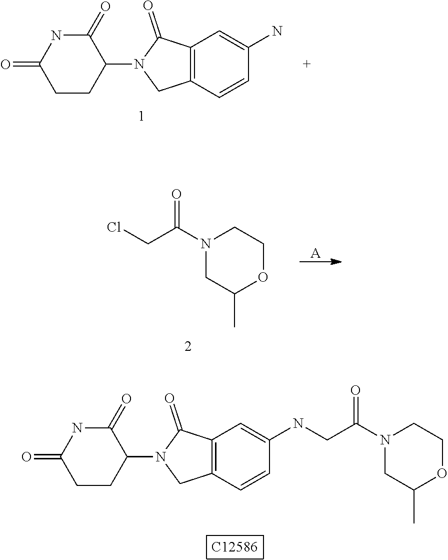

### Step A:

This compound was prepared according to General Procedure A for C12584. Yield: 42%.

### C35856

Synthesis of target compound was carried out following the scheme given below:

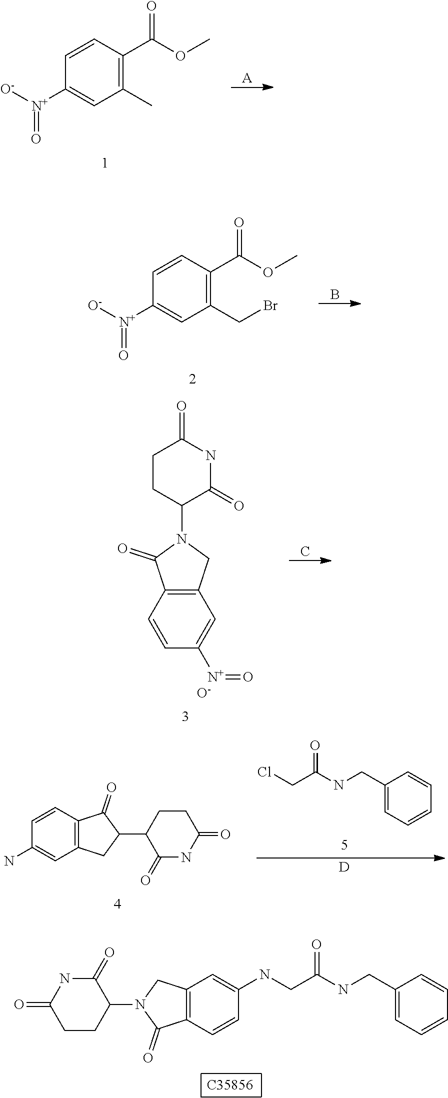

### Step A:

This compound was prepared according to General Procedure B for C47935. Yield: 91%.

### Step B:

This compound was prepared according to General Procedure A for C47959. Yield: 82%.

### Step C:

This compound was prepared according to General Procedure D for C84965. Yield: 8100.

### Step D:

This compound was prepared according to General Procedure A for C12584. Yield: 220%.

### C35751

Synthesis of target compound was carried out following the scheme given below:

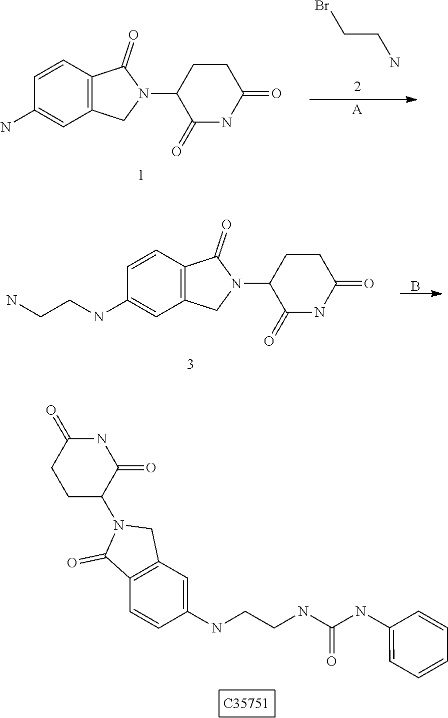

### General Procedure A:

Compound 1 (20 mmol) and K₂CO₃ (24 mmol) were suspended in DMF (20 mL). Compound 2 hydrobromide (100 mmol) was added and the reaction mixture was heated at 90° C. with stirring for 10h. The suspension was filtered, washed with DMF (3×10 ml) and solvent was evaporated to give the compound 3. The crude residue was purified using LC. Yield: 9%.

### General Procedure B:

Compound 3 (1 mmol) was dissolved in acetonitrile (10 mL). PhNCO (1.1 mmol) and 1 drop of NEt₃ were added and the reaction mixture was heated at 60-70° C. with stirring for 3-5h. The suspension was cooled, filtered, washed with acetonitrile (3×10 mL) and solvent was evaporated to give the compound 3. The crude residue was purified using LC. Yield: 46%.

### C48020

Synthesis of target compound was carried out following the scheme given below:

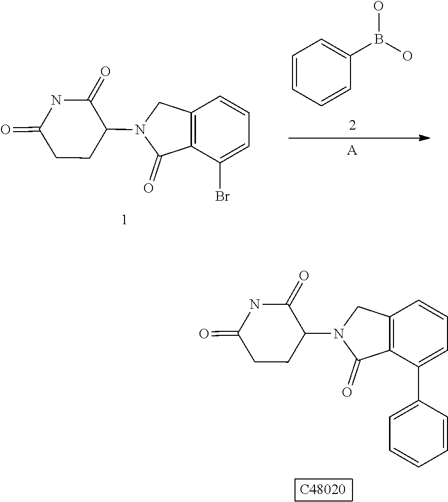

### Step A:

This compound was prepared according to General Procedure A for C48014. Yield: 25%.

### C68126

Synthesis of target compound was carried out following the scheme given below:

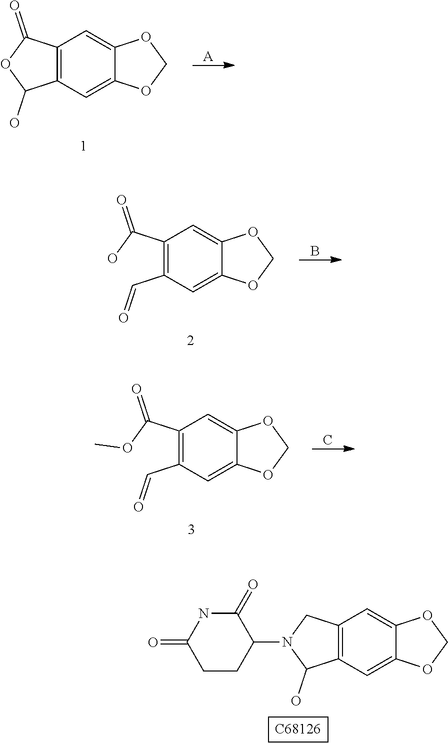

### General Procedure A:

Compound 1 (1 mmol) and K₂C03 (2 mmol) were mixed with water (20 mL). The reaction mixture was heated at 80° C. with stirring overnight, then water was evaporated and Na salt of the product was treatment with 50 ml of mixture EtOAc:THF (4:1), filtered, washed with mixture EtOAc:THF (4:1) and dried. Yield: 44%.

### Step B:

This compound was prepared according to General Procedure A for C47935. Yield: 69%.

### Step C:

This compound was prepared according to General Procedure B for C67858. Yield: 38%.

### C95333

Synthesis of target compound was carried out following the scheme given below:

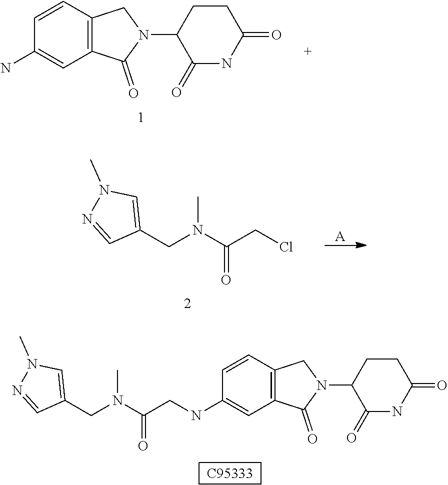

### Step A:

This compound was prepared according to General Procedure A for C12584. Yield: 42%.

### C84966

Synthesis of target compound was carried out following the scheme given below:

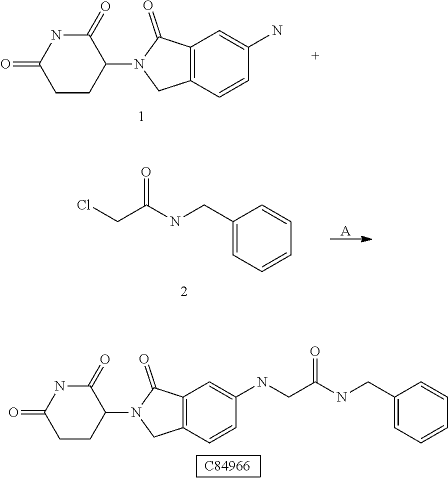

### Step A:

This compound was prepared according to General Procedure A for C12584. Yield: 37%.

### C66979

Synthesis of target compound was carried out following the scheme given below:

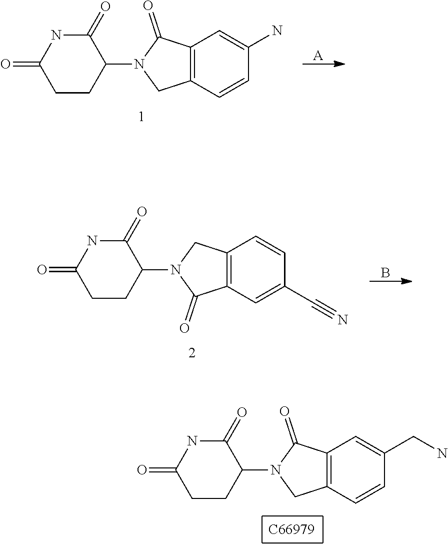

### Step A:

This compound was prepared according to General Procedure A for C48003. Yield: 59%.

### Step B:

To a solution of compound 4 (0.5 mmol) in THF (2 mL) was added palladium carbon (5%, 10 mg), and the mixture was stirred under a hydrogen atmosphere at 80° C. for 20 h. The reaction mixture was filtered through celite. The filtrate was concentrated under reduced pressure and after was purified by using HPLC. Yield: 14%.

### C80389

Synthesis of target compound was carried out following the scheme given below:

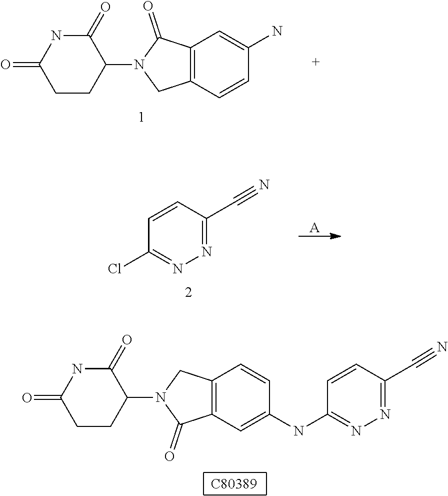

### Step A:

This compound was prepared according to General Procedure B for C80370. Yield: 25%.

### C35833

Synthesis of target compound was carried out following the scheme given below:

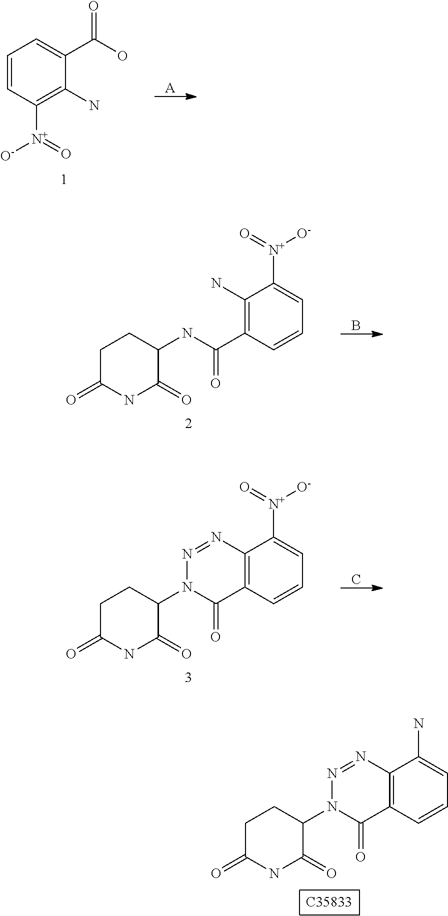

### Step A:

This compound was prepared according to General Procedure B for C47935. Yield: 96%.

### General Procedure B:

Compound 2 (1 mmol) was treated with 10 ml of dilute (1:3) hydrochloric acid and 10 ml of dioxane was added. The resulting suspension was cooled to 10° C. and a solution of NaNO₂ (2 mmol) in 10 ml of water was added during 2-3 min. The mixture was stirred at r.t. for 10h, neutralized with NaHCO₃ and a precipitate was filtered, washed with water (3×25 ml) and recrystallization from mixture DMF/i-PrOH (1/1). Yield: 29%.

### Step C:

This compound was prepared according to General Procedure D for C84965. Yield: 32%.

### C59904

Synthesis of target compound was carried out following the scheme given below:

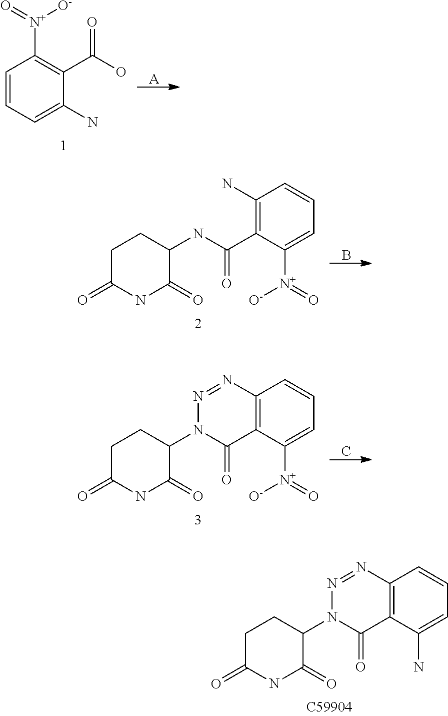

### Step A:

This compound was prepared according to General Procedure B for C47935. Yield: 95%.

### Step A:

This compound was prepared according to General Procedure B for C35833. Yield: 23%.

### Step C:

This compound was prepared according to General Procedure D for C84965. Yield: 37%.

### C80390

Synthesis of target compound was carried out following the scheme given below:

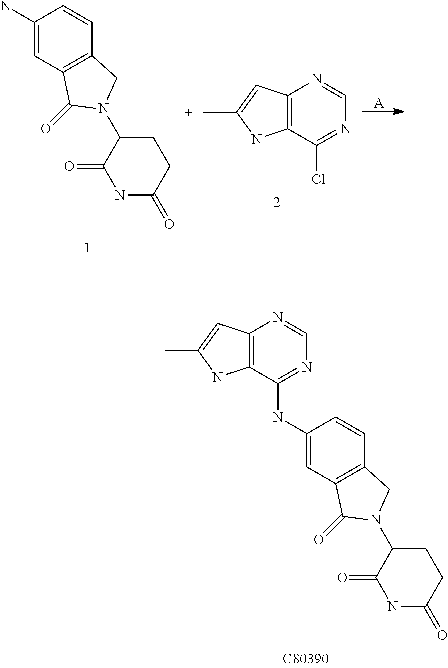

### Step A:

This compound was prepared according to General Procedure B for C80370. Yield: 21%.

### C35754

Synthesis of target compound was carried out following the scheme given below:

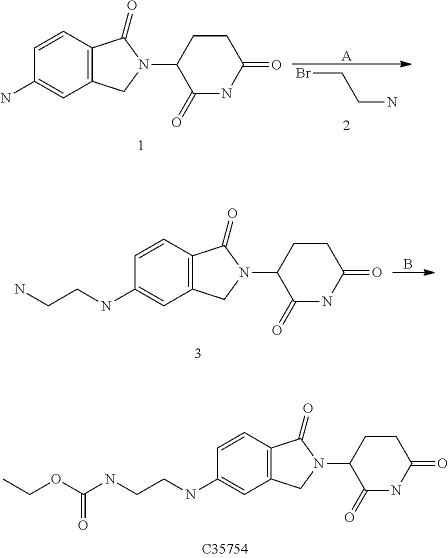

### Step A:

This compound was prepared according to General Procedure A for C35751. Yield: 9%.

### Step B:

This compound was prepared according to General Procedure A for C95330. Yield: 51%.

### C49708

Synthesis of target compound was carried out following the scheme given below:

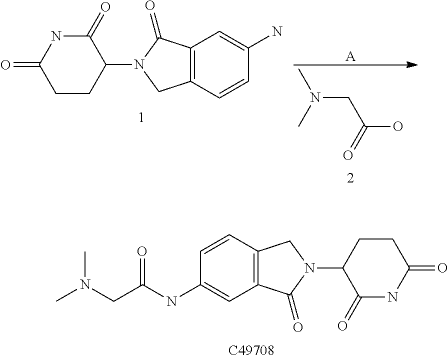

### Step A:

This compound was prepared according to General Procedure A for C80392. Yield: 57%.

### C80380

Synthesis of target compound was carried out following the scheme given below:

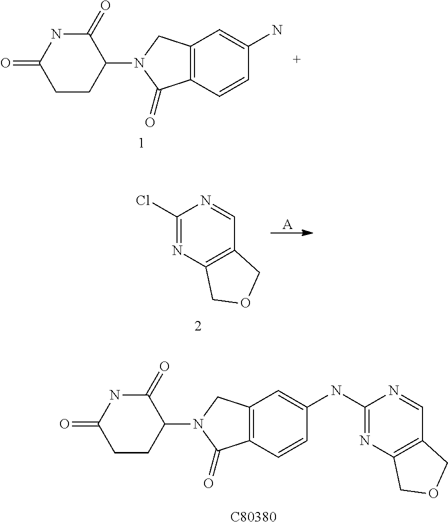

### Step A:

This compound was prepared according to General Procedure A for C80370. Yield: 36%.

### C35797

Synthesis of target compound was carried out following the scheme given below:

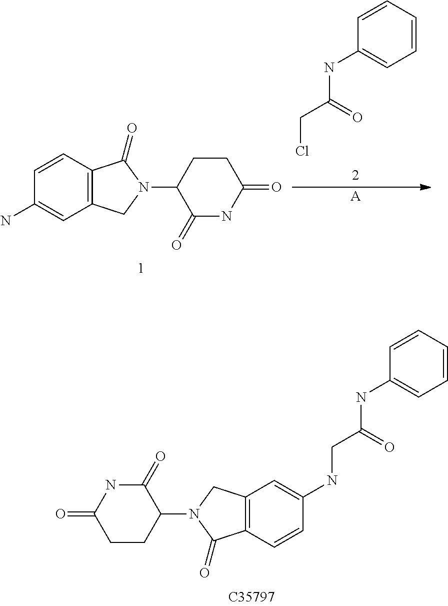

### Step 0:

This compound was prepared according to General Procedure A for C12584. Yield: 39%.

### C80395

Synthesis of target compound was carried out following the scheme given below:

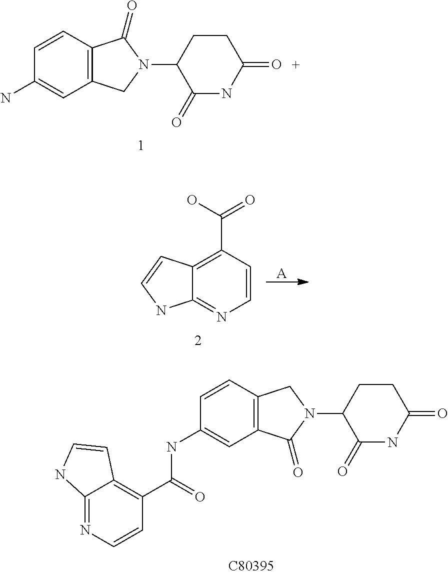

### Step A:

This compound was prepared according to General Procedure A for C80392. Yield: 32%.

### C80373

Synthesis of target compound was carried out following the scheme given below:

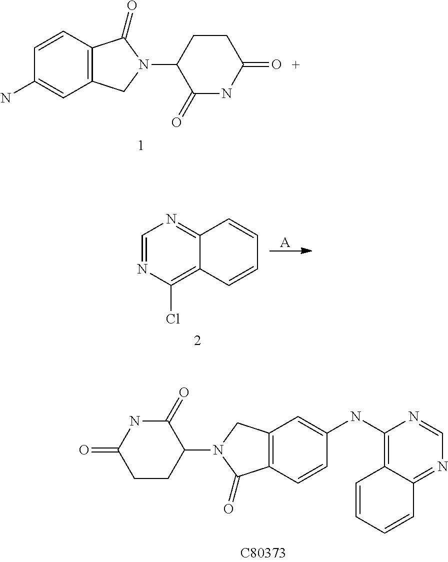

### Step A:

This compound was prepared according to General Procedure A for C80370. Yield: 71%.

### C45748

Synthesis of target compound was carried out following the scheme given below:

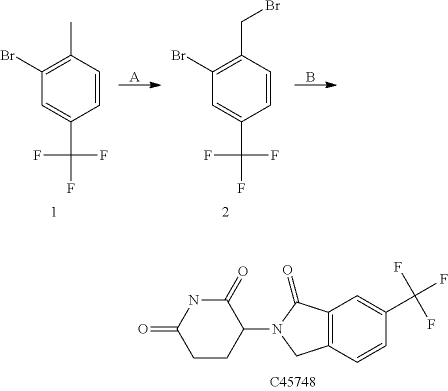

### Step A:

This compound was prepared according to General Procedure B for C47935. Yield: 66%.

### Step B:

This compound was prepared according to General Procedure A for C47959. Yield: 65%.

### C80379

Synthesis of target compound was carried out following the scheme given below:

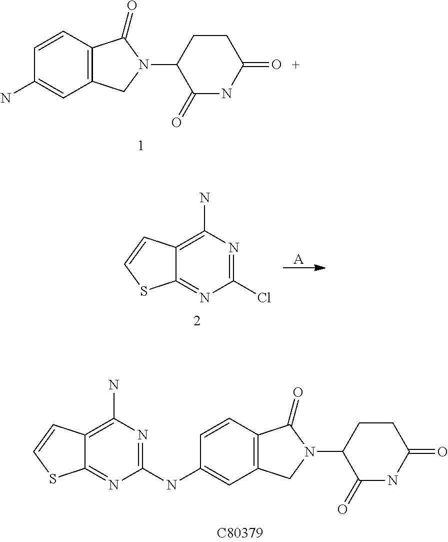

### Step A:

This compound was prepared according to General Procedure B for C80370. Yield: 26%.

### C47933

Synthesis of target compound was carried out following the scheme given below:

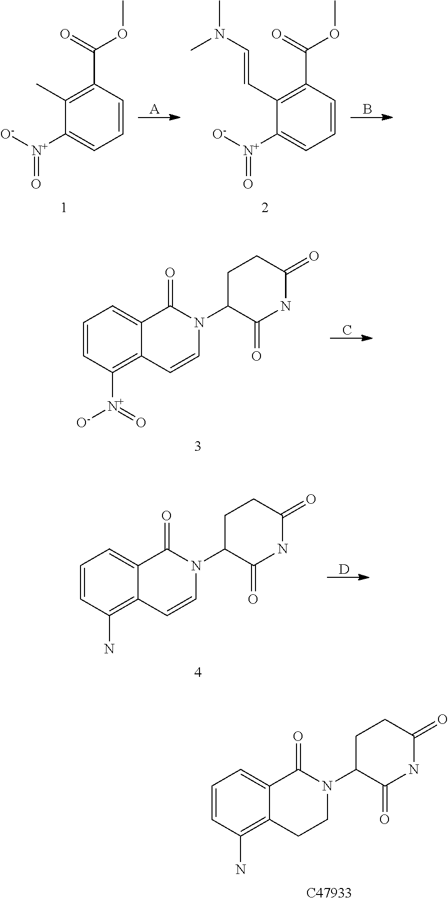

### General Procedure A:

Compound 1 (1 mmol) was dissolved in 10 ml of dry DMF, DMFDMA (1.2 mmol) was added and the reaction mixture was stirred at 50° C. overnight, then diluted with water (50 mL). The solution was extracted with ethyl acetate (2×50 mL), organic phase was washed with brine (3×150 ml), dried with CaCl₂) and evaporated to give the methyl ester 2. Yield: 88%.

### General Procedure B:

Compound 2 (1 mmol) was dissolved in 10 ml of dry DMF, Glutarimide (1.2 mmol) was added and the reaction mixture was stirred at 90° C. 10h, then solvent was evaporated to give the compound 3. The crude residue was recrystallization with use mixture DMF:iPrOH (1:2). Yield: 76%.

### Step C:

This compound was prepared according to General Procedure D for C84965. Yield: 53%.

### Step D:

To a suspension of compound 4 (1 mmol) in dioxane (20 mL) was added palladium carbon (5%, 15 mg), and the mixture was stirred under a hydrogen atmosphere at 90° C. for 15 h. The reaction mixture was filtered through celite. The filtrate was concentrated under reduced pressure and after was purified by using HPLC. Yield: 65%.

### C22548

Synthesis of target compound was carried out following the scheme given below:

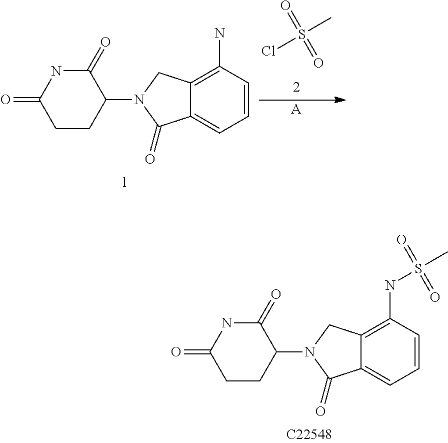

### Step A:

This compound was prepared according to General Procedure A for C95330. Yield: 37%.

### C84963

Synthesis of target compound was carried out following the scheme given below:

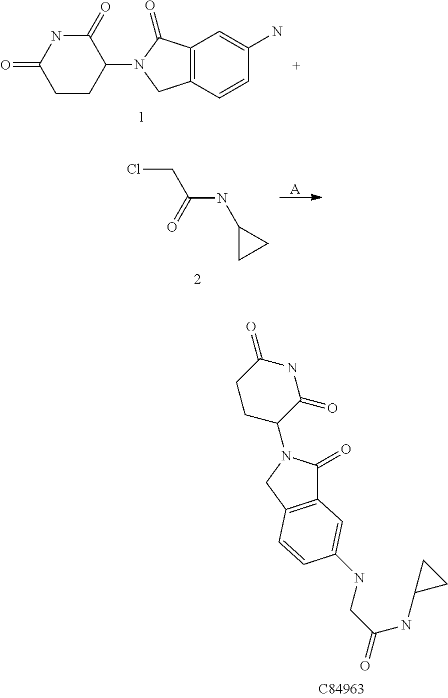

### Step A:

This compound was prepared according to General Procedure A for C12584. Yield: 31%.

### C22586

Synthesis of target compound was carried out following the scheme given below:

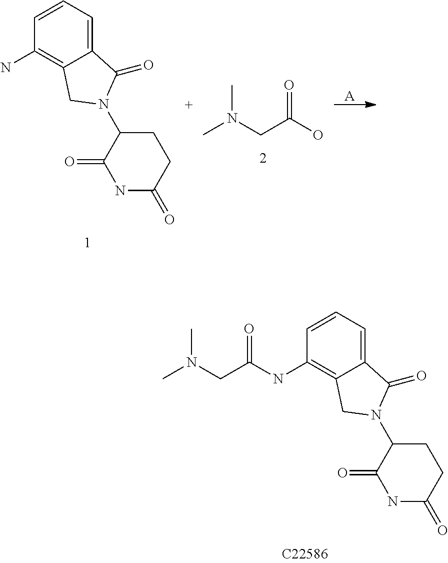

### Step A:

This compound was prepared according to General Procedure A for C80392. Yield: 58%.

### C84971

Synthesis of target compound was carried out following the scheme given below:

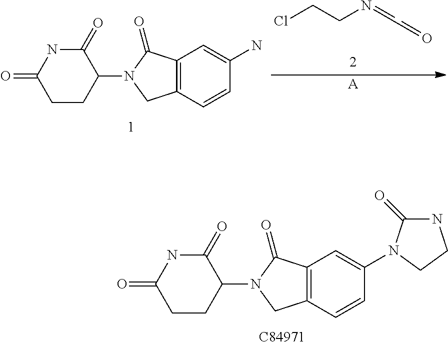

### Step A:

This compound was prepared according to General Procedure B for C35751. Yield: 48%.

### C48007

Synthesis of target compound was carried out following the scheme given below:

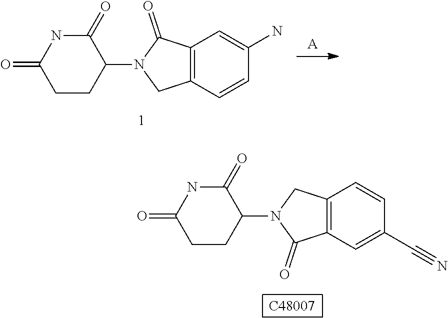

### Step A:

This compound was prepared according to General Procedure A for C48003. Yield: 69%.

### C99884

Synthesis of target compound was carried out following the scheme given below:

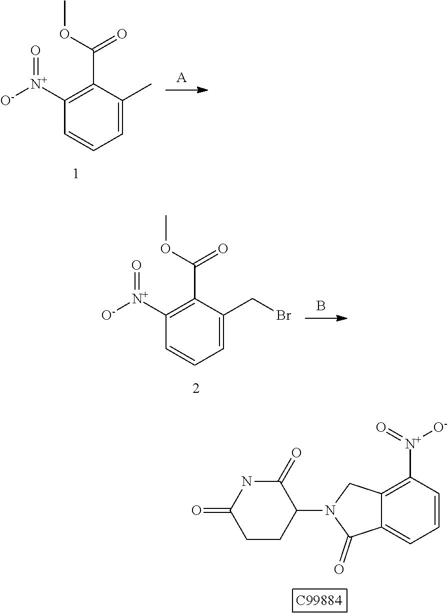

### Step A:

This compound was prepared according to General Procedure B for C47935. Yield: 79%.

### Step B:

This compound was prepared according to General Procedure A for C47959. Yield: 82%.

### C29330

Synthesis of target compound was carried out following the scheme given below:

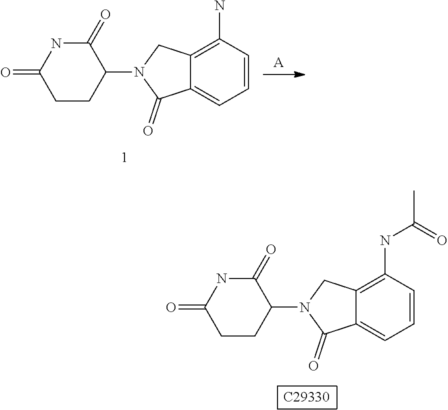

### Step A:

This compound was prepared according to General Procedure A for C80392. Yield: 87%.

### C84970

Synthesis of target compound was carried out following the scheme given below:

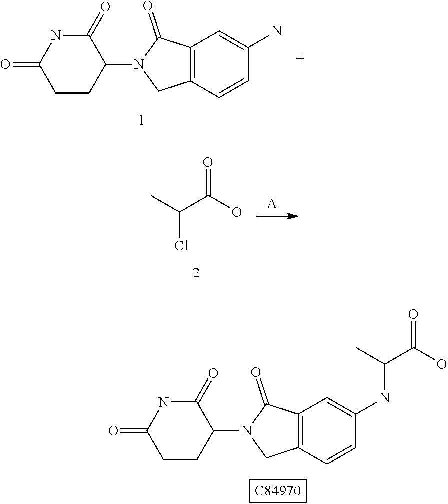

### Step A:

This compound was prepared according to General Procedure A for C12584. Yield: 11%.

In this case we used additional amount of HOAc (3 eq) before separation of the product from reaction mixture.

### C29361

Synthesis of target compound was carried out following the scheme given below:

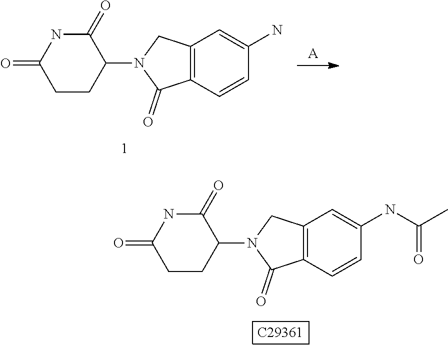

### Step A:

This compound was prepared according to General Procedure A for C80392. Yield: 78%.

### C22564

Synthesis of target compound was carried out following the scheme given below:

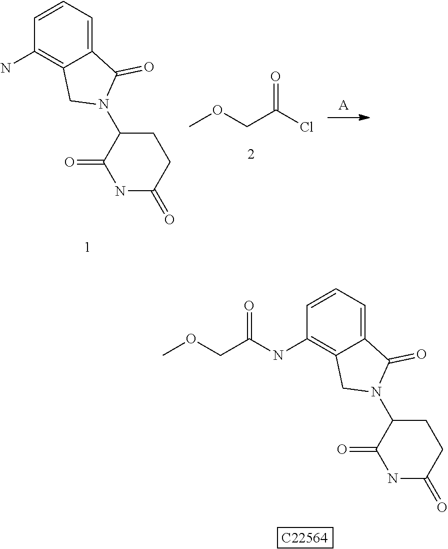

### Step A:

This compound was prepared according to General Procedure A for C95330. Yield: 45%.

### C48005

Synthesis of target compound was carried out following the scheme given below:

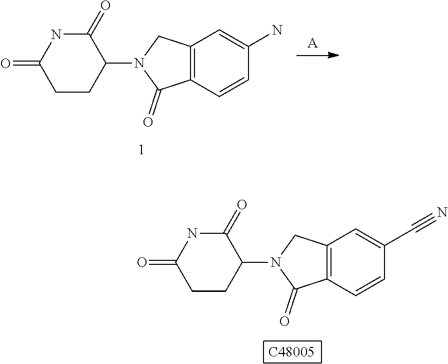

### Step A:

This compound was prepared according to General Procedure A for C48003. Yield: 59%.

### C80375

Synthesis of target compound was carried out following the scheme given below:

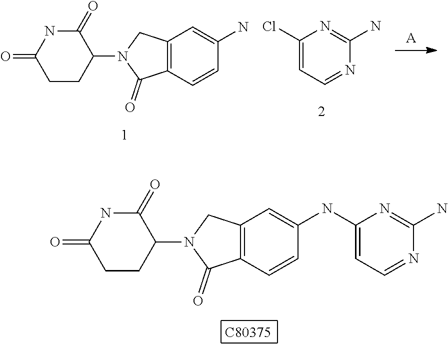

### Step A:

This compound was prepared according to General Procedure A for C80370. Yield: 19%.

### C48009

Synthesis of target compound was carried out following the scheme given below:

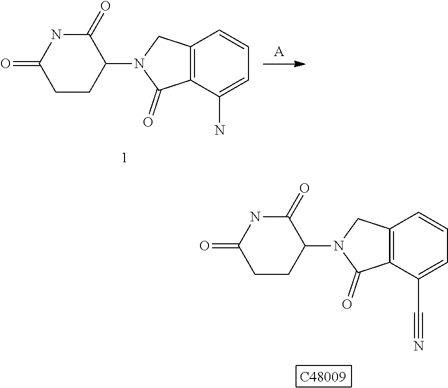

### Step A:

This compound was prepared according to General Procedure A for C48003. Yield: 22%.

### C80378

Synthesis of target compound was carried out following the scheme given below:

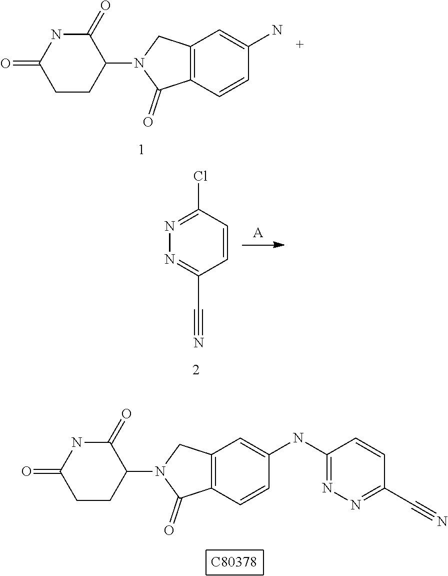

### Step A:

This compound was prepared according to General Procedure A for C80370. Yield: 31%.

### C21223

Synthesis of target compound was carried out following the scheme given below:

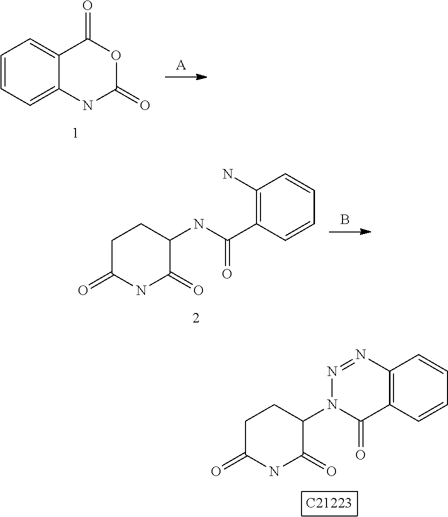

### General Procedure A:

Compound 1 (1.1 mmol) and Glutarimide (1 mmol) were dissolved in dioxane (10 mL) and NEt₃ (1.3 mmol) was added. The reaction mixture was heated at 80° C. with stirring at overnight, then diluted with water (150 mL). The precipitate was filtered, washed with water (3×25 ml) and recrystallization from mixture iPrOH:Water (4:1). Yield: 90%.

### Step B:

This compound was prepared according to General Procedure B for C35833. Yield: 42%.

### C13247

Synthesis of target compound was carried out following the scheme given below:

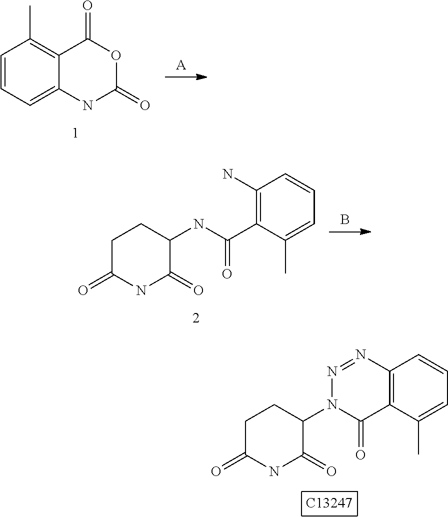

### Step A:

This compound was prepared according to General Procedure A for C21223. Yield: 73%.

### Step B:

This compound was prepared according to General Procedure B for C35833. Yield: 33%.

### C35811

Synthesis of target compound was carried out following the scheme given below:

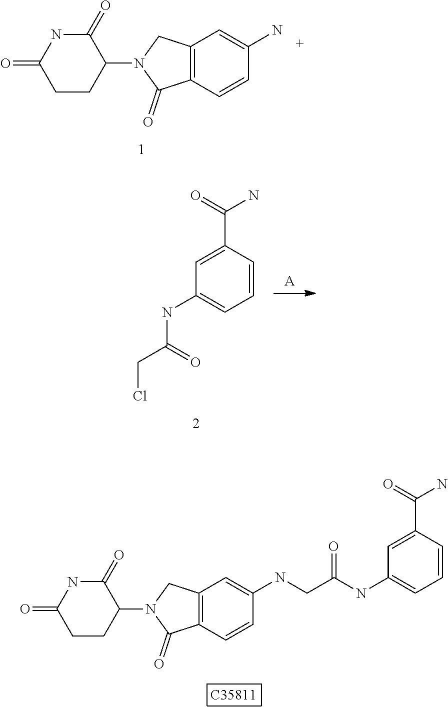

### Step B:

This compound was prepared according to General Procedure A for C12584. Yield: 21%.

### C80396

Synthesis of target compound was carried out following the scheme given below:

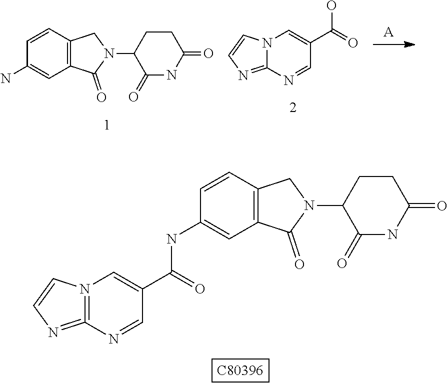

### Step A:

This compound was prepared according to General Procedure A for C80392. Yield: 47%.

### C64324

Synthesis of target compound was carried out following the scheme given below:

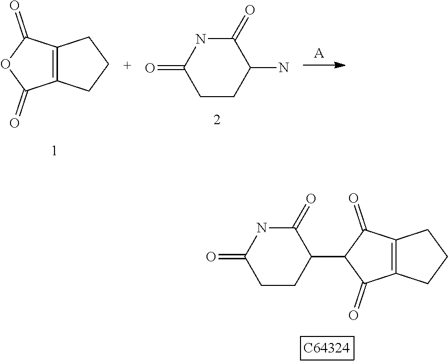

### General Procedure A:

Compound 1 (1 mmol), hydrochloride 2 (1 mmol) and dry NaOAc (5 mmol) were dissolved in 10 ml of dry HOAc, the mixture was heated at 100° C. for 10-24 hours (LCMS control). The reaction mixture was cooled, filtered and the solvent was evaporated. The crude residue was purified using HPLC. Yield: 29%.

### C84967

Synthesis of target compound was carried out following the scheme given below:

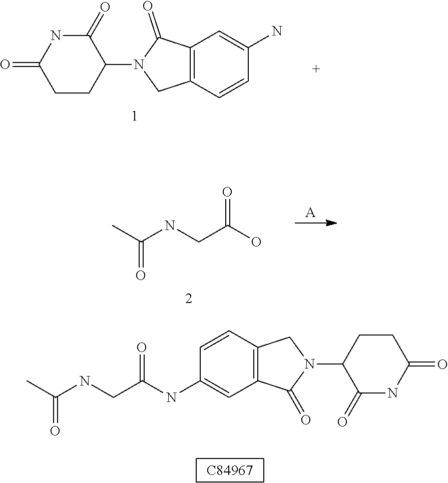

### Step A:

This compound was prepared according to General Procedure A for C80392. Yield: 34%.

### C89940

Synthesis of target compound was carried out following the scheme given below:

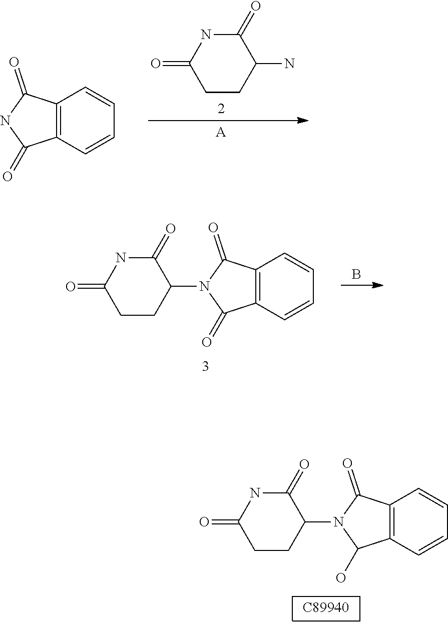

### Step A:

This compound was prepared according to General Procedure A for C64324. Yield: 34%.

### General Procedure B:

Compound 1 (1 mmol) was dissolved in 10 ml of HOAc, NaBH₄ (1 mmol) was added by portions during 15 min at r.t. The mixture was stirred at r.t. for 10 hours. TLC and LCMS control. The reaction mixture was mixture with water (25 ml), then filtered and washed with water (3×25 ml). The crude residue was purified using HPLC. Yield 58%.

### C35830

Synthesis of target compound was carried out following the scheme given below:

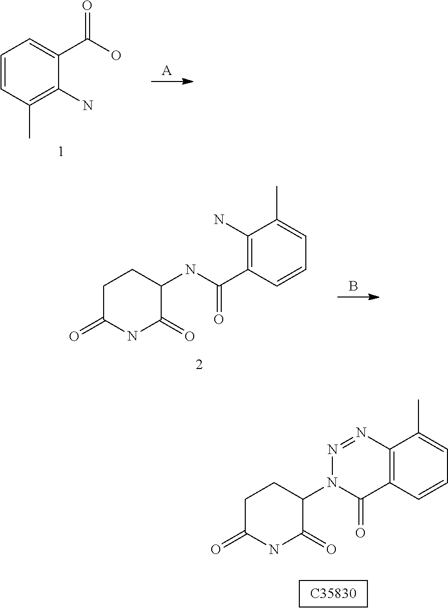

### Step A:

This compound was prepared according to General Procedure B for C35833. Yield: 36%.

### Step B:

This compound was prepared according to General Procedure B for C35833. Yield: 30%.

### C47995

Synthesis of target compound was carried out following the scheme given below:

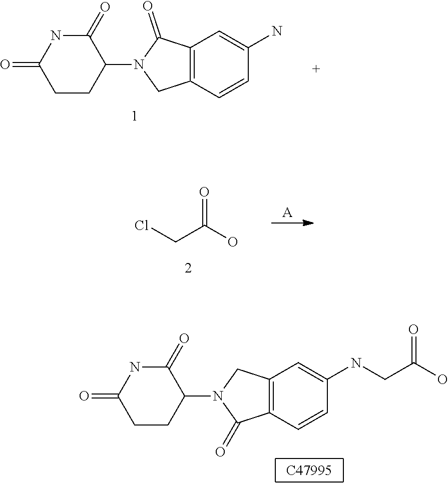

### Step A:

This compound was prepared according to General Procedure A for C12584. Yield: 8%.

In this case we used additional amount of HOAc (3 eq) before separation of the product from reaction mixture.

### C64376

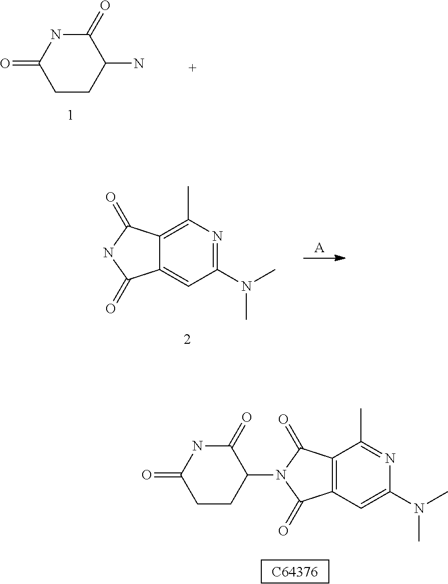

Synthesis of target compound was carried out following the scheme given below:

### Step A:

This compound was prepared according to General Procedure A for C64324. Yield: 31%.

### C84961

Synthesis of target compound was carried out following the scheme given below:

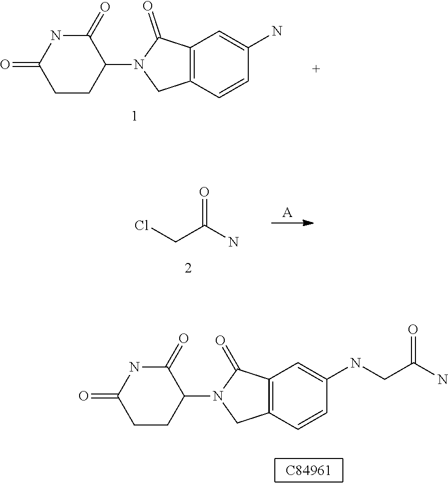

### Step A:

This compound was prepared according to General Procedure A for C12584. Yield: 11%.

### C22594

Synthesis of target compound was carried out following the scheme given below:

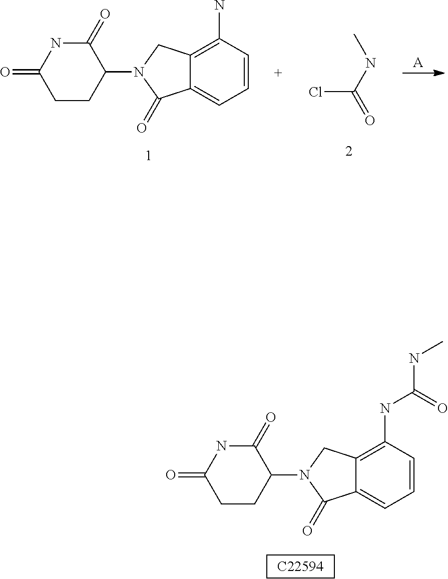

### Step A:

This compound was prepared according to General Procedure A for C95330. Yield: 34%.

### C12598

Synthesis of target compound was carried out following the scheme given below:

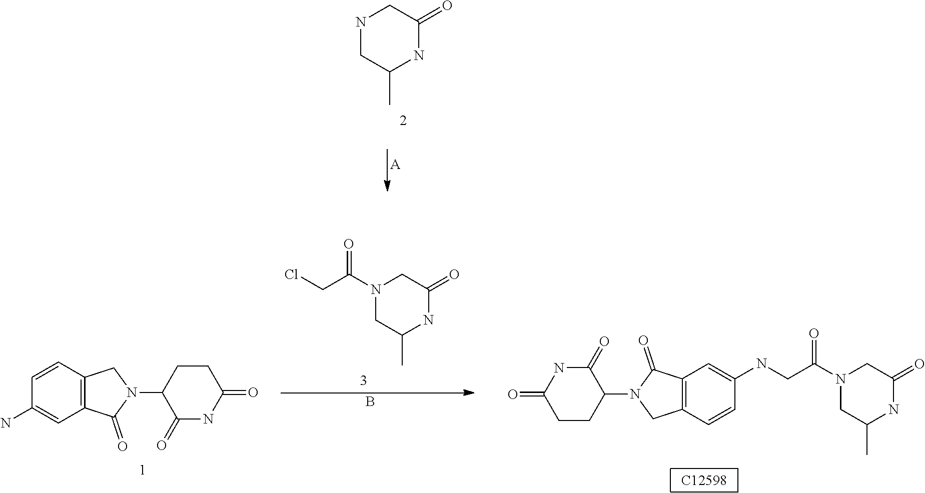

### Step A:

This compound was prepared according to General Procedure A for C95330. Yield: 81%.

### Step B:

This compound was prepared according to General Procedure A for C12584. Yield: 19%.

### C29408

Synthesis of target compound was carried out following the scheme given below:

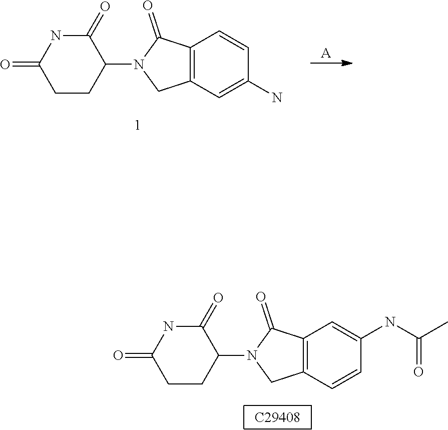

### Step A:

This compound was prepared according to General Procedure A for C80392. Yield: 68%.

### C73349

Synthesis of target compound was carried out following the scheme given below:

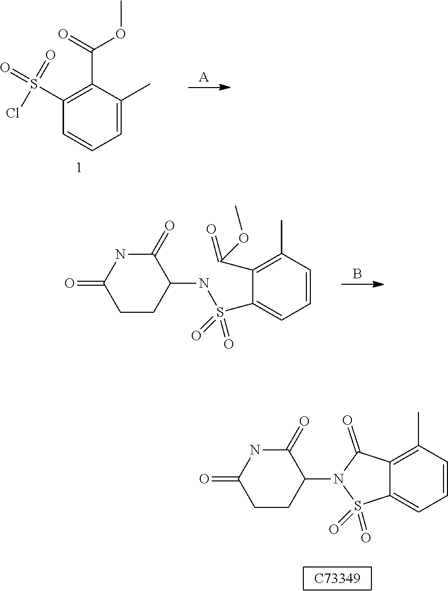

### Step A:

This compound was prepared according to General Procedure A for C95330. Yield: 86%.

### Step B:

Compound 2 (1 mmol), DIPEA (2 mmol) were dissolved in 10 ml of dioxane, the mixture was heated at 90° C. for 20 hours (TLC and LCMS control). The reaction mixture was cooled and the solvent was evaporated. The crude residue was purified using HPLC. Yield: 22%.

### C12597

Synthesis of target compound was carried out following the scheme given below:

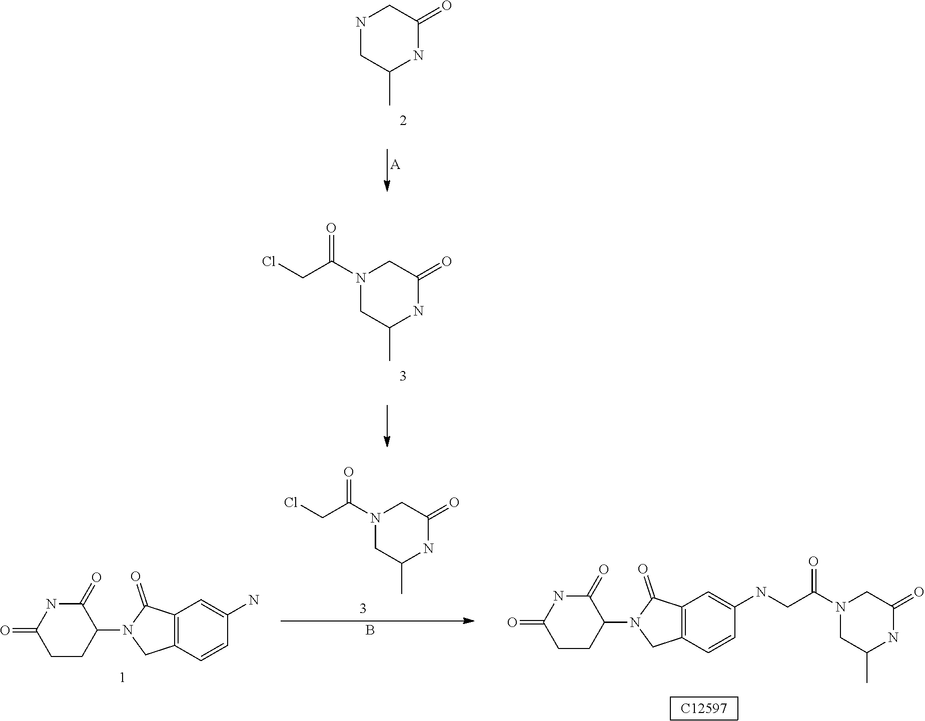

### Step A:

This compound was prepared according to General Procedure A for C95330. Yield: 84%.

### Step B:

This compound was prepared according to General Procedure A for C12584. Yield: 16%.

### C98053

Synthesis of target compound was carried out following the scheme given below:

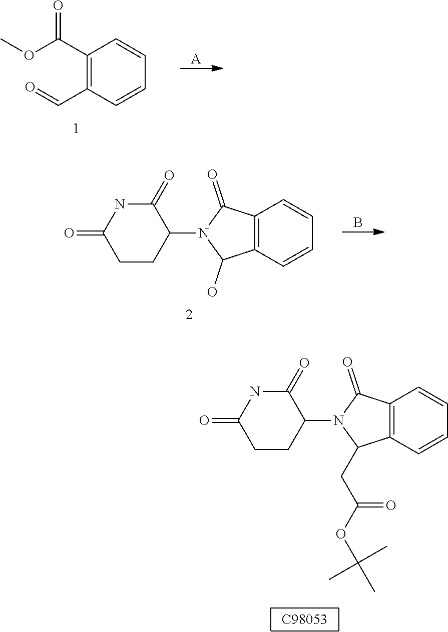

### Step A:

This compound was prepared according to General Procedure B for C67858. Yield: 79%.

### Step B:

Compound 2 (1 mmol), Wittig reagent tertBu ester (1.1 mmol) was refluxed in 20 ml of toluene for 96 hours (TLC and LCMS control). The reaction mixture was cooled, filtered and the solvent was evaporated. The crude residue was purified using HPLC. Yield: 29%.

### C51830

Synthesis of target compound was carried out following the scheme given below:

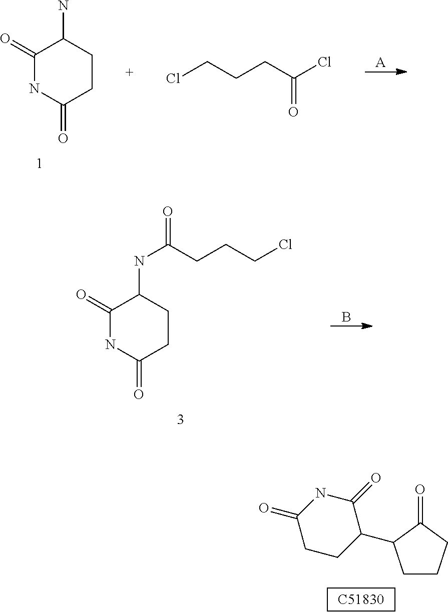

### Step A:

This compound was prepared according to General Procedure A for C95330. Yield: 12%.

### Step B:

Compound 2 (1 mmol), K₂C03 (2 mmol) were dissolved in 10 ml of acetonitrile, the mixture was heated at 80° C. for 12 hours. The reaction mixture was cooled, filtered and the solvent was evaporated. The crude residue was purified using HPLC. Yield: 14%.

### C97402

Synthesis of target compound was carried out following the scheme given below:

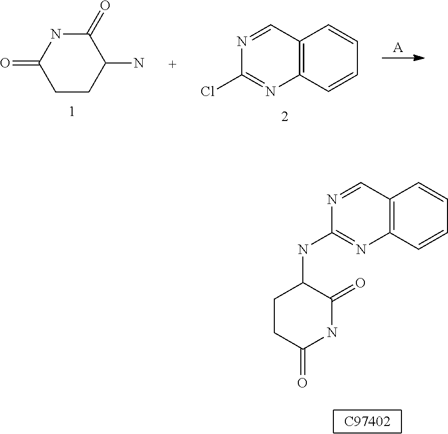

### Step A:

This compound was prepared according to General Procedure A for C80370. Yield: 26%.

### C68121

Synthesis of target compound was carried out following the scheme given below:

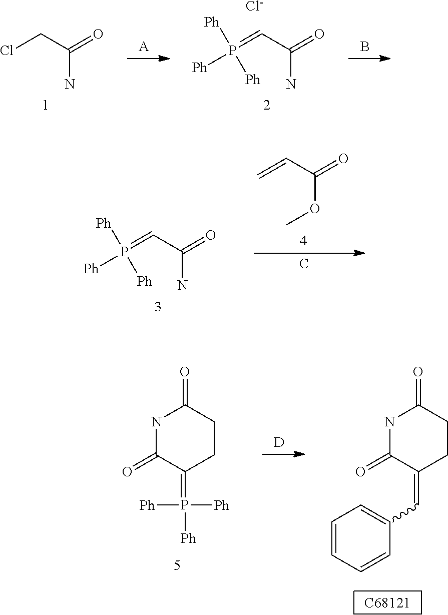

### Step A:

Compound 1 (1 mmol) and PPh3 (1.1 mmol) was heated at 80° C. in toluene (20 ml) for 10 hours (TLC and LCMS control). The reaction mixture was cooled, filtered and the precipitate was washed with toluene (3×25 ml). Yield: 94%.

### Step B:

Compound 2 (1 mmol) was dissolved in THF (20 ml), NaHCO₃ (5 mmol) was added and mixture was stirred at r.t. for 30 min. Then 50 ml of water was added, precipitate was filtered, washed with water and dried. Yield: 54%.

### Step C:

Compound 3 (1 mmol) and 4 (1.2 mmol) was dissolved in 10 ml of THF under Ar. NEt₃ (0.1 mmol) was added at 0° C. The mixture was stirred at r.t. for 1h and then at 50° C. for 10h. HOAc (0.1 mol) was added, solvent was evaporated. The residue was purified with use LC. Yield: 42%.

### Step D:

Compound 5 (1 mmol), Benzaldehyde (1.1 mmol) was refluxed in 20 ml of toluene for 96h hours (TLC and LCMS control). The reaction mixture was cooled, filtered and the solvent was evaporated. The crude residue was purified using HPLC. Yield: 22%.

### C47998

Synthesis of target compound was carried out following the scheme given below:

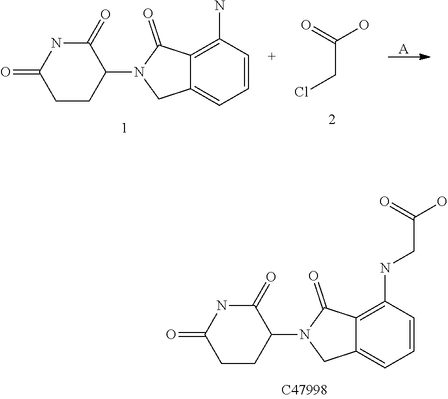

### Step A:

This compound was prepared according to General Procedure A for C12584. Yield: 14%.

In this case we used additional amount of HOAc (3 eq) before separation of the product from reaction mixture.

### C49713

Synthesis of target compound was carried out following the scheme given below:

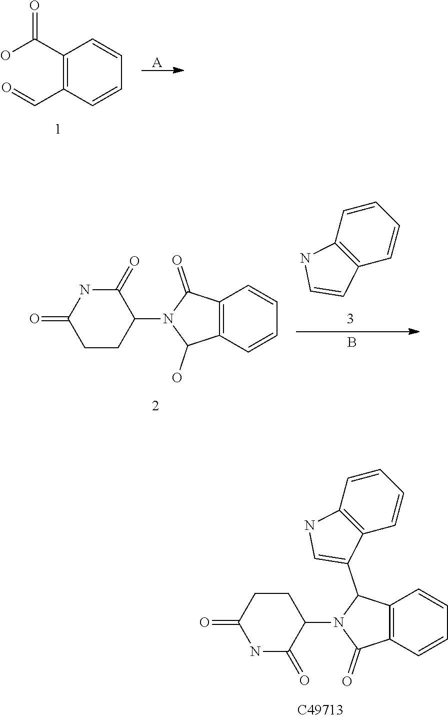

### Step A:

This compound was prepared according to General Procedure A for C80392. Yield: 39%.

### Step B:

Compound 2 (1 mmol) was dissolved in 10 ml of TFA, cooled to 10° C. and solution of 3 in 5 ml of TFA was added under Ar. The reaction mixture was stirred at r.t. for 12h, solvent was evaporated. The crude residue was purified using HPLC. Yield: 64%.

### C36126

Synthesis of target compound was carried out following the scheme given below:

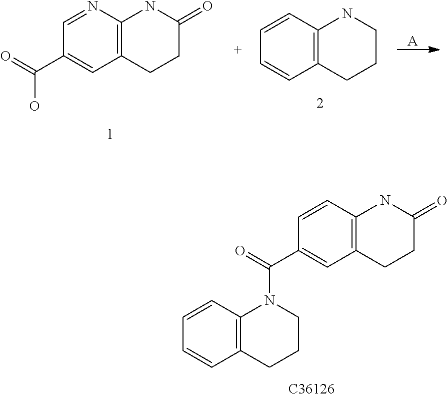

### Step A:

This compound was prepared according to General Procedure A for C80392. Yield: 59%.

### C39453

Synthesis of target compound was carried out following the scheme given below:

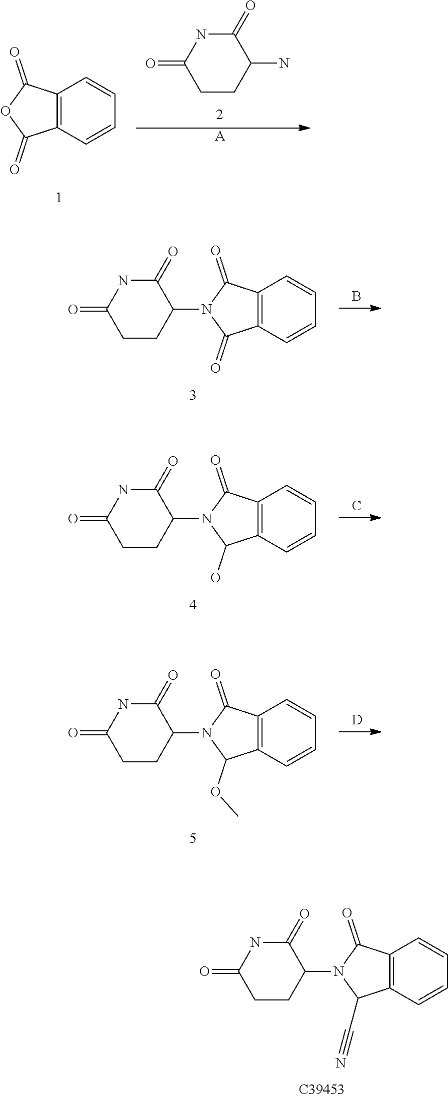

### Step A:

This compound was prepared according to General Procedure A for C64324. Yield: 54%.

### Step B:

This compound was prepared according to General Procedure B for C89940. Yield: 58%.

### Step C:

Compound 4 (1 mmol) was dissolved in 50 ml of methanol and refluxed for 12h. The solvent was evaporated. The crude residue was purified using HPLC. Yield: 83%.

### Step D:

Compound 5 (1 mmol) was dissolved in 10 ml of DMF, NaCN (0.5 mmol) was added. The reaction mixture was heated at 65° C. for 10h and refluxed for 12h. The mixture was filtered, washed with methanol (3×10 ml) and the solvent was evaporated. The crude residue was purified using HPLC. Yield: 12%.

### C10981

Synthesis of target compound was carried out following the scheme given below:

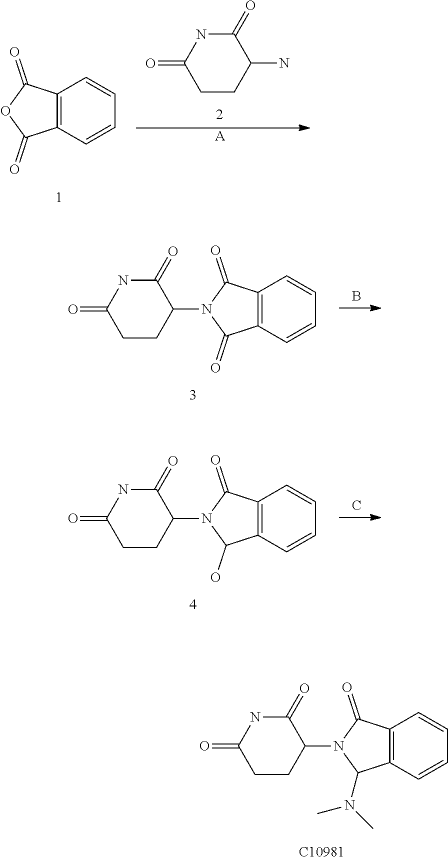

### Step A:

This compound was prepared according to General Procedure A for C64324. Yield: 54%.

### Step B:

This compound was prepared according to General Procedure B for C89940. Yield: 58%.

### Step C:

Compound 4 (1 mmol), dimethylamine 40% water solution (10 mmol) were dissolved in 50 ml toluene and 5 mg of TSA was added. The mixture was refluxed with us Din-Stark 18, cooled, solvent was evaporated. The crude residue was purified using HPLC. Yield: 34%.

### C80394

Synthesis of target compound was carried out following the scheme given below:

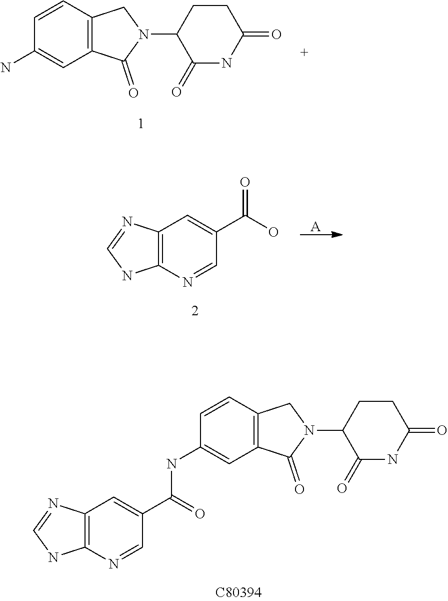

### Step A:

This compound was prepared according to General Procedure A for C80392. Yield: 60%.

### C11892

Synthesis of target compound was carried out following the scheme given below:

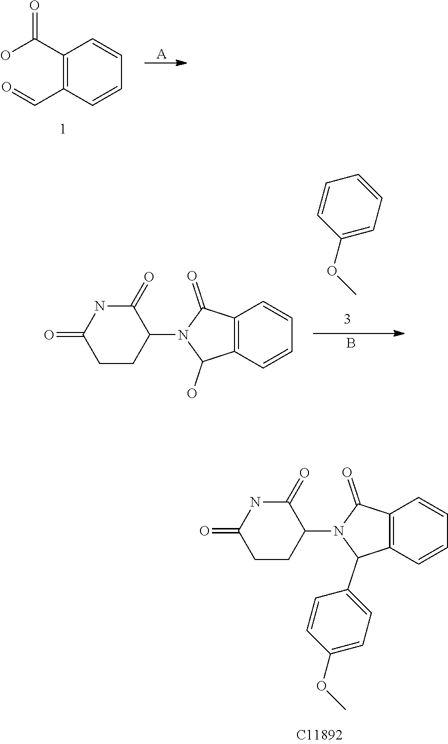

### Step A:

This compound was prepared according to General Procedure A for C80392. Yield: 39%.

### Step B:

This compound was prepared according to General Procedure B for C49713. Yield: 62%.

### C44292

Synthesis of target compound was carried out following the scheme given below:

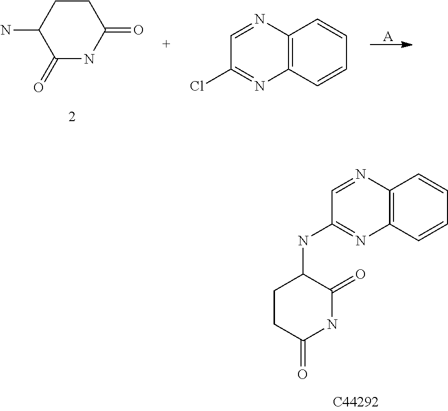

### Step A:

This compound was prepared according to General Procedure B for C80370. Yield: 18%.

### C12693

Synthesis of target compound was carried out following the scheme given below:

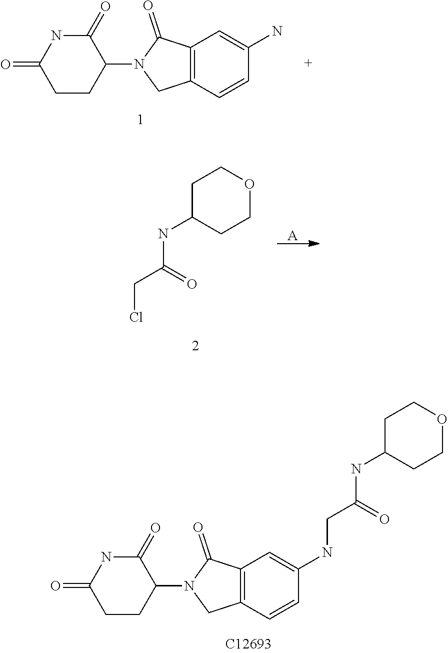

### Step A:

This compound was prepared according to General Procedure A for C12584. Yield: 26%.

### C56572

Synthesis of target compound was carried out following the scheme given below:

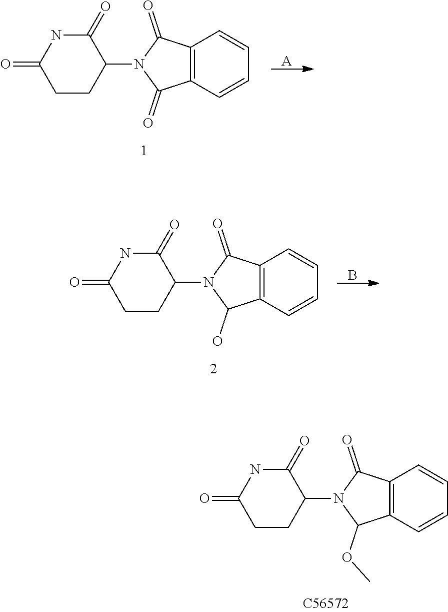

### Step A:

This compound was prepared according to General Procedure B for C89940. Yield: 58%.

### Step B:

Compound 4 (1 mmol) was dissolved in 50 ml of methanol and refluxed for 12h. The solvent was evaporated. The crude residue was purified using HPLC. Yield: 83%.

### C55468

Synthesis of target compound was carried out following the scheme given below:

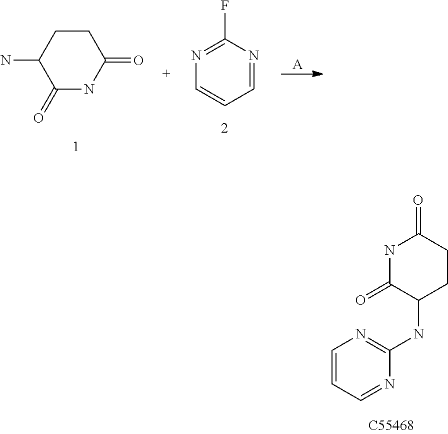

### Step A:

This compound was prepared according to General Procedure A for C80370. Yield: 38%.

### C29490

Synthesis of target compound was carried out following the scheme given below:

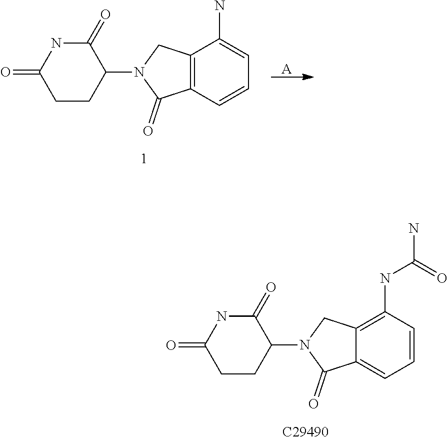

### C47997

Synthesis of target compound was carried out following the scheme given below:

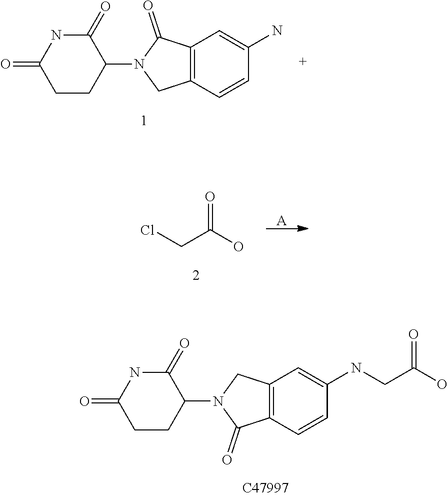

### Step A:

This compound was prepared according to General Procedure A for C12584. Yield: 14%.

In this case we used additional amount of HOAc (3 eq) before separation of the product from reaction mixture.

### C98696

Synthesis of target compound was carried out following the scheme given below:

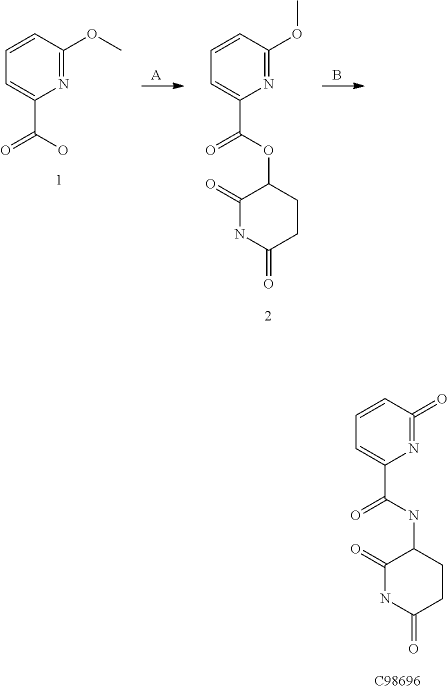

### Step A:

This compound was prepared according to General Procedure A for C80392. Yield: 72%.

### Step B:

Compound 2 (1 mmol) was dissolved in 10 ml of TFA and mixture was heated at 80° C. with stirring for 12h. The reaction mixture was cooled, filtered and the solvent was evaporated. The crude residue was purified using HPLC. Yield: 29%.

### C12581

Synthesis of target compound was carried out following the scheme given below:

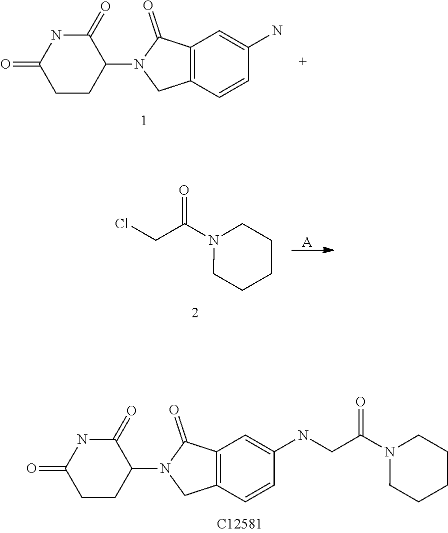

### Step A:

This compound was prepared according to General Procedure A for C12584. Yield: 22%.

### Example 4: Test Results

The following compounds were tested in the Competition Assay and/or Recruitment Assay described herein, and the results are listed below.

TABLE 1
IC50 Competition values were determined using the protocol and reagents described in Example 1.

| | | | | | | Recruit- |
|---|---|---|---|---|---|---|
| | | IC50 | EC50_ | EC50_ | EC50_ | ment |
| | | Competition | IKZF1 | ASS1 | SALL4 | Assess- |
| ID | Structure/Name | (M) | (M) | (M) | (M) | ment |
| C80392 | 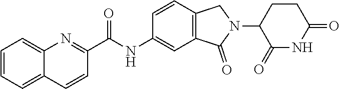 [SMILES: O=C1CCC(N2Cc3ccc(NC(=O)c4ccc5ccccc5n4)cc3C2=O)C(=O)N1] N-[2-(2,6-dioxo-3-piperidyl)-3-oxo-isoindolin-5-yl]quinoline- 2-carboxamide | 2.05303E−11 | NA | NA | NA | FALSE |
| C12584 | 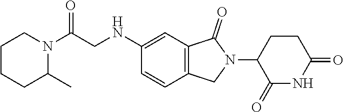 [SMILES: CC1CCCCN1C(=O)CNc1ccc2c(c1)C(=O)N(C1CCC(=O)NC1=O)C2] 3-[6-[[2-(2-methyl-1-piperidyl)-2-oxo-ethyl]amino]- 1-oxo-isoindolin-2-yl]piperidine-2,6-dione | 7.91364E−11 | NA | NA | NA | FALSE |
| CC-885 | 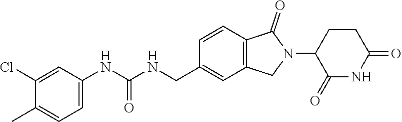 [SMILES: Cc1ccc(NC(=O)NCc2ccc3c(c2)CN(C2CCC(=O)NC2=O)C3=O)cc1Cl] 1-(3-chloro-4-methyl-phenyl)-3-[[2-(2,6-dioxo-3-piperidyl)-1- oxo-isoindolin-5-yl]methyl]urea | 4.65169E−10 | << | 8.01E−08 | NA | TRUE |
| C74668 | 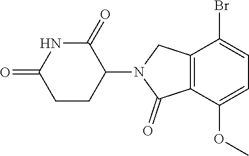 [SMILES: COc1ccc(Br)c2c1C(=O)N(C1CCC(=O)NC1=O)C2] 3-(4-bromo-7-methoxy-1-oxo-isoindolin-2-yl)piperidine-2,6-dione | 1.88601E−09 | NA | NA | NA | FALSE |
| CC-220 | 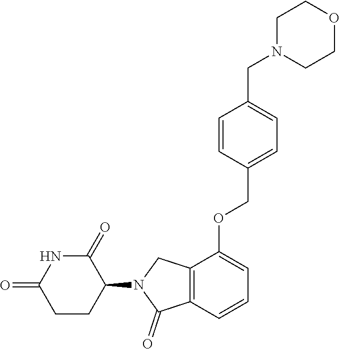 [SMILES: O=C1CC[C@H](N2Cc3c(OCc4ccc(CN5CCOCC5)cc4)cccc3C2=O)C(=O)N1] (3S)-3-[4-[[4-(morpholinomethyl)phenyl]methoxy]-1- oxo-isoindolin-2-yl]piperidine-2,6-dione | 1.000796E−08 | << | 6.24E−08 | << | TRUE |
| C80370 | 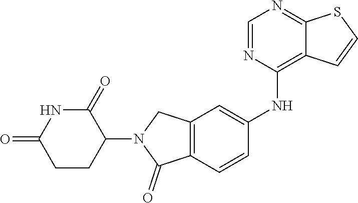 [SMILES: O=C1CCC(N2Cc3cc(Nc4ncnc5sccc45)ccc3C2=O)C(=O)N1] 3-[1-oxo-5-(thieno[2,3-d]pyrimidin-4-ylamino)isoindolin-2-yl] piperidine-2,6-dione | 1.09103E−08 | NA | NA | NA | FALSE |
| C24031 | 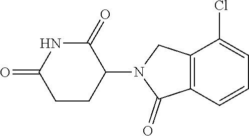 [SMILES: O=C1CCC(N2Cc3c(Cl)cccc3C2=O)C(=O)N1] 3-(4-chloro-1-oxo-isoindolin-2-yl)piperidine-2,6-dione | 1.14592E−08 | 5.87E−08 | 2.59E−07 | << | TRUE |
| C48014 | 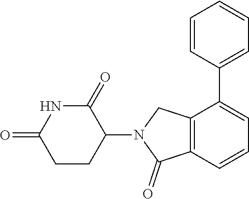 [SMILES: O=C1CCC(N2Cc3c(cccc3-c3ccccc3)C2=O)C(=O)N1] 3-(1-oxo-4-phenyl-isoindolin-2-yl)piperidine-2,6-dione | 2.43314E−08 | 1.24E−06 | 9.21E−07 | 6.21E−08 | TRUE |
| C58181 | 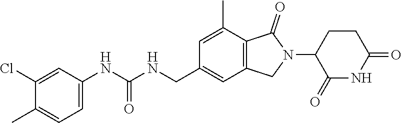 [SMILES: Cc1ccc(NC(=O)NCc2cc(C)c3c(c2)CN(C2CCC(=O)NC2=O)C3=O)cc1Cl] 1-(3-chloro-4-methyl-phenyl)-3-[[2-(2,6-dioxo-3-piperidyl)-7- methyl-1-oxo-isoindolin-5-yl]methyl]urea | 2.91268E−08 | NA | >> | NA | TRUE |
| C95330 | 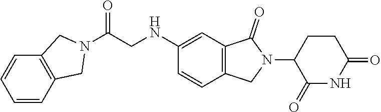 [SMILES: O=C1CCC(N2Cc3ccc(NCC(=O)N4Cc5ccccc5C4)cc3C2=O)C(=O)N1] 3-[6-[(2-isoindolin-2-yl-2-oxo-ethyl)amino]-1-oxo-isoindolin-2-yl] piperidine-2,6-dione | 3.0027E−08 | NA | NA | NA | FALSE |
| C95338 | 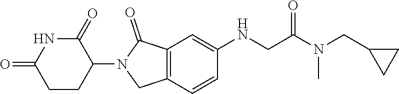 [SMILES: CN(CC1CC1)C(=O)CNc1ccc2c(c1)C(=O)N(C1CCC(=O)NC1=O)C2] N-(cyclopropylmethyl)-2-[[2-(2,6-dioxo-3-piperidyl)-3-oxo- isoindolin-5-yl]amino]-N-methyl-acetamide | 3.20528E−08 | NA | NA | NA | FALSE |
| C80382 acetic acid salt | 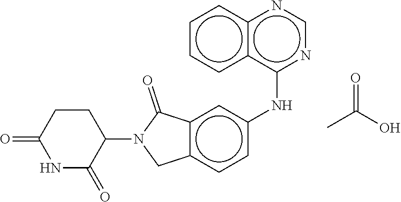 acetic acid; 3-[1-oxo-6-(quinazolin-4-ylamino)isoindolin-2-yl] piperidine-2,6-dione | 3.34174E−08 | NA | NA | NA | FALSE |
| C80369 | 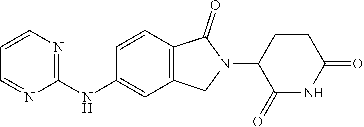 [SMILES: O=C1CCC(N2Cc3cc(Nc4ncccn4)ccc3C2=O)C(=O)N1] 3-[1-oxo-5-(pyrimidin-2-ylamino)isoindolin-2-yl]piperidine-2,6-dione | 4.15416E−08 | >> | 1.29E−06 | NA | TRUE |
| C47935 | 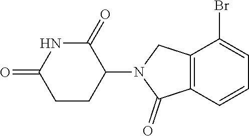 [SMILES: O=C1CCC(N2Cc3c(Br)cccc3C2=O)C(=O)N1] 3-(4-bromo-1-oxo-isoindolin-2-yl)piperidine-2,6-dione | 4.53754E−08 | 1.62E−07 | 6.77E−07 | 2.70E−08 | TRUE |
| C12583 | 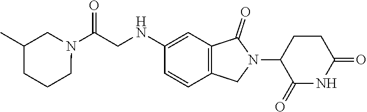 [SMILES: CC1CCCN(C(=O)CNc2ccc3c(c2)C(=O)N(C2CCC(=O)NC2=O)C3)C1] 3-[6-[[2-(3-methyl-1-piperidyl)-2-oxo-ethyl]amino]-1-oxo- isoindolin-2-yl]piperidine-2,6-dione | 4.9194E−08 | NA | NA | NA | FALSE |
| C28558 | 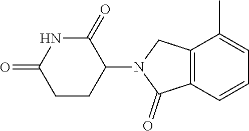 [SMILES: Cc1cccc2c1CN(C1CCC(=O)NC1=O)C2=O] 3-(4-methyl-1-oxo-isoindolin-2-yl)piperidine-2,6-dione | 5.04383E−08 | 7.74E−08 | 3.68E−07 | << | TRUE |
| C80387 | 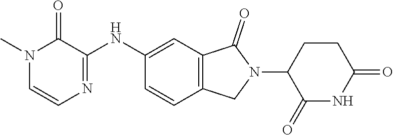 [SMILES: Cn1ccnc(Nc2ccc3c(c2)C(=O)N(C2CCC(=O)NC2=O)C3)c1=O] 3-[6-[(4-methyl-3-oxo-pyrazin-2-yl)amino]-1-oxo-isoindolin-2-yl] piperidine-2,6-dione | 5.41939E−08 | NA | NA | NA | FALSE |
| C80386 | 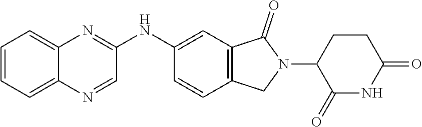 [SMILES: O=C1CCC(N2Cc3ccc(Nc4cnc5ccccc5n4)cc3C2=O)C(=O)N1] 3-[1-oxo-6-(quinoxalin-2-ylamino)isoindolin-2-yl]piperidine-2,6-dione | 6.73708E−08 | NA | NA | NA | FALSE |
| C67858 | 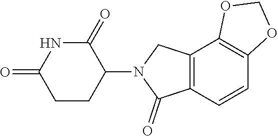 [SMILES: O=C1CCC(N2Cc3c(ccc4c3OCO4)C2=O)C(=O)N1] 3-(6-oxo-8H-[1,3]dioxolo[4,5-e]isoindol-7-yl)piperidine-2,6-dione | 1.06453E−07 | 5.69E−07 | 8.84E−07 | 2.93E−08 | TRUE |
| C28577 | 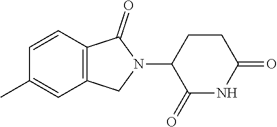 [SMILES: Cc1ccc2c(c1)CN(C1CCC(=O)NC1=O)C2=O] 3-(5-methyl-1-oxo-isoindolin-2-yl)piperidine-2,6-dione | 1.12883E−07 | 1.61E−06 | 6.53E−07 | << | TRUE |
| C89676 | 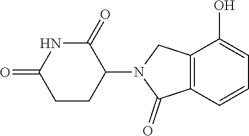 [SMILES: O=C1CCC(N2Cc3c(O)cccc3C2=O)C(=O)N1] 3-(4-hydroxy-1-oxo-isoindolin-2-yl)piperidine-2,6-dione | 1.26858E−07 | 1.13E−07 | 8.32E−07 | << | TRUE |
| C98103 | 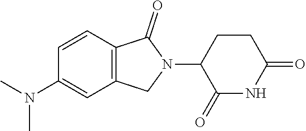 [SMILES: CN(C)c1ccc2c(c1)CN(C1CCC(=O)NC1=O)C2=O] 3-[5-(dimethylamino)-1-oxo-isoindolin-2-yl]piperidine-2,6-dione | 1.44886E−07 | 1.29E−06 | >> | << | TRUE |
| C80383 | 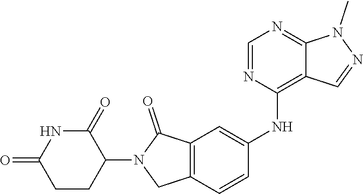 [SMILES: Cn1ncc2c(Nc3ccc4c(c3)C(=O)N(C3CCC(=O)NC3=O)C4)ncnc21] 3-[6-[(1-methylpyrazolo[3,4-d]pyrimidin-4-yl)amino]-1-oxo- isoindolin-2-yl]piperidine-2,6-dione | 1.47987E−07 | NA | NA | NA | FALSE |
| C80391 | 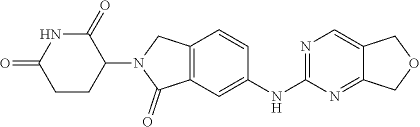 [SMILES: O=C1CCC(N2Cc3ccc(Nc4ncc5c(n4)COC5)cc3C2=O)C(=O)N1] 3-[6-(5,7-dihydrofuro[3,4-d]pyrimidin-2-ylamino)-1-oxo- isoindolin-2-yl]piperidine-2,6-dione | 1.62797E−07 | >> | NA | 1.52E−07 | TRUE |
| C48016 | 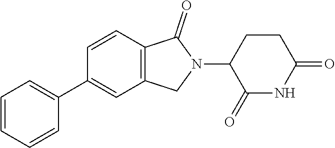 [SMILES: O=C1CCC(N2Cc3cc(-c4ccccc4)ccc3C2=O)C(=O)N1] 3-(1-oxo-5-phenyl-isoindolin-2-yl)piperidine-2,6-dione | 1.73495E−07 | >> | NA | 1.22E−07 | TRUE |
| C05955 | 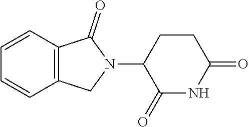 [SMILES: O=C1CCC(N2Cc3ccccc3C2=O)C(=O)N1] 3-(1-oxoisoindolin-2-yl)piperidine-2,6-dione | 1.7519E−07 | 5.21E−07 | 1.55E−06 | << | TRUE |
| C28620 | 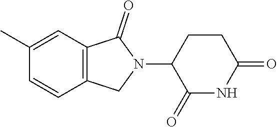 [SMILES: Cc1ccc2c(c1)C(=O)N(C1CCC(=O)NC1=O)C2] 3-(6-methyl-1-oxo-isoindolin-2-yl)piperidine-2,6-dione | 1.78223E−07 | >> | NA | 2.70E−07 | TRUE |
| C80384 | 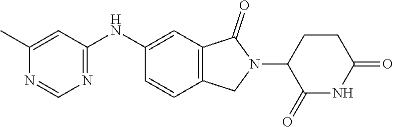 [SMILES: Cc1cc(Nc2ccc3c(c2)C(=O)N(C2CCC(=O)NC2=O)C3)ncn1] 3-[6-[(6-methylpyrimidin-4-yl)amino]-1-oxo-isoindolin-2-yl] piperidine-2,6-dione | 1.78827E−07 | NA | NA | NA | FALSE |
| C51383 | 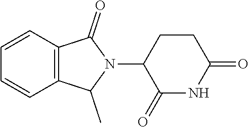 [SMILES: CC1c2ccccc2C(=O)N1C1CCC(=O)NC1=O] 3-(1-methyl-3-oxo-isoindolin-2-yl)piperidine-2,6-dione | 1.90194E−07 | 1.52E−06 | 1.70E−06 | 5.93E−08 | TRUE |
| C84965 | 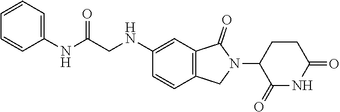 [SMILES: O=C1CCC(N2Cc3ccc(NCC(=O)Nc4ccccc4)cc3C2=O)C(=O)N1] 2-[[2-(2,6-dioxo-3-piperidyl)-3-oxo-isoindolin-5-yl]amino]-N- phenyl-acetamide | 1.98835E−07 | >> | NA | NA | TRUE |
| C12595 | 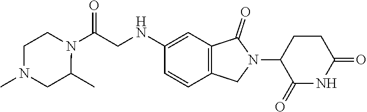 [SMILES: CC1CN(C)CCN1C(=O)CNc1ccc2c(c1)C(=O)N(C1CCC(=O)NC1=O)C2] 3-[6-[[2-(2,4-dimethylpiperazin-1-yl)-2-oxo-ethyl]amino]-1- oxo-isoindolin-2-yl]piperidine-2,6-dione | 2.07712E−07 | NA | NA | NA | FALSE |
| C96622 | 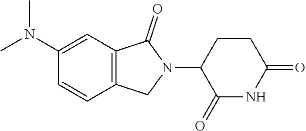 [SMILES: CN(C)c1ccc2c(c1)C(=O)N(C1CCC(=O)NC1=O)C2] 3-[6-(dimethylamino)-1-oxo-isoindolin-2-yl]piperidine-2,6-dione | 2.13914E−07 | NA | NA | NA | FALSE |
| C22622 | 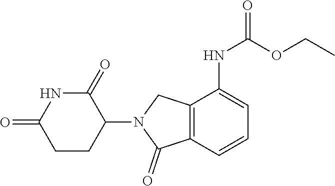 [SMILES: CCOC(=O)Nc1cccc2c1CN(C1CCC(=O)NC1=O)C2=O] ethyl N-[2-(2,6-dioxo-3-piperidyl)-1-oxo-isoindolin-4-yl]carbamate | 2.25027E−07 | >> | >> | 1.21E−07 | TRUE |
| C48018 | 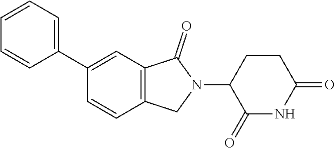 [SMILES: O=C1CCC(N2Cc3ccc(-c4ccccc4)cc3C2=O)C(=O)N1] 3-(1-oxo-6-phenyl-isoindolin-2-yl)piperidine-2,6-dione | 2.28101E−07 | >> | NA | 3.95E−07 | TRUE |
| LEN | 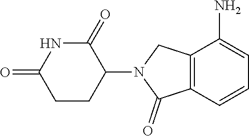 [SMILES: Nc1cccc2c1CN(C1CCC(=O)NC1=O)C2=O] 3-(4-amino-1-oxo-isoindolin-2-yl)piperidine-2,6-dione | 2.43162E−07 | 2.43162E−07 | 6.22E−07 | 1.33E−07 | TRUE |
| C23066 | 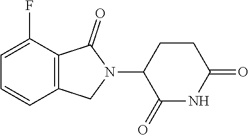 [SMILES: O=C1CCC(N2Cc3cccc(F)c3C2=O)C(=O)N1] 3-(7-fluoro-1-oxo-isoindolin-2-yl)piperidine-2,6-dione | 2.48406E−07 | >> | >> | 8.82E−08 | TRUE |
| C84964 | 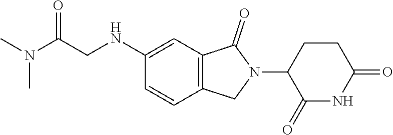 [SMILES: CN(C)C(=O)CNc1ccc2c(c1)C(=O)N(C1CCC(=O)NC1=O)C2] 2-[[2-(2,6-dioxo-3-piperidyl)-3-oxo-isoindolin-5-yl]amino]-N,N- dimethyl-acetamide | 2.65591E−07 | NA | NA | NA | FALSE |
| C29137 | 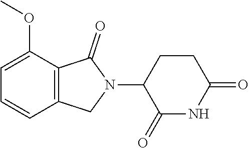 [SMILES: COc1cccc2c1C(=O)N(C1CCC(=O)NC1=O)C2] 3-(7-methoxy-1-oxo-isoindolin-2-yl)piperidine-2,6-dione | 3.04218E−07 | NA | NA | NA | FALSE |
| C12586 | 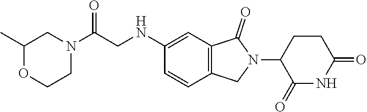 [SMILES: CC1CN(C(=O)CNc2ccc3c(c2)C(=O)N(C2CCC(=O)NC2=O)C3)CCO1] 3-[6-[[2-(2-methylmorpholin-4-yl)-2-oxo-ethyl]amino]-1- oxo-isoindolin-2-yl]piperidine-2,6-dione | 3.82684E−07 | NA | NA | NA | FALSE |
| C28891 | 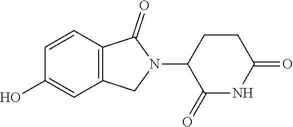 [SMILES: O=C1CCC(N2Cc3cc(O)ccc3C2=O)C(=O)N1] 3-(5-hydroxy-1-oxo-isoindolin-2-yl)piperidine-2,6-dione | 4.79112E−07 | 1.04E−07 | 4.23E−06 | 1.05E−07 | TRUE |
| C35856 | 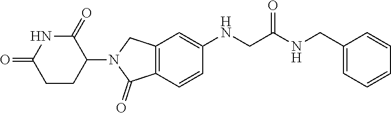 [SMILES: O=C(CNc1ccc2c(c1)CN(C1CCC(=O)NC1=O)C2=O)NCc1ccccc1] N-benzyl-2-[[2-(2,6-dioxo-3-piperidyl)-1-oxo-isoindolin-5-yl] aminolacetamide | 4.84125E−07 | 1.15E−06 | >> | 1.88E−07 | TRUE |
| C35751 | 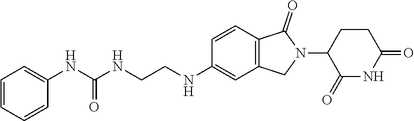 [SMILES: O=C1CCC(N2Cc3cc(NCCNC(=O)Nc4ccccc4)ccc3C2=O)C(=O)N1] 1-[2-[[2-(2,6-dioxo-3-piperidyl)-1-oxo-isoindolin-5-yl]amino] ethyl]-3-phenyl-urea | 4.85851E−07 | >> | 1.45E−06 | 2.32E−07 | TRUE |
| C28928 | 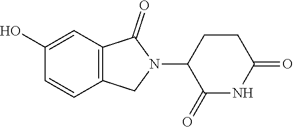 [SMILES: O=C1CCC(N2Cc3ccc(O)cc3C2=O)C(=O)N1] 3-(6-hydroxy-1-oxo-isoindolin-2-yl)piperidine-2,6-dione | 4.88558E−07 | >> | >> | 2.26E−07 | TRUE |
| C28973 | 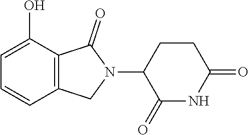 [SMILES: O=C1CCC(N2Cc3cccc(O)c3C2=O)C(=O)N1] 3-(7-hydroxy-1-oxo-isoindolin-2-yl)piperidine-2,6-dione | 5.26297E−07 | >> | NA | 1.50E−07 | TRUE |
| C48020 | 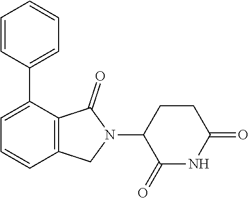 [SMILES: O=C1CCC(N2Cc3cccc(-c4ccccc4)c3C2=O)C(=O)N1] 3-(1-oxo-7-phenyl-isoindolin-2-yl)piperidine-2,6-dione | 5.4615E−07 | NA | NA | NA | FALSE |
| C68126 | 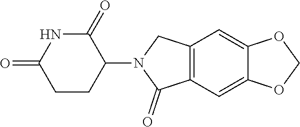 [SMILES: O=C1CCC(N2Cc3cc4c(cc3C2=O)OCO4)C(=O)N1] 3-(7-oxo-5H-[1,3]dioxolo[4,5-f]isoindol-6-yl)piperidine-2,6-dione | 5.64062E−07 | >> | >> | 3.19E−07 | TRUE |
| C07207 | 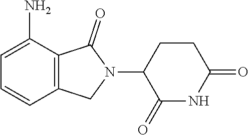 [SMILES: Nc1cccc2c1C(=O)N(C1CCC(=O)NC1=O)C2] 3-(7-amino-1-oxo-isoindolin-2-yl)piperidine-2,6-dione | 5.86861E−07 | >> | NA | 3.07E−07 | TRUE |
| C95333 | 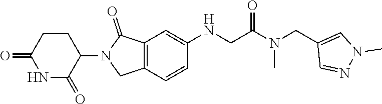 [SMILES: CN(Cc1cnn(C)c1)C(=O)CNc1ccc2c(c1)C(=O)N(C1CCC(=O)NC1=O)C2] 2-[[2-(2,6-dioxo-3-piperidyl)-3-oxo-isoindolin-5-yl]amino]-N- methyl-N-[(1-methylpyrazol-4-yl)met [symbol] | 5.90522E−07 | NA | NA | NA | FALSE |
| C84966 | 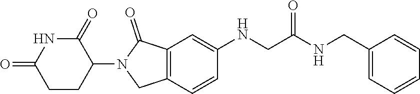 [SMILES: O=C(CNc1ccc2c(c1)C(=O)N(C1CCC(=O)NC1=O)C2)NCc1ccccc1] N-benzyl-2-[[2-(2,6-dioxo-3-piperidyl)-3-oxo-isoindolin- 5-yl]amino]acetaimde | 5.99373E−07 | NA | NA | NA | FALSE |
| C48003 | 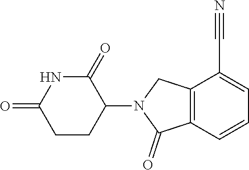 [SMILES: N#Cc1cccc2c1CN(C1CCC(=O)NC1=O)C2=O] 2-(2,6-dioxo-3-piperidyl)-1-oxo-isoindoline-4-carbonitrile | 6.21907E−07 | >> | >> | 5.81E−7 | TRUE |
| C66979 | 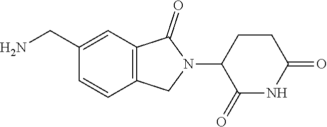 [SMILES: NCc1ccc2c(c1)C(=O)N(C1CCC(=O)NC1=O)C2] 3-[6-(aminomethyl)-1-oxo-isoindolin-2-yl]piperidine-2,6-dione | 6.33015E−07 | NA | NA | NA | FALSE |
| C80389 | 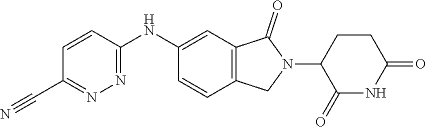 [SMILES: N#Cc1ccc(Nc2ccc3c(c2)C(=O)N(C2CCC(=O)NC2=O)C3)nn1] 6-[[2-(2,6-dioxo-3-piperidyl)-3-oxo-isoindolin-5-yl]amino]pyridazine- 3-carbonitrile | 6.45036E−07 | NA | NA | NA | FALSE |
| C28661 | 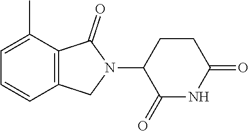 [SMILES: Cc1cccc2c1C(=O)N(C1CCC(=O)NC1=O)C2] 3-(7-methyl-1-oxo-isoindolin-2-yl)piperidine-2,6-dione | 6.54317E−07 | NA | NA | NA | FALSE |
| C35833 | 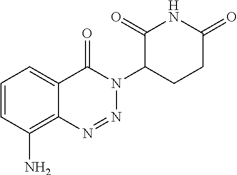 [SMILES: Nc1cccc2c(=O)n(C3CCC(=O)NC3=O)nnc12] 3-(8-amino-4-oxo-1,2,3-benzotriazin-3-yl)piperidine-2,6-dione | 6.83558E−07 | NA | NA | NA | FALSE |
| C59904 | 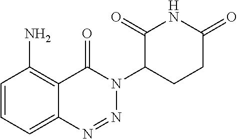 [SMILES: Nc1cccc2nnn(C3CCC(=O)NC3=O)c(=O)c12] 3-(5-amino-4-oxo-1,2,3-benzotriazin-3-yl)piperidine-2,6-dione | 7.03899E−07 | 1.22E−07 | 6.87E−07 | 3.30E−07 | TRUE |
| C80390 | 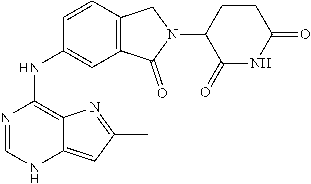 [SMILES: Cc1cc2[nH]cnc(Nc3ccc4c(c3)C(=O)N(C3CCC(=O)NC3=O)C4)c-2n1] 3-[6-[(6-methylpyrrolo[3,2-d]pyrimidin-4-yl)ammo]-1-oxo- isoindolin-2-yl]piperidine-2,6-dione | 7.28895E−07 | >> | NA | NA | TRUE |
| C35754 | 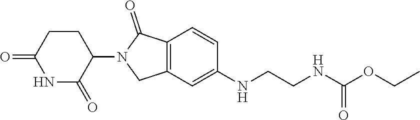 [SMILES: CCOC(=O)NCCNc1ccc2c(c1)CN(C1CCC(=O)NC1=O)C2=O] ethyl N-[2-[[2-(2,6-dioxo-3-piperidyl)-1-oxo-isoindolin-5-yl] amino]ethyl]carbamate | 7.36821E−07 | >> | >> | 2.78E−07 | TRUE |
| C49708 | 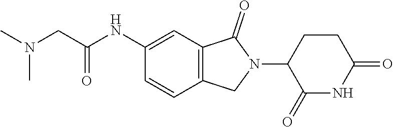 [SMILES: CN(C)CC(=O)Nc1ccc2c(c1)C(=O)N(C1CCC(=O)NC1=O)C2] 2-(dimethylamino)-N-[2-(2,6-dioxo-3-piperidyl)-3-oxo- isoindolin-5-yl]acetamide | 7.5179−07 | NA | NA | NA | FALSE |
| C80380 | 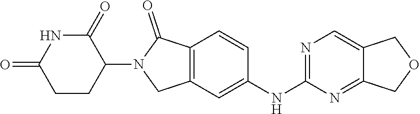 [SMILES: O=C1CCC(N2Cc3cc(Nc4ncc5c(n4)COC5)ccc3C2=O)C(=O)N1] 3-[5-(5,7-dihydrofuro[3,4-d]pyrimidin-2-ylamino)-1-oxo- isoindolin-2-yl]piperidine-2,6-dione | 7.6043E−07 | 1.78E−06 | NA | 9.89E−09 | TRUE |
| C35797 | 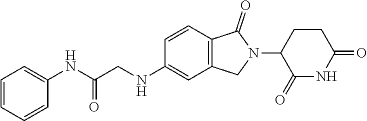 [SMILES: O=C1CCC(N2Cc3cc(NCC(=O)Nc4ccccc4)ccc3C2=O)C(=O)N1] 2-[[2-(2,6-dioxo-3-piperidyl)-1-oxo-isoindolin-5-yl] amino]-N-phenyl-acetamide | 8.50894E−07 | 1.33E−06 | >> | 1.08E−06 | TRUE |
| C80395 | 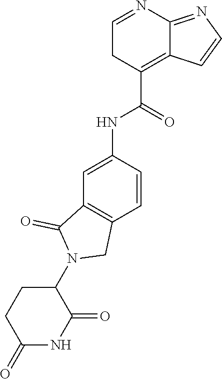 [SMILES: O=C1CCC(N2Cc3ccc(NC(=O)C4=C5C=CN=C5N=CC4)cc3C2=O)C(=O)N1] N-[2-(2,6-dioxo-3-piperidyl)-3-oxo-isoindolin-5-yl]-5H-pyrrolo [2,3-b]pyridine-4-carboxamide | 8.80494E−07 | >> | NA | NA | TRUE |
| C80373 | 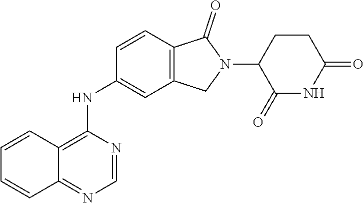 [SMILES: O=C1CCC(N2Cc3cc(Nc4ncnc5ccccc45)ccc3C2=O)C(=O)N1] 3-[-oxo-5-(quinazolin-4-ylamino)isoindolin-2-yl]piperidine-2,6-dione | 8.92014E−07 | >> | >> | NA | TRUE |
| CC-122 | 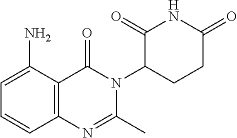 [SMILES: Cc1nc2cccc(N)c2c(=O)n1C1CCC(=O)NC1=O] 3-(5-amino-2-methyl-4-oxo-quinazolin-3-yl)piperidine-2,6-dione | 9.46128E−07 | 2.01E−07 | 1.39E−06 | 1.99E−07 | TRUE |
| C45748 | 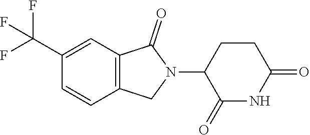 [SMILES: O=C1CCC(N2Cc3ccc(C(F)(F)F)cc3C2=O)C(=O)N1] 3-[1-oxo-6-(trifluoromethyl)isoindolin-2-yl]piperidine-2,6-dione | 9.52933E−07 | NA | NA | NA | FALSE |
| C80379 | 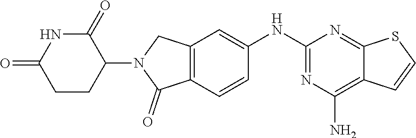 [SMILES: Nc1nc(Nc2ccc3c(c2)CN(C2CCC(=O)NC2=O)C3=O)nc2sccc12] 3-[5-[(4-aminothieno[2,3-d]pyrimidin-2-yl)amino]-1-oxo- isoindolin-2-yl]piperidine-2,6-dione | 9.70979E−07 | >> | NA | NA | TRUE |
| C47933 | 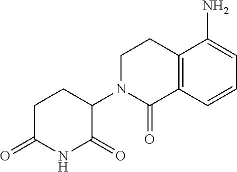 [SMILES: Nc1cccc2c1CCN(C1CCC(=O)NC1=O)C2=O] 3-(5-amino-1-oxo-3,4-dihydroisoquinolin-2-yl)piperidine-2,6-dione | 1.0938E−066 | >> | NA | NA | TRUE |
| POM | 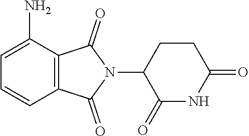 [SMILES: Nc1cccc2c1C(=O)N(C1CCC(=O)NC1=O)C2=O] 4-amino-2-(2,6-dioxo-3-piperidyl)isoindoline-1,3-dione | 1.10121E−06 | 7.27E−07 | 2.07E−06 | 6.77E−08 | TRUE |
| C22548 | 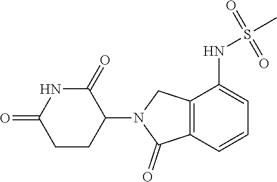 [SMILES: CS(=O)(=O)Nc1cccc2c1CN(C1CCC(=O)NC1=O)C2=O] N-[2-(2,6-dioxo-3-piperidyl)-1-oxo-isoindolin-4-yl] methanesulfonamide | 1.10968E−06 | 4.94E−07 | >> | 6.31E−07 | TRUE |
| C84963 | 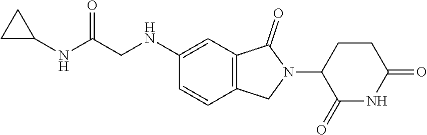 [SMILES: O=C1CCC(N2Cc3ccc(NCC(=O)NC4CC4)cc3C2=O)C(=O)N1] N-cyclopropyl-2-[[2-(2,6-dioxo-3-piperidyl)-3-oxo-isoindolin-5-yl] amino]acetamide | 1.21084E−06 | NA | NA | NA | FALSE |
| C22586 | 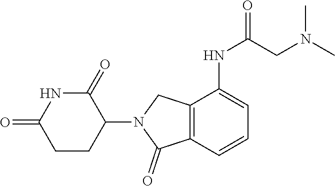 [SMILES: CN(C)CC(=O)Nc1cccc2c1CN(C1CCC(=O)NC1=O)C2=O] 2-(dimethylamino)-N-[2-(2,6-dioxo-3-piperidyl)-1-oxo-isoindolin-4-yl] acetamide | 1.29799E−06 | >> | NA | 1.14E−06 | TRUE |
| C84971 | 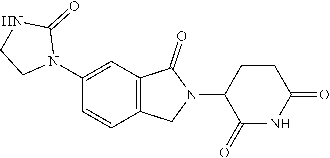 [SMILES: O=C1CCC(N2Cc3ccc(N4CCNC4=O)cc3C2=O)C(=O)N1] 3-[1-oxo-6-(2-oxoimidazolidin-1-yl)isoindolin-2-yl]piperidine-2,6-dione | 1.31113E−06 | NA | NA | NA | FALSE |
| C48007 | 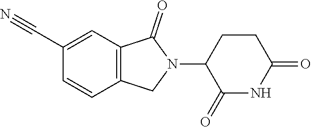 [SMILES: N#Cc1ccc2c(c1)C(=O)N(C1CCC(=O)NC1=O)C2] 2-(2,6-dioxo-3-piperidyl)-3-oxo-isoindoline-5-carbonitrile | 1.34315E−06 | 1.62E−06 | >> | 4.30E−07 | TRUE |
| C99884 | 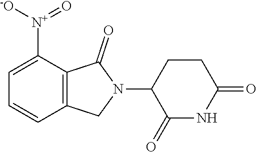 3-(7-nitro-1-oxo-isoindolin-2-yl)piperidine-2,6-dione | 1.36829E−06 | NA | NA | NA | FALSE |
| C29330 | 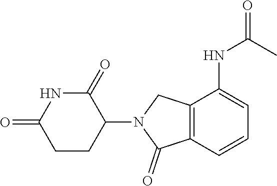 [SMILES: CC(=O)Nc1cccc2c1CN(C1CCC(=O)NC1=O)C2=O] N-[2-(2,6-dioxo-3-piperidyl)-1-oxo-isoindolin-4-yl]acetamide | 1.41494E−06 | >> | >> | 2.50E−07 | TRUE |
| C84970 | 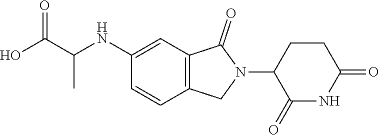 [SMILES: CC(Nc1ccc2c(c1)C(=O)N(C1CCC(=O)NC1=O)C2)C(=O)O] 2-[[2-(2,6-dioxo-3-piperidyl)-3-oxo-isoindolin-5-yl]amino] propanoic acid | 1.42322E−06 | NA | NA | 1.01E−06 | TRUE |
| C29361 | 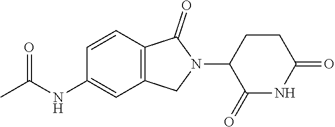 [SMILES: CC(=O)Nc1ccc2c(c1)CN(C1CCC(=O)NC1=O)C2=O] N-[2-(2,6-dioxo-3-piperidyl)-1-oxo-isoindolin-5-yl]acetamide | 1.43168E−06 | >> | >> | 1.86E−07 | TRUE |
| C22564 | 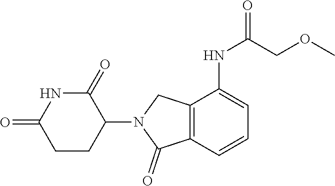 [SMILES: COCC(=O)Nc1cccc2c1CN(C1CCC(=O)NC1=O)C2=O] N-[2-(2,6-dioxo-3-piperidyl)-1-oxo-isoindolin-4-yl]-2- methoxy-acetamide | 1.67347E−06 | >> | >> | 8.73E−07 | TRUE |
| C48005 | 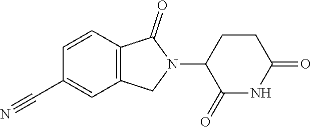 [SMILES: N#Cc1ccc2c(c1)CN(C1CCC(=O)NC1=O)C2=O] 2-(2,6-dioxo-3-piperidyl)-1-oxo-isoindoline-5-carbonitrile | 1.73199E−06 | >> | NA | 5.95E−07 | TRUE |
| C80375 | 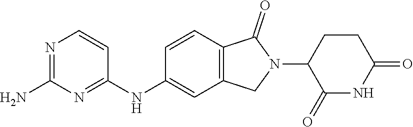 [SMILES: Nc1nccc(Nc2ccc3c(c2)CN(C2CCC(=O)NC2=O)C3=O)n1] 3-[5-[(2-aminopyrimidm-4-yl)amino]-1-oxo-isoindolin-2-yl] piperidine-2,6-dione | 2.05608E−06 | NA | NA | NA | FALSE |
| C48009 | 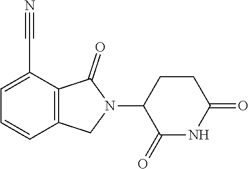 [SMILES: N#Cc1cccc2c1C(=O)N(C1CCC(=O)NC1=O)C2] 2-(2,6-dioxo-3-piperidyl)-3-oxo-isoindoline-4-carbonitrile | 2.18134E−06 | NA | NA | NA | FALSE |
| C80378 | 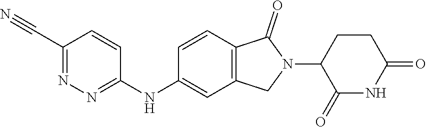 [SMILES: N#Cc1ccc(Nc2ccc3c(c2)CN(C2CCC(=O)NC2=O)C3=O)nn1] 6-[[2-(2,6-dioxo-3-piperidyl)-1-oxo-isoindolin-5-yl]amino] pyridazine-3-carbonitrile | 2.20805E−06 | >> | 9.42E−06 | 3.87E−07 | TRUE |
| C21223 | 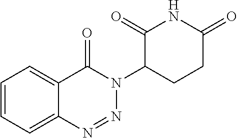 [SMILES: O=C1CCC(n2nnc3ccccc3c2=O)C(=O)N1] 3-(4-oxo-1,2,3-benzotriazin-3-yl)piperidine-2,6-dione | 2.2375E−06 | >> | >> | 5.17E−07 | TRUE |
| C13247 | 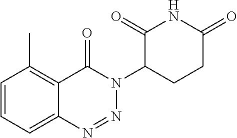 [SMILES: Cc1cccc2nnn(C3CCC(=O)NC3=O)c(=O)c12] 3-(5-methyl-4-oxo-1,2,3-benzotriazin-3-yl)piperidine-2,6-dione | 2.29444E−06 | 2.88E−06 | 8.55E−06 | 4.02E−07 | TRUE |
| C35811 | 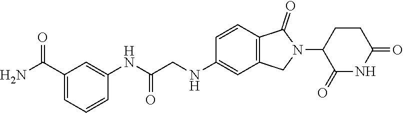 [SMILES: NC(=O)c1cccc(NC(=O)CNc2ccc3c(c2)CN(C2CCC(=O)NC2=O)C3=O)c1] 3-[[2-[[2-(2,6-dioxo-3-piperidyl)-1-oxo-isoindolin-5-yl] amino]acetyl]amino]benzamide | 2.44321E−06 | >> | >> | 6.41E−07 | TRUE |
| C47959 | 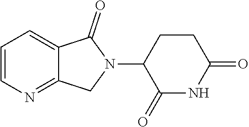 [SMILES: O=C1CCC(N2Cc3ncccc3C2=O)C(=O)N1] 3-(5-oxo-7H-pyrrolo[3,4-b]pyridin-6-yl)piperidine-2,6-dione | 2.5664E−06 | >> | >> | 4.41E−07 | TRUE |
| C80396 | 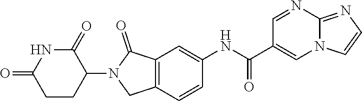 [SMILES: O=C1CCC(N2Cc3ccc(NC(=O)c4cnc5nccn5c4)cc3C2=O)C(=O)N1] N-[2-(2,6-dioxo-3-piperidyl)-3-oxo-isoindolin-5-yl]imidazo[1,2-a] pyrimidine-6-carboxamide | 2.62647E−07 | >> | NA | 1.38E−06 | TRUE |
| C16463 | 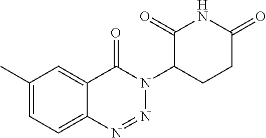 [SMILES: Cc1ccc2nnn(C3CCC(=O)NC3=O)c(=O)c2c1] 3-(6-methyl-4-oxo-1,2,3-benzotriazin-3-yl)piperidine-2,6-dione | 2.67093E−06 | >> | NA | 7.72E−07 | TRUE |
| C64324 | 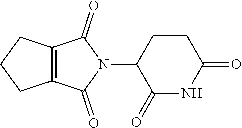 [SMILES: O=C1CCC(N2C(=O)C3=C(CCC3)C2=O)C(=O)N1] 2-(2,6-dioxo-3-piperidyl)-5,6-dihydro-4H-cyclopenta[c] pyrrole-1,3-dione | 2.74862E−06 | NA | NA | NA | FALSE |
| C84967 | 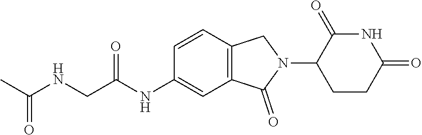 [SMILES: CC(=O)NCC(=O)Nc1ccc2c(c1)C(=O)N(C1CCC(=O)NC1=O)C2] 2-acetamido-N-[2-(2,6-dioxo-3-piperidyl)-3-oxo-isoindolin- 5-yl]acetamide | 3.15182E−06 | NA | NA | >> | TRUE |
| C89940 | 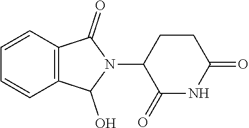 [SMILES: O=C1CCC(N2C(=O)c3ccccc3C2O)C(=O)N1] 3-(1-hydroxy-3-oxo-isoindolin-2-yl)piperidine-2,6-dione | 3.28532E−06 | >> | >> | 4.38E−07 | TRUE |
| C35830 | 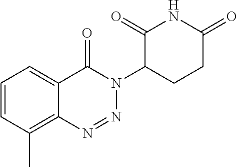 [SMILES: Cc1cccc2c(=O)n(C3CCC(=O)NC3=O)nnc12] 3-(8-methyl-4-oxo-1,2,3-benzotriazin-3-yl)piperidine-2,6-dione | 3.45963E−06 | >> | 5.56E−06 | 5.88E−07 | TRUE |
| C47995 | 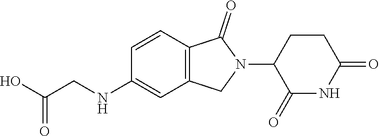 [SMILES: O=C(O)CNc1ccc2c(c1)CN(C1CCC(=O)NC1=O)C2=O] 2-[[2-(2,6-dioxo-3-piperidyl)-1-oxo-isoindolin-5-yl]amino]acetic acid | 3.70808E−06 | >> | >> | 5.18E−07 | TRUE |
| C64376 | 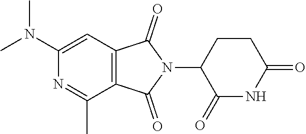 [SMILES: Cc1nc(N(C)C)cc2c1C(=O)N(C1CCC(=O)NC1=O)C2=O] 6-(dimethylamino)-2-(2,6-dioxo-3-piperidyl)-4-methyl-pyrrolo[3,4-c] pyridine-1,3-dione | 3.94892E−06 | NA | NA | NA | FALSE |
| C84961 | 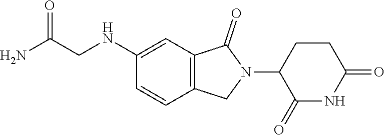 [SMILES: NC(=O)CNc1ccc2c(c1)C(=O)N(C1CCC(=O)NC1=O)C2] 2-[[2-(2,6-dioxo-3-piperidyl)-3-oxo-isoindolin-5-yl]amino]acetamide | 4.00542E−06 | NA | NA | NA | FALSE |
| C22594 | 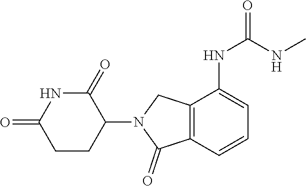 [SMILES: CNC(=O)Nc1cccc2c1CN(C1CCC(=O)NC1=O)C2=O] 1-[2-(2,6-dioxo-3-piperidyl)-1-oxo-isoindolin-4-yl]-3-methyl-urea | 4.49003E−06 | 2.76E−06 | >> | 7.71E−07 | TRUE |
| C12598 | 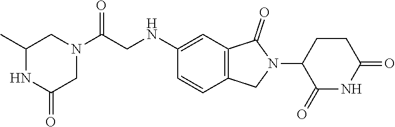 [SMILES: CC1CN(C(=O)CNc2ccc3c(c2)C(=O)N(C2CCC(=O)NC2=O)C3)CC(=O)N1] 3-[6-[[2-(3-methyl-5-oxo-piperazin-1-yl)-2-oxo-ethyl]amino]-1-oxo- isoindolin-2-yl]piperidine-2,6-dione | 4.88466E−06 | NA | NA | NA | FALSE |
| C29408 | 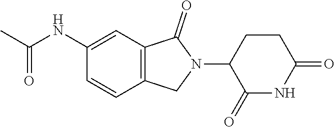 [SMILES: CC(=O)Nc1ccc2c(c1)C(=O)N(C1CCC(=O)NC1=O)C2] N-[2-(2,6-dioxo-3-piperidyl)-3-oxo-isoindolin-5-yl]acetamide | 4.92017E−06 | NA | NA | NA | FALSE |
| C73349 | 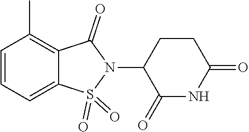 [SMILES: Cc1cccc2c1C(=O)N(C1CCC(=O)NC1=O)S2(=O)=O] 3-(4-methyl-1,1,3-trioxo-1,2-benzothiazol-2-yl)piperidine-2,6-dione | 4.94642E−06 | NA | NA | NA | FALSE |
| 5HPP-33 | 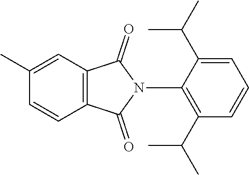 [SMILES: Cc1ccc2c(c1)C(=O)N(c1c(C(C)C)cccc1C(C)C)C2=O] 2-(2,6-diisopropylphenyl)-5-methyl-isoindoline-1,3-dione | 5.1107E−06 | NA | NA | NA | FALSE |
| C12597 | 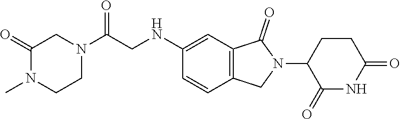 [SMILES: CN1CCN(C(=O)CNc2ccc3c(c2)C(=O)N(C2CCC(=O)NC2=O)C3)CC1=O] 3-[6-[[2-(4-methyl-3-oxo-piperazin-1-yl)-2-oxo-ethyl]amino]-1-oxo- isoindolin-2-yl]piperidine-2,6-dione | 5.50144E−06 | NA | NA | NA | FALSE |
| ZE26-0001 | 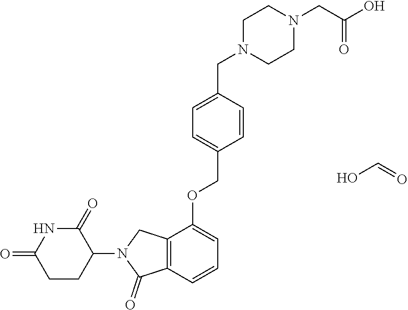 [SMILES: O=C(O)CN1CCN(Cc2ccc(COc3cccc4c3CN(C3CCC(=O)NC3=O)C4=O)cc2)CC1 ; O=CO] 2-[4-[[4-[[2-(2,6-dioxo-3-piperidyl)-1-oxo-isoindolin-4-yl] oxymethyl]phenyl] methyl]piperazin-1-yl]acetic acid; formic acid | 5.76168E−06 | 4.68E−06 | >> | 8.01E−07 | TRUE |
| C98053 | 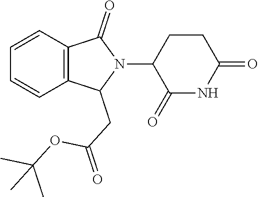 [SMILES: CC(C)(C)OC(=O)CC1c2ccccc2C(=O)N1C1CCC(=O)NC1=O] t-butyl 2-[2-(2,6-dioxo-3-piperidyl)-3-oxo-isoindolin-1-yl]acetate | 5.76611E−06 | NA | NA | NA | FALSE |
| C51830 | 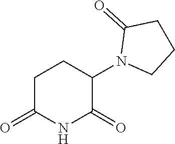 [SMILES: O=C1CCC(N2CCCC2=O)C(=O)N1] 3-(2-oxopyrrolidin-1 yl)piperidine-2,6-dione | 6.03878E−06 | NA | NA | NA | FALSE |
| C97402 | 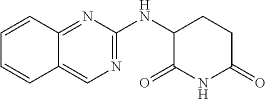 [SMILES: O=C1CCC(Nc2ncc3ccccc3n2)C(=O)N1] 3-(quinazolin-2-ylamino)piperidine-2,6-dione | 6.3232E−06 | NA | NA | NA | FALSE |
| C68121 | 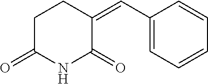 [SMILES: O=C1CC/C(=C/c2ccccc2)C(=O)N1] (3Z)-3-benzylidenepiperidine-2,6-dione | 6.5035E−06 | NA | NA | NA | FALSE |
| C47998 | 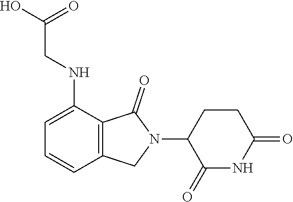 [SMILES: O=C(O)CNc1cccc2c1C(=O)N(C1CCC(=O)NC1=O)C2] 2-[[2-(2,6-dioxo-3-piperidyl)-3-oxo-isoindolin-4-yl]amino]acetic acid | 6.58948E−06 | NA | NA | 2.17E−06 | TRUE |
| C49713 | 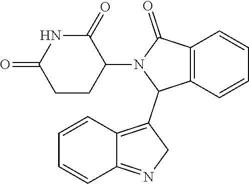 [SMILES: O=C1CCC(N2C(=O)c3ccccc3C2C2=c3ccccc3=NC2)C(=O)N1] 3-[1-(2H-indol-3-yl)-3-oxo-isoindolin-2-yl]piperidine-2,6-dione | 6.73708E−06 | NA | NA | NA | FALSE |
| C36126 | 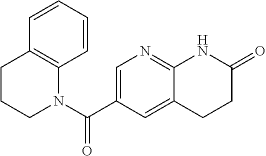 [SMILES: O=C1CCc2cc(C(=O)N3CCCc4ccccc43)cnc2N1] 6-(3,4-dihydro-2H-quinoline-1-carbonyl)-3,4-dihydro-1H-1,8- naphthyridin-2-one | 7.23164E−06 | NA | NA | NA | FALSE |
| C39453 | 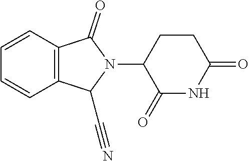 [SMILES: N#CC1c2ccccc2C(=O)N1C1CCC(=O)NC1=O] 2-(2,6-dioxo-3-piperidyl)-3-oxo-isoindoline-1-carbonitrile | 7.51549E−06 | 1.71E−05 | NA | 8.75E−07 | TRUE |
| C10981 | 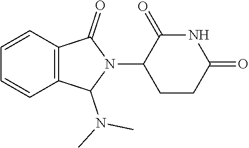 [SMILES: CN(C)C1c2ccccc2C(=O)N1C1CCC(=O)NC1=O] 3-[1-(dimethylamino)-3-oxo-isoindolin-2-yl]piperidine-2,6-dione | 7.80566E−06 | >> | >> | 1.46E−06 | TRUE |
| C80394 | 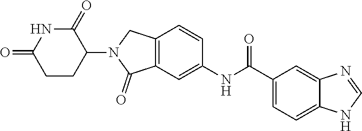 [SMILES: O=C1CCC(N2Cc3ccc(NC(=O)c4ccc5[nH]cnc5c4)cc3C2=O)C(=O)N1] N-[2-(2,6-dioxo-3-piperidyl)-3-oxo-isoindolin-5-yl]-3H-imidazo[4,5-b] pyridine-6-carboxamide | 7.99132E−06 | >> | NA | NA | TRUE |
| C11892 | 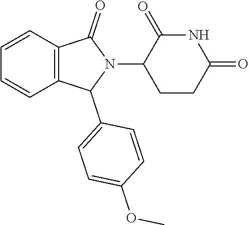 [SMILES: COc1ccc(C2c3ccccc3C(=O)N2C2CCC(=O)NC2=O)cc1] 3-[l-(4-methoxyphenyl)-3-oxo-isoindolin-2-yl1piperidine-2,6-dione | 8.15508E−06 | NA | NA | NA | FALSE |
| C44292 | 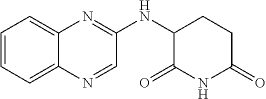 [SMILES: O=C1CCC(Nc2cnc3ccccc3n2)C(=O)N1] 3-(quinoxalin-2-ylamino )piperidine-2,6-dione | 8.32122E−06 | NA | NA | NA | FALSE |
| C12693 | 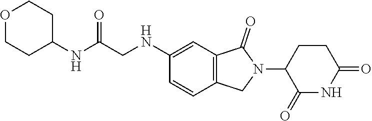 [SMILES: O=C1CCC(N2Cc3ccc(NCC(=O)NC4CCOCC4)cc3C2=O)C(=O)N1] 2-[[2-(2,6-dioxo-3-piperidyl)-3-oxo-isoindolin-5-yl]amino]-N- tetrahydropyran-4-yl-acetamide | 8.32631E−06 | >> | NA | 2.62E−06 | TRUE |
| C56572 | 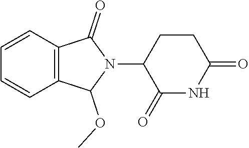 [SMILES: COC1c2ccccc2C(=O)N1C1CCC(=O)NC1=O] 3-(1-methoxy-3-oxo-isoindolin-2-yl)piperidine-2,6-dione | 8.60508E−06 | NA | NA | NA | FALSE |
| C55468 | 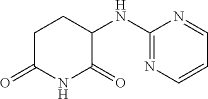 [SMILES: O=C1CCC(Nc2ncccn2)C(=O)N1] 3-(pyrimidin-2-ylamino)piperidine-2,6-dione | 8.68221E−06 | NA | NA | NA | FALSE |
| C29490 | 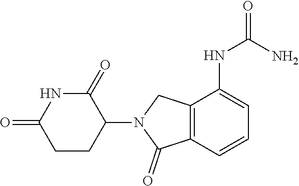 [SMILES: NC(=O)Nc1cccc2c1CN(C1CCC(=O)NC1=O)C2=O] [2-(2,6-dioxo-3-piperidyl)-1-oxo-isoindolin-4-yl]urea | 8.86373E−06 | >>0 | NA | NA | TRUE |
| C47997 | 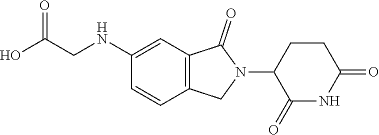 [SMILES: O=C(O)CNc1ccc2c(c1)C(=O)N(C1CCC(=O)NC1=O)C2] 2-[[2-(2,6-dioxo-3-piperidyl)-3-oxo-isoindolin-5-yl]amino]acetic acid | 9.09859E−06 | NA | NA | NA | FALSE |
| C98696 | 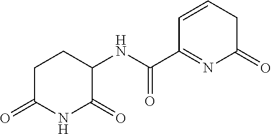 [SMILES: O=C1CC=CC(C(=O)NC2CCC(=O)NC2=O)=N1] N-(2,6-dioxo-3-piperidyl)-2-oxo-3H-pyridine-6-carboxamide | 9.3409E−06 | NA | NA | NA | FALSE |
| C12581 | 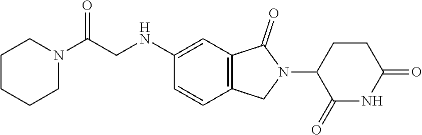 [SMILES: O=C1CCC(N2Cc3ccc(NCC(=O)N4CCCCC4)cc3C2=O)C(=O)N1] 3-[1-oxo-6-[[2-oxo-2-(1-piperidyl)ethyl]amino]isoindolin-2-yl]piperidine- 2,6-dione | 9.99653E−06 | NA | NA | NA | FALSE |
| C12589 | 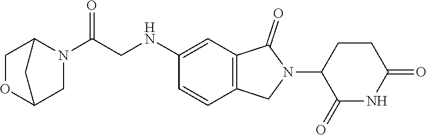 [SMILES: O=C1CCC(N2Cc3ccc(NCC(=O)N4CC5CC4CO5)cc3C2=O)C(=O)N1] 3-[6-[[2-(2-oxa-5-azabicyclo[2.1.1]heptan-5-yl)-2-oxo-ethyl] amino]-1-oxo-isoindolin-2-yl]piperidine [symbol] | 1.02777E−05 | NA | NA | NA | FALSE |
| C15352 | 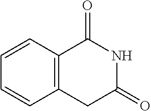 [SMILES: O=C1Cc2ccccc2C(=O)N1] 4H-isoquinoline-1,3-dione | 1.031E−05 | NA | NA | NA | FALSE |
| C95329 | 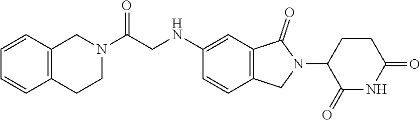 [SMILES: O=C1CCC(N2Cc3ccc(NCC(=O)N4CCc5ccccc5C4)cc3C2=O)C(=O)N1] 3-[6-[[2-(3,4-dihydro-1H-isoquinolin-2-yl)-2-oxo-ethyl] amino]-1-oxo-isoindolin-2-yl]piperidine-2,6- [symbol] | 1.03261E−05 | NA | NA | NA | FALSE |
| C29457 | 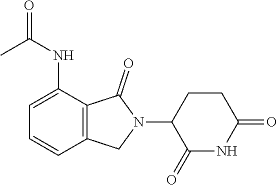 [SMILES: CC(=O)Nc1cccc2c1C(=O)N(C1CCC(=O)NC1=O)C2] N-[2-(2,6-dioxo-3-piperidyl)-3-oxo-isoindolin-4-yl]acetamide | 1.03599E−05 | >> | NA | >> | TRUE |
| C16899 | 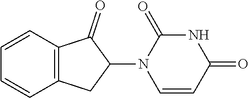 [SMILES: O=C1c2ccccc2CC1n1ccc(=O)[nH]c1=O] 1-(1-oxoindan-2-yl)pyrimidine-2,4-dione | 1.03816E−05 | NA | NA | NA | FALSE |
| C96413 | 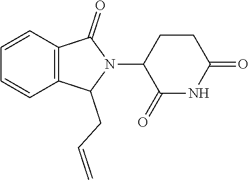 [SMILES: C=CCC1c2ccccc2C(=O)N1C1CCC(=O)NC1=O] 3-(1-allyl-3-oxo-isoindolin-2-yl)piperidine-2,6-dione | 1.03991E−05 | NA | NA | NA | FALSE |
| C40531 | 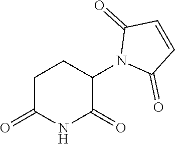 [SMILES: O=C1CCC(N2C(=O)C=CC2=O)C(=O)N1] 3-(2,5-dioxopyrrol-1-yl)piperidine-2,6-dione | 1.10435E−05 | NA | NA | NA | FALSE |
| C75987 | 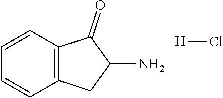 2-aminoindan-1-one; hydrochloride | 1.12251E−05 | NA | NA | NA | FALSE |
| C35745 | 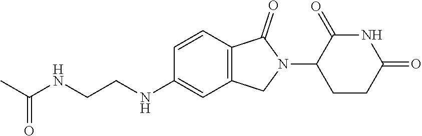 [SMILES: CC(=O)NCCNc1ccc2c(c1)CN(C1CCC(=O)NC1=O)C2=O] N-[2-[[2-(2,6-dioxo-3-piperidyl)-1-oxo-isoindolin-5-yl] amino]ethyl]acetamide | 1.12483E−05 | >> | NA | 2.63E−06 | TRUE |
| C95336 | 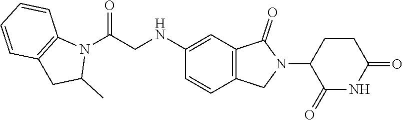 [SMILES: CC1Cc2ccccc2N1C(=O)CNc1ccc2c(c1)C(=O)N(C1CCC(=O)NC1=O)C2] 3-[6-[[2-(2-methylindolin-1-yl)-2-oxo-ethyl]amino]-1-oxo- isoindolin-2-yl]piperidine-2,6-dione | 1.19471E−05 | NA | NA | NA | FALSE |
| C64348 | 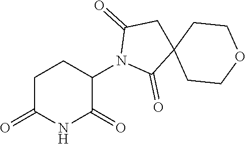 [SMILES: O=C1CCC(N2C(=O)CC3(CCOCC3)C2=O)C(=O)N1] 2-(2,6-dioxo-3-piperidyl)-8-oxa-2-azaspiro[4.5]decane-1,3-dione | 1.29256E−05 | NA | NA | NA | FALSE |
| C02896 | 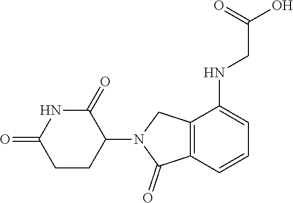 [SMILES: O=C(O)CNc1cccc2c1CN(C1CCC(=O)NC1=O)C2=O] 2-[[2-(2,6-dioxo-3-piperidyl)-1-oxo-isoindolin-4-yl]amino]acetic acid | 1.38151E−05 | NA | NA | NA | FALSE |
| C39772 | 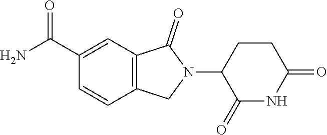 [SMILES: NC(=O)c1ccc2c(c1)C(=O)N(C1CCC(=O)NC1=O)C2] 2-(2,6-dioxo-3-piperidyl)-3-oxo-isoindoline-5-carboxamide | 1.38404E−05 | NA | NA | NA | FALSE |
| C75688 | 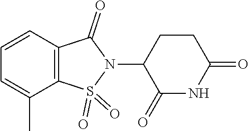 [SMILES: Cc1cccc2c1S(=O)(=O)N(C1CCC(=O)NC1=O)C2=O] 3-(7-methyl-1,1,3-trioxo-1,2-benzothiazol-2-yl)piperidine-2,6-dione | 1.47323E−05 | NA | NA | NA | FALSE |
| C64329 | 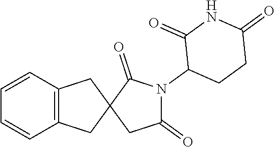 [SMILES: O=C1CCC(N2C(=O)CC3(Cc4ccccc4C3)C2=O)C(=O)N1] 1′-(2,6-dioxo-3-piperidyl)spiro[indane-2,3′-pyrrolidine]-2′,5′-dione | 1.51214E−05 | NA | NA | NA | FALSE |
| C34491 | 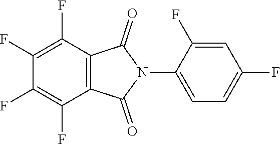 [SMILES: O=C1c2c(F)c(F)c(F)c(F)c2C(=O)N1c1ccc(F)cc1F] 2-(2,4-difluorophenyl)-4,5,6,7-tetrafluoro-isoindoline-1,3-dione | 1.61223E−05 | NA | NA | NA | FALSE |
| 5′-OH-THL | 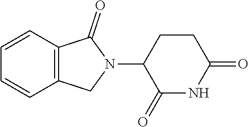 [SMILES: O=C1CCC(N2Cc3ccccc3C2=O)C(=O)N1] 3-(1-oxoisoindolin-2-yl)piperidine-2,6-dione | 1.61621E−05 | NA | NA | NA | FALSE |
| C80374 | 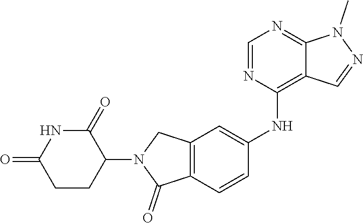 [SMILES: Cn1ncc2c(Nc3ccc4c(c3)CN(C3CCC(=O)NC3=O)C4=O)ncnc21] 3-[5-[(1-methylpyrazolo[3,4-d]pyrimidin-4-yl)amino]-1-oxo- isoindolin-2-yl]piperidine-2,6-dione | 1.65805E−05 | >> | NA | NA | TRUE |
| C24191 | 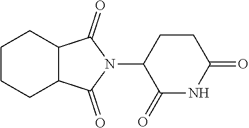 [SMILES: O=C1CCC(N2C(=O)C3CCCCC3C2=O)C(=O)N1] 2-(2,6-dioxo-3-piperidyl)-3a,4,5,6,7,7a-hexahydroisoindole-1,3-dione | 1.7705E−05 | NA | NA | NA | FALSE |
| C87699 | 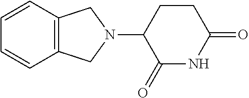 [SMILES: O=C1CCC(N2Cc3ccccc3C2)C(=O)N1] 3-isoindolin-2-ylpiperidine-2.6-dione | 1.96334E−05 | NA | NA | 4.82E−06 | TRUE |
| C36128 | 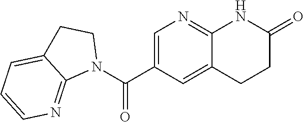 [SMILES: O=C1CCc2cc(C(=O)N3CCc4cccnc43)cnc2N1] 6-(2,3-dihydropyrrolo[2,3-b]pyridine-1-carbonyl)-3,4- dihydro-1H-1,8-naphthyridin-2-one________ | 2.02561E−05 | NA | NA | NA | FALSE |
| C17800 | 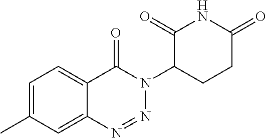 [SMILES: Cc1ccc2c(=O)n(C3CCC(=O)NC3=O)nnc2c1] 3-(7-methyl-4-oxo-1,2,3-benzotriaan-3-yl)piperidirie-2,6-dione | 2.18924E−05 | >> | NA | 9.04E−07 | TRUE |
| C08493 | 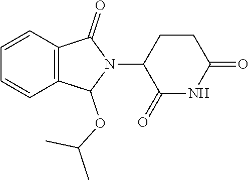 [SMILES: CC(C)OC1c2ccccc2C(=O)N1C1CCC(=O)NC1=O] 3-(1-isopropoxy-3-oxo-isoindolin-2-yl)piperidine-2,6-dione | 2.28752E−05 | >> | NA | 2.70E−06 | TRUE |
| C07128 | 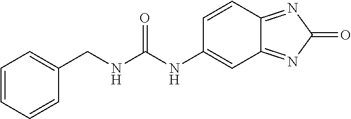 [SMILES: O=C1N=c2ccc(NC(=O)NCc3ccccc3)cc2=N1] 1-benzyl-3-(2-oxobenzimidazol-5-yl)urea | 2.3835E−05 | NA | NA | NA | FALSE |
| C64319 | 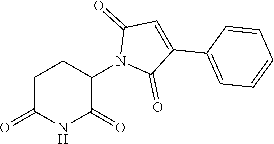 [SMILES: O=C1CCC(N2C(=O)C=C(c3ccccc3)C2=O)C(=O)N1] 3-(2,5-dioxo-3-phenyl-pyrrol-1-yl)piperidine-2,6-dione | 4.28801E−05 | NA | NA | NA | FALSE |
| C49278 | 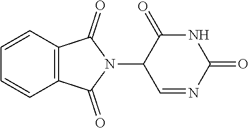 [SMILES: O=C1N=CC(N2C(=O)c3ccccc3C2=O)C(=O)N1] 2-(2,6-dioxo-5H-pyrimidin-5-yl)isoindoline-1,3-dione | 4.53958E−05 | NA | NA | NA | FALSE |
| C33779 | 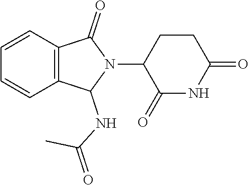 [SMILES: CC(=O)NC1c2ccccc2C(=O)N1C1CCC(=O)NC1=O] N-[2-(2,6-dioxo-3-piperidyl)-3-oxo-isoindolin-1-yl]acetamide | 4.94369E−05 | NA | NA | 3.03E−06 | TRUE |
| C12695 | 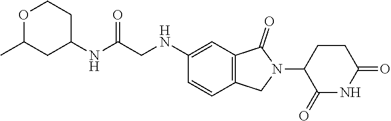 [SMILES: CC1CC(NC(=O)CNc2ccc3c(c2)C(=O)N(C2CCC(=O)NC2=O)C3)CCO1] 2-[[2-(2,6-dioxo-3-piperidyl)-3-oxo-isoindolin-5-yl]amino]- N-(2-methyltetrahydropyran-4-yl)acetamide | 5.79759E−05 | NA | NA | NA | FALSE |
| C09563 | 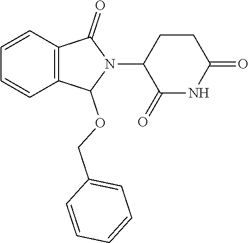 [SMILES: O=C1CCC(N2C(=O)c3ccccc3C2OCc2ccccc2)C(=O)N1] 3-(1-benzyloxy-3-oxo-isoindolin-2-yl)piperidine-2,6-dione | 5.82854E−05 | NA | NA | NA | FALSE |
| glutarimide | 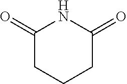 piperidine-2,6-dione | 9.53602E−05 | NA | NA | NA | FALSE |
| C38935 | 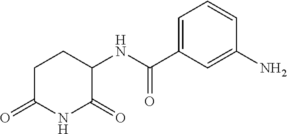 [SMILES: Nc1cccc(C(=O)NC2CCC(=O)NC2=O)c1] 3-amino-N-(2,6-dioxo-3-piperidyl)benzamide | 9.92946E−05 | >> | NA | NA | TRUE |
| C30231 | 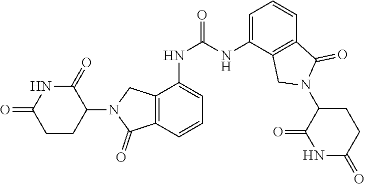 [SMILES: O=C1CCC(N2Cc3c(NC(=O)Nc4cccc5c4CN(C4CCC(=O)NC4=O)C5=O)cccc3C2=O)C(=O)N1] 1,3-bis[2-(2,6-dioxo-3-piperidyl)-1-oxo-isoindolin-4-yl]urea | 0.000247103 | 2.73E−06 | >> | 1.7E−06 | TRUE |
| C64340 | 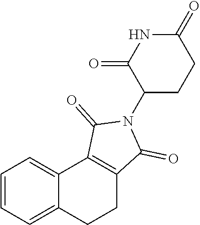 [SMILES: O=C1CCC(N2C(=O)C3=C(C2=O)c2ccccc2CC3)C(=O)N1] 2-(2,6-dioxo-3-piperidyl)-4,5-dihydrobenzo[e]isoindole-1,3-dione | 0.002503261 | >> | NA | 1.49E−06 | TRUE |
| C23258 | 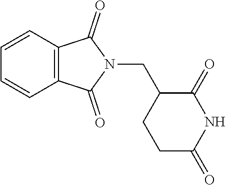 [SMILES: O=C1CCC(CN2C(=O)c3ccccc3C2=O)C(=O)N1] 2-[(2,6-dioxo-3-piperidyl)methyl]isoindoline-1,3-dione | 0.014526546 | NA | NA | NA | FALSE |
| C12582 | 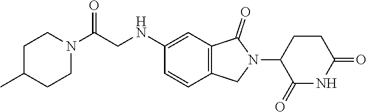 [SMILES: CC1CCN(C(=O)CNc2ccc3c(c2)C(=O)N(C2CCC(=O)NC2=O)C3)CC1] 3-[6-[[2-(4-methyl-1-piperidyl)-2-oxo-ethyl]amino]-1-oxo- isoindolin-2-yl]piperidine-2,6-dione | 0.037396754 | NA | NA | NA | FALSE |
| C60651 | 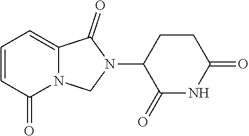 [SMILES: O=C1CCC(N2Cn3c(cccc3=O)C2=O)C(=O)N1] 2-(2,6-dioxo-3-piperidyl)-3H-imidazo[1,5-a]pyridine-1,5-dione | 0.113371238 | NA | NA | NA | FALSE |
| C29737 | 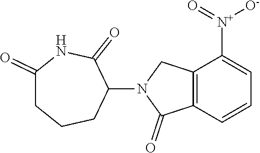 [SMILES: O=C1CCCC(N2Cc3c(cccc3[N+](=O)[O-])C2=O)C(=O)N1] 3-(4-nitro-1-oxo-isoindolin-2-yl)azepane-2,7-dione | 0.122564012 | 1.41E−06 | NA | NA | TRUE |
| C64372 | 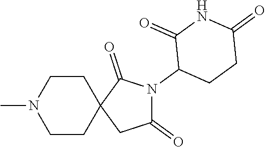 [SMILES: CN1CCC2(CC1)CC(=O)N(C1CCC(=O)NC1=O)C2=O] 2-(2,6-dioxo-3-piperidyl)-8-methyl-2,8-diazaspiro[4.5]decane-1,3-dione | 0.223963175 | NA | NA | NA | FALSE |
| C09063 | 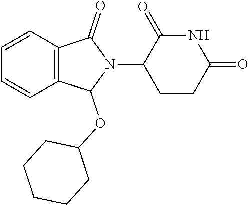 [SMILES: O=C1CCC(N2C(=O)c3ccccc3C2OC2CCCCC2)C(=O)N1] 3-[1-(cyclohexoxy)-3-oxo-isoindolin-2-yl]piperidine-2,6-dione | 0.453327822 | NA | NA | NA | FALSE |
| C95334 | 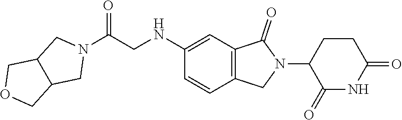 [SMILES: O=C1CCC(N2Cc3ccc(NCC(=O)N4CC5COCC5C4)cc3C2=O)C(=O)N1] 3-[6-[[2-(1,3,3a,4,6,6a-hexahydrofuro[3,4-c]pyrrol-5-yl)-2- oxo-ethyl]amino]-1-oxo-isoindolin-2-yl]piperidine-2.6-dione | 1.594934429 | NA | NA | NA | FALSE |
| C14950 | 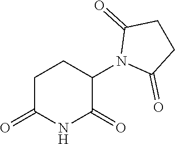 [SMILES: O=C1CCC(N2C(=O)CCC2=O)C(=O)N1] 3-(2,5-dioxopyrrolidin-1-yl)piperidine-2,6-dione | 3.958295874 | NA | NA | NA | FALSE |
| C46003 | 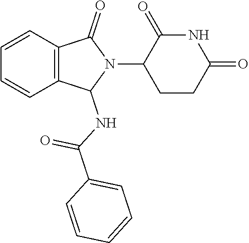 [SMILES: O=C1CCC(N2C(=O)c3ccccc3C2NC(=O)c2ccccc2)C(=O)N1] N-[2-(2,6-dioxo-3-piperidyl)-3-oxo-isoindolin-1-yl]benzamide | 32.56223026 | NA | NA | NA | FALSE |
| C51647 | 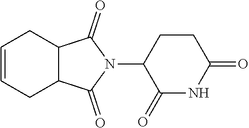 [SMILES: O=C1CCC(N2C(=O)C3CC=CCC3C2=O)C(=O)N1] 2-(2,6-dioxo-3-piperidyl)-3a,4,7,7a-tetrahydroisoindole-1,3-dione | 51.17761807 | NA | NA | NA | FALSE |
| C12697 | 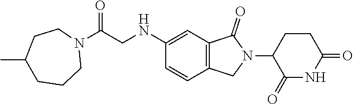 [SMILES: CC1CCCN(C(=O)CNc2ccc3c(c2)C(=O)N(C2CCC(=O)NC2=O)C3)CC1] 3-[6-[[2-(4-methylazepan-1-yl)-2-oxo-ethyl]amino]-1-oxo- isoindolin-2-yl]piperidine-2,6-dione | 51.67955472 | NA | NA | NA | FALSE |
| C64344 | 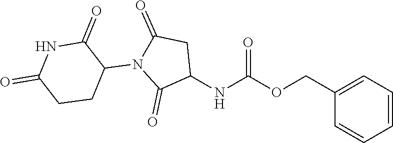 [SMILES: O=C1CCC(N2C(=O)CC(NC(=O)OCc3ccccc3)C2=O)C(=O)N1] benzyl N-[1-(2,6-dioxo-3-piperidyl)-2,5-dioxo-pyrrolidin-3- yl]carbamate | 112.7097496 | NA | NA | NA | FALSE |
| C10537 | 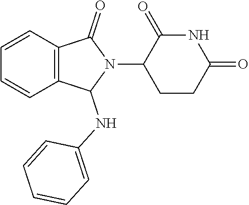 [SMILES: O=C1CCC(N2C(=O)c3ccccc3C2Nc2ccccc2)C(=O)N1] 3-(1-anilino-3-oxo-isoindolin-2-yl)piperidine-2,6-dione | | NA | NA | NA | FALSE |
| C12694 | 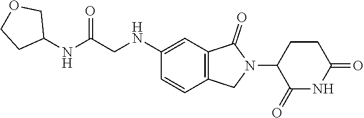 [SMILES: O=C1CCC(N2Cc3ccc(NCC(=O)NC4CCOC4)cc3C2=O)C(=O)N1] 2-[[2-(2,6-dioxo-3-piperidyl)-3-oxo-isoindolin-5-yl]amino]-N- tetrahydrofuran-3-yl-acetamide | | >> | NA | 3.43E−06 | TRUE |
| C38930 | 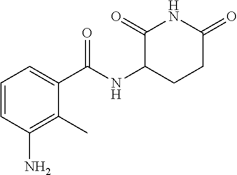 [SMILES: Cc1c(N)cccc1C(=O)NC1CCC(=O)NC1=O] 3-amino-N-(2,6-dioxo-3-piperidyl)-2-methyl-benzamide | | NA | NA | NA | FALSE |
| C80385 | 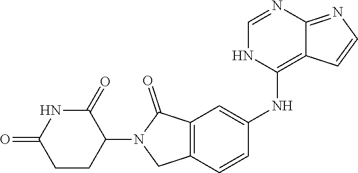 [SMILES: O=C1CCC(N2Cc3ccc(Nc4[nH]cnc5nccc4-5)cc3C2=O)C(=O)N1] 3-[1-oxo-6-(3H-pyrrolo[2,3-d]pyrimidin-4-ylamino)isoindolin-2-yl] piperidine-2,6-dione | | NA | NA | NA | FALSE |
| C80393 | 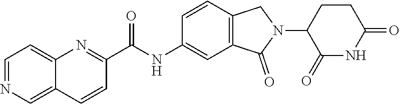 [SMILES: O=C1CCC(N2Cc3ccc(NC(=O)c4ccc5cnccc5n4)cc3C2=O)C(=O)N1] N-[2-(2,6-dioxo-3-piperidyl)-3-oxo-isoindolin-5-yl]-1,6- naphthyridine-2-carboxamide | 7.00E−09 | NA | NA | NA | FALSE |
| C47930 | 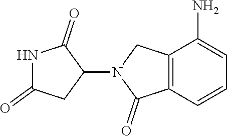 [SMILES: Nc1cccc2c1CN(C1CC(=O)NC1=O)C2=O] 3-(4-amino-1-oxo-isoindolin-2-yl)pyrrolidine-2,5-dione | | 7.84E−07 | NA | NA | TRUE |
| C47927 | 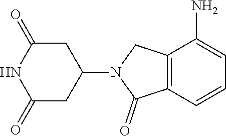 [SMILES: Nc1cccc2c1CN(C1CC(=O)NC(=O)C1)C2=O] 4-(4-amino-1-oxo-isoindolin-2-yl)piperidine-2,6-dione | | >> | NA | NA | TRUE |
| C47932 | 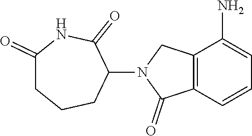 [SMILES: Nc1cccc2c1CN(C1CCCC(=O)NC1=O)C2=O] 3-(4-amino-1-oxo-isoindolin-2-yl)azepane-2,7-dione | | >> | NA | NA | TRUE |
| C47928 | 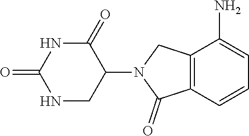 [SMILES: Nc1cccc2c1CN(C1CNC(=O)NC1=O)C2=O] 5-(4-amino-1-oxo-isoindolin-2-yl)hexahydropyrimidine-2,4-dione | | NA | NA | NA | FALSE |
| C10239 | 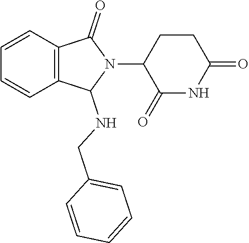 [SMILES: O=C1CCC(N2C(=O)c3ccccc3C2NCc2ccccc2)C(=O)N1] 3-[1-(benzylamino)-3-oxo-isoindolin-2-yl]piperidine-2,6-dione | | >> | NA | 1.16E−06 | TRUE |
| C36124 | 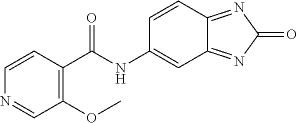 [SMILES: COc1cnccc1C(=O)Nc1ccc2c(c1)=NC(=O)N=2] 3-methoxy-N-(2-oxobenzimidazol-5-yl)pyridine-4-carboxamide | | NA | NA | NA | FALSE |
| C80376 | 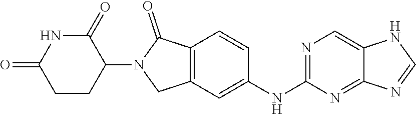 [SMILES: O=C1CCC(N2Cc3cc(Nc4ncc5[nH]cnc5n4)ccc3C2=O)C(=O)N1] 3-[1-oxo-5-(7H-purin-2-ylamino)isoindolin-2-yl]piperidine-2,6-dione | | >> | NA | 5.59E−07 | TRUE |
| C10001 | 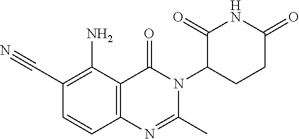 [SMILES: Cc1nc2ccc(C#N)c(N)c2c(=O)n1C1CCC(=O)NC1=O] 5-amino-3-(2,6-dioxo-3-piperidyl)-2-methyl-4-oxo- quinazoline-6-carbonitrile | | >> | >> | 1.25E−06 | TRUE |
| C55859 | 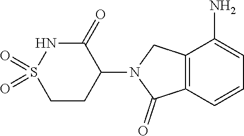 [SMILES: Nc1cccc2c1CN(C1CCS(=O)(=O)NC1=O)C2=O] 4-(4-Amino-1-oxoisoindolin-2yl)-1,2-thiazinan-3-one 1,1-dioxide | | NA | NA | NA | FALSE |
| THL | 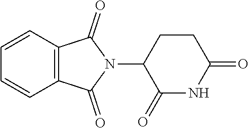 [SMILES: O=C1CCC(N2C(=O)c3ccccc3C2=O)C(=O)N1] 2-(2,6-dioxo-3-piperidyl)isonidoline-1,3-dione | | >> | >> | NA | TRUE |

EC50 values for recruitment of IKZF1, ASS1 and SALL4 were determined using the protocols and reagents described in Example 2. Recruitment assessment was made for each compound, where TRUE = observation of recruitment of any tested substrate at any tested compound concentration from among IKZF1, ASS1 and SALL4, and FALSE = no observed recruitment of any tested substrate at any tested compound concentration from among IKF1, ASS1 and SALL4. NA represents instances where no substrate recruitment was observed at any tested concentration , >> represents instances were an EC50 curve could not be calculated as values did not reach a plateau over the measured concentration range, and << represents instances where an EC50 could not be calculated because a value was read above 50% of control at the first concentration tested for the compound (13.7 nM). [symbol]
indicates data missing or illegible when filed

1. A compound of Formula I: 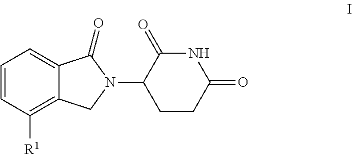
or a pharmaceutically acceptable salt thereof, wherein:
R¹ is aryl, —N(R⁵)—X—R⁶, —SO₂R⁵, or —O(CH₂)ₘR⁵, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

2. The compound of claim 1, wherein:
R¹ is aryl optionally substituted with 1 or more R^{w} groups as allowed by valence.

3. A compound of Formula II, 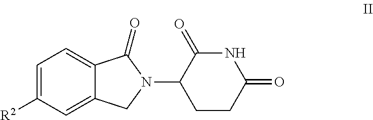
or a pharmaceutically acceptable salt thereof, wherein:
R² is aryl, —NH—(C₃-C₁₀) heteroaryl, or —N(R⁵)—(CH₂)ₘ—X—(CH₂)ₙ—R⁶, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

4. The compound of claim 3, wherein:
R² is —N(R⁵)—(CH₂)ₘ—X—(CH₂)ₙ—R⁶;
R⁵ is H;
R⁶ is OH, (C₁-C₃)alkyl, —(C₁-C₃)alkoxy, —NR⁵R⁵, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl.

5. A compound of Formula III 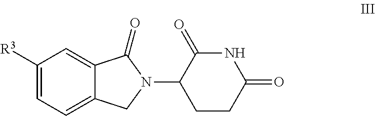
or a pharmaceutically acceptable salt thereof, wherein:
R³ is cyano, aryl, —NH—(C₃-C₁₀) heteroaryl, (C₃-C₁₀)heterocyclo, or —N(R⁵)—(CH₂)ₘ—X—(CH₂)ₙ—R⁶, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

6. The compound of claim 5, wherein
R³ is —N(R⁵)—(CH₂)ₘ—X—(CH₂)ₙ—R⁶;
R⁵ is H;
R⁶ is OH, (C₁-C₃)alkyl, —(C₁-C₃)alkoxy, —NR⁵R⁵, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl.

7. A compound of Formula IV 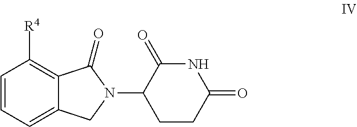
or a pharmaceutically acceptable salt thereof, wherein:
R⁴ is halo, cyano, aryl, OR⁵, or —N(R⁵)—(CH₂)ₘ—X—(CH₂)ₙ—R⁶, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

8. The compound of claim 7, wherein
R⁴ is —N(R⁵)—(CH₂)ₘ—X—(CH₂)ₙ—R⁶;
R⁵ is H;
R⁶ is OH, (C₁-C₃)alkyl, —(C₁-C₃)alkoxy, —NR⁵R⁵, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl.

9. A compound of Formula V 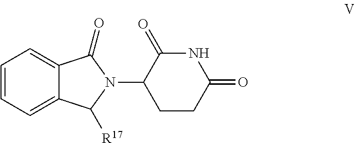
or a pharmaceutically acceptable salt thereof, wherein:
R¹⁷ is cyano, heteroaryl, —(CH₂)ₘ—C(O)O—R⁶, or —N(R⁵)—(CH₂)ₘ—X—(CH₂)ₙ—R⁶, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

10. A compound of Formula VI 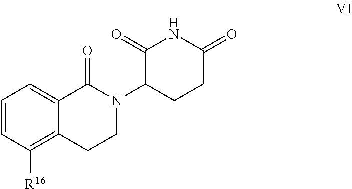
or a pharmaceutically acceptable salt thereof, wherein:
R¹⁶ is NH₂ or —N(R⁵)—(CH₂)ₘ—X—(CH₂)ₙ—R⁶, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

11. A compound of Formula VII 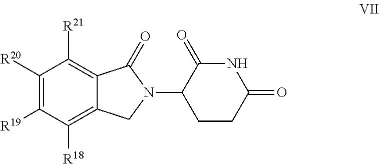
or a pharmaceutically acceptable salt thereof, wherein:
R¹⁸, R¹⁹, R²⁰, R²¹ each independently is H, halo, (C₁-C₃)alkyl, (C₁-C₃)alkoxy, or —N(R⁵)—X—R⁶, with the proviso that no more than two substituents of R¹⁸, R¹⁹, R²⁰, R²¹ are H; or
R¹⁸, R¹⁹ taken together with the carbons they are attached to forming a (C₃-C₁₀)cycloalkyl or a (C₃-C₁₀)heterocyclo, or R¹⁹, R²⁰ taken together with the carbons they are attached to forming a (C₃-C₁₀)cycloalkyl or a (C₃-C₁₀)heterocyclo, or R²⁰, R²¹ taken together with the carbons they are attached to forming a (C₃-C₁₀)cycloalkyl or a (C₃-C₁₀)heterocyclo, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

12. A compound of Formula VIII 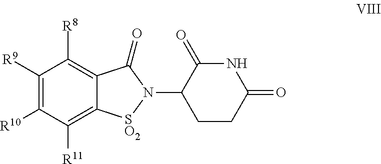
or a pharmaceutically acceptable salt thereof, wherein:
R⁸, R⁹, R¹⁰, R¹¹ each independently is H, halo, OH, cyano, (C₁-C₃)alkyl, (C₁-C₃)alkoxy, aryl, or heteroaryl, any of which may be optionally substituted with 1 or more R^{w} groups as allowed by valence;
R^{w} at each occurrence is independently, H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
n is 0, 1, 2, 3, or 4.

13. A compound of Formula IX 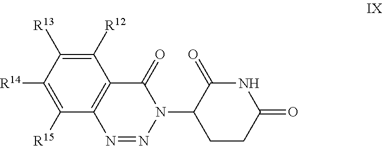
or a pharmaceutically acceptable salt thereof, wherein:
R¹², R¹³, R¹⁴, R¹⁵ each is independently H, NH₂, (C₁-C₃)alkyl, —N(R⁵)—(CH₂)ₘ—N(R⁵)—X—R⁶, with proviso that no more than three substituents out of R¹², R¹³, R¹⁴, and R¹⁵ are H, any of which may be optionally substituted with 1 or more R^{w} groups as allowed by valence;
R⁵ at each occurrence is independently H, (C₁-C₃)alkyl, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, or heteroaryl, any of which may be optionally substituted with 1 or more R^{w} groups as allowed by valence;
R⁶ at each occurrence is independently OH, (C₁-C₃)alkyl, —(C₁-C₃)alkoxy, (C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, heteroaryl, or R⁵ and R⁶ taken together with the atoms they are attached to forming a nitrogen containing (C₃-C₁₀)heterocyclic ring, any of which may be optionally substituted with 1 or more R^{w} groups as allowed by valence;
R^{w} at each occurrence is independently H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
X is a bond, —SO₂—, —(CH₂)ₙC(O)(CH₂)ₘ—, —C(O)NH—, —C(O)N(R^{w})—, —NHC(O)NH—, or —(CH₂)ₙ—;
m is 0, 1, 2, 3, or 4;
n is 0, 1, 2, 3, or 4.

14. A compound of Formula X 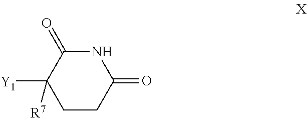
or a pharmaceutically acceptable salt thereof, wherein:
Y₁ is —NHR³³, —NHC(O)R³³, or —CHR³³R³⁴;
R⁷ is H, (C₁-C₃)alkyl, or R⁷ and R³⁴ taken together with the carbons they are attached to forming a carbon carbon double bond;
R³³ is aryl, heteroaryl, or (C₃-C₁₀)heterocyclo, any of which may be optionally substituted with 1 or more R^{w} groups as allowed by valence;
R^{w} at each occurrence is independently, H, halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, wherein said alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more groups selected from the group consisting of halo, cyano, oxo(C₃-C₁₀)heterocyclo, (C₃-C₁₀)cycloalkyl, —(CH₂)ₙ—(C₃-C₁₀) cycloalkyl, —(CH₂)ₙ—(C₃-C₁₀)heterocyclo, —(CH₂)ₙ-aryl, —(CH₂)ₙ-heteroaryl, aryl, and heteroaryl;
n is 0, 1, 2, 3, or 4.

15. A compound selected from the group consisting of:
3-[1-oxo-5-(quinazolin-4-ylamino)isoindolin-2-yl]piperidine-2,6-dione;
3-[5-[(4-aminothieno[2,3-d]pyrimidin-2-yl)amino]-1-oxo-isoindolin-2-yl]piperidine-2,6-dione;
N-[2-(2,6-dioxo-3-piperidyl)-1-oxo-isoindolin-5-yl]acetamide;
3-[5-[(2-aminopyrimidin-4-yl)amino]-1-oxo-isoindolin-2-yl]piperidine-2,6-dione;
6-[[2-(2,6-dioxo-3-piperidyl)-1-oxo-isoindolin-5-yl]amino]pyridazine-3-carbonitrile;
3-[[2-[[2-(2,6-dioxo-3-piperidyl)-1-oxo-isoindolin-5-yl]amino]acetyl]amino]benzamide;
2-[[2-(2,6-dioxo-3-piperidyl)-1-oxo-isoindolin-5-yl]amino]acetic acid;
N-[2-(2,6-dioxo-3-piperidyl)-3-oxo-isoindolin-5-yl]quinoline-2-carboxamide;
3-[6-[[2-(2-methyl-1-piperidyl)-2-oxo-ethyl]amino]-1-oxo-isoindolin-2-yl]piperidine-2,6-dione;
3-[6-[(2-isoindolin-2-yl-2-oxo-ethyl)amino]-1-oxo-isoindolin-2-yl]piperidine-2,6-dione;
N-(cyclopropylmethyl)-2-[[2-(2,6-dioxo-3-piperidyl)-3-oxo-isoindolin-5-yl]amino]-N-methyl-acetamide;
acetic acid; 3-[1-oxo-6-(quinazolin-4-ylamino)isoindolin-2-yl]piperidine-2,6-dione;
3-[6-[[2-(3-methyl-1-piperidyl)-2-oxo-ethyl]amino]-1-oxo-isoindolin-2-yl]piperidine-2,6-dione;
3-[6-[(4-methyl-3-oxo-pyrazin-2-yl)amino]-1-oxo-isoindolin-2-yl]piperidine-2,6-dione;
3-[1-oxo-6-(quinoxalin-2-ylamino)isoindolin-2-yl]piperidine-2,6-dione;
3-[6-[(1-methylpyrazolo[3,4-d]pyrimidin-4-yl)amino]-1-oxo-isoindolin-2-yl]piperidine-2,6-dione;
3-[6-(5,7-dihydrofuro[3,4-d]pyrimidin-2-ylamino)-1-oxo-isoindolin-2-yl]piperidine-2,6-dione;
3-[6-[(6-methylpyrimidin-4-yl)amino]-1-oxo-isoindolin-2-yl]piperidine-2,6-dione;
2-[[2-(2,6-dioxo-3-piperidyl)-3-oxo-isoindolin-5-yl]amino]-N-phenyl-acetamide;
3-[6-[[2-(2,4-dimethylpiperazin-1-yl)-2-oxo-ethyl]amino]-1-oxo-isoindolin-2-yl]piperidine-2,6-dione;
3-[6-(dimethylamino)-1-oxo-isoindolin-2-yl]piperidine-2,6-dione;
3-(1-oxo-6-phenyl-isoindolin-2-yl)piperidine-2,6-dione;
2-[[2-(2,6-dioxo-3-piperidyl)-3-oxo-isoindolin-5-yl]amino]-N,N-dimethyl-acetamide;
3-[6-[[2-(2-methylmorpholin-4-yl)-2-oxo-ethyl]amino]-1-oxo-isoindolin-2-yl]piperidine-2,6-dione;
2-[[2-(2,6-dioxo-3-piperidyl)-3-oxo-isoindolin-5-yl]amino]-N-methyl-N-[(1-methylpyrazol-4-yl)methyl]acetamide;
N-benzyl-2-[[2-(2,6-dioxo-3-piperidyl)-3-oxo-isoindolin-5-yl]amino]acetamide;
6-[[2-(2,6-dioxo-3-piperidyl)-3-oxo-isoindolin-5-yl]amino]pyridazine-3-carbonitrile;
3-[6-[(6-methylpyrrolo[3,2-d]pyrimidin-4-yl)amino]-1-oxo-isoindolin-2-yl]piperidine-2,6-dione;
2-(dimethylamino)-N-[2-(2,6-dioxo-3-piperidyl)-3-oxo-isoindolin-5-yl]acetamide;
N-[2-(2,6-dioxo-3-piperidyl)-3-oxo-isoindolin-5-yl]-5H-pyrrolo[2,3-b]pyridine-4-carboxamide;
N-cyclopropyl-2-[[2-(2,6-dioxo-3-piperidyl)-3-oxo-isoindolin-5-yl]amino]acetamide;
3-[1-oxo-6-(2-oxoimidazolidin-1-yl)isoindolin-2-yl]piperidine-2,6-dione;
2-(2,6-dioxo-3-piperidyl)-3-oxo-isoindoline-5-carbonitrile;
2-[[2-(2,6-dioxo-3-piperidyl)-3-oxo-isoindolin-5-yl]amino]propanoic acid;
2-acetamido-N-[2-(2,6-dioxo-3-piperidyl)-3-oxo-isoindolin-5-yl]acetamide;
2-[[2-(2,6-dioxo-3-piperidyl)-3-oxo-isoindolin-5-yl]amino]acetamide;
3-[6-[[2-(3-methyl-5-oxo-piperazin-1-yl)-2-oxo-ethyl]amino]-1-oxo-isoindolin-2-yl]piperidine-2,6-dione;
N-[2-(2,6-dioxo-3-piperidyl)-3-oxo-isoindolin-5-yl]acetamide;
3-[6-[[2-(4-methyl-3-oxo-piperazin-1-yl)-2-oxo-ethyl]amino]-1-oxo-isoindolin-2-yl]piperidine-2,6-dione;
N-[2-(2,6-dioxo-3-piperidyl)-3-oxo-isoindolin-5-yl]-3H-imidazo[4,5-b]pyridine-6-carboxamide;
2-[[2-(2,6-dioxo-3-piperidyl)-3-oxo-isoindolin-5-yl]amino]-N-tetrahydropyran-4-yl-acetamide;
2-[[2-(2,6-dioxo-3-piperidyl)-3-oxo-isoindolin-5-yl]amino]acetic acid;
3-[1-oxo-6-[[2-oxo-2-(1-piperidyl)ethyl]amino]isoindolin-2-yl]piperidine-2,6-dione;
3-(1-oxo-7-phenyl-isoindolin-2-yl)piperidine-2,6-dione;
2-(2,6-dioxo-3-piperidyl)-3-oxo-isoindoline-4-carbonitrile;
2-[[2-(2,6-dioxo-3-piperidyl)-3-oxo-isoindolin-4-yl]amino]acetic acid;
3-(7-fluoro-1-oxo-isoindolin-2-yl)piperidine-2,6-dione;
3-(5-amino-1-oxo-3,4-dihydroisoquinolin-2-yl)piperidine-2,6-dione;
t-butyl 2-[2-(2,6-dioxo-3-piperidyl)-3-oxo-isoindolin-1-yl]acetate;
3-[1-(2H-indol-3-yl)-3-oxo-isoindolin-2-yl]piperidine-2,6-dione;
2-(2,6-dioxo-3-piperidyl)-3-oxo-isoindoline-1-carbonitrile;
3-[1-(dimethylamino)-3-oxo-isoindolin-2-yl]piperidine-2,6-dione;
3-(2-oxopyrrolidin-1-yl)piperidine-2,6-dione;
3-(quinazolin-2-ylamino)piperidine-2,6-dione;
(3Z)-3-benzylidenepiperidine-2,6-dione;
3-(quinoxalin-2-ylamino)piperidine-2,6-dione;
3-(pyrimidin-2-ylamino)piperidine-2,6-dione;
N-(2,6-dioxo-3-piperidyl)-2-oxo-3H-pyridine-6-carboxamide;
3-(4-methyl-1,1,3-trioxo-1,2-benzothiazol-2-yl)piperidine-2,6-dione;
3-(8-amino-4-oxo-1,2,3-benzotriazin-3-yl)piperidine-2,6-dione;
3-(5-amino-4-oxo-1,2,3-benzotriazin-3-yl)piperidine-2,6-dione;
3-(4-oxo-1,2,3-benzotriazin-3-yl)piperidine-2,6-dione;
3-(5-methyl-4-oxo-1,2,3-benzotriazin-3-yl)piperidine-2,6-dione;
3-(6-methyl-4-oxo-1,2,3-benzotriazin-3-yl)piperidine-2,6-dione; and
3-(8-methyl-4-oxo-1,2,3-benzotriazin-3-yl)piperidine-2,6-dione.

16. A composition comprising a pharmaceutically effective amount of the compound of any of the preceding claims and a pharmaceutically acceptable carrier.

17. A method of treating a disease comprising administering the composition of claim 16 to a subject in need thereof.

18. The method of claim 17, wherein the disease is a cancer.

19. The method of claim 18, wherein the cancer is selected from the group consisting of squamous-cell carcinoma, basal cell carcinoma, adenocarcinoma, hepatocellular carcinomas, renal cell carcinomas, bladder cancer, bowel cancer, breast cancer, cervical cancer, colon cancer, esophageal cancer, head cancer, kidney cancer, liver cancer, lung cancer, neck cancer, ovarian cancer, pancreatic cancer, prostate cancer, stomach cancer, uterine cancer, leukemias, lymphomas, Burkitt's lymphoma, Non-Hodgkin's lymphoma, melanomas, myeloproliferative diseases, multiple myeloma, sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, Schwannomas, testicular cancer, thyroid cancer, astrocytoma, Hodgkin's disease, Wilms' tumor, and teratocarcinomas.

20. The method of claim 19, wherein the cancer is multiple myeloma.

21. The method of claim 17, wherein the disease is an autoimmune disease or disorder.

22. The method of claim 21, wherein the the autoimmune disease or disorder is selected from, such as multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome;
transplantation rejection (e.g., prevention of allograft rejection) pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, and other autoimmune diseases or disorders.

23. A method of modulating cereblon comprising administering the composition of claim 16 to a subject in need thereof.

24. A method of modulating proteasomal degradation of a protein comprising administering the composition of claim 16 to a subject in need thereof.

25. A method of modulating sequestration of a protein to the proteasome comprising administering the composition of claim 16 to a subject in need thereof.
